(19) United States
(12) Patent Application Publication
(10) Pub. No.: US 2020/0111967 A1
(43) Pub. Date: Apr. 9, 2020

(54) COMPOSITION FOR ORGANIC OPTOELECTRONIC DEVICE AND ORGANIC OPTOELECTRONIC DEVICE AND DISPLAY DEVICE

(71) Applicants: SAMSUNG SDI CO., LTD., Yongin-si (KR); SAMSUNG ELECTRONICS CO., LTD., Suwon-si (KR)

(72) Inventors: Namheon LEE, Suwon-si (KR); Dong Min KANG, Suwon-si (KR); Dongyeong KIM, Suwon-si (KR); Jun Seok KIM, Suwon-si (KR); Byoungkwan LEE, Suwon-si (KR); Sangshin LEE, Suwon-si (KR); Eun Sun YU, Suwon-si (KR); Ho Kuk JUNG, Suwon-si (KR); Jongwoo CHOI, Suwon-si (KR)

(21) Appl. No.: 16/590,456

(22) Filed: Oct. 2, 2019

(30) Foreign Application Priority Data
Oct. 4, 2018 (KR) 10-2018-0118385

(51) Int. Cl. H01L 51/00

(52) U.S. Cl. CPC H01L 51/0067; H01L 51/5206; H01L 51/0054

(57) ABSTRACT

A composition, an organic optoelectronic device, and a display device, the composition including a first compound represented by a combination of Chemical Formula 1 and Chemical Formula 2, and a second compound represented by Chemical Formula 3:

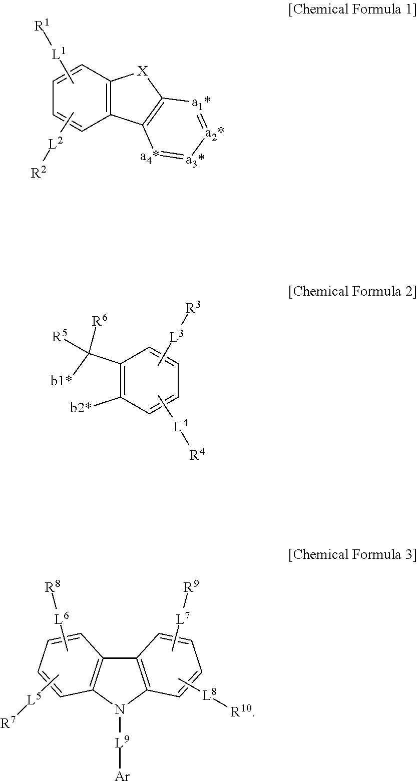

### CROSS-REFERENCE TO RELATED APPLICATION

Korean Patent Application No. 10-2018-0118385, filed on Oct. 4, 2018, in the Korean Intellectual Property Office, and entitled: “Composition for Organic Optoelectronic Device and Organic Optoelectronic Device and Display Device,” is incorporated by reference herein in its entirety.

### BACKGROUND

### 1. Field

Embodiments relate to a composition for an organic optoelectronic device, an organic optoelectronic device, and a display device.

### 2. Description of the Related Art

An organic optoelectronic device is a device that converts electrical energy into photoenergy, and vice versa.

An organic optoelectronic device may be classified as follows in accordance with its driving principles. One is a photoelectric device where excitons generated by photoenergy are separated into electrons and holes and the electrons and holes are transferred to different electrodes respectively and electrical energy is generated, and the other is a light emitting device to generate photoenergy from electrical energy by supplying a voltage or a current to electrodes.

Examples of the organic optoelectronic device may include an organic photoelectric device, an organic light emitting diode, an organic solar cell, and an organic photo conductor drum.

Among them, the organic light emitting diode (OLED) has recently drawn attention due to an increase in demand for flat panel displays. The organic light emitting diode may convert electrical energy into light, and the performance of organic light emitting diode may be influenced by the organic materials between electrodes.

### SUMMARY

The embodiments may be realized by providing a composition for an organic optoelectronic device, the composition including a first compound represented by a combination of Chemical Formula 1 and Chemical Formula 2, and a second compound represented by Chemical Formula 3:

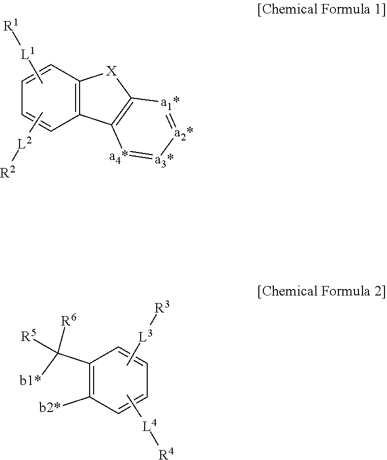

wherein, in Chemical Formula 1 and Chemical Formula 2, X is O or S, adjacent two of a₁* to a₄* are C linked with b₁* and b₂* respectively, the other two of a₁* to a₄* not linked with b₁* and b₂* are each independently C-L^{a}-R^{a}, L^{a} and L¹ to L⁴ are each independently a single bond, a substituted or unsubstituted C6 to C20 arylene group, a substituted or unsubstituted C2 to C20 heterocyclic group, or a combination thereof, R^{a} and R¹ to R⁶ are each independently hydrogen, deuterium, a cyano group, a substituted or unsubstituted amine group, a substituted or unsubstituted C1 to C30 alkyl group, a substituted or unsubstituted C6 to C30 aryl group, a substituted or unsubstituted C2 to C30 heterocyclic group, or a combination thereof, and at least one of R¹ to R⁴ is a substituted amine group represented by Chemical Formula a,

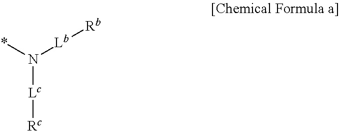

wherein, in Chemical Formula a, L^{b} and L^{c} are each independently a single bond, a substituted or unsubstituted C6 to C20 arylene group, a substituted or unsubstituted C2 to C20 heterocyclic group, or a combination thereof, R^{b} and R^{c} are each independently a substituted or unsubstituted C6 to C30 aryl group, a substituted or unsubstituted C2 to C30 heterocyclic group, or a combination thereof, and * is a linking point with L¹, L², L³, or L⁴;

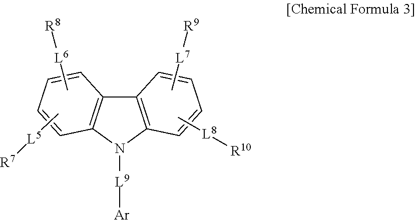

wherein, in Chemical Formula 3, L⁵ to L⁹ are each independently a single bond, a substituted or unsubstituted C6 to C20 arylene group, a substituted or unsubstituted C2 to C20 heterocyclic group, or a combination thereof, Ar is a substituted or unsubstituted C6 to C30 aryl group, a substituted or unsubstituted C2 to C30 heterocyclic group, or a combination thereof, R⁷ to R¹⁰ are each independently hydrogen, deuterium, a substituted or unsubstituted C1 to C30 alkyl group, a substituted or unsubstituted C6 to C30 aryl group, a substituted or unsubstituted C2 to C30 heterocyclic group, a substituted or unsubstituted silyl group, a substituted or unsubstituted amine group, a halogen, a cyano group, or a combination thereof, R⁷ to R¹⁰ are separately present or adjacent ones thereof are linked with each other to form a substituted or unsubstituted aliphatic monocyclic ring or a substituted or unsubstituted aliphatic polycyclic ring, a substituted or unsubstituted aromatic monocyclic ring or a substituted or unsubstituted aromatic polycyclic ring, or a substituted or unsubstituted heteroaromatic monocyclic ring or a substituted or unsubstituted heteroaromatic polycyclic ring, and at least one of Ar and R⁷ to R¹⁰ is a group represented by Chemical Formula b,

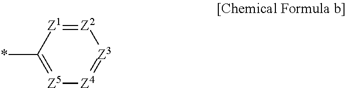

wherein, in Chemical Formula b, Z¹ to Z⁵ are each independently N or C-L^{d}-R^{d}, at least two of Z¹ to Z⁵ are N, each L^{d} is independently a single bond, a substituted or unsubstituted C6 to C20 arylene group, a substituted or unsubstituted C2 to C20 heterocyclic group, or a combination thereof, each R^{d} is independently hydrogen, deuterium, a substituted or unsubstituted C1 to C30 alkyl group, a substituted or unsubstituted C6 to C30 aryl group, a substituted or unsubstituted C2 to C30 heterocyclic group, a substituted or unsubstituted silyl group, a substituted or unsubstituted amine group, a halogen, a cyano group, or a combination thereof, each R^{d} is separately present or adjacent ones thereof are linked with each other to form a substituted or unsubstituted aliphatic monocyclic ring or a substituted or unsubstituted aliphatic polycyclic ring, a substituted or unsubstituted aromatic monocyclic ring or a substituted or unsubstituted aromatic polycyclic ring, or a substituted or unsubstituted heteroaromatic monocyclic ring or a substituted or unsubstituted heteroaromatic polycyclic ring, and * is a linking point with L⁵, L⁶, L⁷, L⁸, or L⁹.

The first compound may be represented by one of Chemical Formula 1A to Chemical Formula 1F:

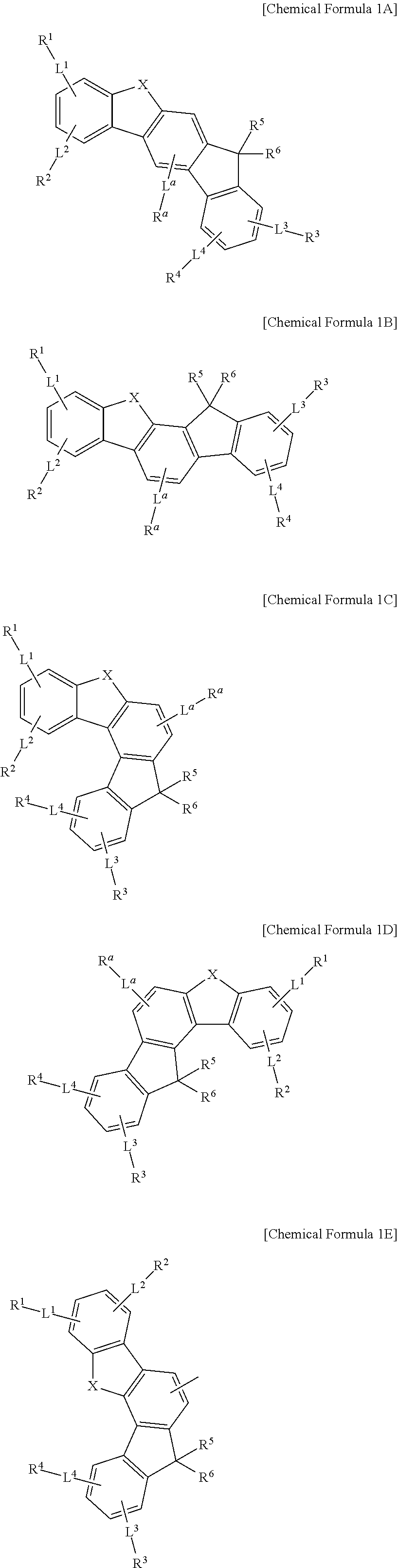

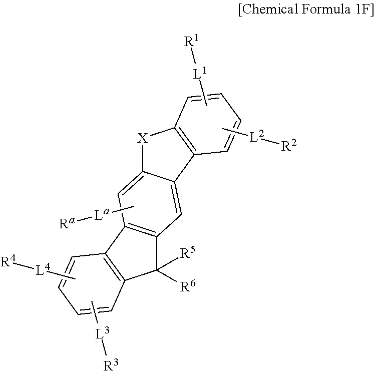

wherein, in Chemical Formula 1A to Chemical Formula 1F, X is O or S, L^{a} and L¹ to L⁴ are each independently a single bond, a substituted or unsubstituted C6 to C20 arylene group, a substituted or unsubstituted C2 to C20 heterocyclic group, or a combination thereof, R^{a} and R¹ to R⁶ are each independently hydrogen, deuterium, a cyano group, a substituted or unsubstituted amine group, a substituted or unsubstituted C1 to C30 alkyl group, a substituted or unsubstituted C6 to C30 aryl group, a substituted or unsubstituted C2 to C30 heterocyclic group, or a combination thereof, and at least one of R¹ to R⁴ is a substituted amine group represented by Chemical Formula a,

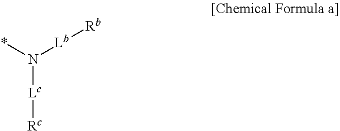

wherein, in Chemical Formula a, L^{b} and L^{c} are each independently a single bond, a substituted or unsubstituted C6 to C20 arylene group, a substituted or unsubstituted C2 to C20 heterocyclic group, or a combination thereof, R^{b} and R^{c} are each independently a substituted or unsubstituted C6 to C30 aryl group, a substituted or unsubstituted C2 to C30 heterocyclic group, or a combination thereof, and

* is a linking point with L¹, L², L³, or L⁴.

The first compound may be represented by Chemical Formula 1E-1-1 or Chemical Formula 1E-2-2:

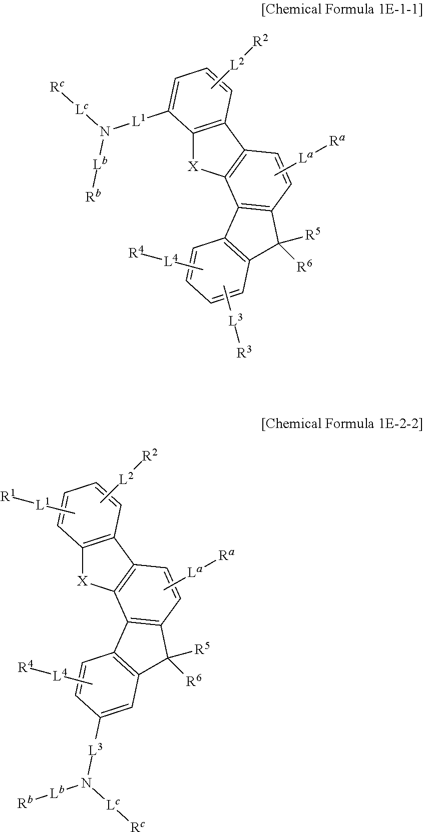

wherein, in Chemical Formula 1E-1-1 and Chemical Formula 1E-2-2, X is O or S, L^{a} and L¹ to L⁴ are each independently a single bond, a substituted or unsubstituted C6 to C20 arylene group, a substituted or unsubstituted C2 to C20 heterocyclic group, or a combination thereof, R^{a} and R¹ to R⁶ are each independently hydrogen, deuterium, a cyano group, a substituted or unsubstituted amine group, a substituted or unsubstituted C1 to C30 alkyl group, a substituted or unsubstituted C6 to C30 aryl group, a substituted or unsubstituted C2 to C30 heterocyclic group, or a combination thereof, L^{b} and L^{c} are each independently a single bond, a substituted or unsubstituted C6 to C20 arylene group, a substituted or unsubstituted C2 to C20 heterocyclic group, or a combination thereof, and R^{b} and R^{c} are each independently a substituted or unsubstituted phenyl group, a substituted or unsubstituted biphenyl group, a substituted or unsubstituted terphenyl group, a substituted or unsubstituted anthracenyl group, a substituted or unsubstituted naphthyl group, a substituted or unsubstituted phenanthrenyl group, a substituted or unsubstituted triphenylene group, a substituted or unsubstituted fluorenyl group, a substituted or unsubstituted carbazolyl group, a substituted or unsubstituted dibenzofuranyl group, a substituted or unsubstituted dibenzothiophenyl group, or a monovalent fused ring group of a compound represented by a combination of Chemical Formula 1 and Chemical Formula 2.

The second compound may be represented by one of Chemical Formula 3A to Chemical Formula 3R:

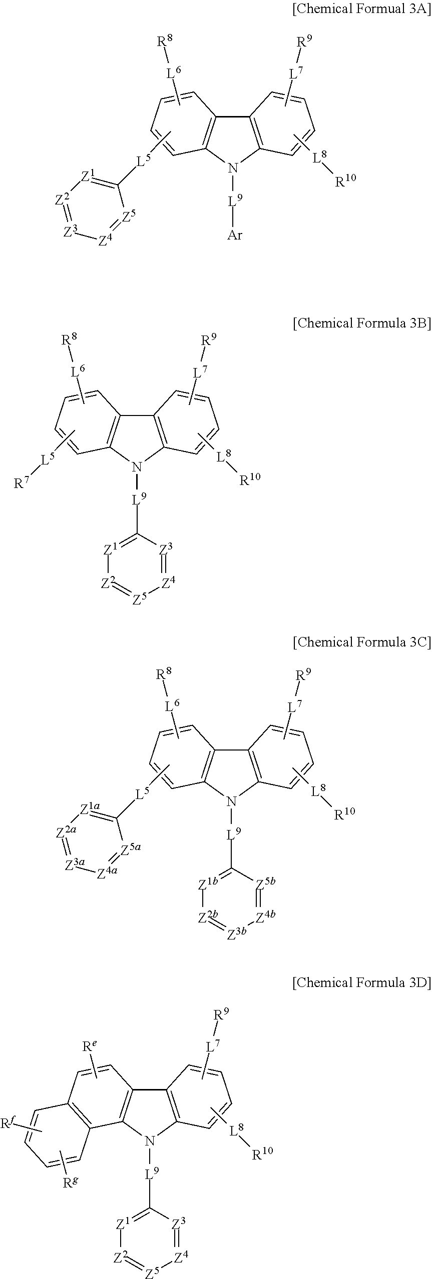

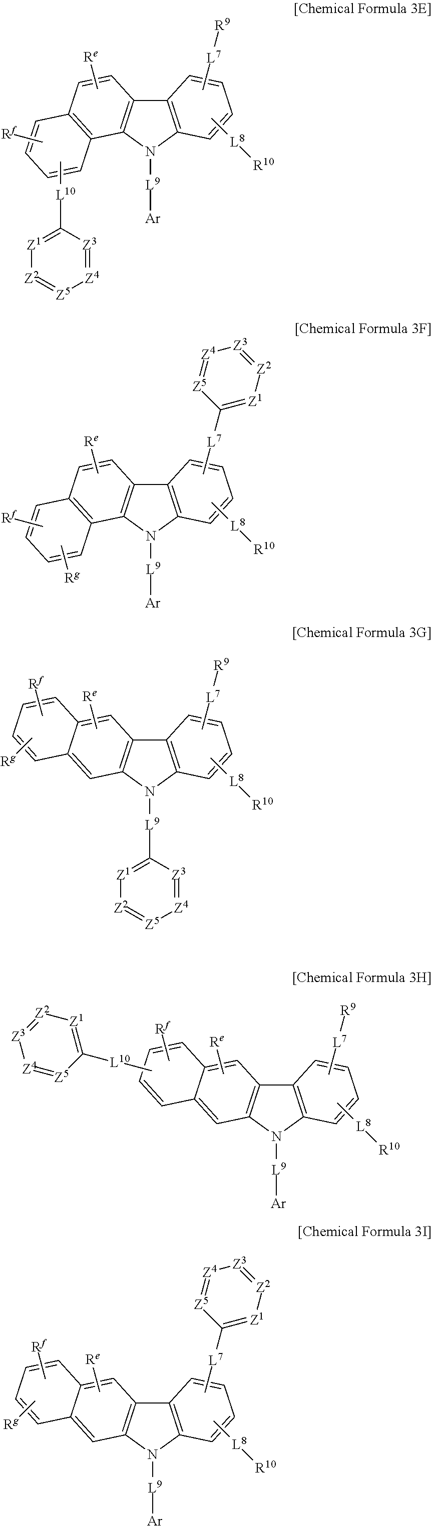

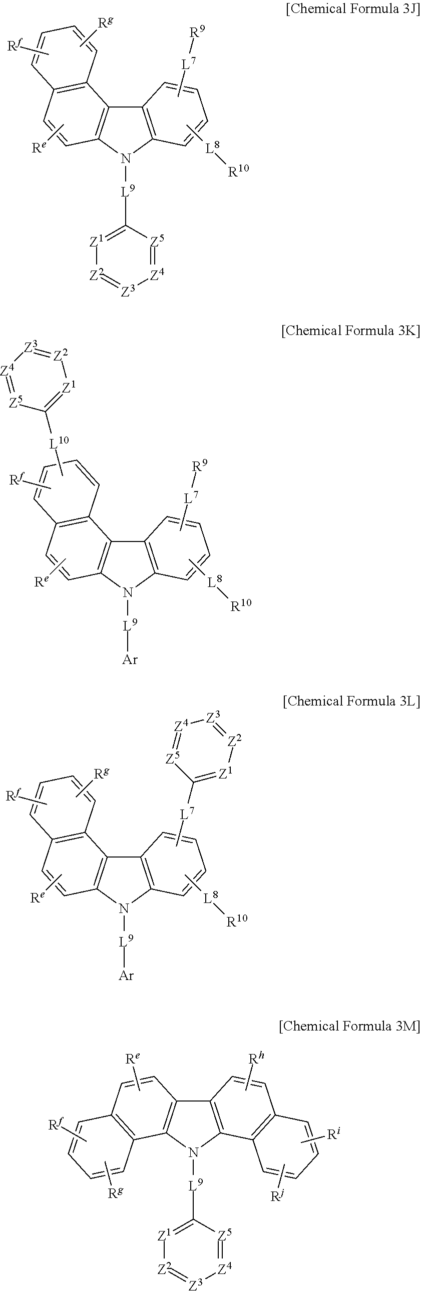

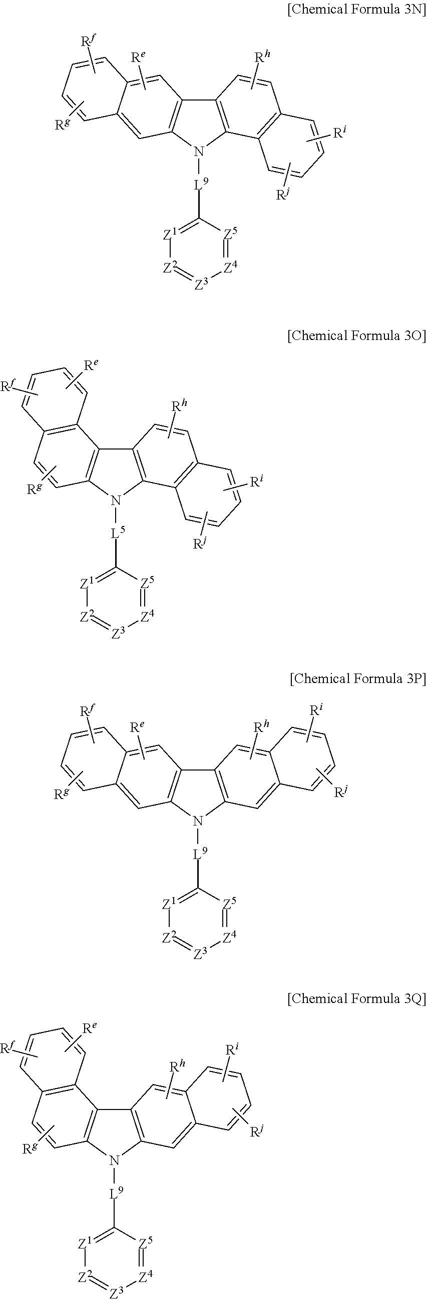

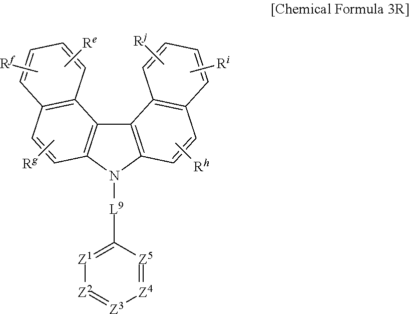

wherein, in Chemical Formula 3A to Chemical Formula 3R, L⁵ to L¹⁰ are each independently a single bond, a substituted or unsubstituted C6 to C20 arylene group, a substituted or unsubstituted C2 to C20 heterocyclic group, or a combination thereof, Ar is a substituted or unsubstituted C6 to C30 aryl group, R⁷ to R¹⁰, R^{e}, R^{f}, R^{g}, R^{h}, Rⁱ, and R^{j} are each independently hydrogen, deuterium, a substituted or unsubstituted C1 to C30 alkyl group, a substituted or unsubstituted C6 to C30 aryl group, a substituted or unsubstituted C2 to C30 heterocyclic group, a substituted or unsubstituted silyl group, a substituted or unsubstituted amine group, a halogen, a cyano group, or a combination thereof, Z¹ to Z⁵, Z^{1a} to Z^{5a}, and Z^{1b} to Z^{5b} are each independently N or C-L^{d}-R^{d}, at least two of Z¹ to Z⁵ are N, at least two of Z^{1a} to Z^{5a} are N, at least two of Z^{1b} to Z^{5b} are N, each L^{d} is independently a single bond, a substituted or unsubstituted C6 to C20 arylene group, a substituted or unsubstituted C2 to C20 heterocyclic group, or a combination thereof, each R^{d} is independently hydrogen, deuterium, a substituted or unsubstituted C1 to C30 alkyl group, a substituted or unsubstituted C6 to C30 aryl group, a substituted or unsubstituted C2 to C30 heterocyclic group, a substituted or unsubstituted silyl group, a substituted or unsubstituted amine group, a halogen, a cyano group, or a combination thereof, and each R^{d} is separately present or adjacent ones thereof are linked with each other to form a substituted or unsubstituted aliphatic monocyclic ring or a substituted or unsubstituted aliphatic polycyclic ring, a substituted or unsubstituted aromatic monocyclic ring or a substituted or unsubstituted aromatic polycyclic ring, or a substituted or unsubstituted heteroaromatic monocyclic ring or a substituted or unsubstituted heteroaromatic polycyclic ring.

The second compound may be represented by one of Chemical Formula 3A, Chemical Formula 3B, Chemical Formula 3E, and Chemical Formula 3J.

The second compound may be represented by Chemical Formula 3A or Chemical Formula 3E, the compound represented by Chemical Formula 3A may be represented by Chemical Formula 3A-2 or Chemical Formula 3A-4, and the compound represented by Chemical Formula 3E may be represented by Chemical Formula 3E-2:

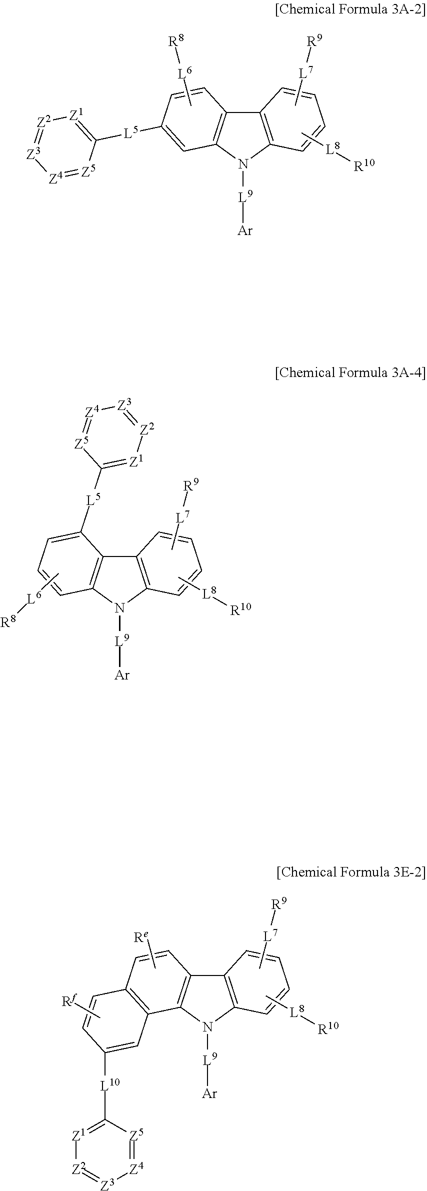

wherein, in Chemical Formula 3A-2, Chemical Formula 3A-4, and Chemical Formula 3E-2, L⁵ to L¹⁰ are each independently a single bond, a substituted or unsubstituted C6 to C20 arylene group, a substituted or unsubstituted C2 to C20 heterocyclic group, or a combination thereof, Ar is a substituted or unsubstituted C6 to C30 aryl group, R⁸ to R¹⁰, R^{e} and R^{f} are each independently hydrogen, deuterium, a substituted or unsubstituted C1 to C30 alkyl group, a substituted or unsubstituted C6 to C30 aryl group, a substituted or unsubstituted C2 to C30 heterocyclic group, a substituted or unsubstituted silyl group, a substituted or unsubstituted amine group, a halogen, a cyano group, or a combination thereof, Z¹ to Z⁵ are each independently N or C-L^{d}-R^{d}, at least two of Z¹ to Z⁵ are N, each L^{d} is independently a single bond, a substituted or unsubstituted C6 to C20 arylene group, a substituted or unsubstituted C2 to C20 heterocyclic group, or a combination thereof, each R^{d} is independently hydrogen, deuterium, a substituted or unsubstituted C1 to C30 alkyl group, a substituted or unsubstituted C6 to C30 aryl group, a substituted or unsubstituted C2 to C30 heterocyclic group, a substituted or unsubstituted silyl group, a substituted or unsubstituted amine group, a halogen, a cyano group, or a combination thereof, and each R^{d} is separately present or adjacent ones thereof are linked with each other to form a substituted or unsubstituted aliphatic monocyclic ring or a substituted or unsubstituted aliphatic polycyclic ring, a substituted or unsubstituted aromatic monocyclic ring or a substituted or unsubstituted aromatic polycyclic ring, or a substituted or unsubstituted heteroaromatic monocyclic ring or a substituted or unsubstituted heteroaromatic polycyclic ring.

The group represented by Chemical Formula b may be a substituted or unsubstituted pyrimidinyl group, a substituted or unsubstituted triazinyl group, a substituted or unsubstituted quinazolinyl group, a substituted or unsubstituted quinoxalinyl group, or a substituted or unsubstituted naphthyridinyl group.

The group represented by Chemical Formula b may be represented by one of Chemical Formula b-1 to Chemical Formula b-5:

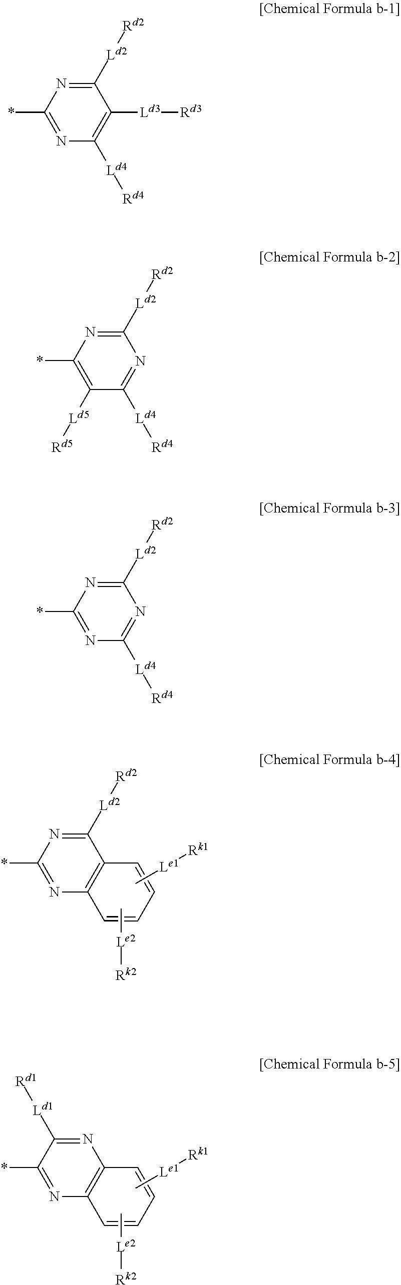

wherein, in Chemical Formula b-1 to Chemical Formula b-5, L^{d1} to L^{d5}, L^{e1}, and L^{e2} are each independently a single bond, a substituted or unsubstituted C6 to C20 arylene group, a substituted or unsubstituted C2 to C20 heterocyclic group, or a combination thereof, R^{d2} to R^{d5}, R^{k1}, and R^{k2} are each independently hydrogen, deuterium, a cyano group, a substituted or unsubstituted amine group, a substituted or unsubstituted C1 to C10 alkyl group, a substituted or unsubstituted phenyl group, a substituted or unsubstituted biphenyl group, a substituted or unsubstituted naphthyl group, a substituted or unsubstituted terphenyl group, a substituted or unsubstituted anthracenyl group, a substituted or unsubstituted phenanthrenyl group, a substituted or unsubstituted fluorenyl group, a substituted or unsubstituted triphenylene group, a substituted or unsubstituted dibenzofuranyl group, or a substituted or unsubstituted dibenzothiophenyl group, and * is a linking point with L⁵, L⁶, L⁷, L⁸, or L⁹.

The group represented by Chemical Formula b may be a group of the following Group I:

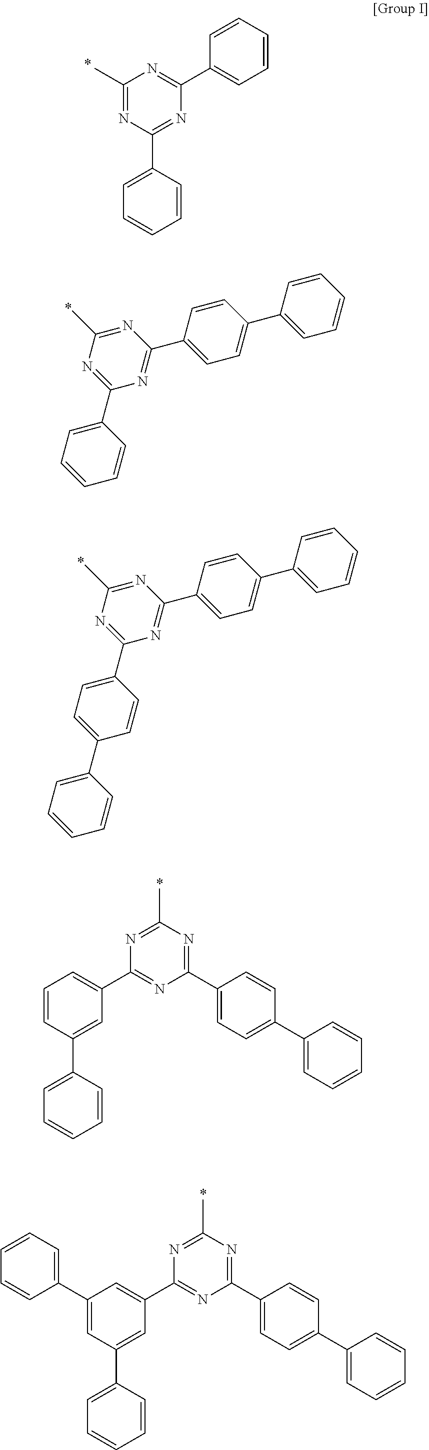

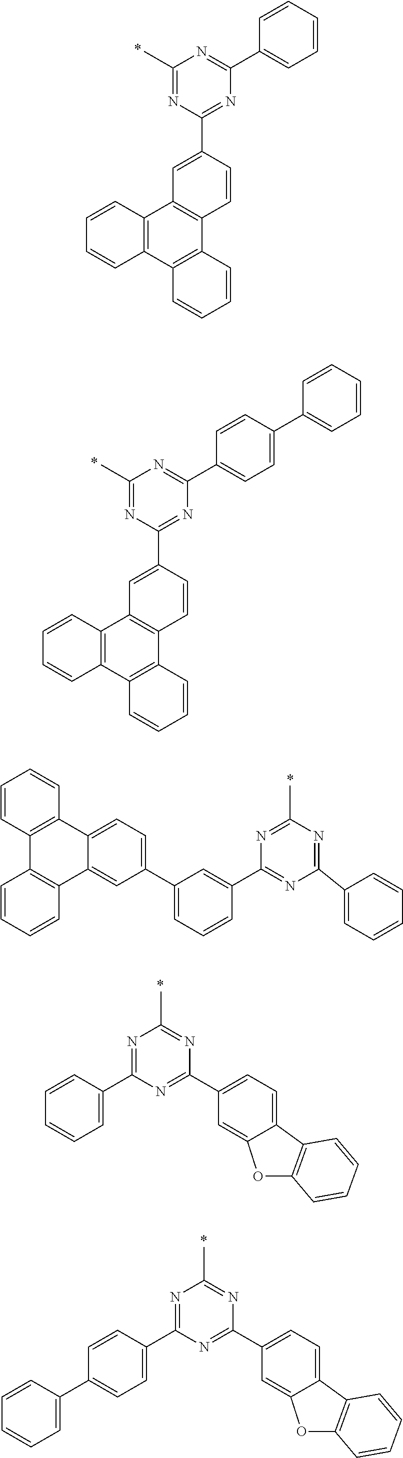

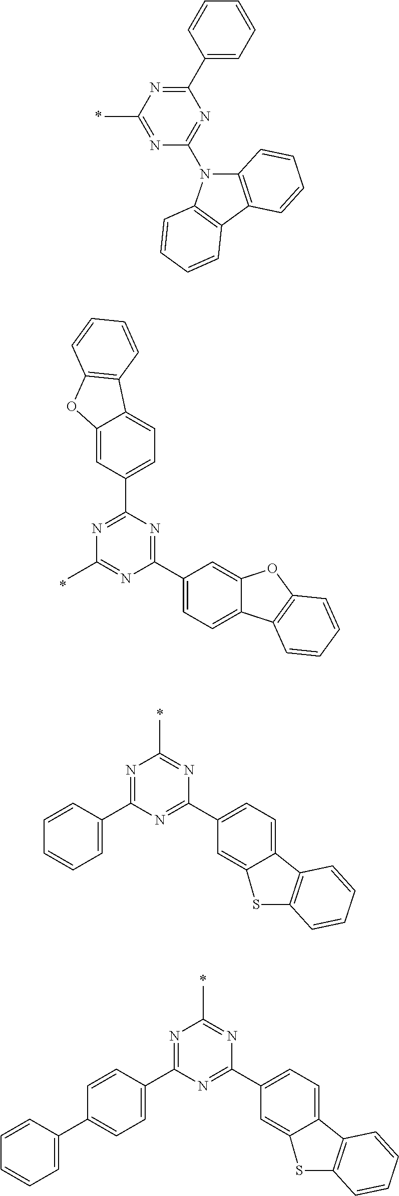

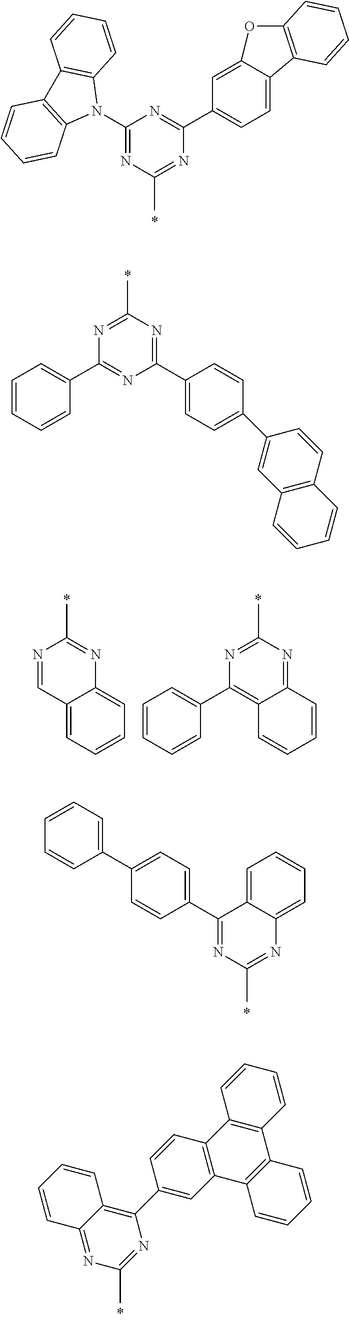

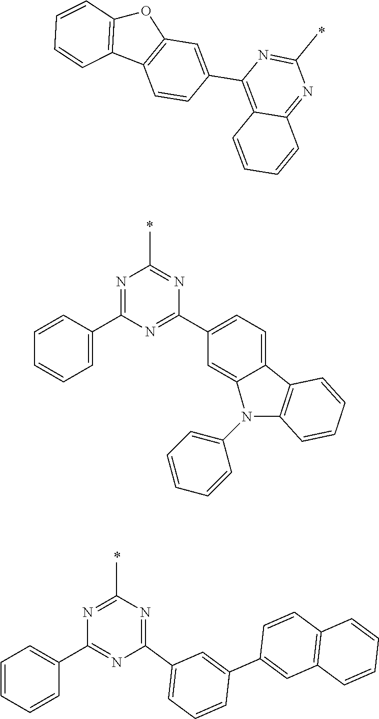

wherein, in Group I, * is a linking point.

The first compound may be represented by Chemical Formula 1E-2-2, and the second compound may be represented by one of Chemical Formula 3A-2, Chemical Formula 3B, Chemical Formula 3E-2, and Chemical Formula 3J:

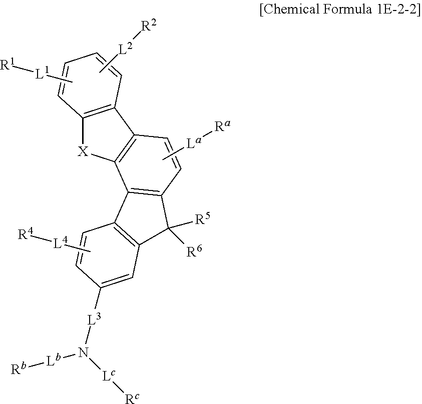

wherein, in Chemical Formula 1E-2-2, X is O or S, L^{a}, L^{b}, L^{c}, and L¹ to L⁴ are each independently a single bond, a substituted or unsubstituted phenylene group, a substituted or unsubstituted biphenylene group, a substituted or unsubstituted terphenylene group, or a substituted or unsubstituted naphthylene group, R^{a}, R¹, R², and R⁴ are each independently hydrogen, deuterium, a cyano group, a substituted or unsubstituted C1 to C30 alkyl group, a substituted or unsubstituted C6 to C30 aryl group, a substituted or unsubstituted C2 to C30 heterocyclic group, or a combination thereof, R⁵ and R⁶ are each independently a substituted or unsubstituted C1 to C10 alkyl group or a substituted or unsubstituted C6 to C20 aryl group, and R^{b} and R^{c} are each independently a substituted or unsubstituted phenyl group, a substituted or unsubstituted biphenyl group, a substituted or unsubstituted anthracenyl group, a substituted or unsubstituted naphthyl group, a substituted or unsubstituted phenanthrenyl group, a substituted or unsubstituted triphenylene group, a substituted or unsubstituted fluorenyl group, a substituted or unsubstituted carbazolyl group, a substituted or unsubstituted dibenzofuranyl group, a substituted or unsubstituted dibenzothiophenyl group, or a monovalent fused ring group of a compound represented by a combination of Chemical Formula 1 and Chemical Formula 2;

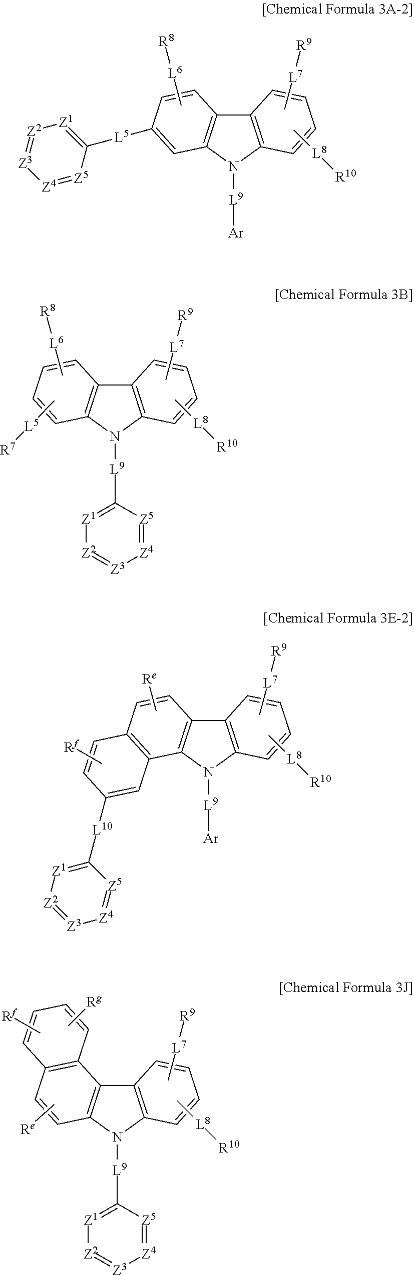

wherein, in Chemical Formula 3A-2, Chemical Formula 3B, Chemical Formula 3E-2, and Chemical Formula 3J, L⁵ to L¹⁰ are each independently a single bond, a substituted or unsubstituted phenylene group, a substituted or unsubstituted biphenylene group, a substituted or unsubstituted terphenylene group, a substituted or unsubstituted naphthylene group, a substituted or unsubstituted dibenzofuranylene group, a substituted or unsubstituted dibenzothiophenylene group, a substituted or unsubstituted carbazolylene group, a substituted or unsubstituted fused dibenzofuranylene group, a substituted or unsubstituted fused dibenzothiophenylene group, or a combination thereof, Ar is a substituted or unsubstituted phenyl group, a substituted or unsubstituted biphenyl group, or a substituted or unsubstituted naphthyl group, or a substituted or unsubstituted fluorenyl group. R⁷ to R¹⁰, R^{e}, R^{f} and R^{g} are each independently hydrogen, deuterium, a cyano group, a substituted or unsubstituted phenyl group, a substituted or unsubstituted biphenyl group, a substituted or unsubstituted terphenyl group, a substituted or unsubstituted naphthyl group, a substituted or unsubstituted triphenylene group, a substituted or unsubstituted dibenzofuranyl group, a substituted or unsubstituted dibenzothiophenyl group, or a combination thereof, Z¹ to Z⁵ are each independently N or C-L^{d}-R^{d}, at least two of Z¹ to Z⁵ are N, each L^{d} is independently a single bond or a substituted or unsubstituted C6 to C20 arylene group, each R^{d} is independently a substituted or unsubstituted phenyl group, a substituted or unsubstituted biphenyl group, a substituted or unsubstituted terphenyl group, a substituted or unsubstituted anthracenyl group, a substituted or unsubstituted naphthyl group, a substituted or unsubstituted phenanthrenyl group, a substituted or unsubstituted triphenylene group, a substituted or unsubstituted fluorenyl group, a substituted or unsubstituted carbazolyl group, a substituted or unsubstituted dibenzofuranyl group or a substituted or unsubstituted dibenzothiophenyl group, and each R^{d} is separately present or adjacent ones thereof are linked with each other to form a substituted or unsubstituted quinazolinyl group or a substituted or unsubstituted quinoxalinyl group.

The composition may further include a dopant.

The embodiments may be realized by providing an organic optoelectronic device including an anode and a cathode facing each other, at least one organic layer between the anode and the cathode, wherein the organic layer includes the composition for an organic optoelectronic device according to an embodiment.

The organic layer may include a light emitting layer, and the light emitting layer may include the composition.

The first compound and the second compound may be phosphorescent hosts of the light emitting layer.

The composition may be a red light emitting composition.

The embodiments may be realized by providing a display device comprising the organic optoelectronic device according to an embodiment.

### BRIEF DESCRIPTION OF THE DRAWINGS

Features will be apparent to those of skill in the art by describing in detail exemplary embodiments with reference to the attached drawings in which:

FIGS. 1 and 2 illustrate cross-sectional views of organic light emitting diodes according to embodiments.

### DETAILED DESCRIPTION

Example embodiments will now be described more fully hereinafter with reference to the accompanying drawings; however, they may be embodied in different forms and should not be construed as limited to the embodiments set forth herein. Rather, these embodiments are provided so that this disclosure will be thorough and complete, and will fully convey exemplary implementations to those skilled in the art.

In the drawing figures, the dimensions of layers and regions may be exaggerated for clarity of illustration. It will also be understood that when a layer or element is referred to as being “on” another layer or element, it can be directly on the other layer or element, or intervening layers may also be present. In addition, it will also be understood that when a layer is referred to as being “between” two layers, it can be the only layer between the two layers, or one or more intervening layers may also be present. Like reference numerals refer to like elements throughout.

As used herein, when a definition is not otherwise provided, “substituted” may refer to replacement of at least one hydrogen of a substituent or a compound by deuterium, a halogen, a hydroxyl group, an amino group, a substituted or unsubstituted C1 to C30 amine group, a nitro group, a substituted or unsubstituted C1 to C40 silyl group, a C1 to C30 alkyl group, a C1 to C10 alkylsilyl group, a C6 to C30 arylsilyl group, a C3 to C30 cycloalkyl group, a C3 to C30 heterocycloalkyl group, a C6 to C30 aryl group, a C2 to C30 heteroaryl group, a C1 to C20 alkoxy group, a C1 to C10 trifluoroalkyl group, a cyano group, or a combination thereof.

In one example, “substituted” may refer to replacement of at least one hydrogen of a substituent or a compound by deuterium, a C1 to C30 alkyl group, a C1 to C10 alkylsilyl group, a C6 to C30 arylsilyl group, a C3 to C30 cycloalkyl group, a C3 to C30 heterocycloalkyl group, a C6 to C30 aryl group, or a C2 to C30 heteroaryl group. In addition, in specific examples, “substituted” may refer to replacement of at least one hydrogen of a substituent or a compound by deuterium, a C1 to C20 alkyl group, a C6 to C30 aryl group, or a C2 to C30 heteroaryl group. In addition, in specific examples, “substituted” may refer to replacement of at least one hydrogen of a substituent or a compound by deuterium, a C1 to C5 alkyl group, a C6 to C18 aryl group, a pyridinyl group, a quinolinyl group, an isoquinolinyl group, a dibenzofuranyl group, a dibenzothiophenyl group, or a carbazolyl group. In addition, in specific examples, “substituted” may refer to replacement of at least one hydrogen of a substituent or a compound by deuterium, a C1 to C5 alkyl group, a C6 to C18 aryl group, a dibenzofuranyl group, or a dibenzothiophenyl group. In addition, in specific, “substituted” may refer to replacement of at least one hydrogen of a substituent or a compound by deuterium, a methyl group, an ethyl group, a propanyl group, a butyl group, a phenyl group, a biphenyl group, a terphenyl group, a naphthyl group, a triphenyl group, a dibenzofuranyl group, or a dibenzothiophenyl group.

As used herein, when a definition is not otherwise provided, “hetero” may refer to one including one to three heteroatoms selected from N, O, S, P, and Si, and remaining carbons in one functional group.

In the present specification, “aryl group” may refer to a group including at least one hydrocarbon aromatic moiety, and may include a group in which all elements of the hydrocarbon aromatic moiety have p-orbitals which form conjugation, for example a phenyl group, a naphthyl group, and the like, a group in which two or more hydrocarbon aromatic moieties may be linked by a sigma bond, for example a biphenyl group, a terphenyl group, a quarterphenyl group, and the like, and a group in which two or more hydrocarbon aromatic moieties are fused directly or indirectly to provide a non-aromatic fused ring, for example a fluorenyl group, and the like.

The aryl group may include a monocyclic, polycyclic or fused ring polycyclic (i.e., rings sharing adjacent pairs of carbon atoms) functional group.

In the present specification, “heterocyclic group” may refer to a generic concept of a heteroaryl group, and may include at least one heteroatom selected from N, O, S, P, and Si instead of carbon (C) in a cyclic compound such as an aryl group, a cycloalkyl group, a fused ring thereof, or a combination thereof. When the heterocyclic group is a fused ring, the entire ring or each ring of the heterocyclic group may include one or more heteroatoms.

For example, “heteroaryl group” may refer to an aryl group including at least one heteroatom selected from N, O, S, P, and Si. Two or more heteroaryl groups are linked by a sigma bond directly, or when the heteroaryl group includes two or more rings, the two or more rings may be fused. When the heteroaryl group is a fused ring, each ring may include one to three heteroatoms.

More specifically, the substituted or unsubstituted C6 to C30 aryl group may be a substituted or unsubstituted phenyl group, a substituted or unsubstituted naphthyl group, a substituted or unsubstituted anthracenyl group, a substituted or unsubstituted phenanthrenyl group, a substituted or unsubstituted naphthacenyl group, a substituted or unsubstituted pyrenyl group, a substituted or unsubstituted biphenyl group, a substituted or unsubstituted p-terphenyl group, a substituted or unsubstituted m-terphenyl group, a substituted or unsubstituted o-terphenyl group, a substituted or unsubstituted chrysenyl group, a substituted or unsubstituted triphenylene group, a substituted or unsubstituted perylenyl group, a substituted or unsubstituted fluorenyl group, a substituted or unsubstituted indenyl group, or a combination thereof, but is not limited thereto.

More specifically, the substituted or unsubstituted C2 to C30 heterocyclic group may be a substituted or unsubstituted furanyl group, a substituted or unsubstituted thiophenyl group, a substituted or unsubstituted pyrrolyl group, a substituted or unsubstituted pyrazolyl group, a substituted or unsubstituted imidazolyl group, a substituted or unsubstituted triazolyl group, a substituted or unsubstituted oxazolyl group, a substituted or unsubstituted thiazolyl group, a substituted or unsubstituted oxadiazolyl group, a substituted or unsubstituted thiadiazolyl group, a substituted or unsubstituted pyridyl group, a substituted or unsubstituted pyrimidinyl group, a substituted or unsubstituted pyrazinyl group, a substituted or unsubstituted triazinyl group, a substituted or unsubstituted benzofuranyl group, a substituted or unsubstituted benzothiophenyl group, a substituted or unsubstituted benzimidazolyl group, a substituted or unsubstituted indolyl group, a substituted or unsubstituted quinolinyl group, a substituted or unsubstituted isoquinolinyl group, a substituted or unsubstituted quinazolinyl group, a substituted or unsubstituted quinoxalinyl group, a substituted or unsubstituted naphthyridinyl group, a substituted or unsubstituted benzoxazinyl group, a substituted or unsubstituted benzthiazinyl group, a substituted or unsubstituted acridinyl group, a substituted or unsubstituted phenazinyl group, a substituted or unsubstituted phenothiazinyl group, a substituted or unsubstituted phenoxazinyl group, a substituted or unsubstituted dibenzofuranyl group, or a substituted or unsubstituted dibenzothiophenyl group, or a combination thereof, but is not limited thereto.

As used herein, the description that adjacent groups or ones thereof are linked with each other to form a substituted or unsubstituted aromatic monocyclic or polycyclic ring, or a substituted or unsubstituted aromatic monocyclic or polycyclic heterocyclic ring may mean that any two adjacent substituents directly substituting an aromatic ring or an aromatic heterocyclic ring with a single bond without a linking group are linked to form an additional ring.

For example, adjacent groups are linked with each other to form a substituted or unsubstituted aromatic monocyclic additional ring or a substituted or unsubstituted aromatic polycyclic additional ring and examples may be a substituted or unsubstituted aromatic monocyclic additional ring.

For example, any two substituents directly substituting the benzene ring of carbazole are linked with each other to form an additional ring, and thereby a substituted or unsubstituted benzocarbazole group or a substituted or unsubstituted dibenzocarbazole group may be formed together with the benzene ring of carbazole.

For example, any two substituents directly substituting the nitrogen-containing hexagonal ring are linked with each other to form an additional ring, and thereby a substituted or unsubstituted quinazolinyl group or a substituted or unsubstituted quinoxalinyl group may be formed together with the nitrogen-containing hexagonal ring.

In the present specification, hole characteristics refer to an ability to donate an electron to form a hole when an electric field is applied and that a hole formed in the anode may be easily injected into the light emitting layer and transported in the light emitting layer due to conductive characteristics according to a highest occupied molecular orbital (HOMO) level.

In addition, electron characteristics refer to an ability to accept an electron when an electric field is applied and that electron formed in the cathode may be easily injected into the light emitting layer and transported in the light emitting layer due to conductive characteristics according to a lowest unoccupied molecular orbital (LUMO) level.

Hereinafter, a composition for an organic optoelectronic device according to an embodiment is described.

The composition for an organic optoelectronic device may include, e.g., a first compound (for an organic optoelectronic device) having hole characteristics and a second compound (for an organic optoelectronic device) having electron characteristics.

The first compound may be represented by a combination of Chemical Formula 1 and Chemical Formula 2.

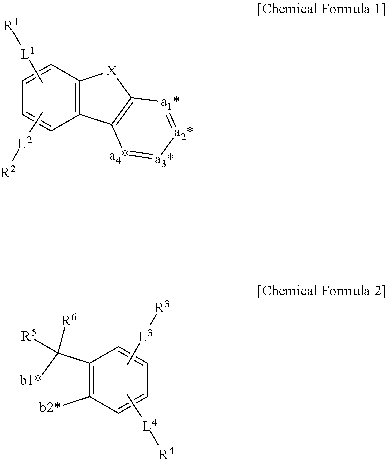

In Chemical Formula 1 and Chemical Formula 2,

X may be, e.g., O or S,

adjacent two of a₁* to a₄* may be C linked with b₁* and b₂* respectively,

the remainders (e.g., the other two of) of a₁* to a₄* not linked with b₁* and b₂* may each independently be, e.g., C-L^{a}-R^{a},

L^{a} and L¹ to L⁴ may each independently be or include, e.g., a single bond, a substituted or unsubstituted C6 to C20 arylene group, a substituted or unsubstituted C2 to C20 heterocyclic group, or a combination thereof, and

R^{a} and R¹ to R⁶ may each independently be or include, e.g., hydrogen, deuterium, a cyano group, a substituted or unsubstituted amine group, a substituted or unsubstituted C1 to C30 alkyl group, a substituted or unsubstituted C6 to C30 aryl group, a substituted or unsubstituted C2 to C30 heterocyclic group, or a combination thereof. For example, remaining carbon atoms of the rings that include -L¹-R¹, -L²-R², -L³-R³, and -L⁴-R⁴ thereon may have a hydrogen atom bonded thereto.

In an implementation, at least one of R¹ to R⁴ may be, e.g., a (substituted amine) group represented by Chemical Formula a.

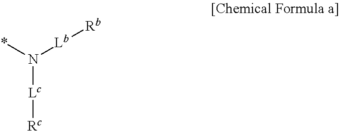

wherein, in Chemical Formula a,

L^{b} and L^{c} may each independently be or include, e.g., a single bond, a substituted or unsubstituted C6 to C20 arylene group, a substituted or unsubstituted C2 to C20 heterocyclic group, or a combination thereof.

R^{b} and R^{c} may each independently be or include, e.g., a substituted or unsubstituted C6 to C30 aryl group, a substituted or unsubstituted C2 to C30 heterocyclic group, or a combination thereof (e.g., a monovalent group of a fused ring represented by a combination of Chemical Formula 1 and Chemical Formula 2).

* is a linking point with L¹ to L⁴.

The first compound may have a structure of an amine (substituted with an aryl group and/or a heteroaryl group) linked to a fused heterocycle of 6 membered ring-5 membered ring-6 membered ring-5 membered ring-6 membered ring and may have high HOMO energy. For example, a HOMO electron cloud may expand from the amine to the fused heterocycle, and the compound may exhibit excellent hole injection and transfer characteristics.

In addition, the fused heterocycle of 6 membered ring-5 membered ring-6 membered ring-5 membered ring-6 membered ring may have relatively high HOMO energy compared with bicarbazole and indolocarbazole, and a device having a low driving voltage may be realized by applying the structure of the amine linked to the fused heterocycle.

In addition, bicarbazole and indolocarbazole may have high T1 energy and may not be appropriate or suitable as a red host. In contrast, the structure of the amine linked to the fused heterocycle (e.g., according to an embodiment) may have appropriate T1 energy as a red host.

The first compound may include the fused heterocycle and may exhibit a decreased symmetry in the molecule, and crystallization among the compounds may be suppressed. For example, a dark spot generated due to the crystallization of the compounds during deposition of a material in a process of manufacturing a device may be suppressed, and accordingly, a life-span of the device may be improved.

For example, a device manufactured with the first compound according to an embodiment may realize high efficiency/long life-span characteristics.

In an implementation, the first compound may be included with the second compound, may exhibit satisfactory interface characteristics and transportation capability of holes and electrons and accordingly, and may help lower a driving voltage of a device manufactured by applying the same.

In an implementation, L^{b} and L^{c} may each independently be, e.g., a single bond or a substituted or unsubstituted C6 to C12 arylene group.

In an implementation, L^{b} and L^{c} may each independently be, e.g., a single bond, a substituted or unsubstituted phenylene group, or a substituted or unsubstituted biphenylene group.

In an implementation, R^{b} and R^{c} may each independently be or include, e.g., a substituted or unsubstituted phenyl group, a substituted or unsubstituted biphenyl group, a substituted or unsubstituted terphenyl group, a substituted or unsubstituted anthracenyl group, a substituted or unsubstituted naphthyl group, a substituted or unsubstituted phenanthrenyl group, a substituted or unsubstituted triphenylene group, a substituted or unsubstituted fluorenyl group, a substituted or unsubstituted carbazolyl group, a substituted or unsubstituted dibenzofuranyl group, a substituted or unsubstituted dibenzothiophenyl group, or a monovalent group of a fused ring represented by a combination of Chemical Formula 1 and Chemical Formula 2. For example, R^{b} and/or R^{c} may be a monovalent group that is a fused combination of a substituted or unsubstituted C6 to C30 aryl group and a substituted or unsubstituted C2 to C30 heterocyclic group that forms a monovalent fused ring group of the compound represented by a combination of Chemical Formula 1 and Chemical Formula 2. For example, R^{b} and/or R^{c} may be a monovalent group of a fused heterocycle of 6 membered ring-5 membered ring-6 membered ring-5 membered ring-6 membered ring. For example, a hydrogen of the combination of Chemical Formula 1 and Chemical Formula 2 may be replaced with a bonding location to L^{b}, L^{c}, and/or N in order to form a monovalent group for R^{b} and/or R^{c}.

In an implementation, R^{b} and R^{c} may each independently be or include, e.g., a substituted or unsubstituted phenyl group, a substituted or unsubstituted biphenyl group, a substituted or unsubstituted naphthyl group, a substituted or unsubstituted fluorenyl group, a substituted or unsubstituted carbazolyl group, a substituted or unsubstituted dibenzofuranyl group, a substituted or unsubstituted dibenzothiophenyl group, or a fused ring represented by a combination of Chemical Formula 1 and Chemical Formula 2.

In an implementation, R^{b} and R^{c} may each independently be or include, e.g., a substituted or unsubstituted phenyl group, a substituted or unsubstituted biphenyl group, a substituted or unsubstituted naphthyl group, or a substituted or unsubstituted fluorenyl group.

In an implementation, L^{a} and L¹ to L⁴ may each independently be or include, e.g., a single bond or a substituted or unsubstituted C6 to C20 arylene group.

In an implementation, L^{a} and L¹ to L⁴ may each independently be or include, e.g., a single bond, a substituted or unsubstituted phenylene group, a substituted or unsubstituted biphenylene group, or a substituted or unsubstituted naphthylene group.

In an implementation, L^{a} and L¹ to L⁴ may each independently be or include, e.g., a single bond or a substituted or unsubstituted p-phenylene group.

In an implementation, R^{a} and R¹ to R⁴ may each independently be or include, e.g., hydrogen, deuterium, a cyano group, a substituted or unsubstituted C1 to C10 alkyl group, or a substituted or unsubstituted C6 to C20 aryl group.

In an implementation, R^{a} and R¹ to R⁴ may each be, e.g., hydrogen.

In an implementation, R⁵ and R⁶ may each independently be or include, e.g., a substituted or unsubstituted C1 to C10 alkyl group or a substituted or unsubstituted C6 to C20 aryl group.

In an implementation, R⁵ and R⁶ may each independently be or include, e.g., a substituted or unsubstituted C1 to C4 alkyl group or a substituted or unsubstituted C6 to C12 aryl group.

In an implementation, the first compound may be a compound represented by one of Chemical Formula 1A to Chemical Formula 1F (e.g., according to a combination point of Chemical Formula 1 and Chemical Formula 2).

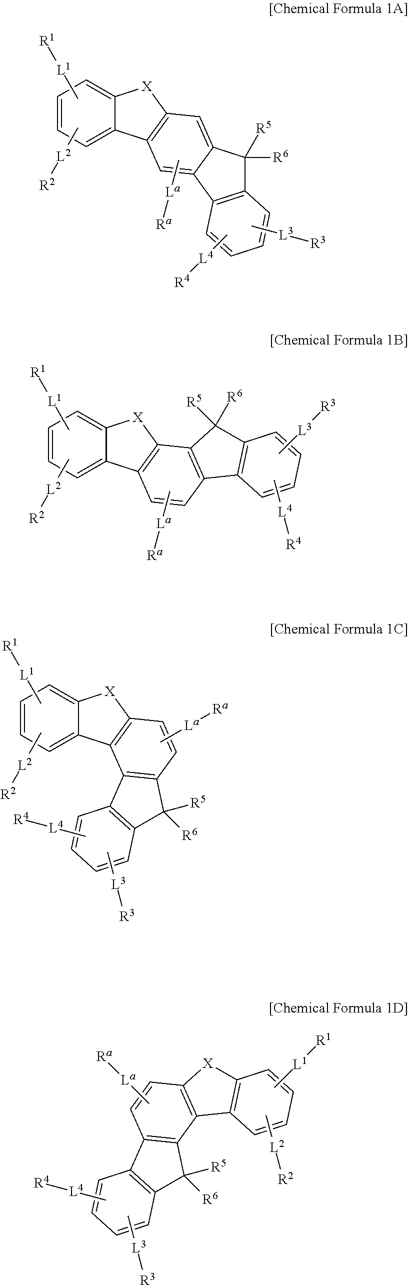

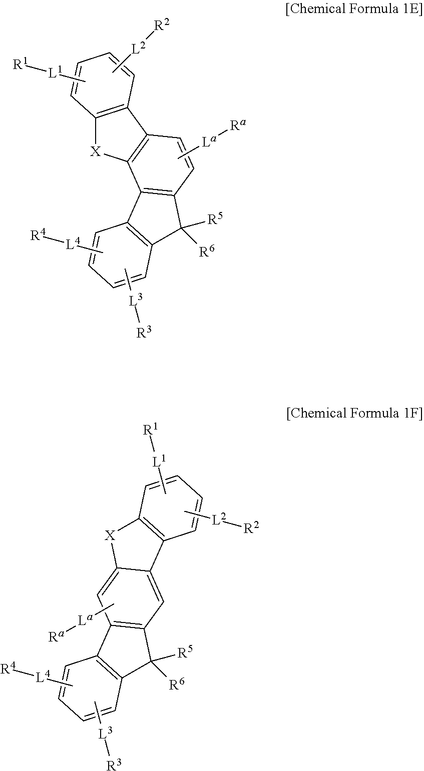

In Chemical Formula 1A to Chemical Formula 1F, X, L^{a} and L¹ to L⁴ and R^{a} and R¹ to R⁶ may be defined the same as those of Chemical Formulae 1 and 2.

In an implementation, the first compound represented by Chemical Formula 1A may be represented by Chemical Formula 1A-1 or Chemical Formula 1A-2 (e.g., according to a substitution direction of the group represented by Chemical Formula a).

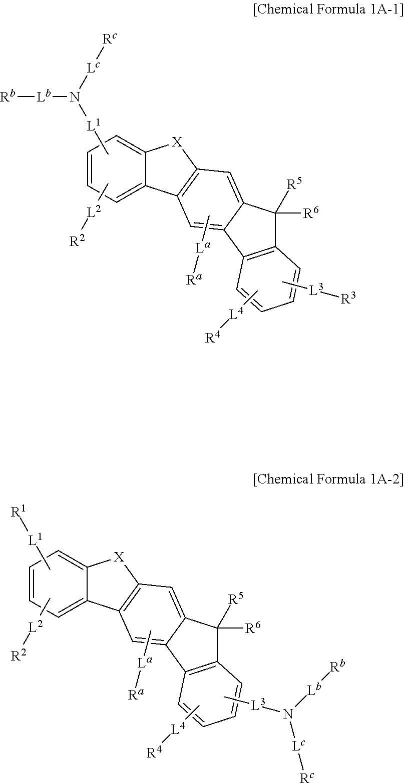

In Chemical Formula 1A-1 and Chemical Formula 1A-2, X, L^{a}, L^{b}, L^{c}, L¹ to L⁴, R^{a}, R¹ to R⁶, R^{b}, and R^{c} may be defined the same as those of Chemical Formulae 1 and 2.

In an implementation, the first compound represented by Chemical Formula 1A-1 may be represented by one of Chemical Formula 1A-1-1 to Chemical Formula 1A-1-4 (e.g., according to a specific substitution position of the group represented by Chemical Formula a).

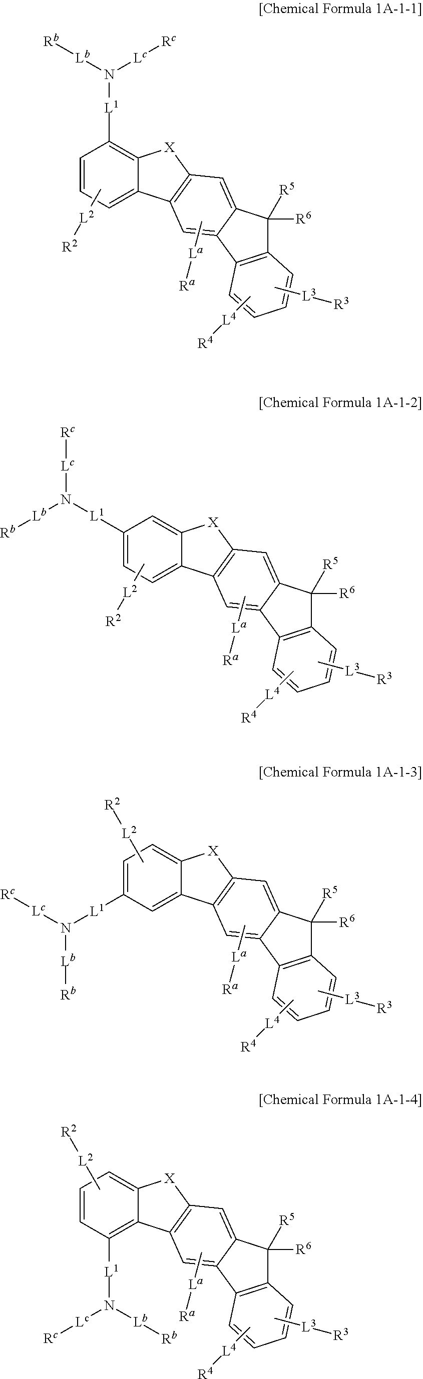

In Chemical Formula 1A-1-1 to Chemical Formula 1A-1-4, X, L^{a}, L^{b}, L^{c}, L¹ to L⁴, R^{a}, R¹ to R⁶, R^{b}, and R^{c} may be defined the same as those of Chemical Formulae 1 and 2.

In an implementation, the first compound represented by Chemical Formula 1A-2 may be represented by one of Chemical Formula 1A-2-1 to Chemical Formula 1A-2-4 (e.g., according to a specific substitution position of the group represented by Chemical Formula a).

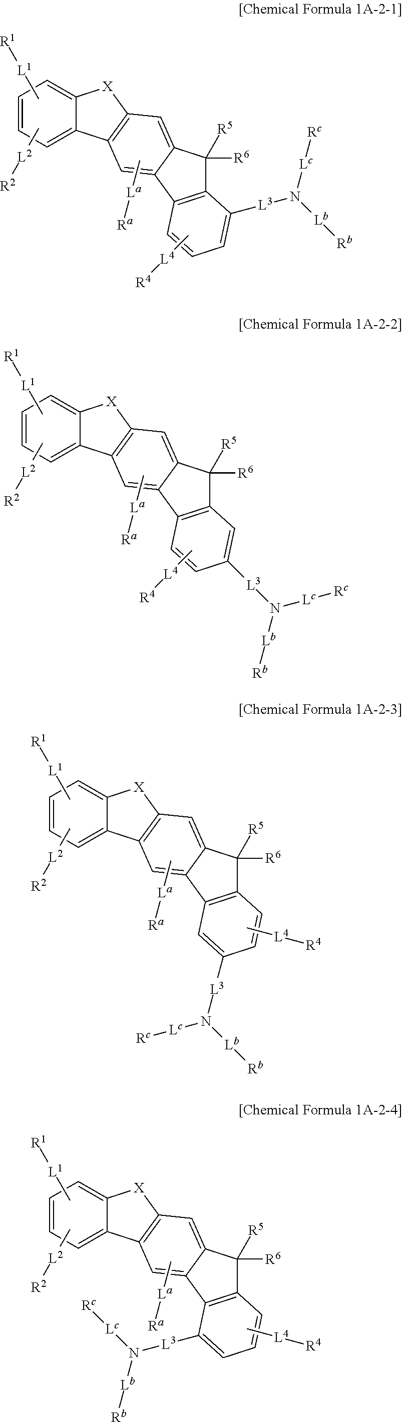

In Chemical Formula 1A-2-1 to Chemical Formula 1A-2-4, X, L^{a}, L^{b}, L^{c} and L¹ to L⁴ and R¹ to R⁶ and R^{b} and R^{c} may be defined the same as those of Chemical Formulae 1 and 2.

In an implementation, the first compound represented by Chemical Formula 1A may be represented by one of Chemical Formula 1A-1-1, Chemical Formula 1A-2-2, and Chemical Formula 1A-2-3.

In an implementation, the first compound represented by Chemical Formula 1B may be represented by Chemical Formula 1B-1 or Chemical Formula 1B-2 (e.g., according to a substitution direction of the group represented by Chemical Formula a).

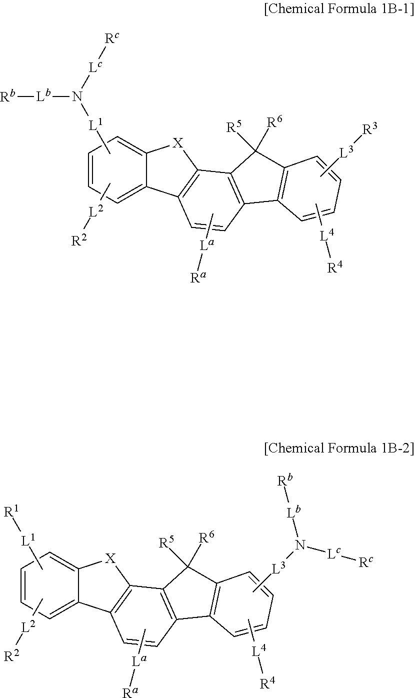

In Chemical Formula 1B-1 and Chemical Formula 1B-2, X, L^{a}, L^{b}, L^{c}, L¹ to L⁴. R^{a}, R¹ to R⁶, R^{b}, and R^{c} may be defined the same as those of Chemical Formulae 1 and 2.

In an implementation, the first compound represented by Chemical Formula 1B-1 may be represented by one of Chemical Formula 1B-1-1 to Chemical Formula 1B-1-4 (e.g., according to a specific substitution position of the group represented by Chemical Formula a).

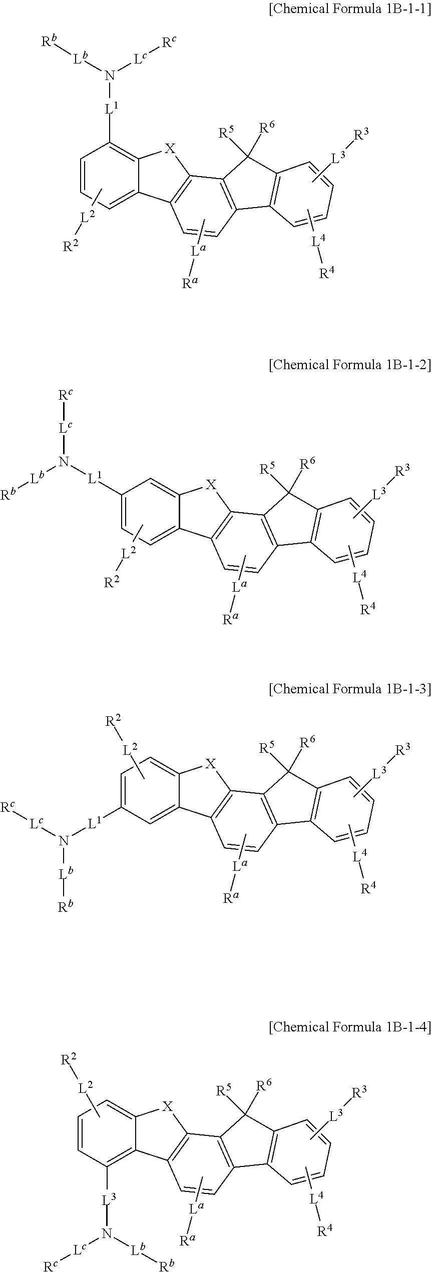

In Chemical Formula 1B-1-1 to Chemical Formula 1B-1-4, X, L^{a}, L^{b}, L^{c}, L¹ to L⁴, R^{a}, R¹ to R⁶, R^{b}, and R^{c} may be defined the same as those of Chemical Formulae 1 and 2.

In an implementation, the first compound represented by Chemical Formula 1B-2 may be represented by one of Chemical Formula 1B-2-1 to Chemical Formula 1B-2-4 (e.g., according to a substitution position of the group represented by Chemical Formula a).

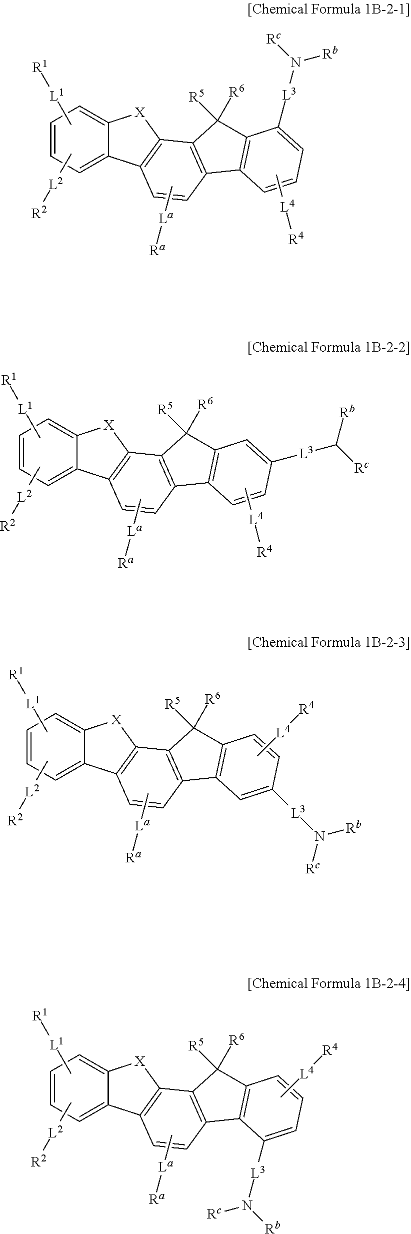

In Chemical Formula 1B-2-1 to Chemical Formula 1B-2-4, X, L^{a}, L^{b}, L^{c}, L¹ to L⁴, R^{a}, R¹ to R⁶, R^{b}, and R^{c} may be defined the same as those of Chemical Formulae 1 and 2.

In an implementation, the first compound represented by Chemical Formula 1B may be represented by one of Chemical Formula 1B-1-1, Chemical Formula 1B-2-2, and Chemical Formula 1B-2-3.

In an implementation, the first compound represented by Chemical Formula 1C may be represented by Chemical Formula 1C-1 or Chemical Formula 1C-2 (e.g., according to a substitution direction of the group represented by Chemical Formula a).

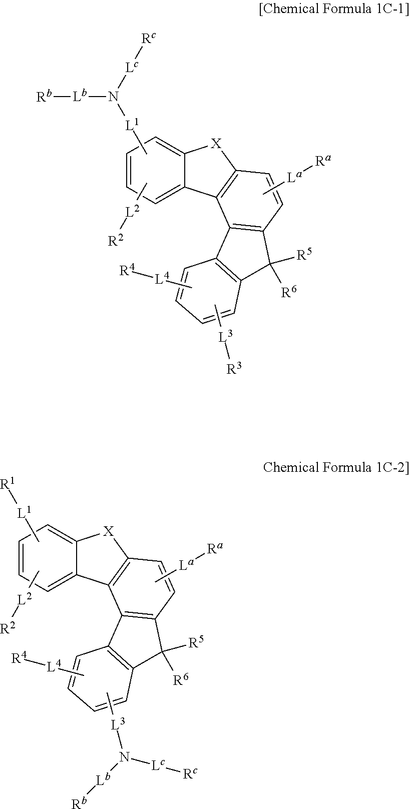

In Chemical Formula 1C-1 and Chemical Formula 1C-2, X, L^{a}, L^{b}, L^{c}, L¹ to L⁴, R^{a}, R¹ to R⁶, R^{b}, and R^{c} may be defined the same as those of Chemical Formulae 1 and 2.

In an implementation, the first compound represented by Chemical Formula 1C-1 may be represented by one of Chemical Formula 1C-1-1 to Chemical Formula 1C-1-4 (e.g., according to a specific substitution position of the group represented by Chemical Formula a).

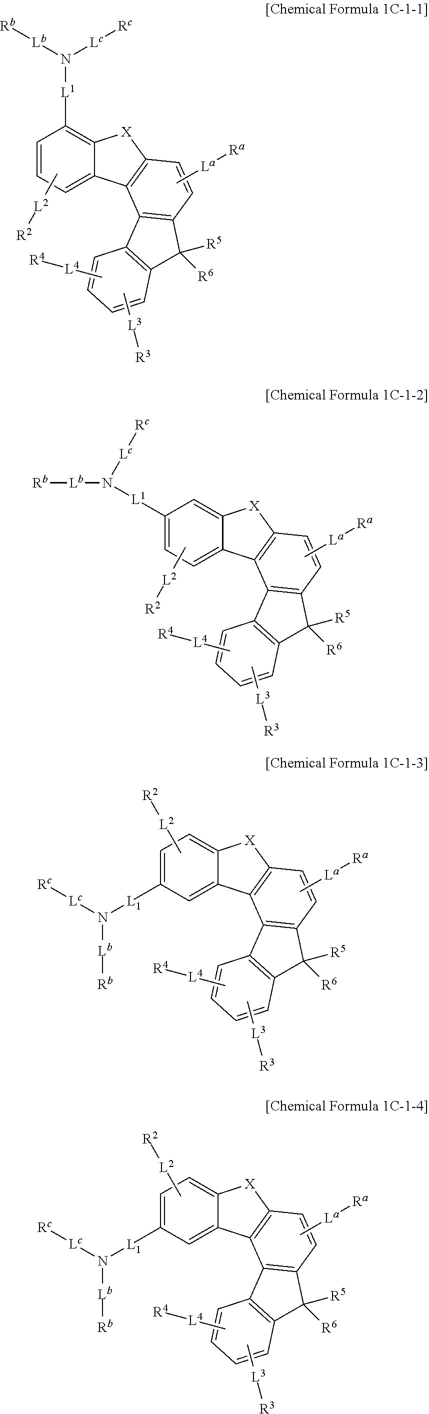

In Chemical Formula 1C-1-1 to Chemical Formula 1C-1-4, X, L^{a}, L^{b}, L^{c}, L¹ to L⁴, R^{a}, R¹ to R⁶, R^{b}, and R^{c} may be defined the same as those of Chemical Formulae 1 and 2.

In an implementation, the first compound represented by Chemical Formula 1C-2 may be represented by one of Chemical Formula 1C-2-1 to Chemical Formula 1C-2-4 (e.g., according to a specific substitution position of the group represented by Chemical Formula a).

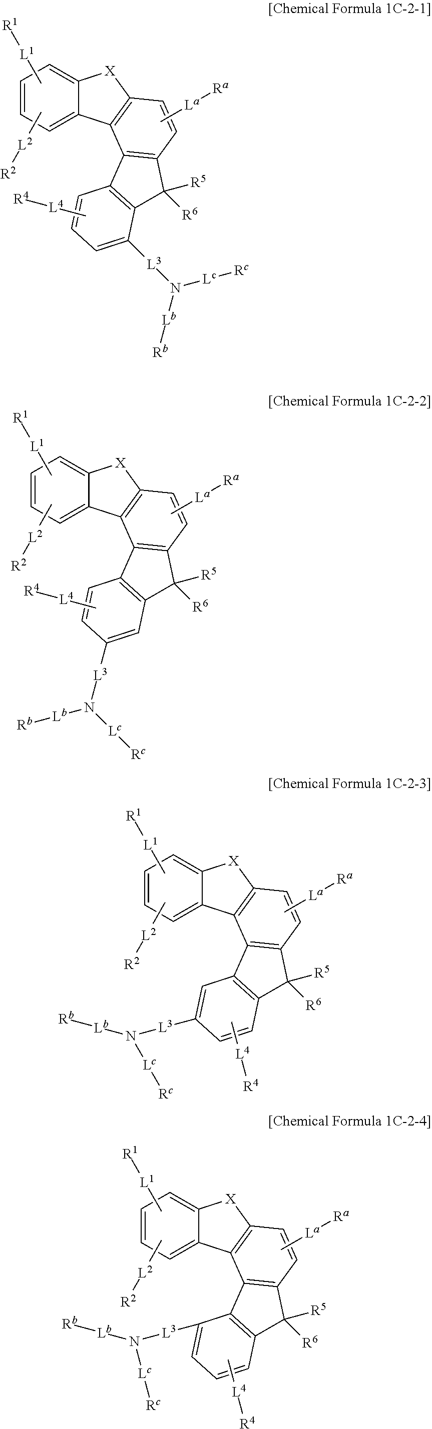

In Chemical Formula 1C-2-1 to Chemical Formula 1C-2-4, X, L^{a}, L^{b}, L^{c}, L¹ to L⁴, R^{a}, R¹ to R⁶, R^{b}, and R^{c} may be defined the same as those of Chemical Formulae 1 and 2.

In an implementation, the first compound represented by Chemical Formula 1C may be represented by one of Chemical Formula 1C-1-1, Chemical Formula 1C-2-2, and Chemical Formula 1C-2-3.

In an implementation, the first compound represented by Chemical Formula 1D may be represented by Chemical Formula 1D-1 or Chemical Formula 1 D-2 (e.g., according to a substitution direction of the group represented by Chemical Formula a).

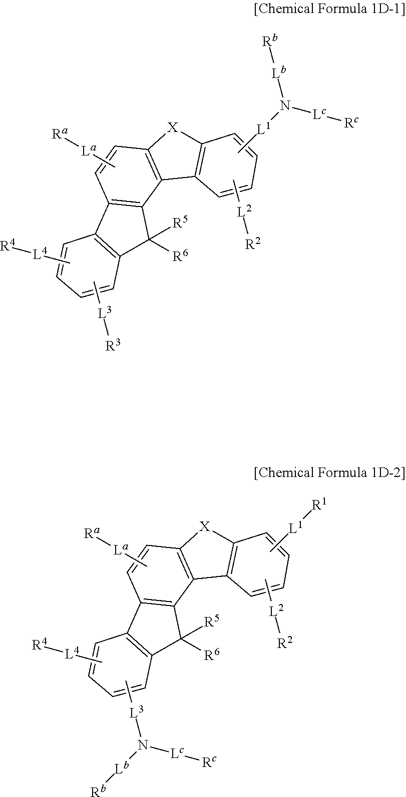

In Chemical Formula 1 D- and Chemical Formula 1D-2, X, L^{a}, L^{b}, L^{c}, L¹ to L⁴, R¹ to R⁶, R^{b}, and R^{c} may be defined the same as those of Chemical Formulae 1 and 2.

In an implementation, the first compound represented by Chemical Formula 1D-1 may be represented by one of Chemical Formula 1D-1-1 to Chemical Formula 1 D-1-4 (e.g., according to a specific substitution position of the group represented by Chemical Formula a).

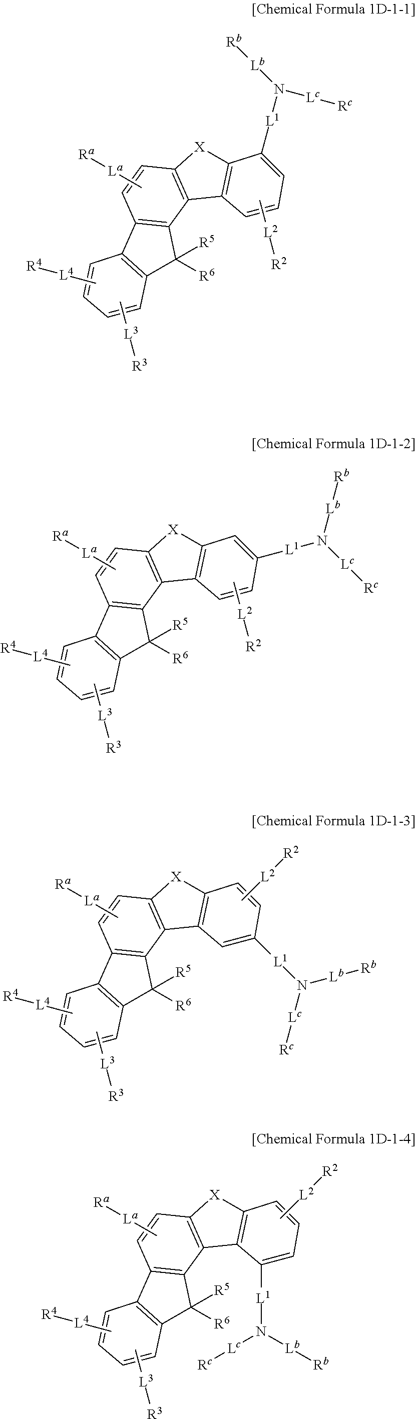

In Chemical Formula 1D-1-1 to Chemical Formula 1D-1-4, X, L^{a}, L^{b}, L^{c}, L¹ to L⁴, R¹ to R⁶, R^{b}, and R^{c} may be defined the same as those of Chemical Formulae 1 and 2.

In an implementation, the first compound represented by Chemical Formula 1D-2 may be represented by one of Chemical Formula 1D-2-1 to Chemical Formula 1D-2-4 (e.g., according to a specific substitution position of the group represented by Chemical Formula a).

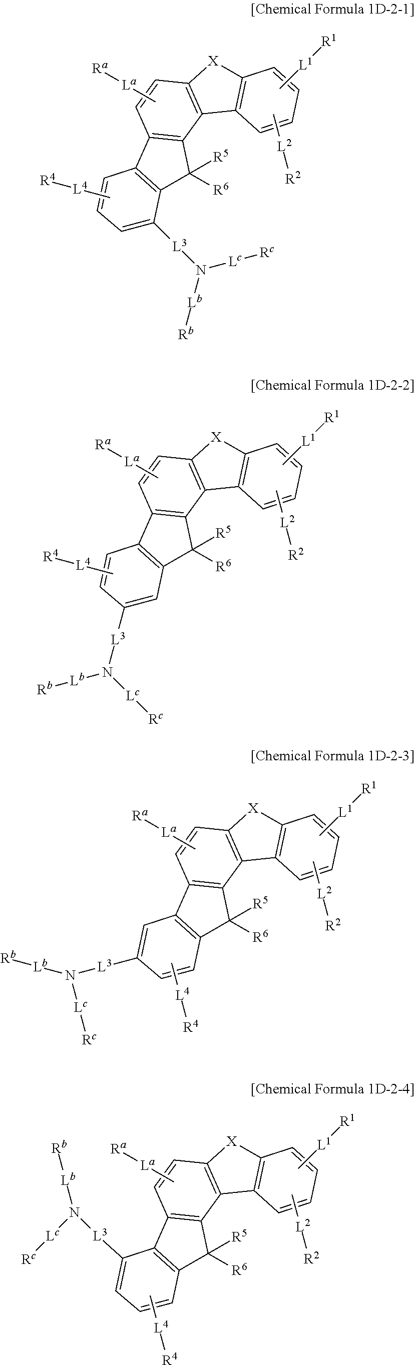

In Chemical Formula 1D-2-1 to Chemical Formula 1D-2-4, X, L^{a}, L^{b}, L^{c}, L¹ to L⁴, R¹ to R⁶, R^{b}, and R^{c} may be defined the same as those of Chemical Formulae 1 and 2.

In an implementation, the first compound represented by Chemical Formula 1D may be represented by one of Chemical Formula 1D-1-1, Chemical Formula 1D-2-2, and Chemical Formula 1D-2-3.

In an implementation, the first compound represented by Chemical Formula 1E may be represented by one of Chemical Formula 1E-1 or Chemical Formula 1E-2 (e.g., according to a substitution direction of the group represented by Chemical Formula a).

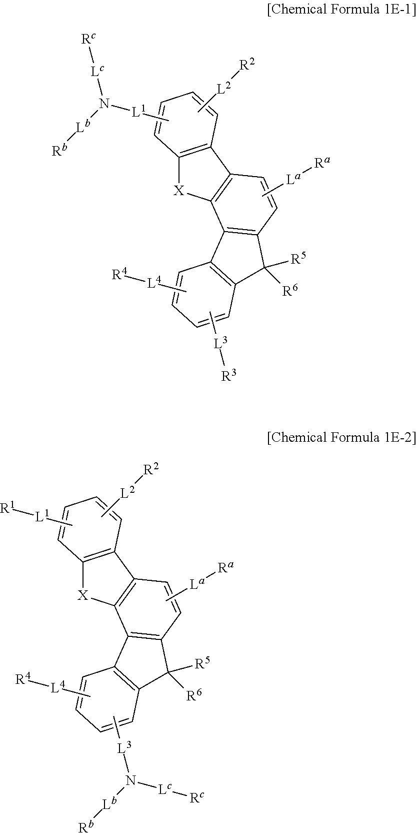

In Chemical Formula 1E-1 and Chemical Formula 1E-2, X, L^{a}, L^{b}, L^{c}, L¹ to L⁴, R¹ to R⁶, R^{b}, and R^{c} may be defined the same as those of Chemical Formulae 1 and 2.

In an implementation, the first compound represented by Chemical Formula 1E-1 may be represented by one of Chemical Formula 1E-1-1 to Chemical Formula 1E-1-4 (e.g., according to a specific substitution position of the group represented by Chemical Formula a).

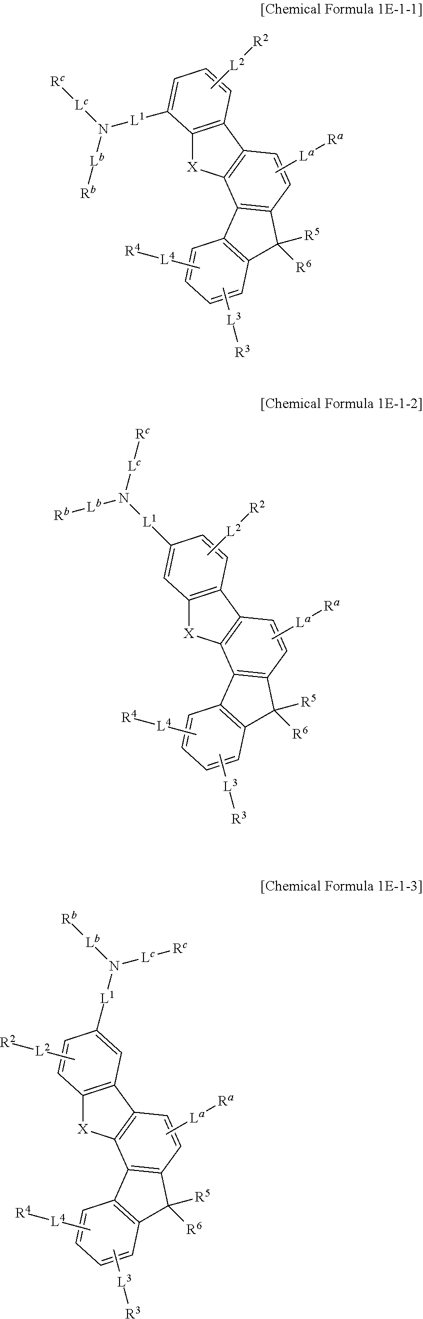

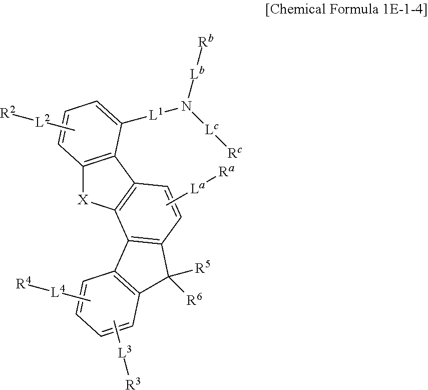

In Chemical Formula 1E-1-1 to Chemical Formula 1E-1-4, X, L^{a}, L^{b}, L^{c}, L¹ to L⁴, R¹ to R⁶, R^{b}, and R^{c} may be defined the same as those of Chemical Formulae 1 and 2.

In an implementation, the first compound represented by Chemical Formula 1E-2 may be represented by one of Chemical Formula 1E-2-1 to Chemical Formula 1E-2-4 (e.g., according to a specific substitution position of the group represented by Chemical Formula a).

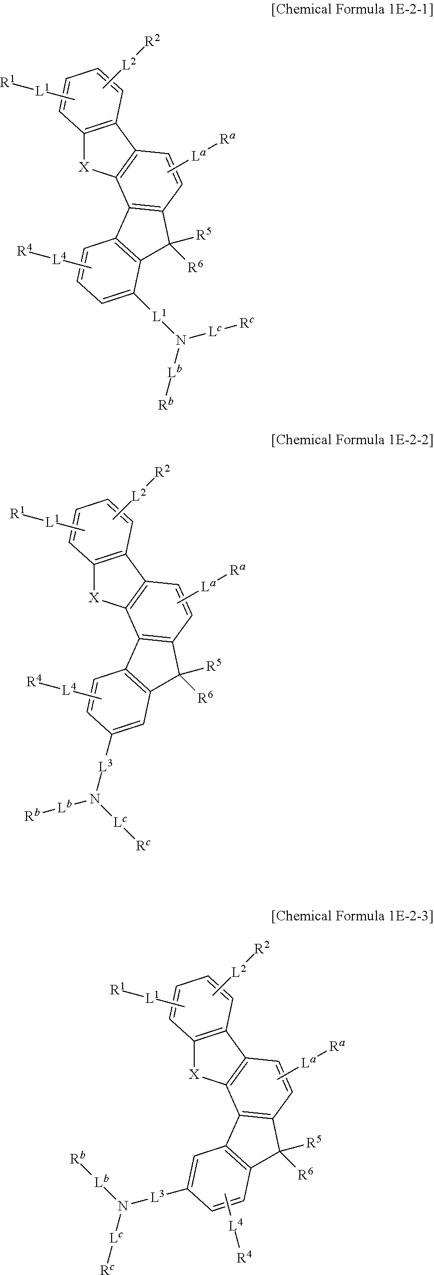

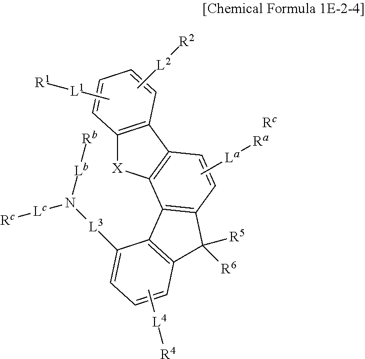

In Chemical Formula 1E-2-1 to Chemical Formula 1E-2-4, X, L^{a}, L^{b}, L^{c}, L¹ to L⁴, R¹ to R⁶, R^{b}, and R^{c} may be defined the same as those of Chemical Formulae 1 and 2.

In an implementation, the first compound represented by Chemical Formula 1E may be represented by one of Chemical Formula 1E-1-1 to Chemical Formula 1E-1-4, and Chemical Formula 1E-2-1 to Chemical Formula 1E-2-4.

In an implementation, the first compound represented by Chemical Formula 1F may be represented by Chemical Formula 1F-1 or Chemical Formula 1F-2 (e.g., according to a substitution direction of the group represented by Chemical Formula a).

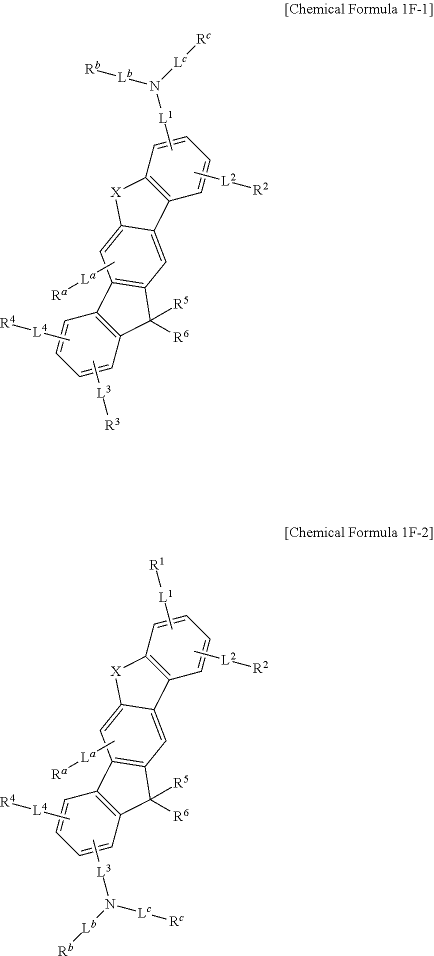

In Chemical Formula 1F-1 and Chemical Formula 1F-2, X, L^{a}, L^{b}, L^{c}, L¹ to L⁴, R¹ to R⁶, R^{b}, and R^{c} may be defined the same as those of Chemical Formulae 1 and 2.

In an implementation, the first compound represented by Chemical Formula 1F-1 may be represented by one of Chemical Formula 1F-1-1 to Chemical Formula 1F-1-4 (e.g., according to a specific substitution position of the group represented by Chemical Formula a).

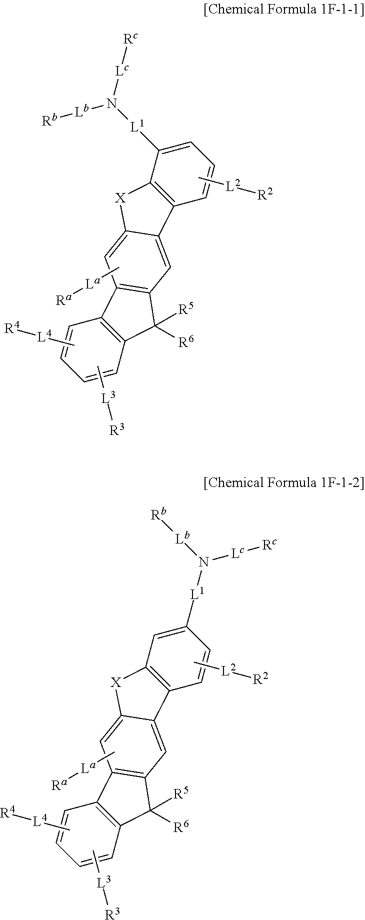

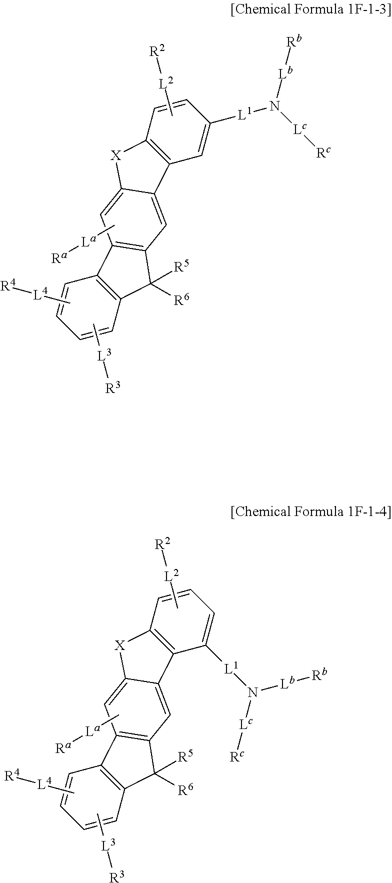

In Chemical Formula 1F-1-1 to Chemical Formula 1F-1-4, X, L^{a}, L^{b}, L^{c}, L¹ to L⁴, R¹ to R⁶, R^{b}, and R^{c} may be defined the same as those of Chemical Formulae 1 and 2.

In an implementation, the first compound represented by Chemical Formula 1F-2 may be represented by one of Chemical Formula 1F-2-1 to Chemical Formula 1F-2-4 (e.g., according to a specific substitution position of the group represented by Chemical Formula a).

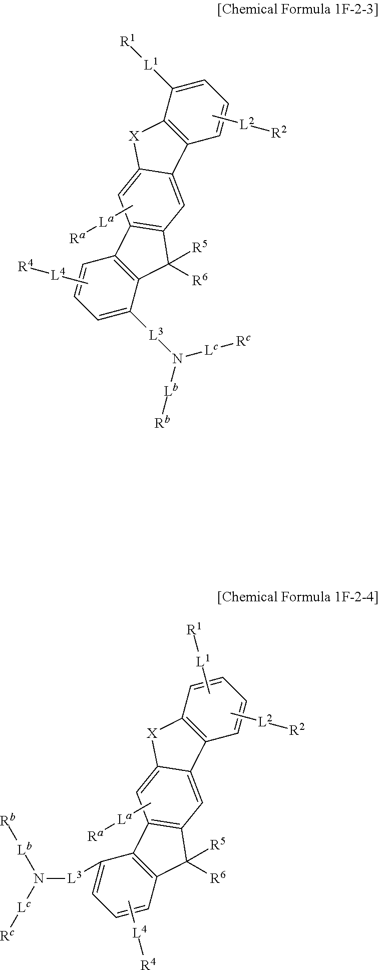

In Chemical Formula 1F-2-1 to Chemical Formula 1F-2-4, X, L^{a}, L^{b}, L^{c}, L¹ to L⁴, R¹ to R⁶. R^{h}, and R may be defined the same as those of Chemical Formulae 1 and 2.

In an implementation, the first compound represented by Chemical Formula 1F may be represented by one of Chemical Formula 1F-1-1, Chemical Formula 1F-2-2, and Chemical Formula 1F-2-3.

In an implementation, the first compound may be represented by Chemical Formula 1E-1-1 or Chemical Formula 1E-2-2, and may be, e.g., represented by Chemical Formula 1E-2-2.

In an implementation, the first compound may be, e.g., a compound of the following Group 1.

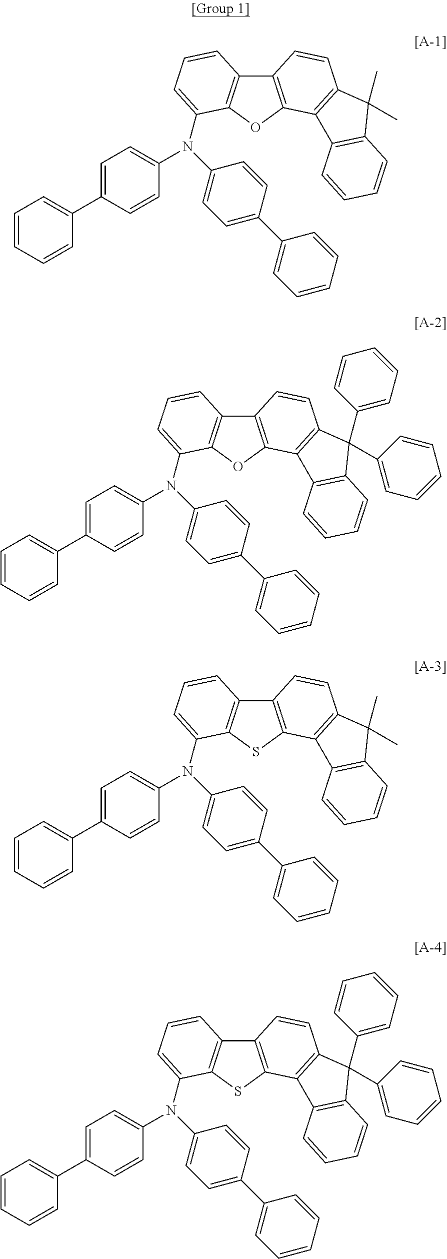

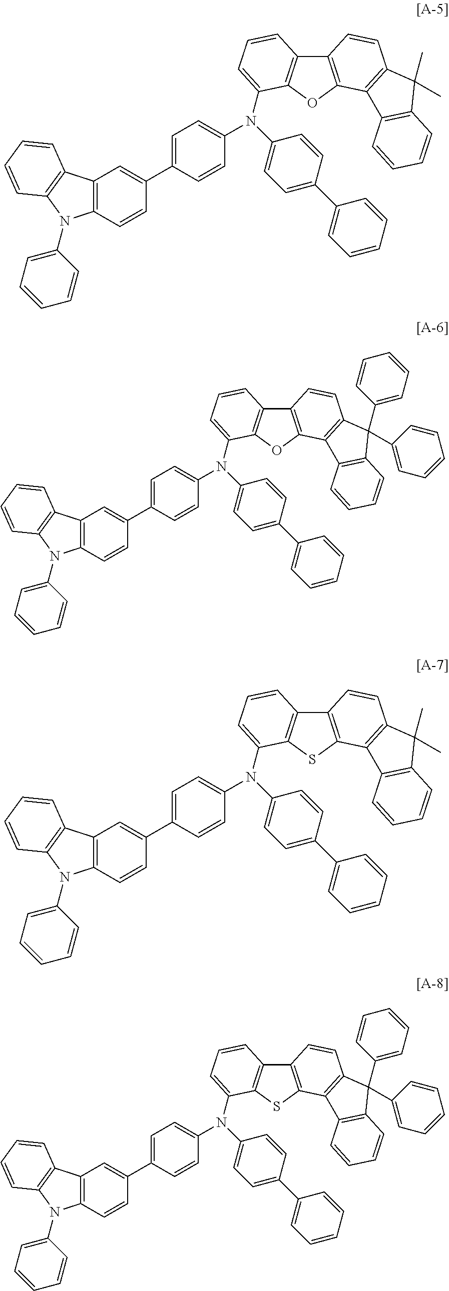 [SMILES: CC1(C)c2ccccc2-c2c1ccc1c2oc2c(N(c3ccc(-c4ccccc4)cc3)c3ccc(-c4ccc5c(c4)c4ccccc4n5-c4ccccc4)cc3)cccc21 ; c1ccc(-c2ccc(N(c3ccc(-c4ccc5c(c4)c4ccccc4n5-c4ccccc4)cc3)c3cccc4c3oc3c5c(ccc34)C(c3ccccc3)(c3ccccc3)c3ccccc3-5)cc2)cc1 ; CC1(C)c2ccccc2-c2c1ccc1c2sc2c(N(c3ccc(-c4ccccc4)cc3)c3ccc(-c4ccc5c(c4)c4ccccc4n5-c4ccccc4)cc3)cccc21 ; c1ccc(-c2ccc(N(c3ccc(-c4ccc5c(c4)c4ccccc4n5-c4ccccc4)cc3)c3cccc4c3sc3c5c(ccc34)C(c3ccccc3)(c3ccccc3)c3ccccc3-5)cc2)cc1]

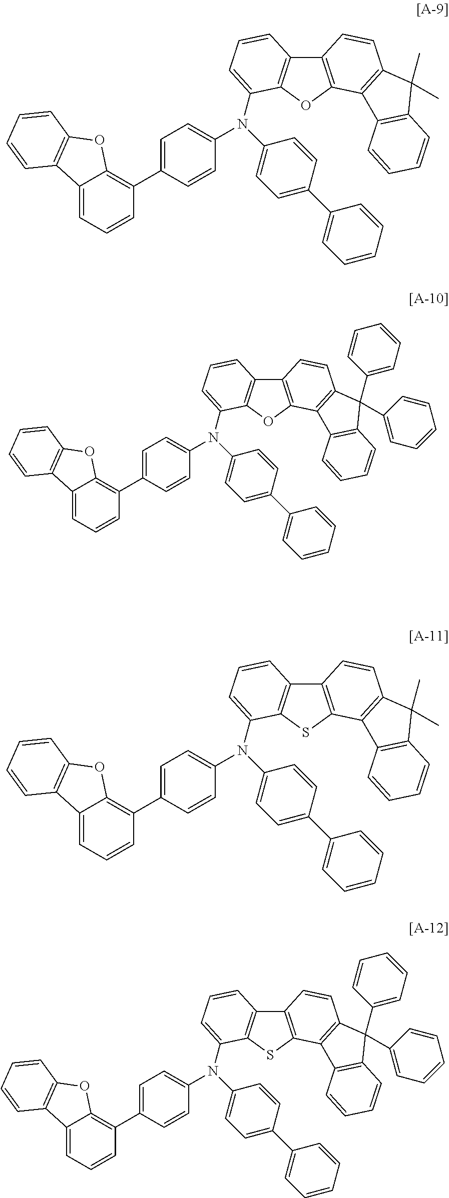 [SMILES: CC1(C)c2ccccc2-c2c1ccc1c2oc2c(N(c3ccc(-c4ccccc4)cc3)c3ccc(-c4cccc5c4oc4ccccc45)cc3)cccc21 ; c1ccc(-c2ccc(N(c3ccc(-c4cccc5c4oc4ccccc45)cc3)c3cccc4c3oc3c5c(ccc34)C(c3ccccc3)(c3ccccc3)c3ccccc3-5)cc2)cc1 ; CC1(C)c2ccccc2-c2c1ccc1c2sc2c(N(c3ccc(-c4ccccc4)cc3)c3ccc(-c4cccc5c4oc4ccccc45)cc3)cccc21 ; c1ccc(-c2ccc(N(c3ccc(-c4cccc5c4oc4ccccc45)cc3)c3cccc4c3sc3c5c(ccc34)C(c3ccccc3)(c3ccccc3)c3ccccc3-5)cc2)cc1]

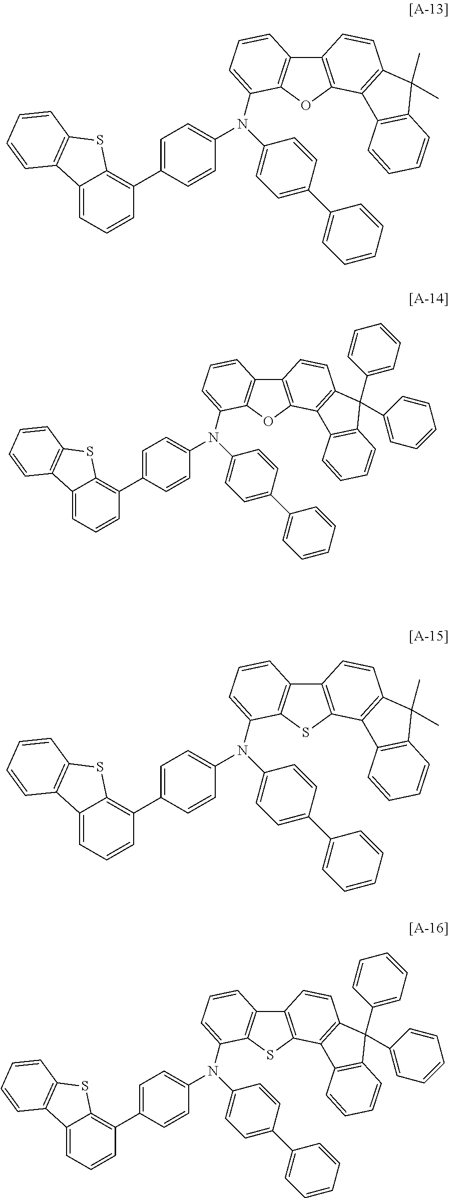 [SMILES: CC1(C)c2ccccc2-c2c1ccc1c2oc2c(N(c3ccc(-c4ccccc4)cc3)c3ccc(-c4cccc5c4sc4ccccc45)cc3)cccc21 ; c1ccc(-c2ccc(N(c3ccc(-c4cccc5c4sc4ccccc45)cc3)c3cccc4c3oc3c5c(ccc34)C(c3ccccc3)(c3ccccc3)c3ccccc3-5)cc2)cc1 ; CC1(C)c2ccccc2-c2c1ccc1c2sc2c(N(c3ccc(-c4ccccc4)cc3)c3ccc(-c4cccc5c4sc4ccccc45)cc3)cccc21 ; c1ccc(-c2ccc(N(c3ccc(-c4cccc5c4sc4ccccc45)cc3)c3cccc4c3sc3c5c(ccc34)C(c3ccccc3)(c3ccccc3)c3ccccc3-5)cc2)cc1]

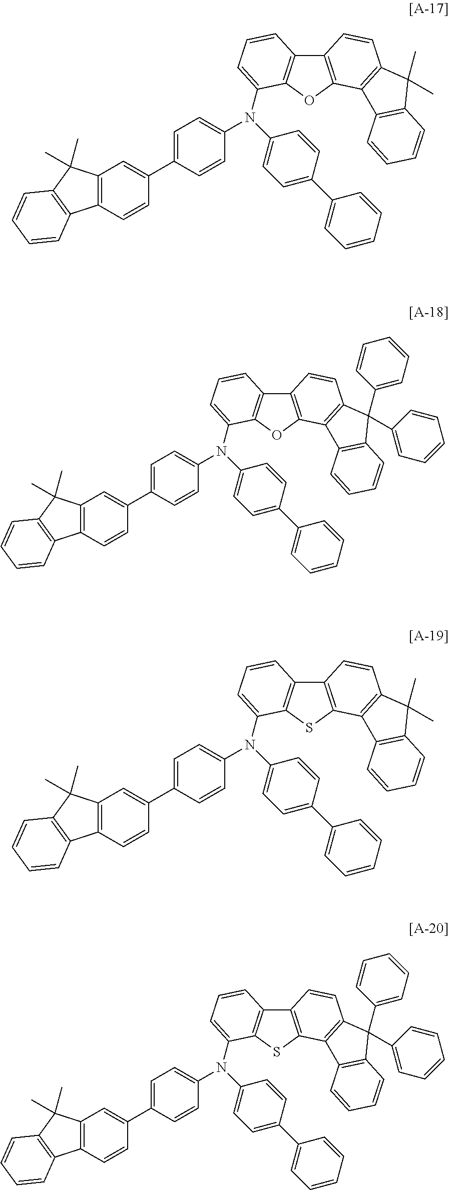 [SMILES: CC1(C)c2ccccc2-c2ccc(-c3ccc(N(c4ccc(-c5ccccc5)cc4)c4cccc5c4oc4c6c(ccc45)C(C)(C)c4ccccc4-6)cc3)cc21 ; CC1(C)c2ccccc2-c2ccc(-c3ccc(N(c4ccc(-c5ccccc5)cc4)c4cccc5c4oc4c6c(ccc45)C(c4ccccc4)(c4ccccc4)c4ccccc4-6)cc3)cc21 ; CC1(C)c2ccccc2-c2ccc(-c3ccc(N(c4ccc(-c5ccccc5)cc4)c4cccc5c4sc4c6c(ccc45)C(C)(C)c4ccccc4-6)cc3)cc21 ; CC1(C)c2ccccc2-c2ccc(-c3ccc(N(c4ccc(-c5ccccc5)cc4)c4cccc5c4sc4c6c(ccc45)C(c4ccccc4)(c4ccccc4)c4ccccc4-6)cc3)cc21]

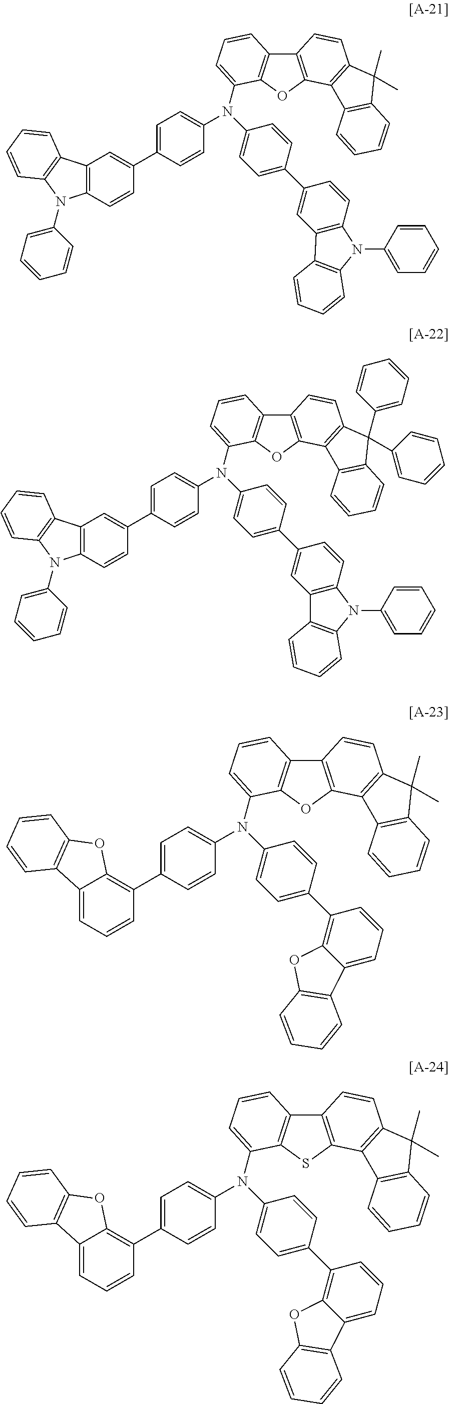 [SMILES: CC1(C)c2ccccc2-c2c1ccc1c2oc2c(N(c3ccc(-c4ccc5c(c4)c4ccccc4n5-c4ccccc4)cc3)c3ccc(-c4ccc5c(c4)c4ccccc4n5-c4ccccc4)cc3)cccc21 ; c1ccc(-n2c3ccccc3c3cc(-c4ccc(N(c5ccc(-c6ccc7c(c6)c6ccccc6n7-c6ccccc6)cc5)c5cccc6c5oc5c7c(ccc56)C(c5ccccc5)(c5ccccc5)c5ccccc5-7)cc4)ccc32)cc1 ; CC1(C)c2ccccc2-c2c1ccc1c2oc2c(N(c3ccc(-c4cccc5c4oc4ccccc45)cc3)c3ccc(-c4cccc5c4oc4ccccc45)cc3)cccc21 ; CC1(C)c2ccccc2-c2c1ccc1c2sc2c(N(c3ccc(-c4cccc5c4oc4ccccc45)cc3)c3ccc(-c4cccc5c4oc4ccccc45)cc3)cccc21]

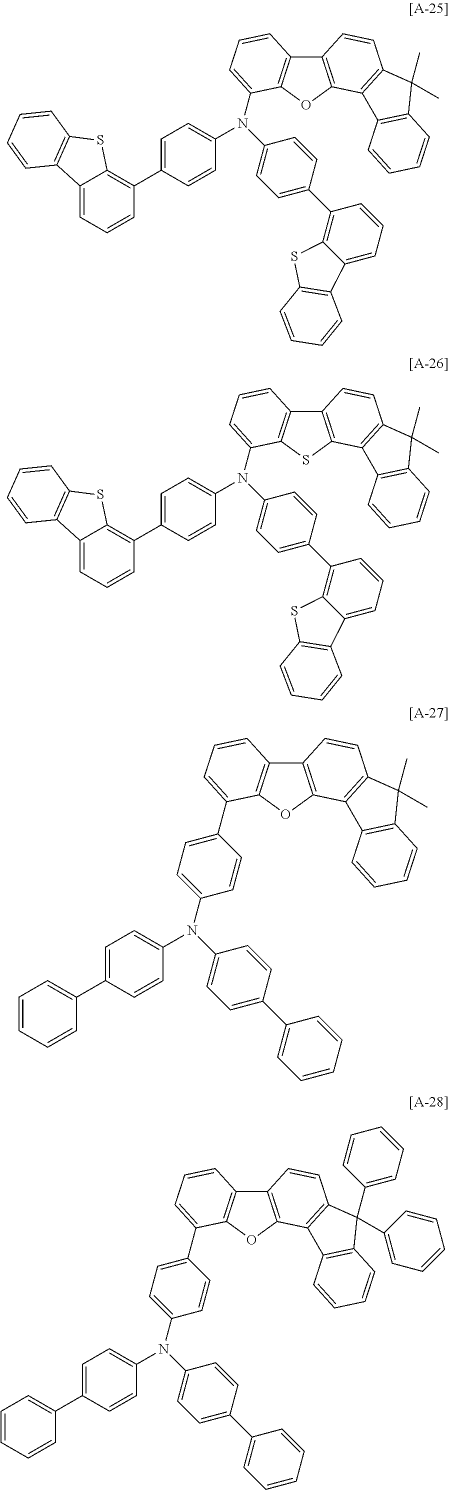

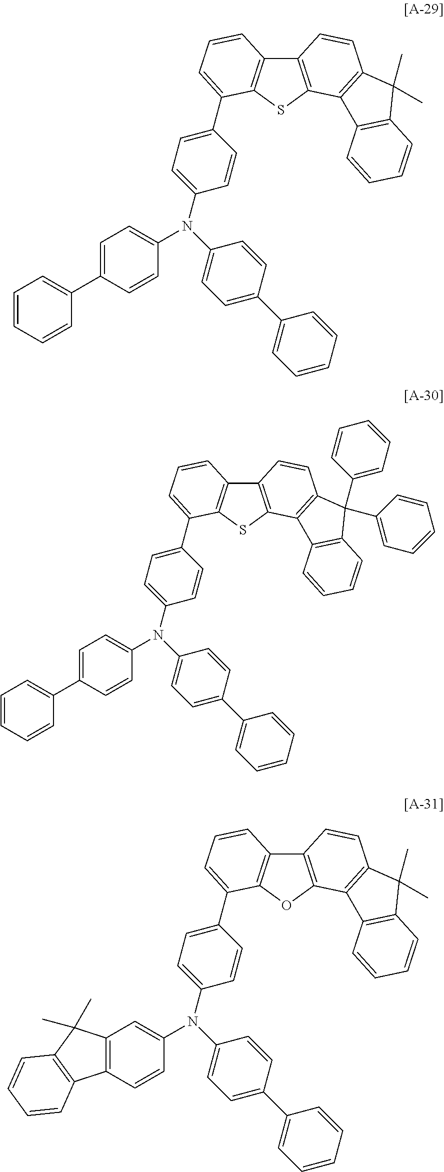 [SMILES: CC1(C)c2ccccc2-c2c1ccc1c2sc2c(-c3ccc(N(c4ccc(-c5ccccc5)cc4)c4ccc(-c5ccccc5)cc4)cc3)cccc21 ; c1ccc(-c2ccc(N(c3ccc(-c4ccccc4)cc3)c3ccc(-c4cccc5c4sc4c6c(ccc45)C(c4ccccc4)(c4ccccc4)c4ccccc4-6)cc3)cc2)cc1 ; CC1(C)c2ccccc2-c2ccc(N(c3ccc(-c4ccccc4)cc3)c3ccc(-c4cccc5c4oc4c6c(ccc45)C(C)(C)c4ccccc4-6)cc3)cc21]

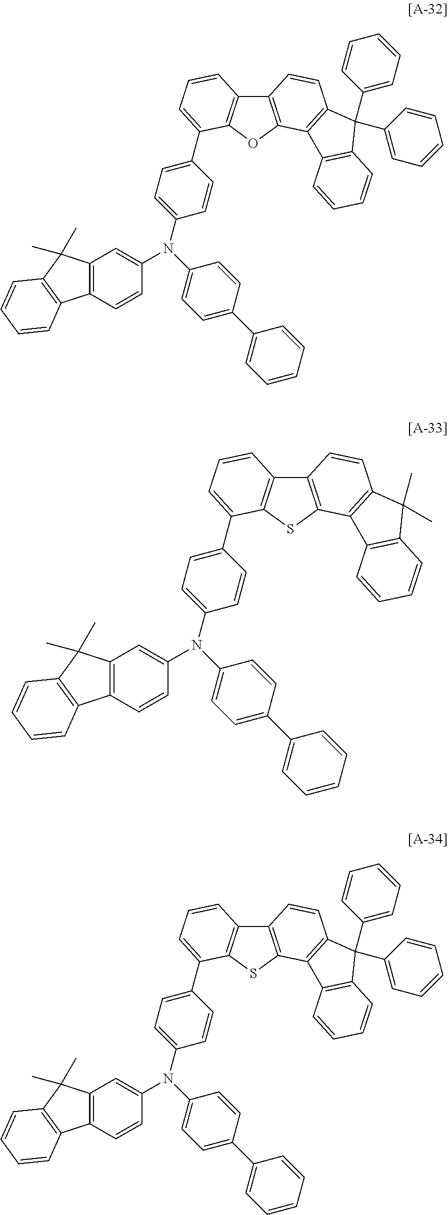 [SMILES: CC1(C)c2ccccc2-c2ccc(N(c3ccc(-c4ccccc4)cc3)c3ccc(-c4cccc5c4oc4c6c(ccc45)C(c4ccccc4)(c4ccccc4)c4ccccc4-6)cc3)cc21 ; CC1(C)c2ccccc2-c2ccc(N(c3ccc(-c4ccccc4)cc3)c3ccc(-c4cccc5c4sc4c6c(ccc45)C(C)(C)c4ccccc4-6)cc3)cc21 ; CC1(C)c2ccccc2-c2ccc(N(c3ccc(-c4ccccc4)cc3)c3ccc(-c4cccc5c4sc4c6c(ccc45)C(c4ccccc4)(c4ccccc4)c4ccccc4-6)cc3)cc21]

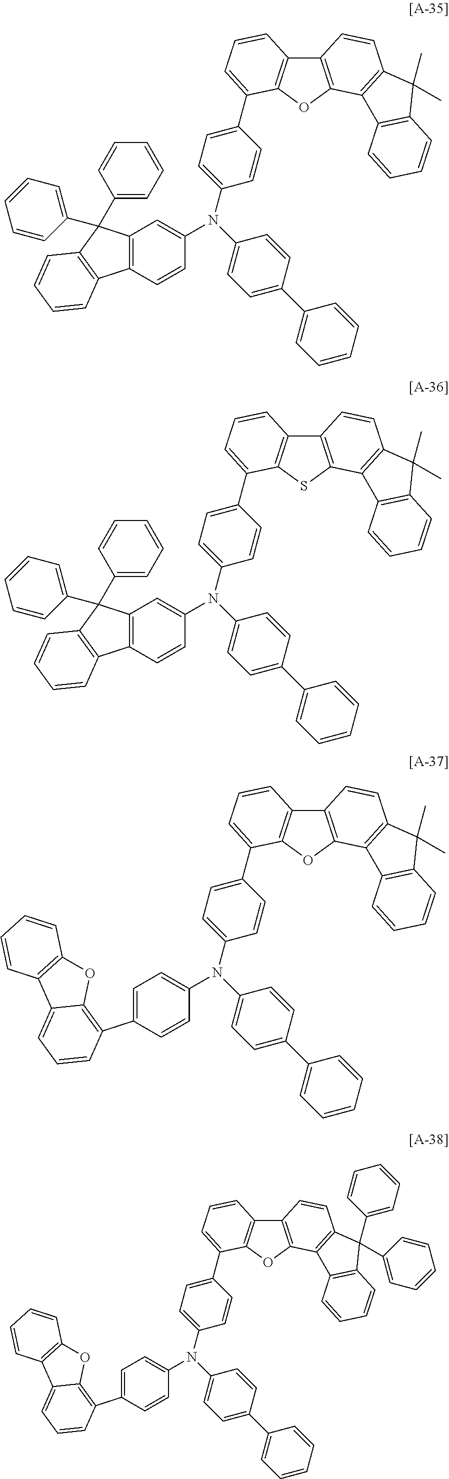 [SMILES: CC1(C)c2ccccc2-c2c1ccc1c2oc2c(-c3ccc(N(c4ccc(-c5ccccc5)cc4)c4ccc5c(c4)C(c4ccccc4)(c4ccccc4)c4ccccc4-5)cc3)cccc21 ; CC1(C)c2ccccc2-c2c1ccc1c2sc2c(-c3ccc(N(c4ccc(-c5ccccc5)cc4)c4ccc5c(c4)C(c4ccccc4)(c4ccccc4)c4ccccc4-5)cc3)cccc21 ; CC1(C)c2ccccc2-c2c1ccc1c2oc2c(-c3ccc(N(c4ccc(-c5ccccc5)cc4)c4ccc(-c5cccc6c5oc5ccccc56)cc4)cc3)cccc21 ; c1ccc(-c2ccc(N(c3ccc(-c4cccc5c4oc4ccccc45)cc3)c3ccc(-c4cccc5c4oc4c6c(ccc45)C(c4ccccc4)(c4ccccc4)c4ccccc4-6)cc3)cc2)cc1]

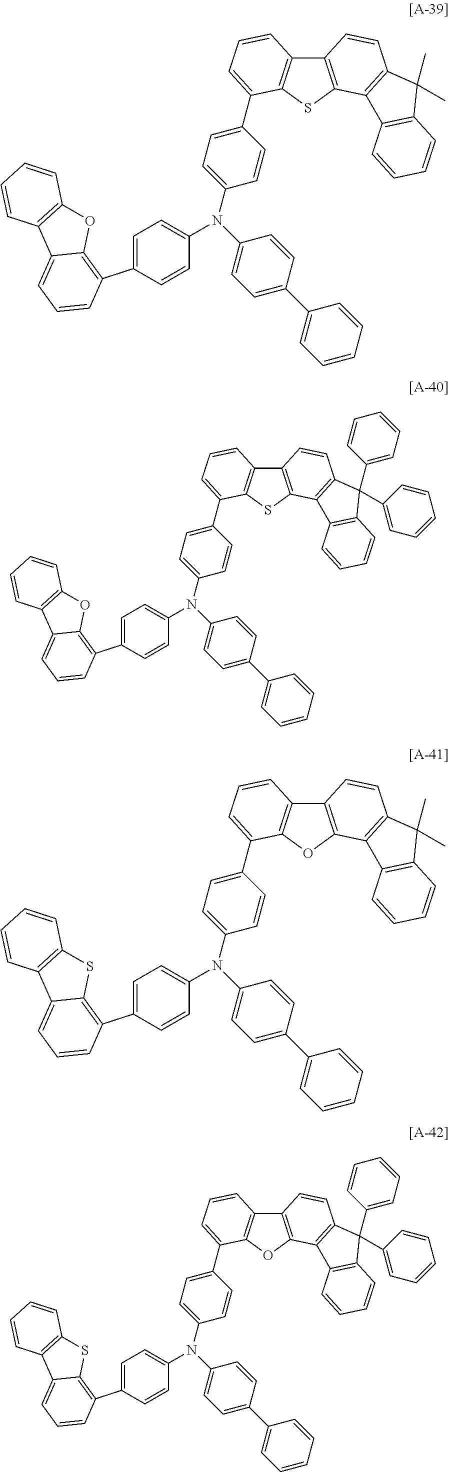 [SMILES: CC1(C)c2ccccc2-c2c1ccc1c2sc2c(-c3ccc(N(c4ccc(-c5ccccc5)cc4)c4ccc(-c5cccc6c5oc5ccccc56)cc4)cc3)cccc21 ; c1ccc(-c2ccc(N(c3ccc(-c4cccc5c4oc4ccccc45)cc3)c3ccc(-c4cccc5c4sc4c6c(ccc45)C(c4ccccc4)(c4ccccc4)c4ccccc4-6)cc3)cc2)cc1 ; CC1(C)c2ccccc2-c2c1ccc1c2oc2c(-c3ccc(N(c4ccc(-c5ccccc5)cc4)c4ccc(-c5cccc6c5sc5ccccc56)cc4)cc3)cccc21 ; c1ccc(-c2ccc(N(c3ccc(-c4cccc5c4oc4c6c(ccc45)C(c4ccccc4)(c4ccccc4)c4ccccc4-6)cc3)c3ccc(-c4cccc5c4sc4ccccc45)cc3)cc2)cc1]

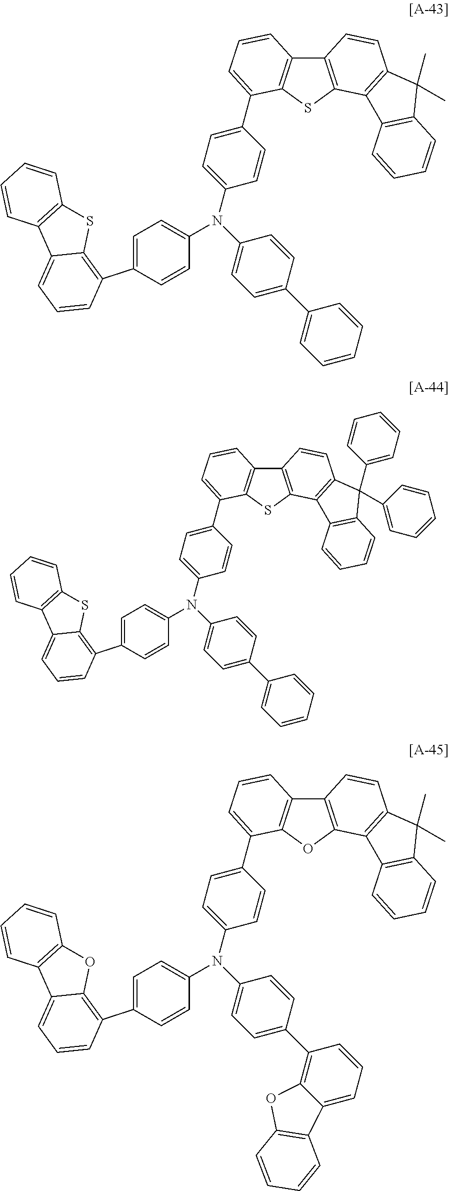 [SMILES: CC1(C)c2ccccc2-c2c1ccc1c2sc2c(-c3ccc(N(c4ccc(-c5ccccc5)cc4)c4ccc(-c5cccc6c5sc5ccccc56)cc4)cc3)cccc21 ; c1ccc(-c2ccc(N(c3ccc(-c4cccc5c4sc4ccccc45)cc3)c3ccc(-c4cccc5c4sc4c6c(ccc45)C(c4ccccc4)(c4ccccc4)c4ccccc4-6)cc3)cc2)cc1 ; CC1(C)c2ccccc2-c2c1ccc1c2oc2c(-c3ccc(N(c4ccc(-c5cccc6c5oc5ccccc56)cc4)c4ccc(-c5cccc6c5oc5ccccc56)cc4)cc3)cccc21]

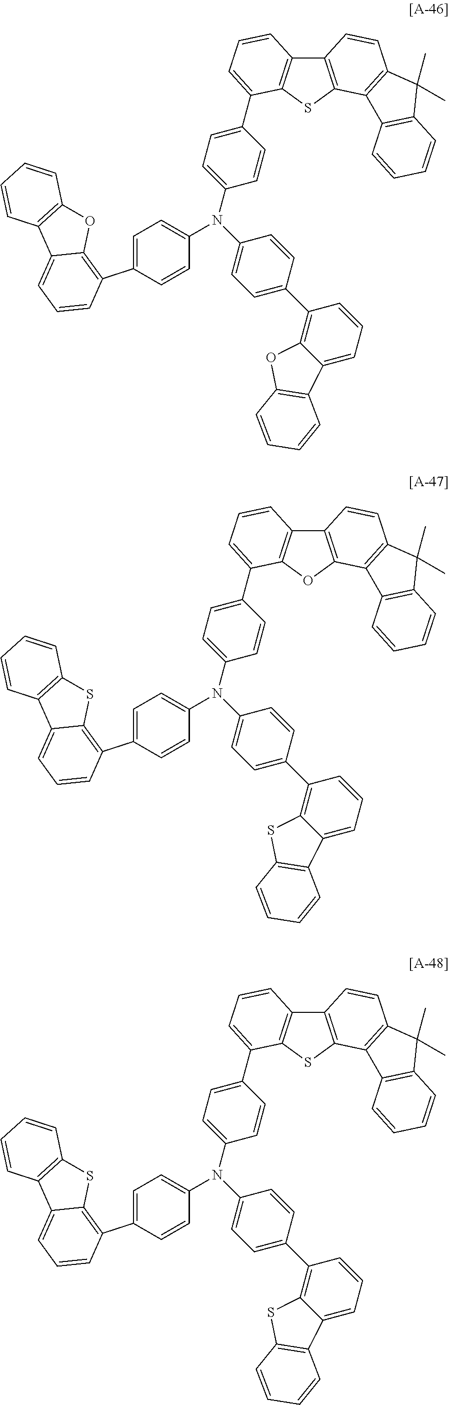 [SMILES: CC1(C)c2ccccc2-c2c1ccc1c2sc2c(-c3ccc(N(c4ccc(-c5cccc6c5oc5ccccc56)cc4)c4ccc(-c5cccc6c5oc5ccccc56)cc4)cc3)cccc21 ; CC1(C)c2ccccc2-c2c1ccc1c2oc2c(-c3ccc(N(c4ccc(-c5cccc6c5sc5ccccc56)cc4)c4ccc(-c5cccc6c5sc5ccccc56)cc4)cc3)cccc21 ; CC1(C)c2ccccc2-c2c1ccc1c2sc2c(-c3ccc(N(c4ccc(-c5cccc6c5sc5ccccc56)cc4)c4ccc(-c5cccc6c5sc5ccccc56)cc4)cc3)cccc21]

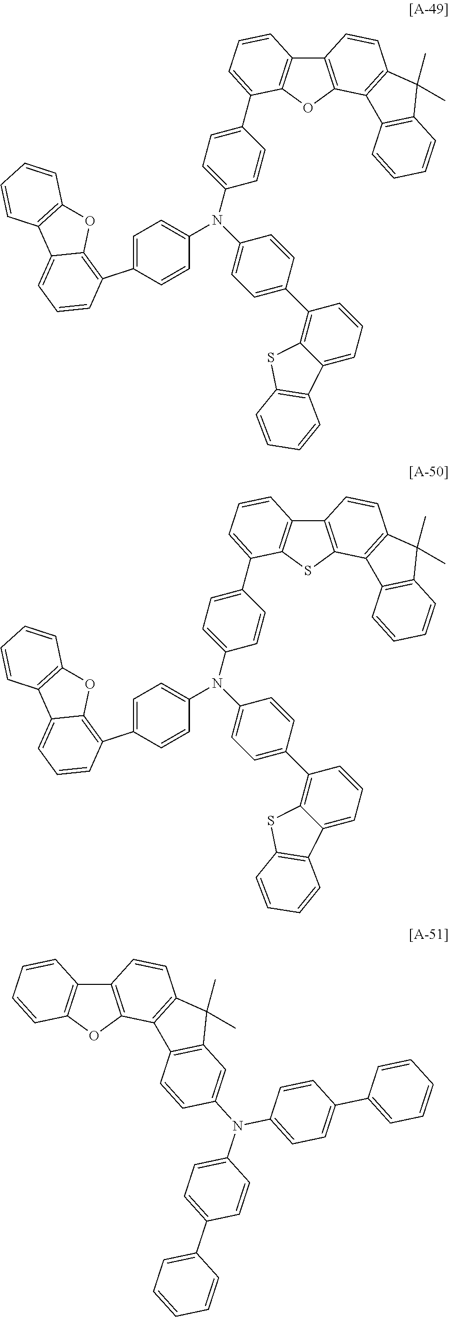 [SMILES: CC1(C)c2ccccc2-c2c1ccc1c2oc2c(-c3ccc(N(c4ccc(-c5cccc6c5oc5ccccc56)cc4)c4ccc(-c5cccc6c5sc5ccccc56)cc4)cc3)cccc21 ; CC1(C)c2ccccc2-c2c1ccc1c2sc2c(-c3ccc(N(c4ccc(-c5cccc6c5oc5ccccc56)cc4)c4ccc(-c5cccc6c5sc5ccccc56)cc4)cc3)cccc21 ; CC1(C)c2cc(N(c3ccc(-c4ccccc4)cc3)c3ccc(-c4ccccc4)cc3)ccc2-c2c1ccc1c2oc2ccccc21]

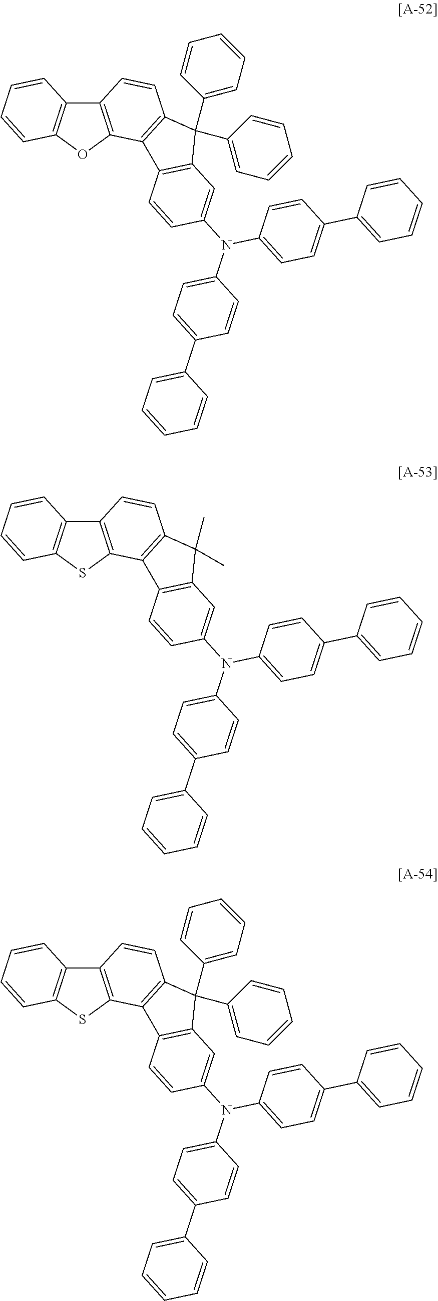 [SMILES: c1ccc(-c2ccc(N(c3ccc(-c4ccccc4)cc3)c3ccc4c(c3)C(c3ccccc3)(c3ccccc3)c3ccc5c(oc6ccccc65)c3-4)cc2)cc1 ; CC1(C)c2cc(N(c3ccc(-c4ccccc4)cc3)c3ccc(-c4ccccc4)cc3)ccc2-c2c1ccc1c2sc2ccccc21 ; c1ccc(-c2ccc(N(c3ccc(-c4ccccc4)cc3)c3ccc4c(c3)C(c3ccccc3)(c3ccccc3)c3ccc5c(sc6ccccc65)c3-4)cc2)cc1]

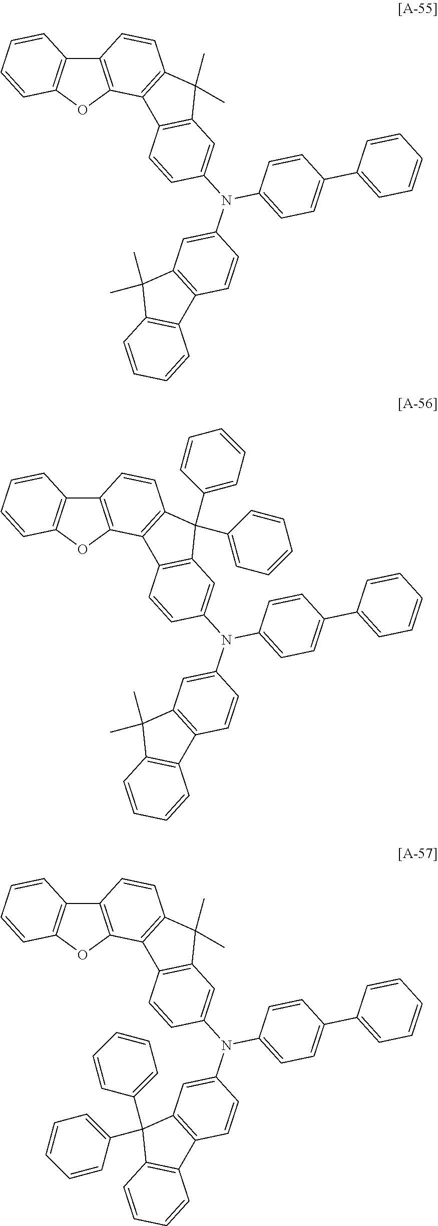 [SMILES: CC1(C)c2ccccc2-c2ccc(N(c3ccc(-c4ccccc4)cc3)c3ccc4c(c3)C(C)(C)c3ccc5c(oc6ccccc65)c3-4)cc21 ; CC1(C)c2ccccc2-c2ccc(N(c3ccc(-c4ccccc4)cc3)c3ccc4c(c3)C(c3ccccc3)(c3ccccc3)c3ccc5c(oc6ccccc65)c3-4)cc21 ; CC1(C)c2cc(N(c3ccc(-c4ccccc4)cc3)c3ccc4c(c3)C(c3ccccc3)(c3ccccc3)c3ccccc3-4)ccc2-c2c1ccc1c2oc2ccccc21]

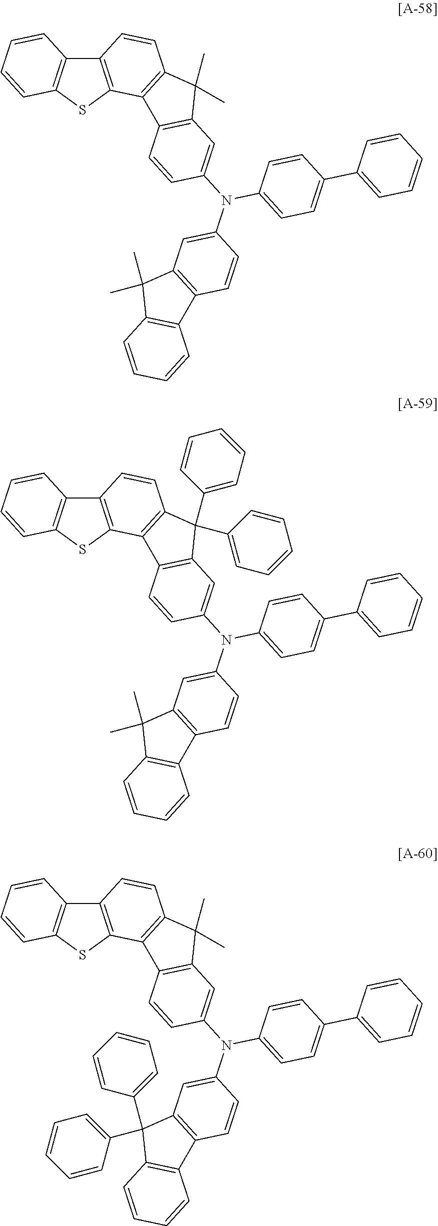 [SMILES: CC1(C)c2ccccc2-c2ccc(N(c3ccc(-c4ccccc4)cc3)c3ccc4c(c3)C(C)(C)c3ccc5c(sc6ccccc65)c3-4)cc21 ; CC1(C)c2ccccc2-c2ccc(N(c3ccc(-c4ccccc4)cc3)c3ccc4c(c3)C(c3ccccc3)(c3ccccc3)c3ccc5c(sc6ccccc65)c3-4)cc21 ; CC1(C)c2cc(N(c3ccc(-c4ccccc4)cc3)c3ccc4c(c3)C(c3ccccc3)(c3ccccc3)c3ccccc3-4)ccc2-c2c1ccc1c2sc2ccccc21]

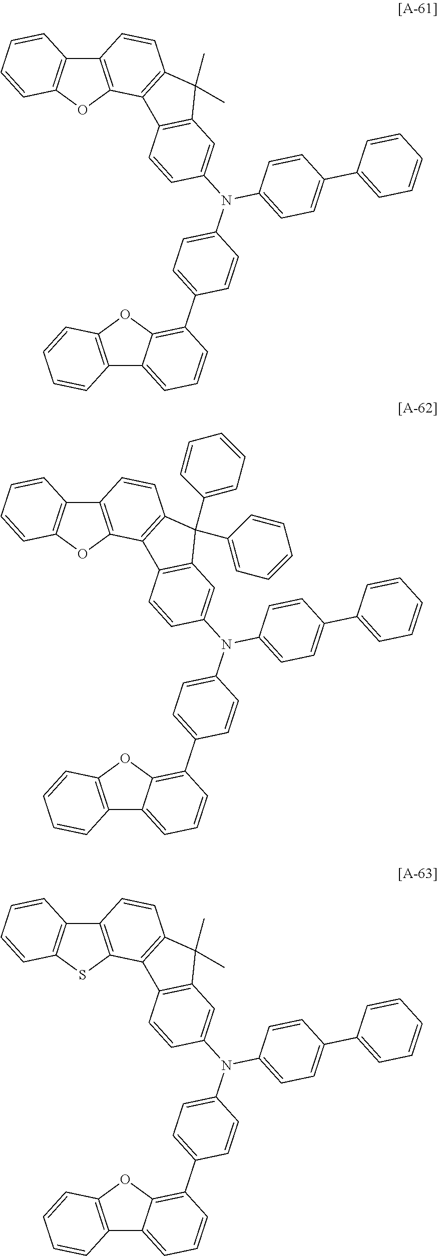 [SMILES: CC1(C)c2cc(N(c3ccc(-c4ccccc4)cc3)c3ccc(-c4cccc5c4oc4ccccc45)cc3)ccc2-c2c1ccc1c2oc2ccccc21 ; c1ccc(-c2ccc(N(c3ccc(-c4cccc5c4oc4ccccc45)cc3)c3ccc4c(c3)C(c3ccccc3)(c3ccccc3)c3ccc5c(oc6ccccc65)c3-4)cc2)cc1 ; CC1(C)c2cc(N(c3ccc(-c4ccccc4)cc3)c3ccc(-c4cccc5c4oc4ccccc45)cc3)ccc2-c2c1ccc1c2sc2ccccc21]

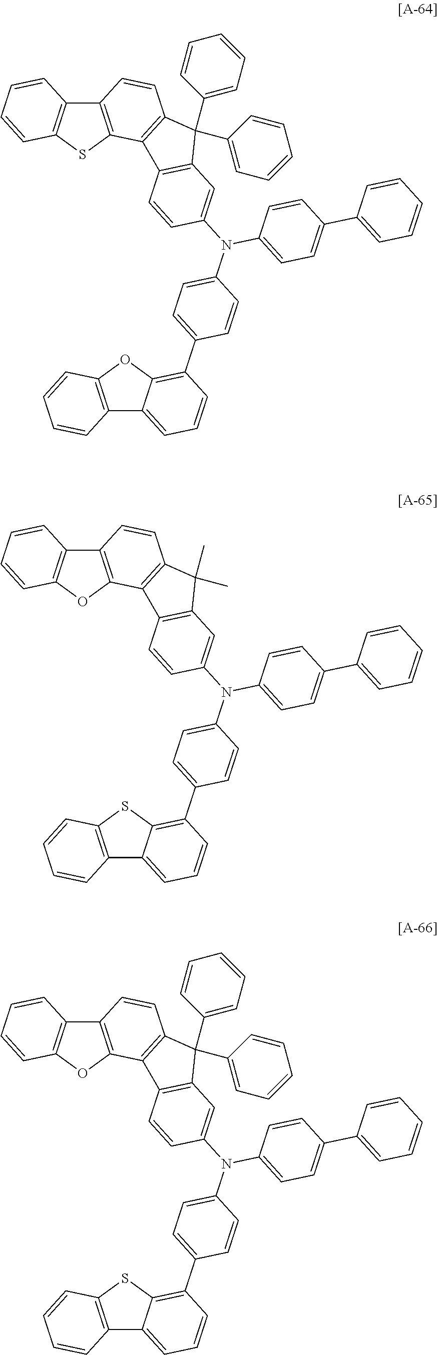 [SMILES: c1ccc(-c2ccc(N(c3ccc(-c4cccc5c4oc4ccccc45)cc3)c3ccc4c(c3)C(c3ccccc3)(c3ccccc3)c3ccc5c(sc6ccccc65)c3-4)cc2)cc1 ; CC1(C)c2cc(N(c3ccc(-c4ccccc4)cc3)c3ccc(-c4cccc5c4sc4ccccc45)cc3)ccc2-c2c1ccc1c2oc2ccccc21 ; c1ccc(-c2ccc(N(c3ccc(-c4cccc5c4sc4ccccc45)cc3)c3ccc4c(c3)C(c3ccccc3)(c3ccccc3)c3ccc5c(oc6ccccc65)c3-4)cc2)cc1]

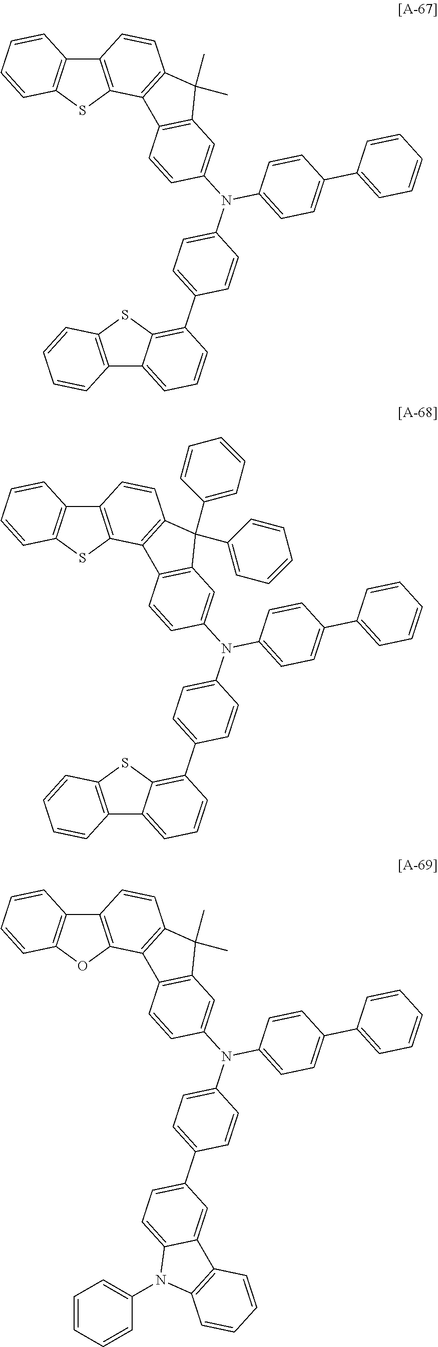 [SMILES: CC1(C)c2cc(N(c3ccc(-c4ccccc4)cc3)c3ccc(-c4cccc5c4sc4ccccc45)cc3)ccc2-c2c1ccc1c2sc2ccccc21 ; c1ccc(-c2ccc(N(c3ccc(-c4cccc5c4sc4ccccc45)cc3)c3ccc4c(c3)C(c3ccccc3)(c3ccccc3)c3ccc5c(sc6ccccc65)c3-4)cc2)cc1 ; CC1(C)c2cc(N(c3ccc(-c4ccccc4)cc3)c3ccc(-c4ccc5c(c4)c4ccccc4n5-c4ccccc4)cc3)ccc2-c2c1ccc1c2oc2ccccc21]

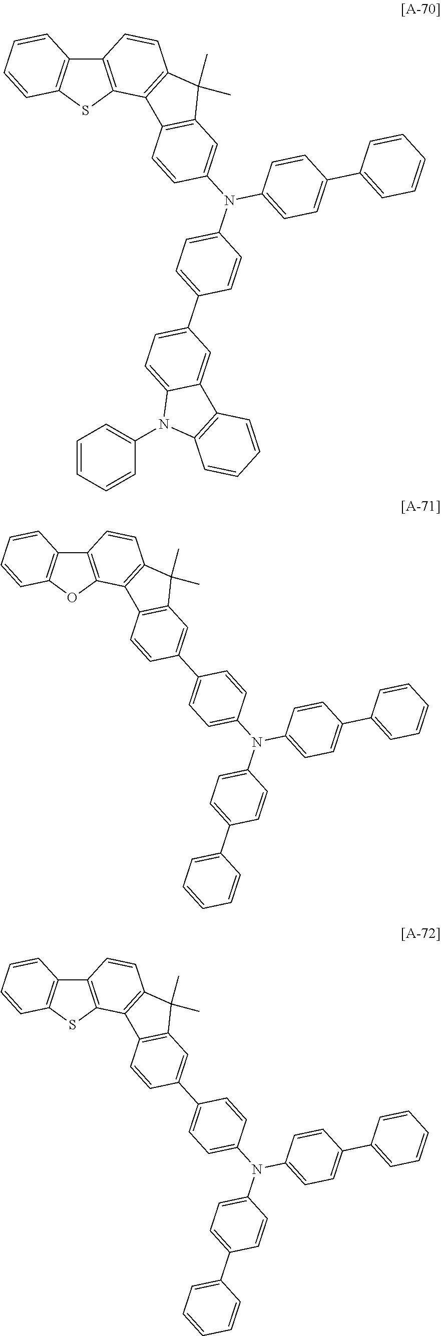

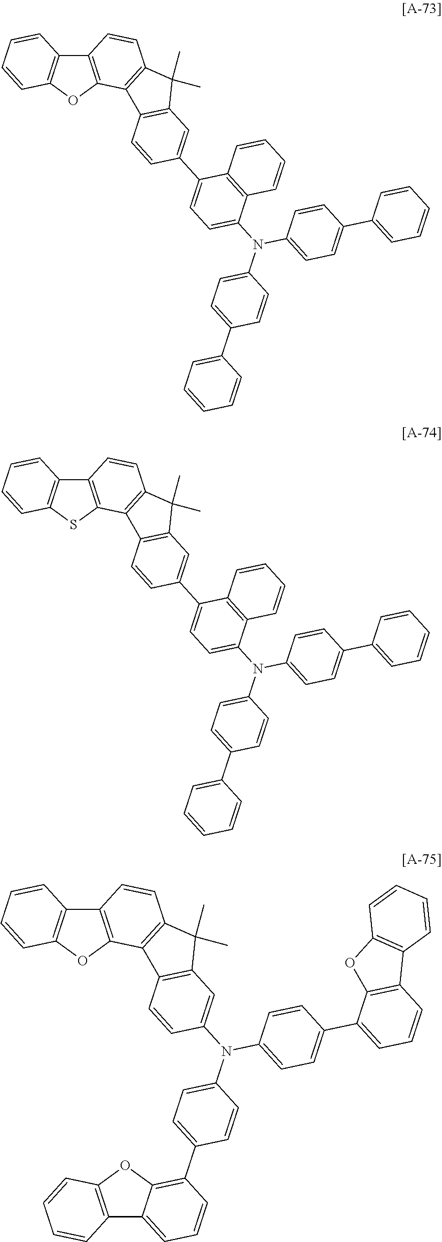 [SMILES: CC1(C)c2cc(-c3ccc(N(c4ccc(-c5ccccc5)cc4)c4ccc(-c5ccccc5)cc4)c4ccccc34)ccc2-c2c1ccc1c2oc2ccccc21 ; CC1(C)c2cc(-c3ccc(N(c4ccc(-c5ccccc5)cc4)c4ccc(-c5ccccc5)cc4)c4ccccc34)ccc2-c2c1ccc1c2sc2ccccc21 ; CC1(C)c2cc(N(c3ccc(-c4cccc5c4oc4ccccc45)cc3)c3ccc(-c4cccc5c4oc4ccccc45)cc3)ccc2-c2c1ccc1c2oc2ccccc21]

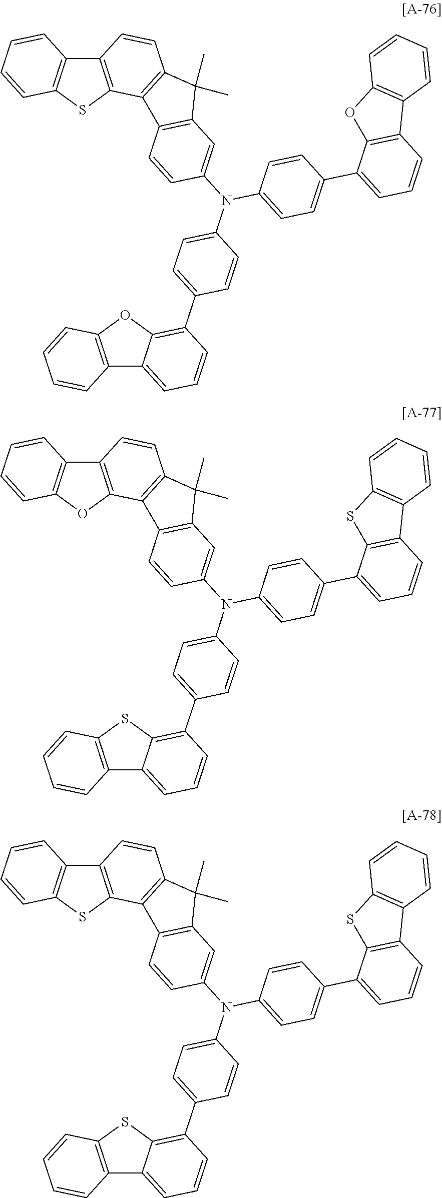 [SMILES: CC1(C)c2cc(N(c3ccc(-c4cccc5c4oc4ccccc45)cc3)c3ccc(-c4cccc5c4oc4ccccc45)cc3)ccc2-c2c1ccc1c2sc2ccccc21 ; CC1(C)c2cc(N(c3ccc(-c4cccc5c4sc4ccccc45)cc3)c3ccc(-c4cccc5c4sc4ccccc45)cc3)ccc2-c2c1ccc1c2oc2ccccc21 ; CC1(C)c2cc(N(c3ccc(-c4cccc5c4sc4ccccc45)cc3)c3ccc(-c4cccc5c4sc4ccccc45)cc3)ccc2-c2c1ccc1c2sc2ccccc21]

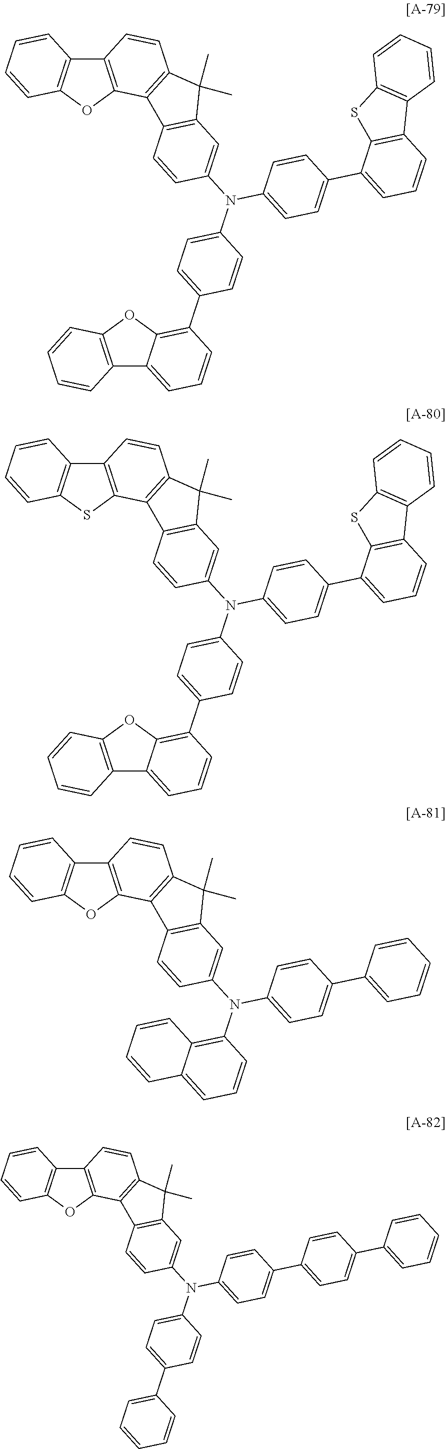 [SMILES: CC1(C)c2cc(N(c3ccc(-c4cccc5c4oc4ccccc45)cc3)c3ccc(-c4cccc5c4sc4ccccc45)cc3)ccc2-c2c1ccc1c2oc2ccccc21 ; CC1(C)c2cc(N(c3ccc(-c4cccc5c4oc4ccccc45)cc3)c3ccc(-c4cccc5c4sc4ccccc45)cc3)ccc2-c2c1ccc1c2sc2ccccc21 ; CC1(C)c2cc(N(c3ccc(-c4ccccc4)cc3)c3cccc4ccccc34)ccc2-c2c1ccc1c2oc2ccccc21 ; CC1(C)c2cc(N(c3ccc(-c4ccccc4)cc3)c3ccc(-c4ccc(-c5ccccc5)cc4)cc3)ccc2-c2c1ccc1c2oc2ccccc21]

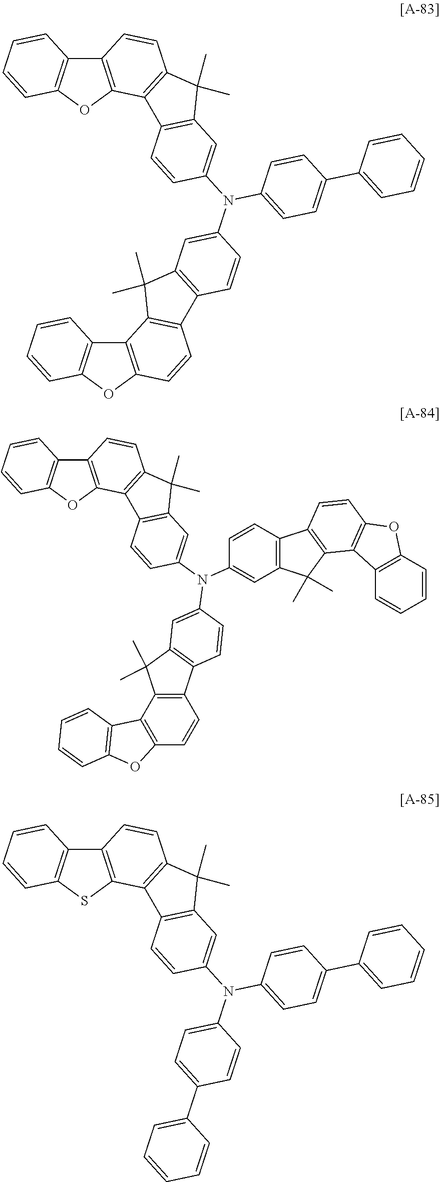

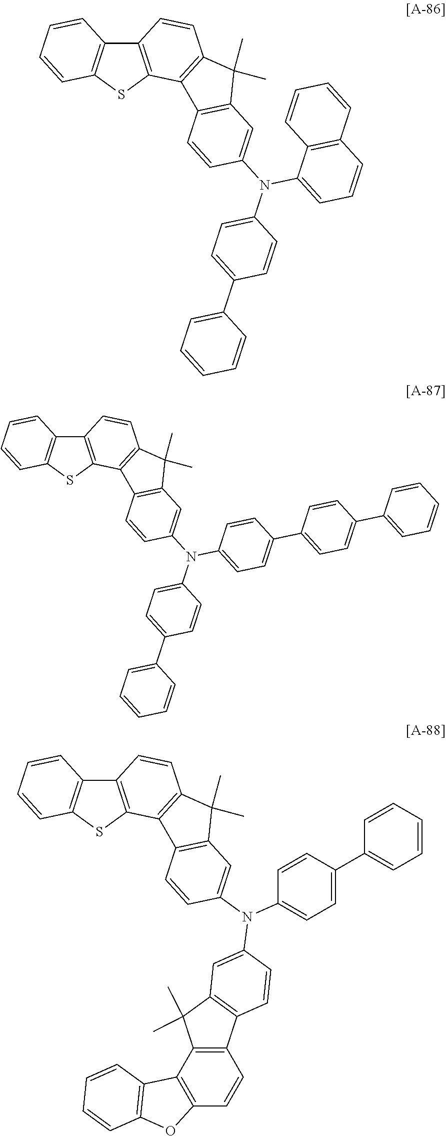

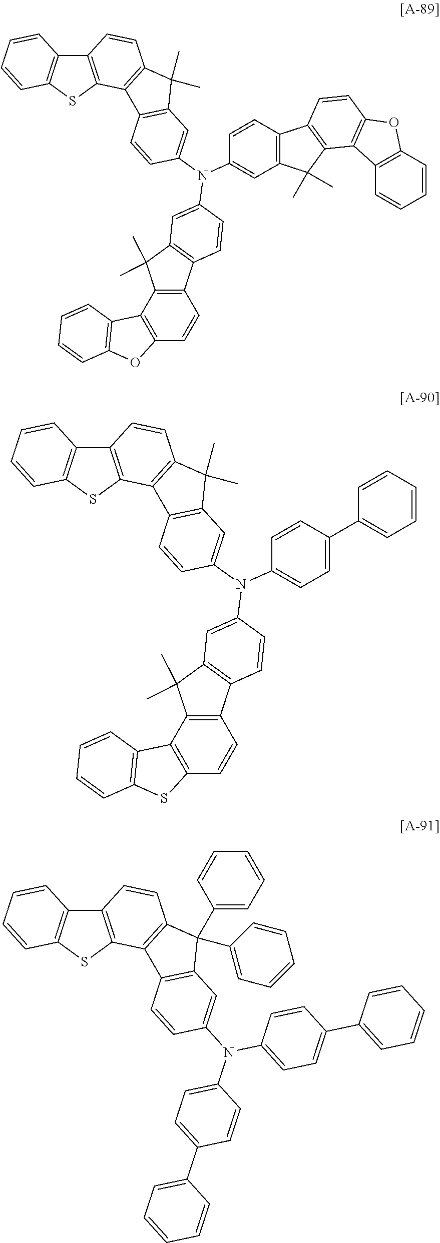 [SMILES: CC1(C)c2cc(N(c3ccc4c(c3)C(C)(C)c3c-4ccc4oc5ccccc5c34)c3ccc4c(c3)C(C)(C)c3c-4ccc4oc5ccccc5c34)ccc2-c2c1ccc1c2sc2ccccc21 ; CC1(C)c2cc(N(c3ccc(-c4ccccc4)cc3)c3ccc4c(c3)C(C)(C)c3c-4ccc4sc5ccccc5c34)ccc2-c2c1ccc1c2sc2ccccc21 ; c1ccc(-c2ccc(N(c3ccc(-c4ccccc4)cc3)c3ccc4c(c3)C(c3ccccc3)(c3ccccc3)c3ccc5c(sc6ccccc65)c3-4)cc2)cc1]

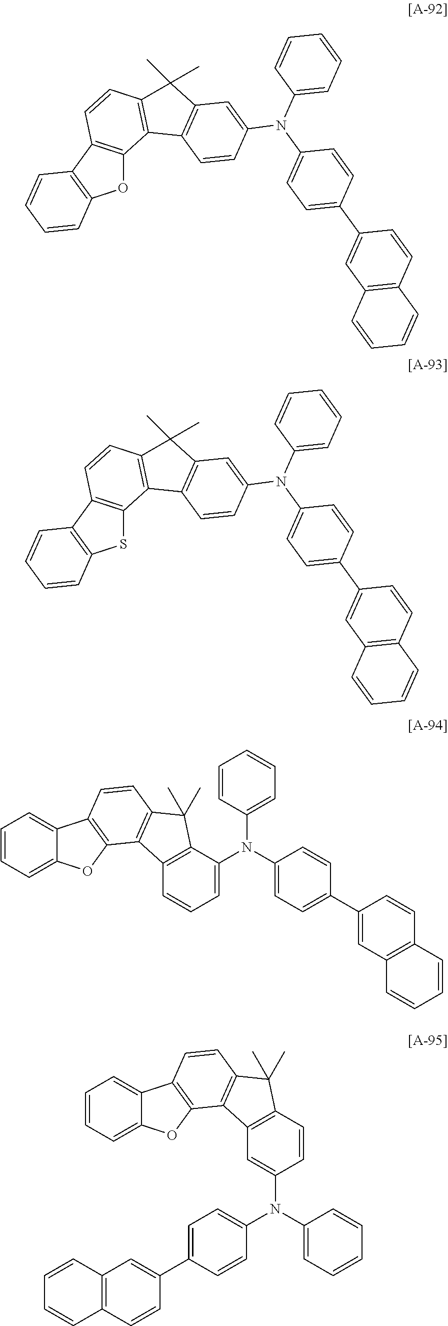 [SMILES: CC1(C)c2cc(N(c3ccccc3)c3ccc(-c4ccc5ccccc5c4)cc3)ccc2-c2c1ccc1c2oc2ccccc21 ; CC1(C)c2cc(N(c3ccccc3)c3ccc(-c4ccc5ccccc5c4)cc3)ccc2-c2c1ccc1c2sc2ccccc21 ; CC1(C)c2ccc3c(oc4ccccc43)c2-c2cccc(N(c3ccccc3)c3ccc(-c4ccc5ccccc5c4)cc3)c21 ; CC1(C)c2ccc(N(c3ccccc3)c3ccc(-c4ccc5ccccc5c4)cc3)cc2-c2c1ccc1c2oc2ccccc21]

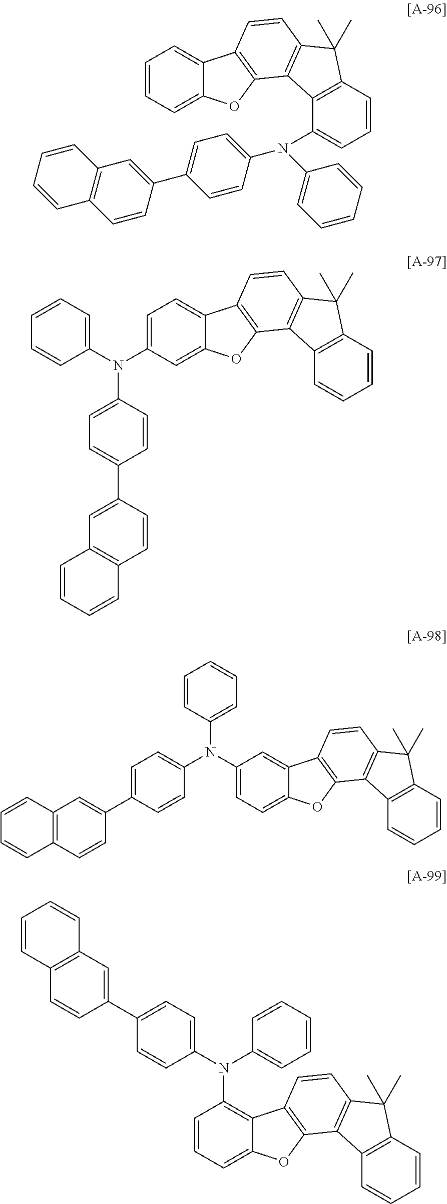

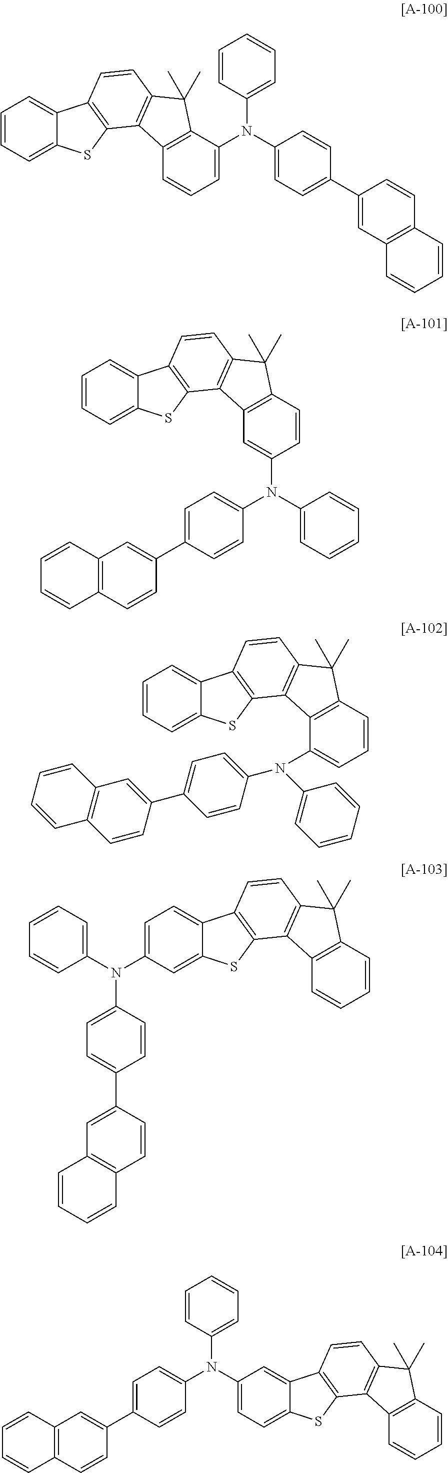

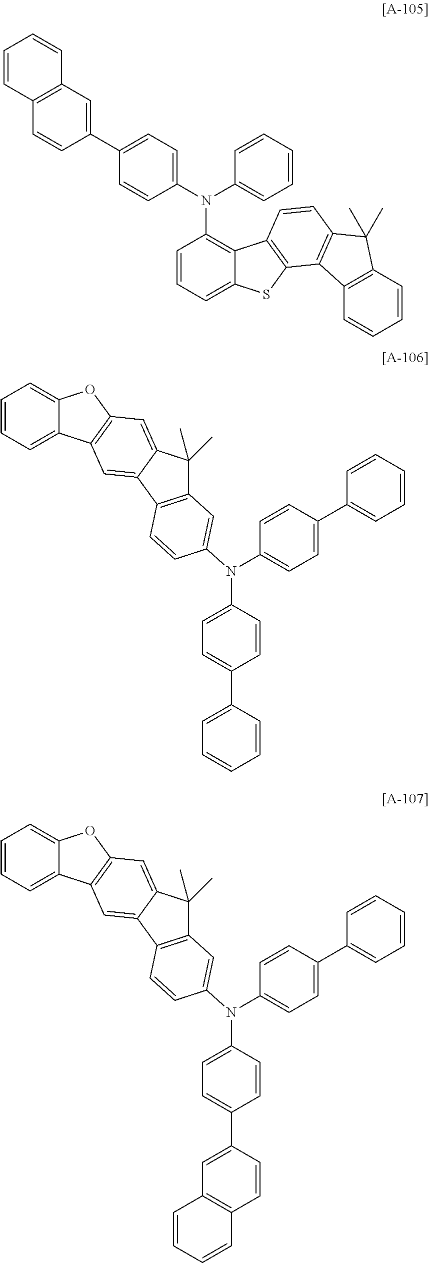 [SMILES: CC1(C)c2ccccc2-c2c1ccc1c2sc2cccc(N(c3ccccc3)c3ccc(-c4ccc5ccccc5c4)cc3)c21 ; CC1(C)c2cc(N(c3ccc(-c4ccccc4)cc3)c3ccc(-c4ccccc4)cc3)ccc2-c2cc3c(cc21)oc1ccccc13 ; CC1(C)c2cc(N(c3ccc(-c4ccccc4)cc3)c3ccc(-c4ccc5ccccc5c4)cc3)ccc2-c2cc3c(cc21)oc1ccccc13]

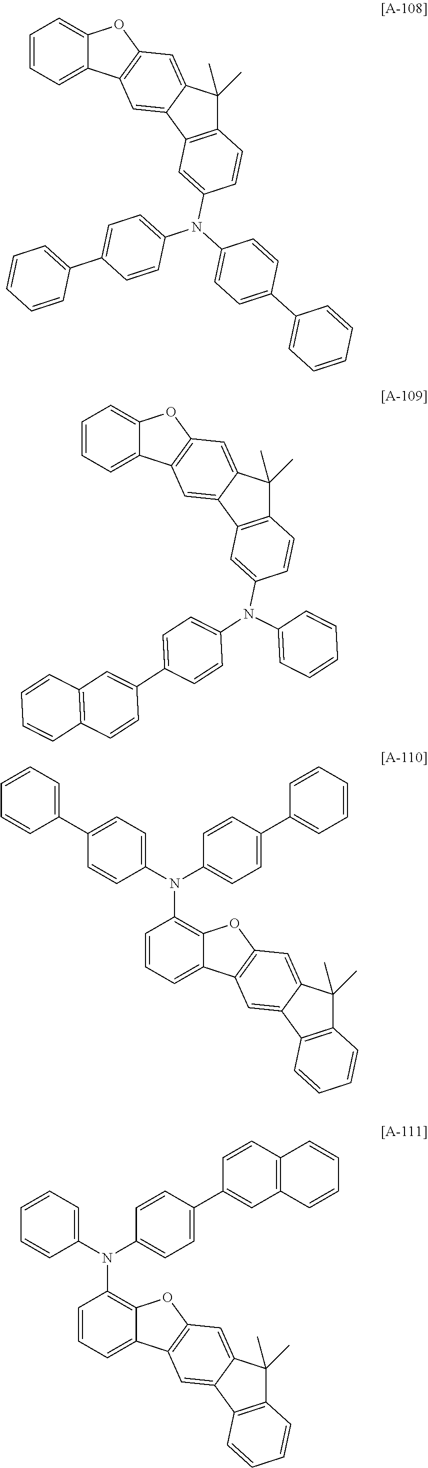

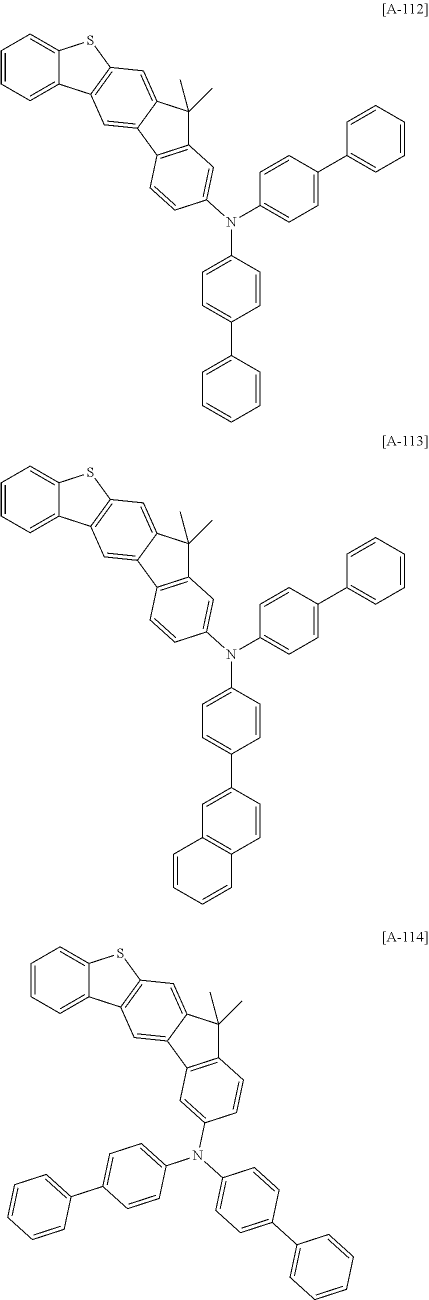 [SMILES: CC1(C)c2cc(N(c3ccc(-c4ccccc4)cc3)c3ccc(-c4ccccc4)cc3)ccc2-c2cc3c(cc21)sc1ccccc13 ; CC1(C)c2cc(N(c3ccc(-c4ccccc4)cc3)c3ccc(-c4ccc5ccccc5c4)cc3)ccc2-c2cc3c(cc21)sc1ccccc13 ; CC1(C)c2ccc(N(c3ccc(-c4ccccc4)cc3)c3ccc(-c4ccccc4)cc3)cc2-c2cc3c(cc21)sc1ccccc13]

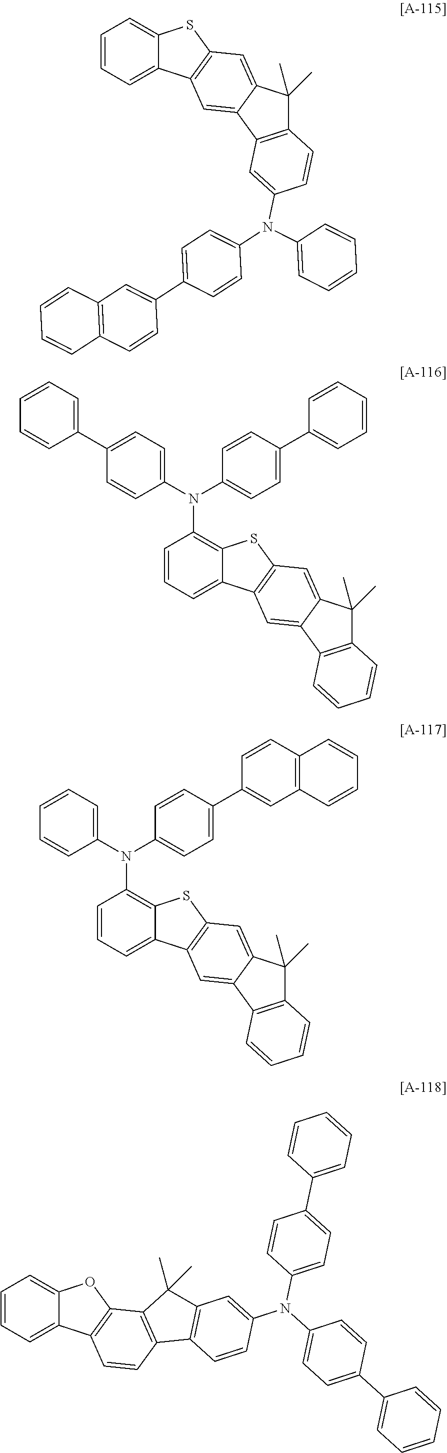

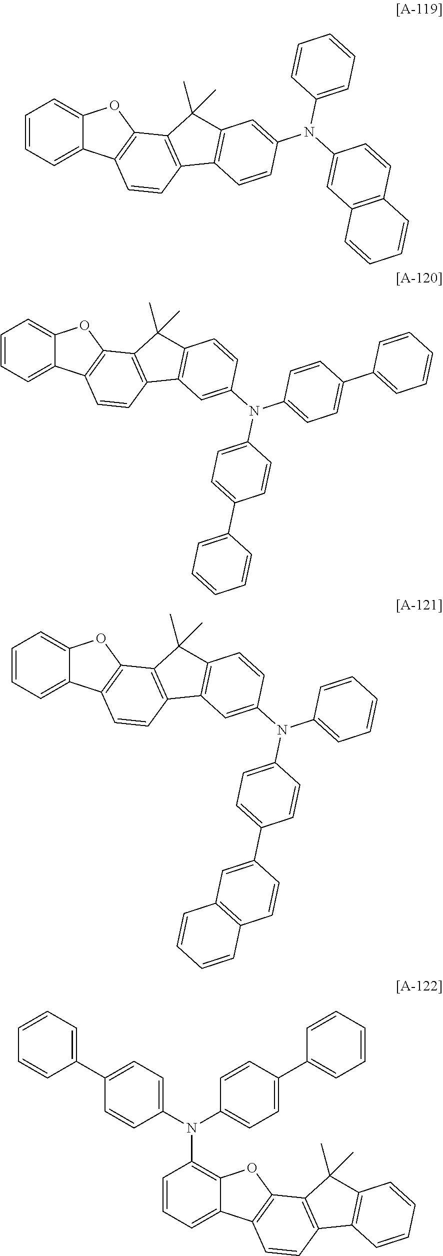

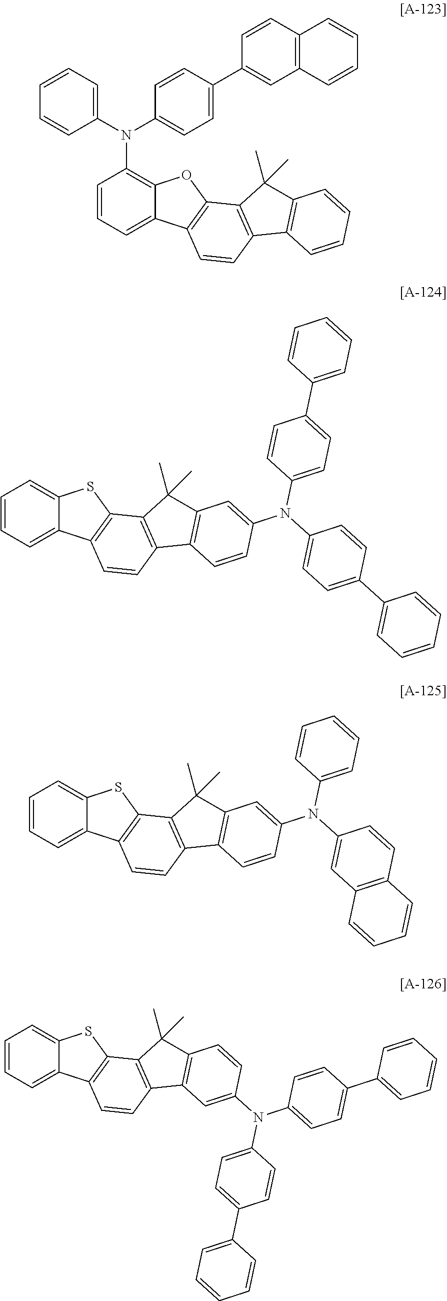 [SMILES: CC1(C)c2ccccc2-c2ccc3c(oc4c(N(c5ccccc5)c5ccc(-c6ccc7ccccc7c6)cc5)cccc43)c21 ; CC1(C)c2cc(N(c3ccc(-c4ccccc4)cc3)c3ccc(-c4ccccc4)cc3)ccc2-c2ccc3c(sc4ccccc43)c21 ; CC1(C)c2cc(N(c3ccccc3)c3ccc4ccccc4c3)ccc2-c2ccc3c(sc4ccccc43)c21 ; CC1(C)c2ccc(N(c3ccc(-c4ccccc4)cc3)c3ccc(-c4ccccc4)cc3)cc2-c2ccc3c(sc4ccccc43)c21]

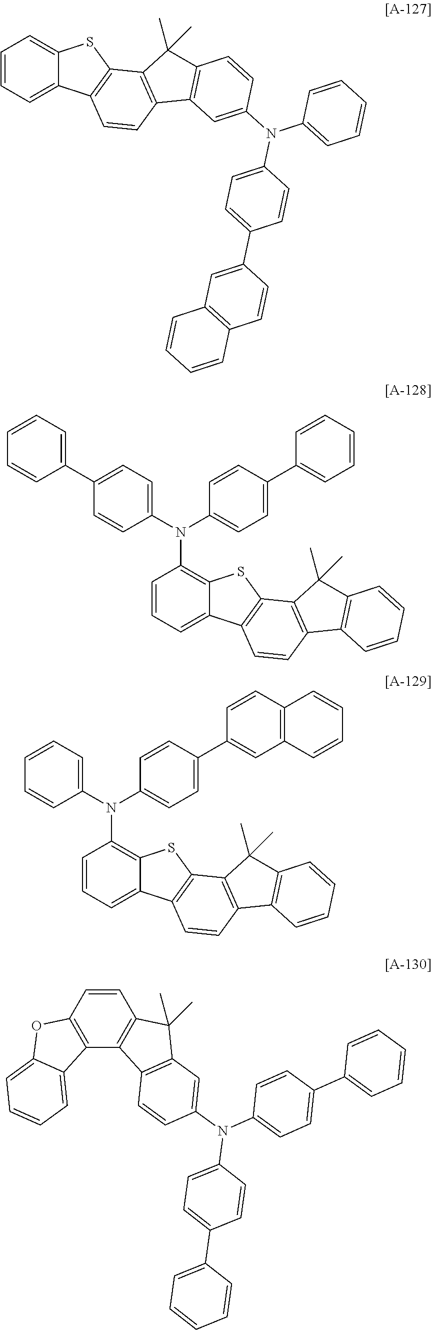

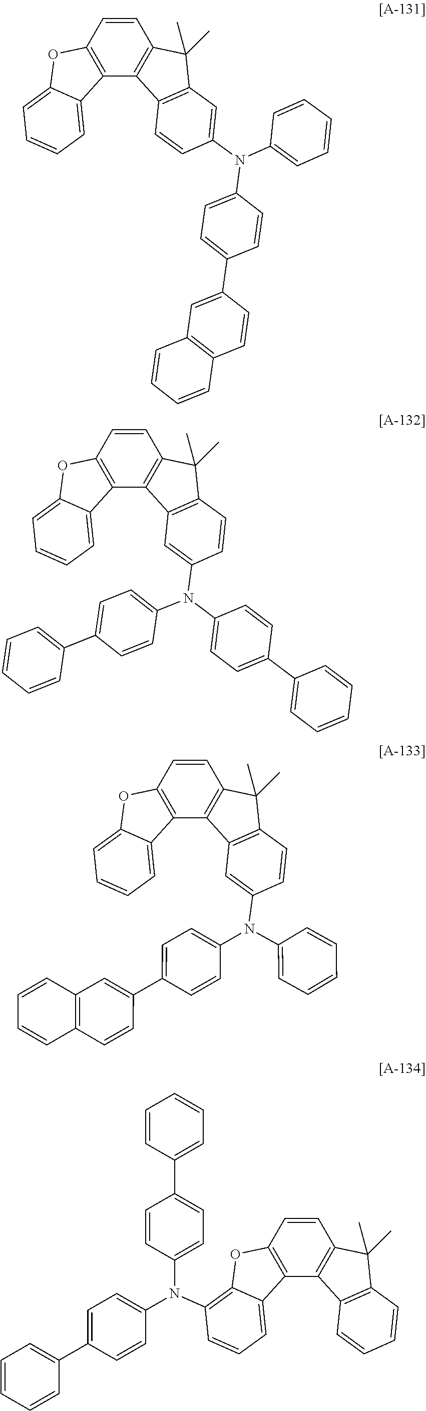

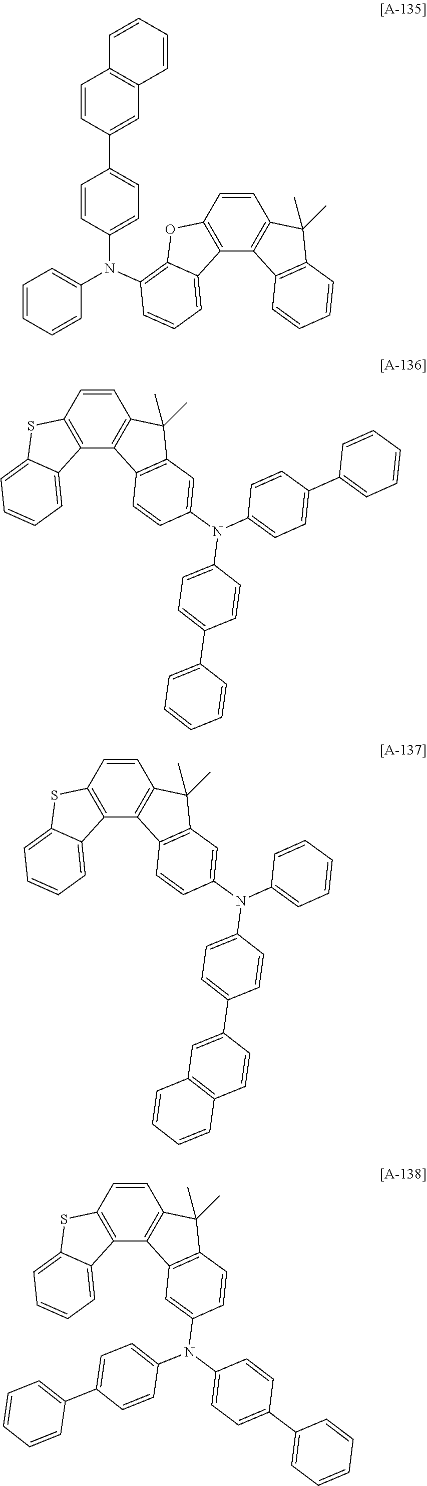

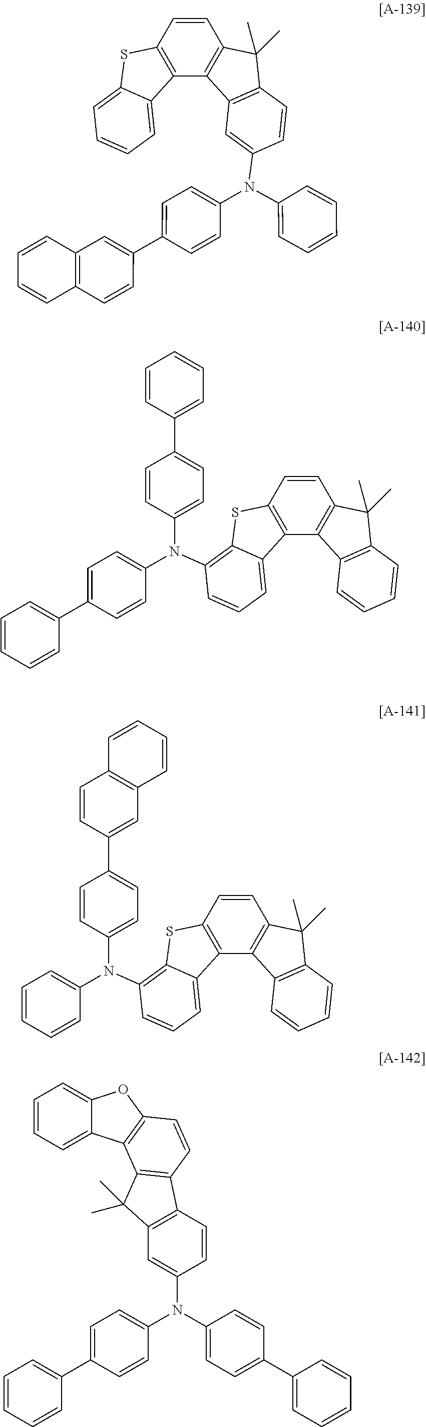 [SMILES: CC1(C)c2ccc(N(c3ccccc3)c3ccc(-c4ccc5ccccc5c4)cc3)cc2-c2c1ccc1sc3ccccc3c21 ; CC1(C)c2ccccc2-c2c1ccc1sc3c(N(c4ccc(-c5ccccc5)cc4)c4ccc(-c5ccccc5)cc4)cccc3c21 ; CC1(C)c2ccccc2-c2c1ccc1sc3c(N(c4ccccc4)c4ccc(-c5ccc6ccccc6c5)cc4)cccc3c21 ; CC1(C)c2cc(N(c3ccc(-c4ccccc4)cc3)c3ccc(-c4ccccc4)cc3)ccc2-c2ccc3oc4ccccc4c3c21]

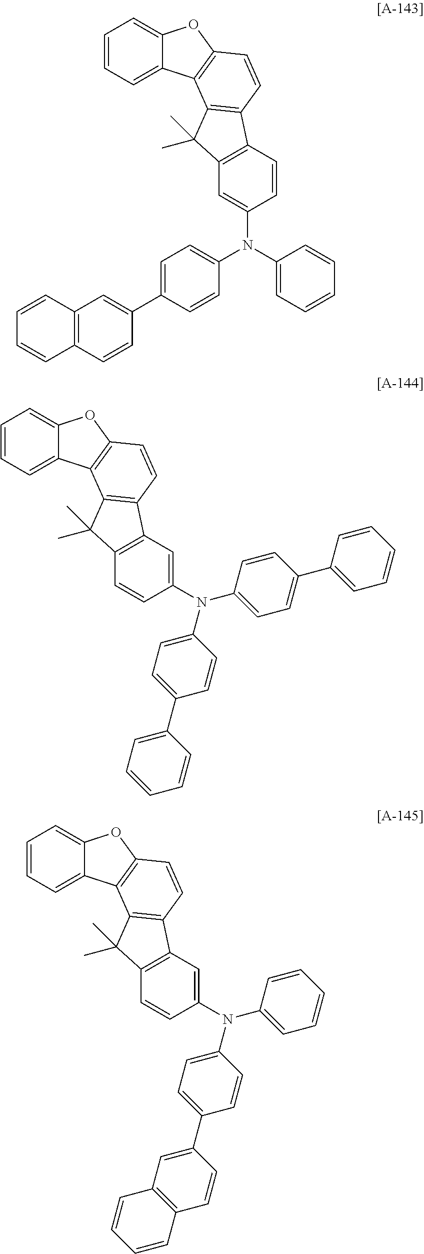 [SMILES: CC1(C)c2cc(N(c3ccccc3)c3ccc(-c4ccc5ccccc5c4)cc3)ccc2-c2ccc3oc4ccccc4c3c21 ; CC1(C)c2ccc(N(c3ccc(-c4ccccc4)cc3)c3ccc(-c4ccccc4)cc3)cc2-c2ccc3oc4ccccc4c3c21 ; CC1(C)c2ccc(N(c3ccccc3)c3ccc(-c4ccc5ccccc5c4)cc3)cc2-c2ccc3oc4ccccc4c3c21]

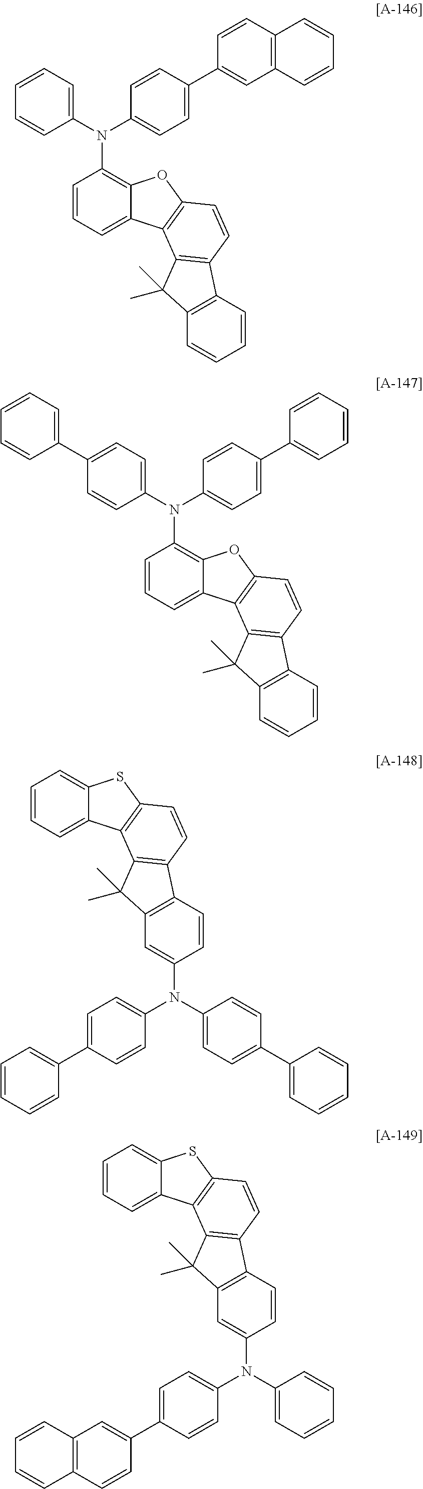 [SMILES: CC1(C)c2ccccc2-c2ccc3oc4c(N(c5ccccc5)c5ccc(-c6ccc7ccccc7c6)cc5)cccc4c3c21 ; CC1(C)c2ccccc2-c2ccc3oc4c(N(c5ccc(-c6ccccc6)cc5)c5ccc(-c6ccccc6)cc5)cccc4c3c21 ; CC1(C)c2cc(N(c3ccc(-c4ccccc4)cc3)c3ccc(-c4ccccc4)cc3)ccc2-c2ccc3sc4ccccc4c3c21 ; CC1(C)c2cc(N(c3ccccc3)c3ccc(-c4ccc5ccccc5c4)cc3)ccc2-c2ccc3sc4ccccc4c3c21]

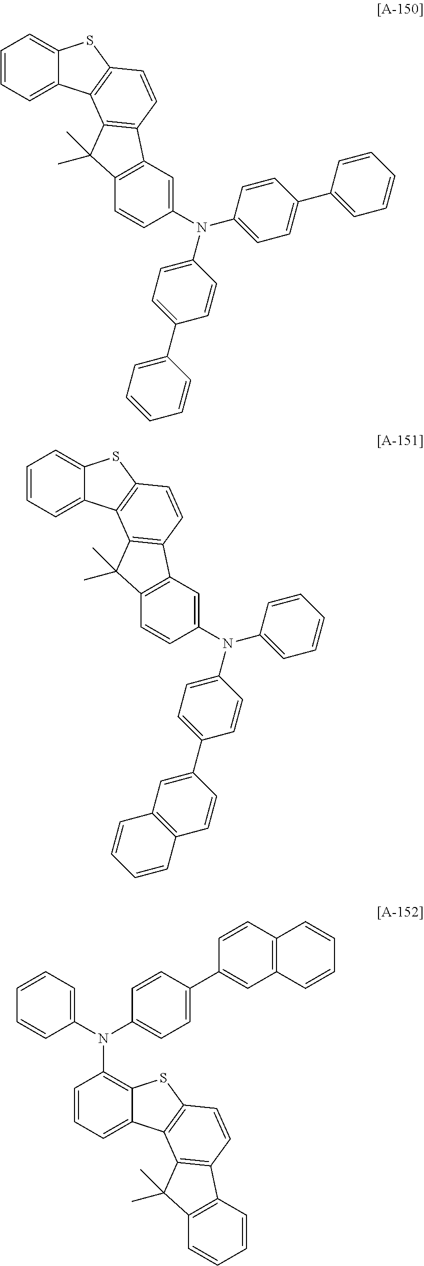 [SMILES: CC1(C)c2ccc(N(c3ccc(-c4ccccc4)cc3)c3ccc(-c4ccccc4)cc3)cc2-c2ccc3sc4ccccc4c3c21 ; CC1(C)c2ccc(N(c3ccccc3)c3ccc(-c4ccc5ccccc5c4)cc3)cc2-c2ccc3sc4ccccc4c3c21 ; CC1(C)c2ccccc2-c2ccc3sc4c(N(c5ccccc5)c5ccc(-c6ccc7ccccc7c6)cc5)cccc4c3c21]

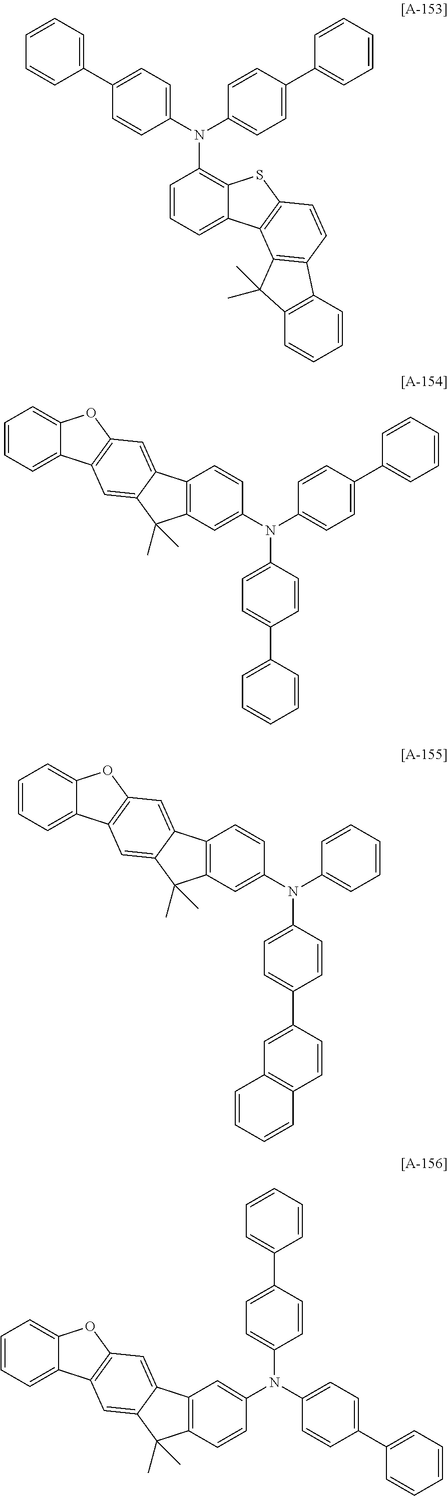 [SMILES: CC1(C)c2ccccc2-c2ccc3sc4c(N(c5ccc(-c6ccccc6)cc5)c5ccc(-c6ccccc6)cc5)cccc4c3c21 ; CC1(C)c2cc(N(c3ccc(-c4ccccc4)cc3)c3ccc(-c4ccccc4)cc3)ccc2-c2cc3oc4ccccc4c3cc21 ; CC1(C)c2cc(N(c3ccccc3)c3ccc(-c4ccc5ccccc5c4)cc3)ccc2-c2cc3oc4ccccc4c3cc21 ; CC1(C)c2ccc(N(c3ccc(-c4ccccc4)cc3)c3ccc(-c4ccccc4)cc3)cc2-c2cc3oc4ccccc4c3cc21]

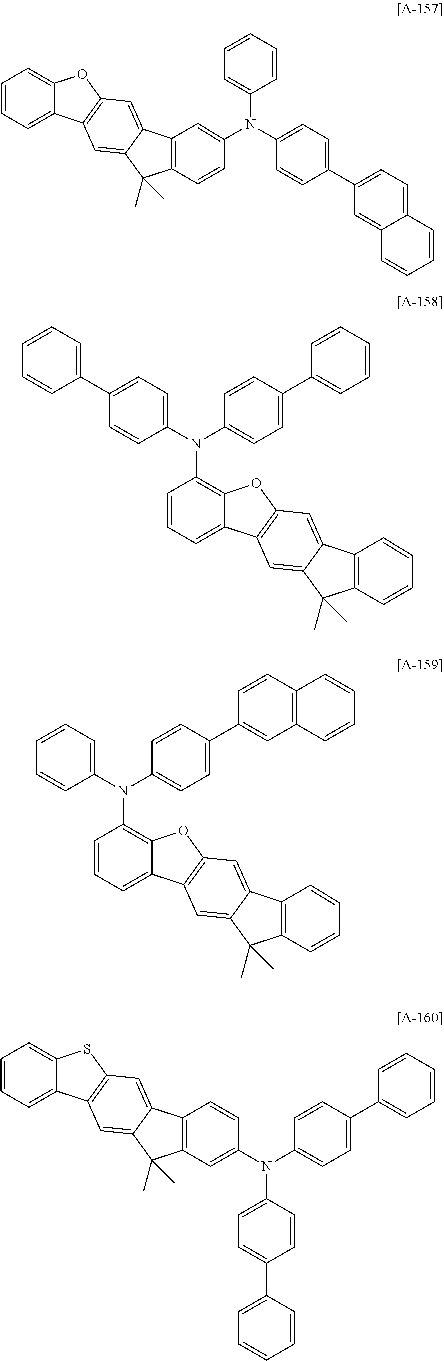 [SMILES: CC1(C)c2ccc(N(c3ccccc3)c3ccc(-c4ccc5ccccc5c4)cc3)cc2-c2cc3oc4ccccc4c3cc21 ; CC1(C)c2ccccc2-c2cc3oc4c(N(c5ccc(-c6ccccc6)cc5)c5ccc(-c6ccccc6)cc5)cccc4c3cc21 ; CC1(C)c2ccccc2-c2cc3oc4c(N(c5ccccc5)c5ccc(-c6ccc7ccccc7c6)cc5)cccc4c3cc21 ; CC1(C)c2cc(N(c3ccc(-c4ccccc4)cc3)c3ccc(-c4ccccc4)cc3)ccc2-c2cc3sc4ccccc4c3cc21]

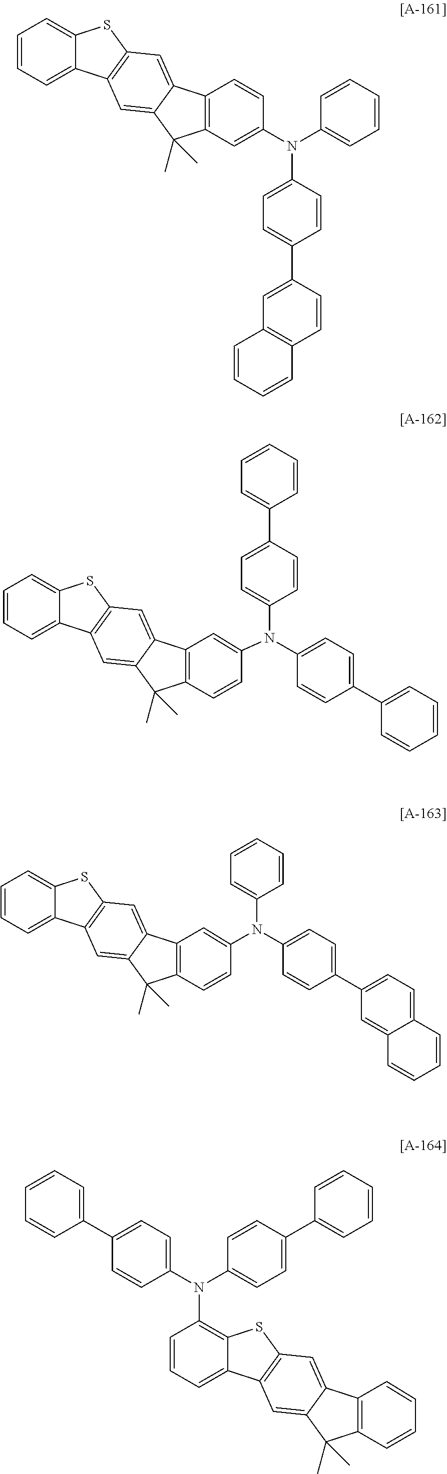 [SMILES: CC1(C)c2cc(N(c3ccccc3)c3ccc(-c4ccc5ccccc5c4)cc3)ccc2-c2cc3sc4ccccc4c3cc21 ; CC1(C)c2ccc(N(c3ccc(-c4ccccc4)cc3)c3ccc(-c4ccccc4)cc3)cc2-c2cc3sc4ccccc4c3cc21 ; CC1(C)c2ccc(N(c3ccccc3)c3ccc(-c4ccc5ccccc5c4)cc3)cc2-c2cc3sc4ccccc4c3cc21 ; CC1(C)c2ccccc2-c2cc3sc4c(N(c5ccc(-c6ccccc6)cc5)c5ccc(-c6ccccc6)cc5)cccc4c3cc21]

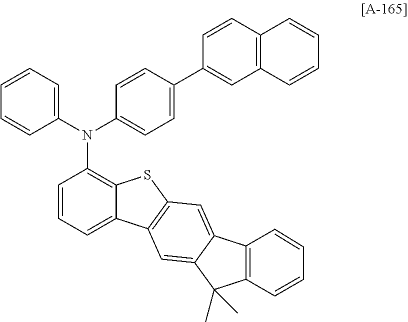 [SMILES: CC1(C)c2ccccc2-c2cc3sc4c(N(c5ccccc5)c5ccc(-c6ccc7ccccc7c6)cc5)cccc4c3cc21]

The second compound may be represented by Chemical Formula 3.

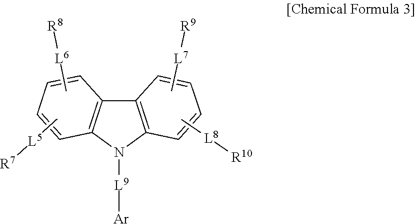

In Chemical Formula 3,

L⁵ to L⁹ may each independently be or include, e.g., a single bond, a substituted or unsubstituted C6 to C20 arylene group, a substituted or unsubstituted C2 to C20 heterocyclic group, or a combination thereof,

Ar may be or may include, e.g., a substituted or unsubstituted C6 to C30 aryl group, a substituted or unsubstituted C2 to C30 heterocyclic group, or a combination thereof,

R⁷ to R¹⁰ may each independently be or include, e.g., hydrogen, deuterium, a substituted or unsubstituted C1 to C30 alkyl group, a substituted or unsubstituted C6 to C30 aryl group, a substituted or unsubstituted C2 to C30 heterocyclic group, a substituted or unsubstituted silyl group, a substituted or unsubstituted amine group, a halogen, a cyano group, or a combination thereof

In an implementation, R⁷ to R¹⁰ may be separately present or adjacent ones thereof may be linked with each other to form a substituted or unsubstituted aliphatic monocyclic ring or a substituted or unsubstituted aliphatic polycyclic ring, a substituted or unsubstituted aromatic monocyclic ring or a substituted or unsubstituted aromatic polycyclic ring, or a substituted or unsubstituted heteroaromatic monocyclic ring or a substituted or unsubstituted heteroaromatic polycyclic ring.

In an implementation, at least one of Ar and R⁷ to R¹⁰ may be, e.g., a group represented by Chemical Formula b.

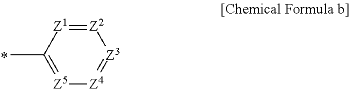

In Chemical Formula b, Z¹ to Z⁵ may each independently be, e.g., N or C-L^{d}-R^{d}. In an implementation, at least two of Z¹ to Z⁵ are N.

In an implementation, each L^{d} may independently be or include, e.g., a single bond, a substituted or unsubstituted C6 to C20 arylene group, a substituted or unsubstituted C2 to C20 heterocyclic group, or a combination thereof.

In an implementation, each R^{d} may independently be or include, e.g., hydrogen, deuterium, a substituted or unsubstituted C1 to C30 alkyl group, a substituted or unsubstituted C6 to C30 aryl group, a substituted or unsubstituted C2 to C30 heterocyclic group, a substituted or unsubstituted silyl group, a substituted or unsubstituted amine group, a halogen, a cyano group, or a combination thereof.

In an implementation, each R^{d} may be separately present or adjacent ones thereof are linked with each other to form a substituted or unsubstituted aliphatic monocyclic ring or a substituted or unsubstituted aliphatic polycyclic ring, a substituted or unsubstituted aromatic monocyclic ring or a substituted or unsubstituted aromatic polycyclic ring, or a substituted or unsubstituted heteroaromatic monocyclic ring or a substituted or unsubstituted heteroaromatic polycyclic ring.

* is a linking point with one of L⁵ to L⁹ (e.g., L⁵, L⁶, L⁷, L⁸, or L⁹).

The second compound may be a compound having characteristics of accepting both holes and electrons, e.g., bipolar characteristics. For example, the second compound may have a structure in which the carbazole core represented by Chemical Formula 3 is substituted with a ring including at least two nitrogen (e.g., a pyrimidine or triazine ring), and a glass transition temperature relative to a molecular weight may be improved, so that the heat resistance may be secured.

In addition, the second compound may have fast and stable electron transport characteristics, and it may be included together with the aforementioned first compound (having fast and stable hole transport characteristics) to balance holes and electrons in a device and to lower a driving voltage of the organic optoelectronic device including same.

In an implementation, L⁵ to L⁹ may each independently be, e.g., a single bond, a substituted or unsubstituted C6 to C20 arylene group, or a substituted or unsubstituted C2 to C20 heterocyclic group.

In an implementation, L⁵ to L⁹ may each independently be, e.g., a single bond, a substituted or unsubstituted phenylene group, a substituted or unsubstituted biphenylene group, a substituted or unsubstituted terphenylene group, a substituted or unsubstituted naphthylene group, a substituted or unsubstituted dibenzofuranylene group, a substituted or unsubstituted dibenzothiophenylene group, a substituted or unsubstituted fused dibenzofuranylene group, a substituted or unsubstituted fused dibenzothiophenylene group, or a combination thereof.

In an implementation, L⁵ to L⁹ may each independently be, e.g., a single bond, a substituted or unsubstituted m-phenylene group, a substituted or unsubstituted p-phenylene group, a substituted or unsubstituted biphenylene group, or a substituted or unsubstituted terphenylene group.

In an implementation, Ar may be, e.g., a substituted or unsubstituted phenyl group, a substituted or unsubstituted biphenyl group, a substituted or unsubstituted terphenyl group, a substituted or unsubstituted naphthyl group, a substituted or unsubstituted anthracenyl group, a substituted or unsubstituted phenanthrenyl group, a substituted or unsubstituted triphenylene group, a substituted or unsubstituted fluorenyl group, a group represented by Chemical Formula b, or a combination thereof.

In an implementation, Ar may be, e.g., a substituted or unsubstituted phenyl group, a substituted or unsubstituted biphenyl group, a substituted or unsubstituted naphthyl group, a substituted or unsubstituted fluorenyl group, or a group represented by Chemical Formula b.

In an implementation, R⁷ to R¹⁰ may each independently be, e.g., hydrogen, deuterium, a cyano group, a substituted or unsubstituted phenyl group, a substituted or unsubstituted biphenyl group, a substituted or unsubstituted terphenyl group, a substituted or unsubstituted naphthyl group, a substituted or unsubstituted anthracenyl group, a substituted or unsubstituted phenanthrenyl group, a substituted or unsubstituted triphenylene group, a substituted or unsubstituted fluorenyl group, a substituted or unsubstituted dibenzofuranyl group, a substituted or unsubstituted dibenzothiophenyl group, a group represented by Chemical Formula b, or a combination thereof.

In an implementation, R⁷ to R¹⁰ may each independently be, e.g., hydrogen, deuterium, a cyano group, a substituted or unsubstituted phenyl group, a substituted or unsubstituted biphenyl group, a substituted or unsubstituted terphenyl group, a substituted or unsubstituted naphthyl group, a substituted or unsubstituted triphenylene group, a substituted or unsubstituted dibenzofuranyl group, a substituted or unsubstituted dibenzothiophenyl group, or a group represented by Chemical Formula b.

In an implementation, the second compound represented by Chemical Formula 3 may be represented by one of Chemical Formula 3A to Chemical Formula 3R (e.g, according to a bonding or linking position of the group represented by Chemical Formula b).

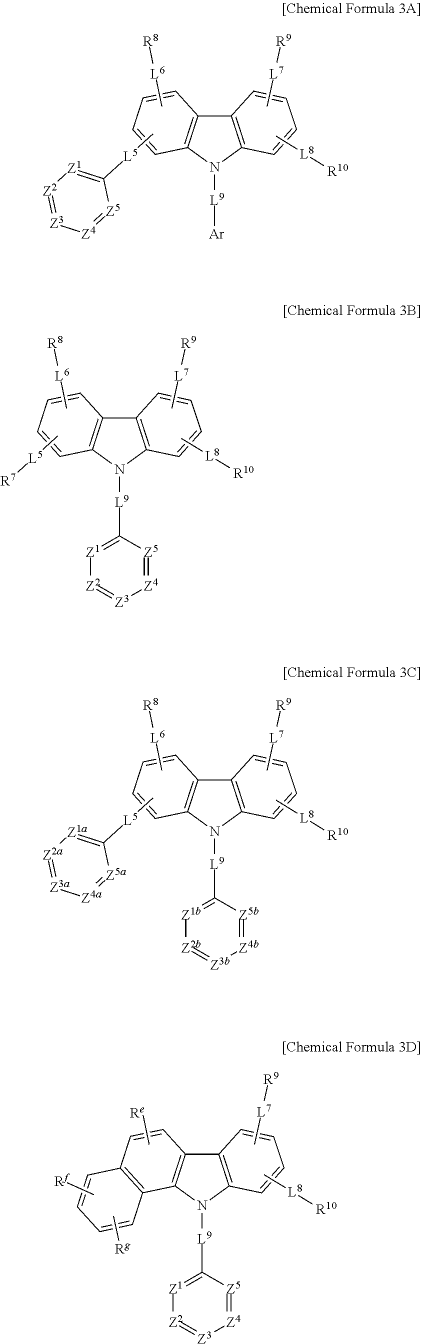

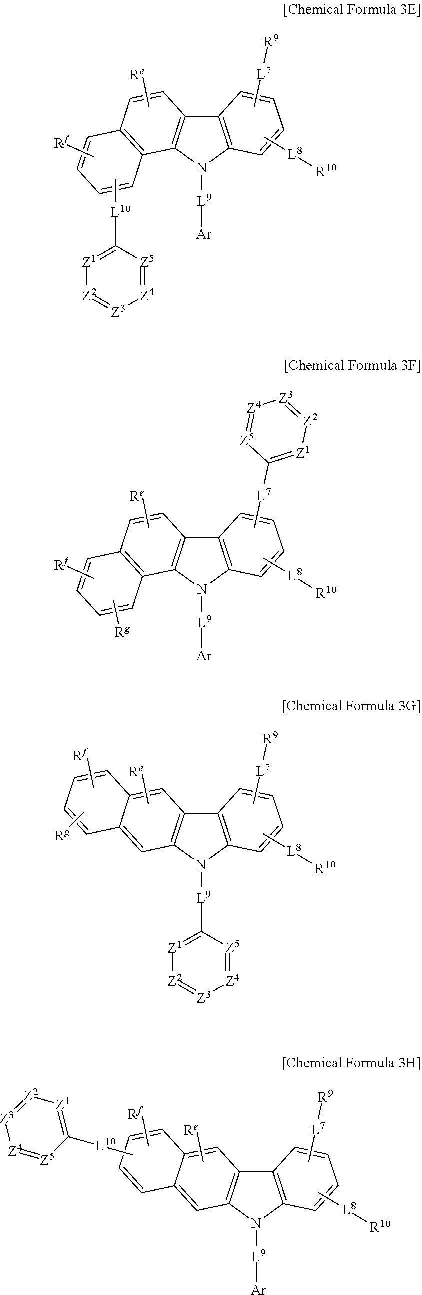

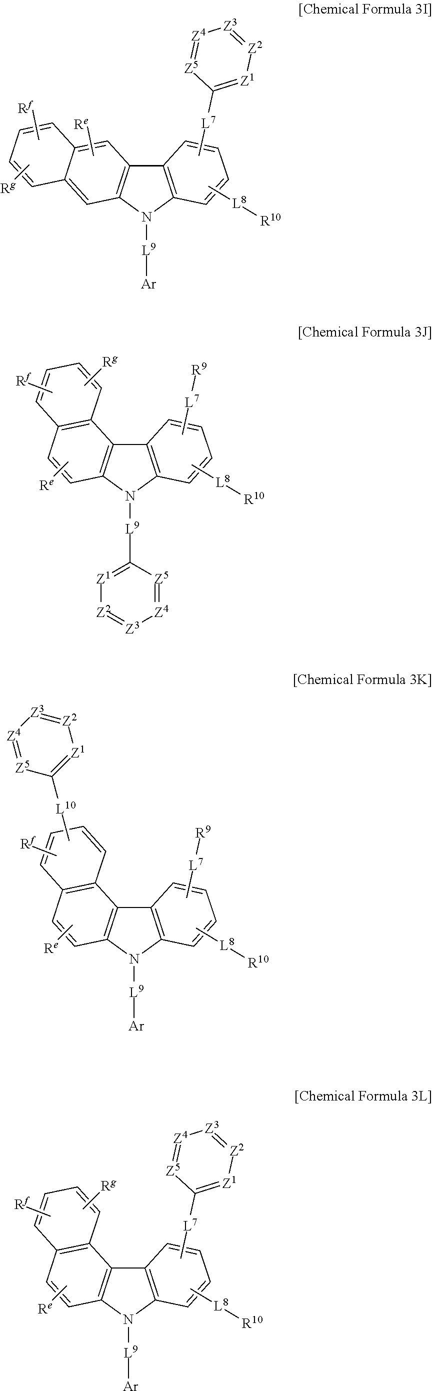

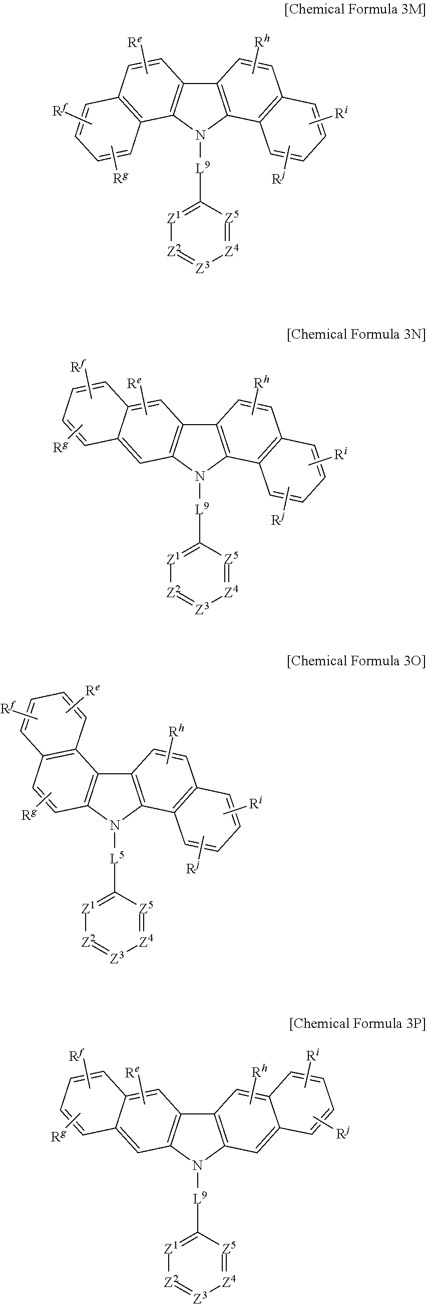

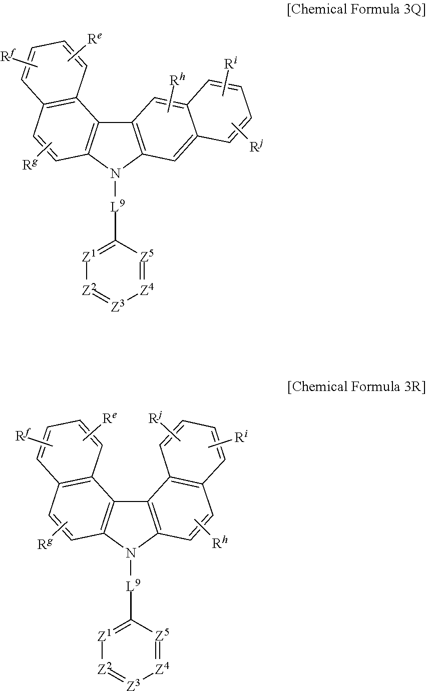

In Chemical Formula 3A to Chemical Formula 3R, L⁵ to L⁹, Ar, R⁷ to R¹⁰, and Z¹ to Z⁵ may be defined the same as those of Chemical Formula 3 and Chemical Formula b.

L¹⁰ may be defined the same as L⁵ to L⁹ of Chemical Formula 3.

R^{e}, R^{f}, R^{g}, R^{h}, Rⁱ, and R^{j} may be defined the same as R⁷ to R¹⁰ of Chemical Formula 3.

Z^{1a} to Z^{5a} and Z^{1b} to Z^{5b} may be defined the same as Z¹ to Z⁵ of Chemical Formula b.

In an implementation, each L^{d} may independently be, e.g., a single bond, a substituted or unsubstituted C6 to C20 arylene group, a substituted or unsubstituted C2 to C20 heterocyclic group, or a combination thereof.

In an implementation, each L^{d} may independently be, e.g., a single bond, a substituted or unsubstituted phenylene group, a substituted or unsubstituted biphenylene group, a substituted or unsubstituted terphenylene group, a substituted or unsubstituted fluorenylene group, a substituted or unsubstituted dibenzofuranylene group, or a substituted or unsubstituted dibenzothiophenylene group.

In an implementation, each L^{d} may independently be, e.g., a single bond, a substituted or unsubstituted m-phenylene group, or a substituted or unsubstituted p-phenylene group.

In an implementation, each R^{d} may independently be, e.g., hydrogen, deuterium, a cyano group, a substituted or unsubstituted C1 to C20 alkyl group, a substituted or unsubstituted C6 to C20 aryl group, a substituted or unsubstituted C2 to C20 heterocyclic group, or a combination thereof.

In an implementation, each R^{d} may independently be, e.g., hydrogen, deuterium, a cyano group, a substituted or unsubstituted phenyl group, a substituted or unsubstituted biphenyl group, a substituted or unsubstituted naphthyl group, a substituted or unsubstituted terphenyl group, a substituted or unsubstituted anthracenyl group, a substituted or unsubstituted phenanthrene group, a substituted or unsubstituted triphenylene group, a substituted or unsubstituted fluorenyl group, a substituted or unsubstituted carbazolyl group, a substituted or unsubstituted dibenzofuranyl group, or a substituted or unsubstituted dibenzothiophenyl group.

In an implementation, each R^{d} may independently be, e.g., hydrogen, deuterium, a cyano group, a substituted or unsubstituted phenyl group, a substituted or unsubstituted m-biphenyl group, a substituted or unsubstituted p-biphenyl group, a substituted or unsubstituted triphenylene group, a substituted or unsubstituted fluorenyl group, a substituted or unsubstituted dibenzofuranyl group, or a substituted or unsubstituted dibenzothiophenyl group.

In an implementation, each R^{d} may be separately present or adjacent ones thereof may be linked with each other to form, e.g., a substituted or unsubstituted aliphatic monocyclic ring or a substituted or unsubstituted aliphatic polycyclic ring, a substituted or unsubstituted aromatic monocyclic ring or a substituted or unsubstituted aromatic polycyclic ring, or a substituted or unsubstituted heteroaromatic monocyclic ring or a substituted or unsubstituted heteroaromatic polycyclic ring.

In an implementation, in the group represented by Chemical Formula b, at least two of Z¹ to Z⁵ may be N and each R^{d} may be separately present.

In an implementation, the group represented by Chemical Formula b may be, e.g., a substituted or unsubstituted pyrimidinyl group or a substituted or unsubstituted triazinyl group.

In an implementation, Z¹ and Z³ may be N, Z², Z⁴, and Z¹ may each independently be C-L^{d}-R^{d}; Z³ and Z⁵ may be N and Z¹, Z², and Z⁴ may each independently be C-L^{d}-R^{d}; or Z² and Z⁴ may be N and Z¹, Z³, and Z⁵ may each independently be C-L^{d}-R^{d}. In this case, L^{d} and R^{d} are the same as described above.

In an implementation, Z¹, Z³, and Z¹ may be N and Z² and Z⁴ may each independently be C-L^{d}-R^{d}. In this case, L^{d} and R^{d} are the same as described above.

In an implementation, in the group represented by Chemical Formula b, at least two of Z¹ to Z⁵ may be N and adjacent R^{d}s may be linked with each other to form a substituted or unsubstituted aromatic monocyclic ring, or a substituted or unsubstituted aromatic monocyclic heterocyclic ring.

In this case, the group represented by Chemical Formula b may be, e.g., a substituted or unsubstituted quinazolinyl group, a substituted or unsubstituted quinoxalinyl group, or a substituted or unsubstituted naphthyridinyl group.

In an implementation, Z³ and Z⁴ may each independently be, e.g., C—R^{d} in which adjacent R^{d}s are linked with each other to form a benzene ring, and two of Z¹, Z², and Z⁵ may be N.

In an implementation, Z³ and Z⁴ may each independently be, e.g., C—R^{d} in which adjacent R^{d}s are linked with each other to form a benzene ring, and each Z¹, Z², and Z⁵ may be N.

In an implementation, the group represented by Chemical Formula b may be represented by, e.g., one of Chemical Formulae b-1 to b-5.

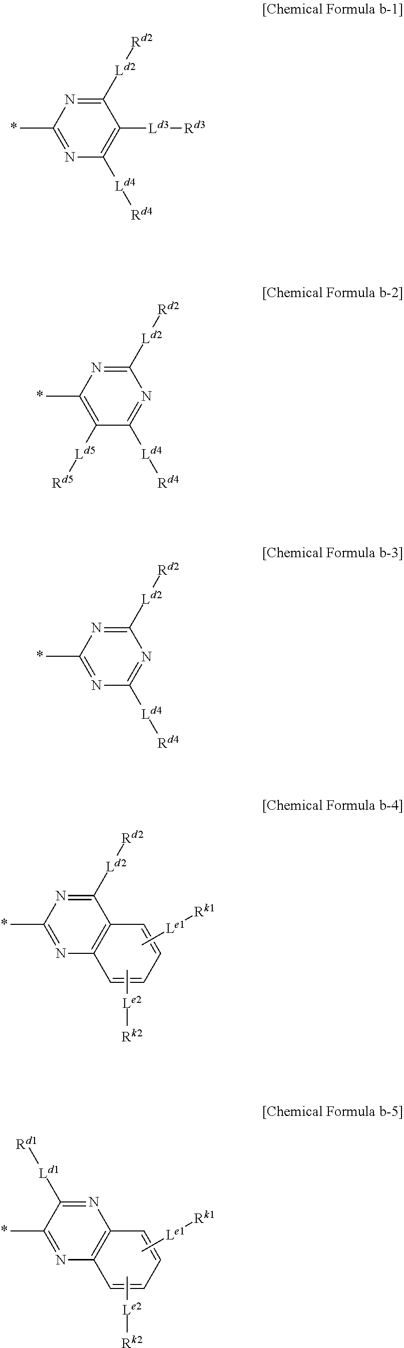

In Chemical Formula b-1 to Chemical Formula b-5, L^{d2} to L^{d5} and L^{e1} and L^{e2} may be defined the same as L^{d}, and R^{d1} to R^{d}, R^{k1}, and R^{k2} are the same as definitions of the aforementioned R^{d} of Chemical Formula b.

In an implementation, the group represented by Chemical Formula b may be, e.g., a substituted or unsubstituted pyrimidinyl group, a substituted or unsubstituted triazinyl group, a substituted or unsubstituted quinoxalinyl group, a substituted or unsubstituted quinazolinyl group, or a substituted or unsubstituted naphthyridinyl group.

In an implementation, the group represented by Chemical Formula b may be, e.g., a group of the following Group I.

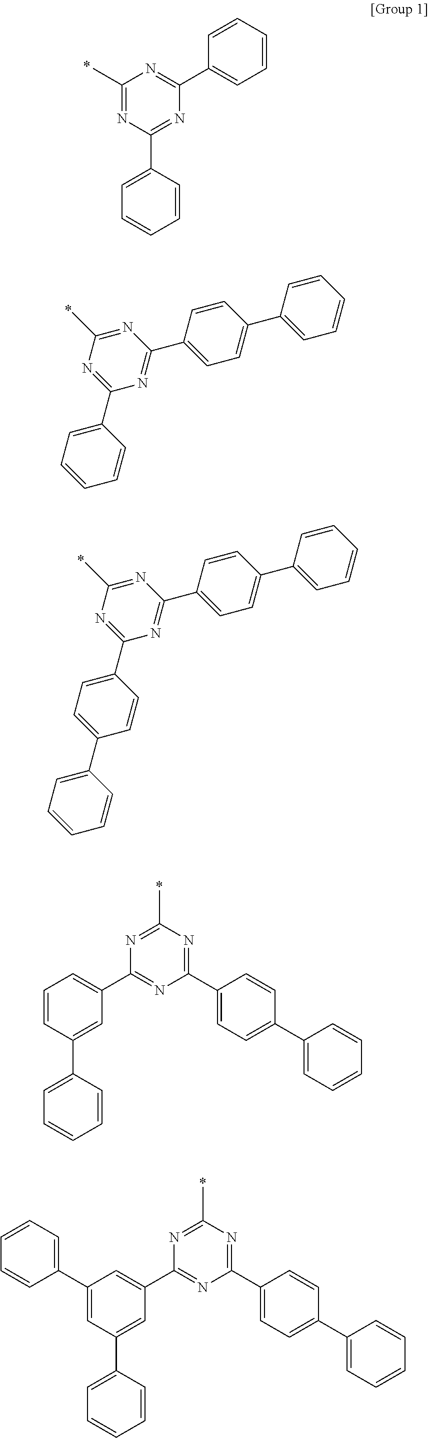 [SMILES: Cc1nc(-c2ccccc2)nc(-c2ccccc2)n1 ; Cc1nc(-c2ccccc2)nc(-c2ccc(-c3ccccc3)cc2)n1 ; Cc1nc(-c2ccc(-c3ccccc3)cc2)nc(-c2ccc(-c3ccccc3)cc2)n1 ; Cc1nc(-c2ccc(-c3ccccc3)cc2)nc(-c2cccc(-c3ccccc3)c2)n1 ; Cc1nc(-c2ccc(-c3ccccc3)cc2)nc(-c2cc(-c3ccccc3)cc(-c3ccccc3)c2)n1]

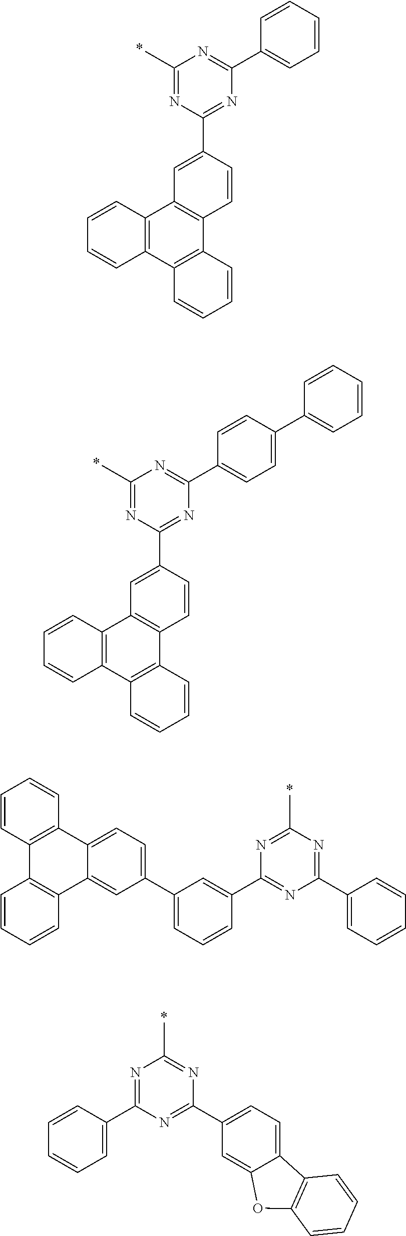 [SMILES: Cc1nc(-c2ccccc2)nc(-c2ccc3c4ccccc4c4ccccc4c3c2)n1 ; Cc1nc(-c2ccc(-c3ccccc3)cc2)nc(-c2ccc3c4ccccc4c4ccccc4c3c2)n1 ; Cc1nc(-c2ccccc2)nc(-c2cccc(-c3ccc4c5ccccc5c5ccccc5c4c3)c2)n1 ; Cc1nc(-c2ccccc2)nc(-c2ccc3c(c2)oc2ccccc23)n1]

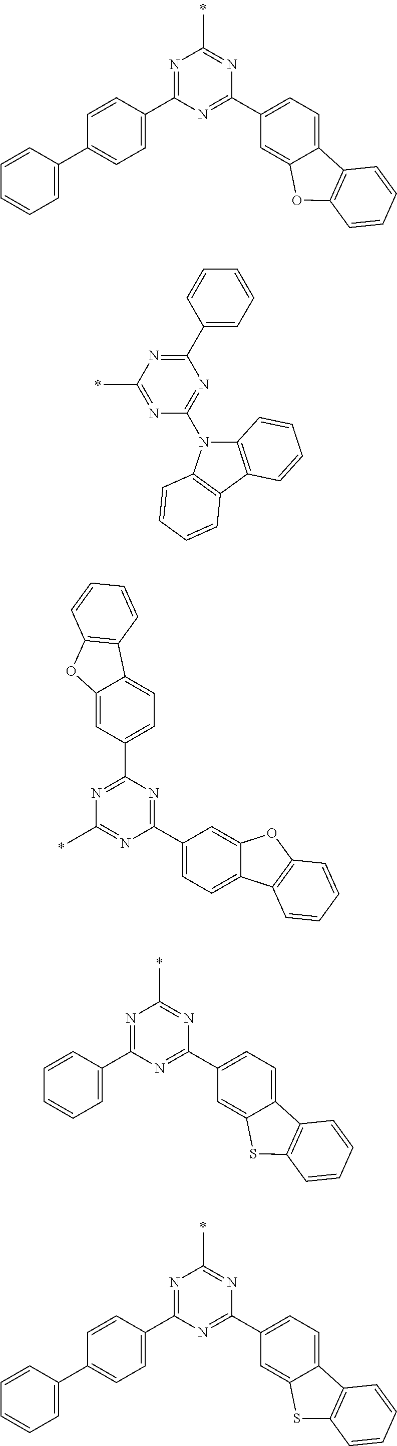 [SMILES: Cc1nc(-c2ccc(-c3ccccc3)cc2)nc(-c2ccc3c(c2)oc2ccccc23)n1 ; Cc1nc(-c2ccccc2)nc(-n2c3ccccc3c3ccccc32)n1 ; Cc1nc(-c2ccc3c(c2)oc2ccccc23)nc(-c2ccc3c(c2)oc2ccccc23)n1 ; Cc1nc(-c2ccccc2)nc(-c2ccc3c(c2)sc2ccccc23)n1 ; Cc1nc(-c2ccc(-c3ccccc3)cc2)nc(-c2ccc3c(c2)sc2ccccc23)n1]

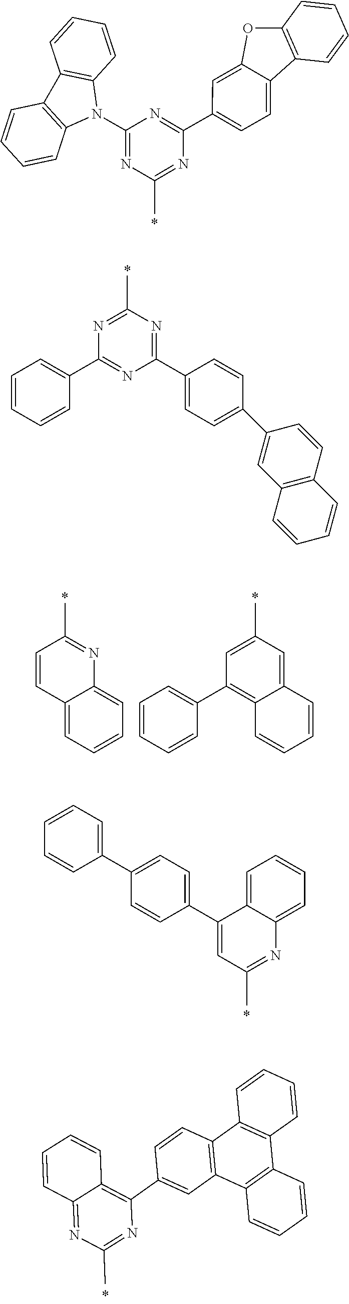

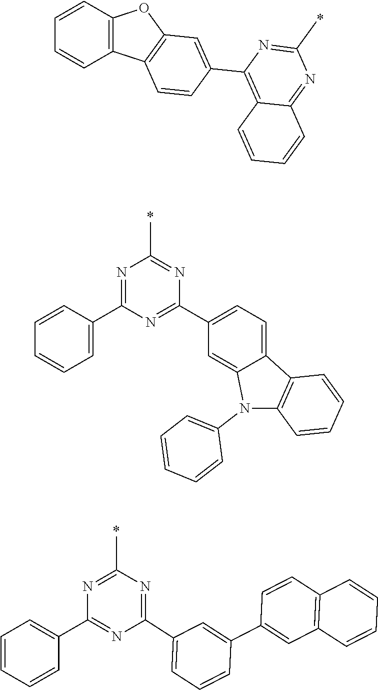 [SMILES: Cc1nc(-c2ccc3c(c2)oc2ccccc23)c2ccccc2n1 ; Cc1nc(-c2ccccc2)nc(-c2ccc3c4ccccc4n(-c4ccccc4)c3c2)n1 ; Cc1nc(-c2ccccc2)nc(-c2cccc(-c3ccc4ccccc4c3)c2)n1]

In an implementation, the second compound represented by Chemical Formula 3A may be represented by, e.g., one of Chemical Formula 3A-1 to Chemical Formula 3A-4 (e.g., according to a specific bonding position of the group represented by Chemical Formula b).

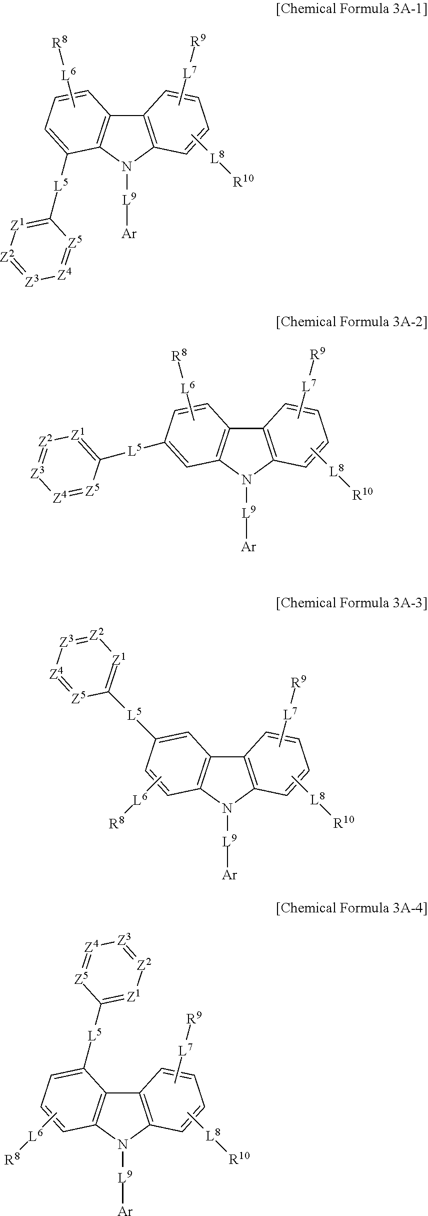

In Chemical Formula 3A-1 to Chemical Formula 3A-4, L⁵ to L⁹, Ar, R⁷ to R¹⁰, and Z¹ to Z⁵ may be defined the same as those of Chemical Formula 3.

For example, Chemical Formula 3A may be represented by Chemical Formula 3A-2 or Chemical Formula 3A-4.

In an implementation, the second compound represented by Chemical Formula 3C may be represented by, e.g., one of Chemical Formula 3C-1 to Chemical Formula 3C-4 (e.g., according to a specific bonding position of the group represented by Chemical Formula b).

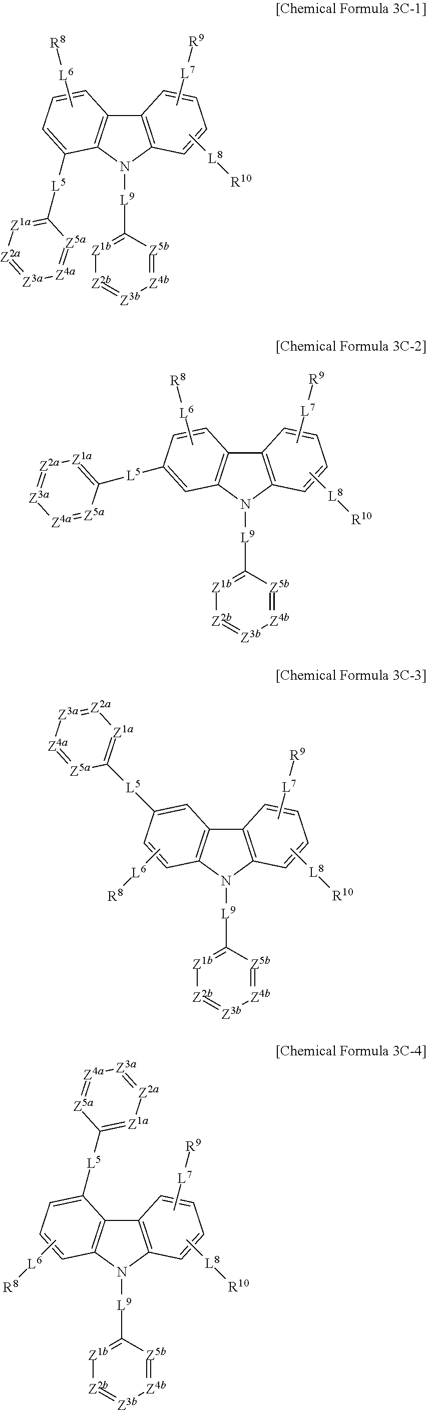

In Chemical Formula 3C-1 to Chemical Formula 3C-4, L⁵ to L⁹, R⁸ to R¹⁰, Z^{1a} to Z^{5a}, and Z^{1b} to Z^{5b} may be defined the same as those of Chemical Formula 3C.

In an implementation, the second compound represented by Chemical Formula 3C may be represented by Chemical Formula 3C-1 or Chemical Formula 3C-4.

In an implementation, the second compound represented by Chemical Formula 3E may be represented by, e.g., one of Chemical Formula 3E-1 to Chemical Formula 3E-4 (e.g., according to a specific bonding position of the group represented by Chemical Formula b).

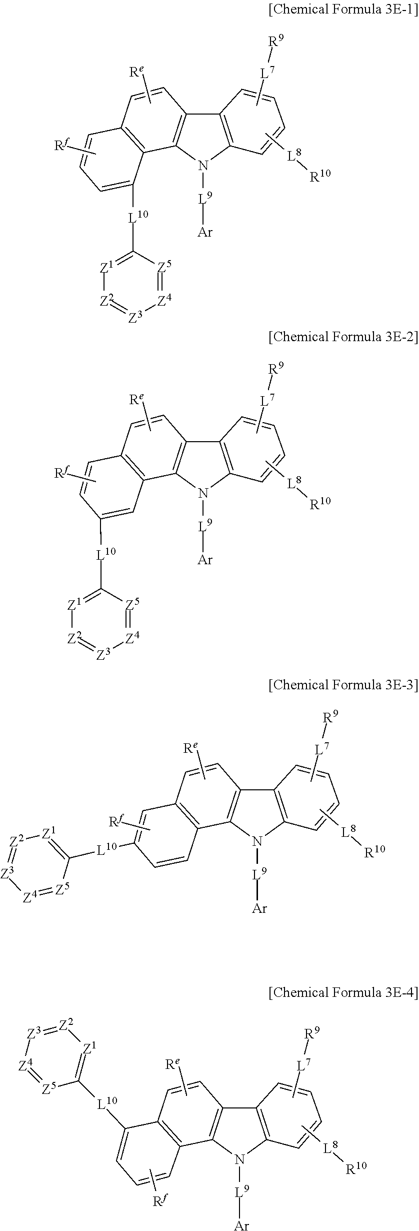

In Chemical Formula 3E-1 to Chemical Formula 3E-4, L⁷ to L¹⁰, R⁹, R¹⁰, Z¹ to Z⁵, and Ar may be defined the same as those of Chemical Formula 3.

In an implementation, the second compound represented by Chemical Formula 3E may be represented by Chemical Formula 3E-2.

In an implementation, the second compound represented by Chemical Formula 3F may be represented by, e.g., one of Chemical Formula 3F-1 to Chemical Formula 3F-4 (e.g., according to a specific bonding position of the group represented by Chemical Formula b).

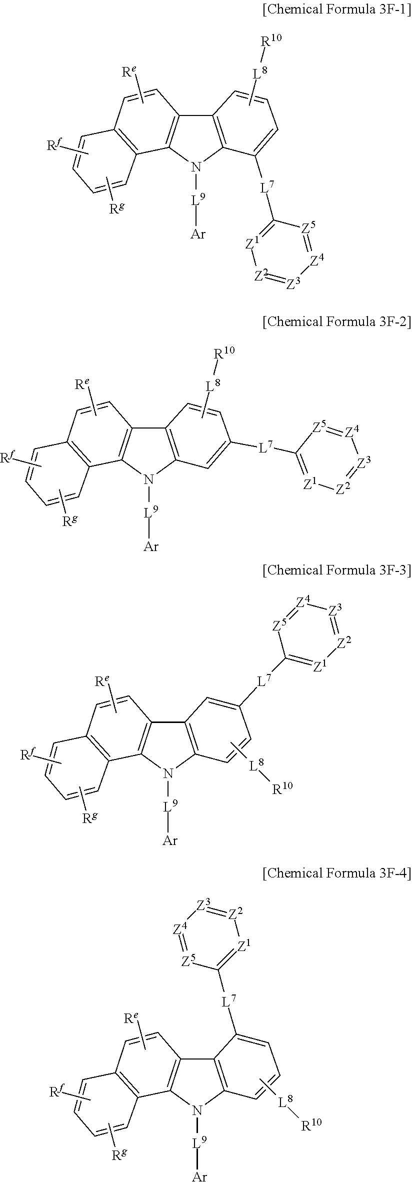

In Chemical Formula 3F-1 to Chemical Formula 3F-4, L⁷ to L⁹, R¹⁰, R^{e}, R^{f}, R^{g}, Z¹ to Z⁵, and Ar may be defined the same as those of Chemical Formula 3F.

In an implementation, the second compound represented by Chemical Formula 3F may be represented by Chemical Formula 3F-3 or Chemical Formula 3F-4.

In an implementation, the second compound represented by Chemical Formula 3H may be represented by, e.g., one of Chemical Formula 3H-1 to Chemical Formula 3H-4 (e.g., according to a specific bonding position of the group represented by Chemical Formula b).

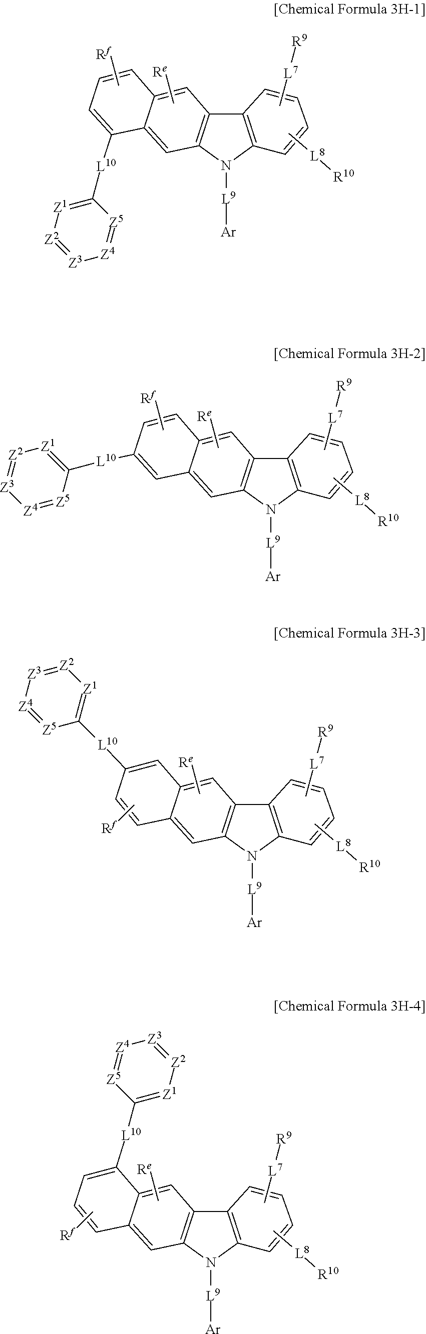

In Chemical Formula 3H-1 to Chemical Formula 3H-4, L⁷ to L¹⁰, R^{e}, R^{f}, R⁹, R¹⁰, Z¹ to Z⁵, and Ar may be defined the same as those of Chemical Formula 3H.

In an implementation, the second compound represented by Chemical Formula 3I may be represented by, e.g., one of Chemical Formula 3I-1 to Chemical Formula 3I-4 (e.g., according to a specific bonding position of the group represented by Chemical Formula b).

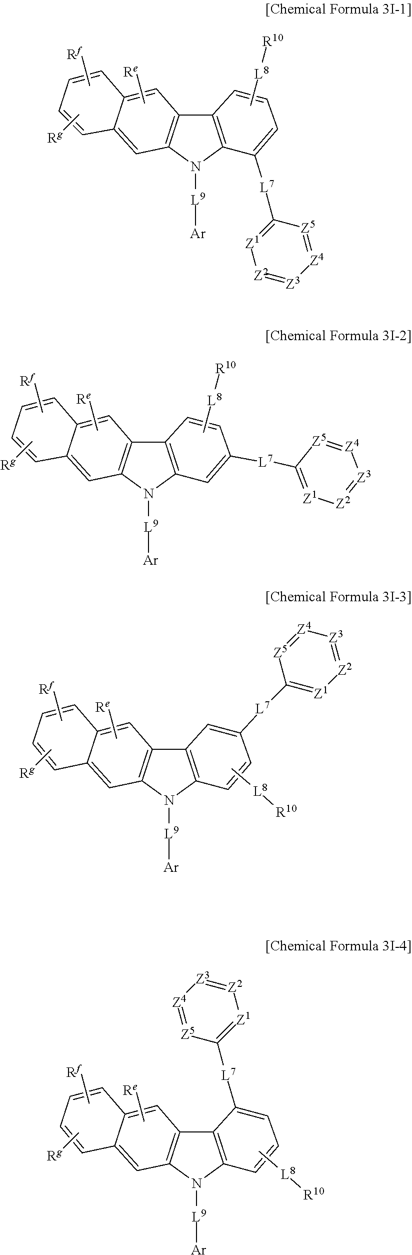

In Chemical Formula 3I-1 to Chemical Formula 3I-4, L⁷ to L⁹, R^{e}, R^{f}, R^{g}, R¹⁰, Z¹ to Z⁵, and Ar may be defined the same as those of Chemical Formula 3I.

In an implementation, the second compound represented by Chemical Formula 3K may be represented by, e.g., one of Chemical Formula 3K-1 to Chemical Formula 3K-4 (e.g., according to a specific bonding position of the group represented by Chemical Formula b).

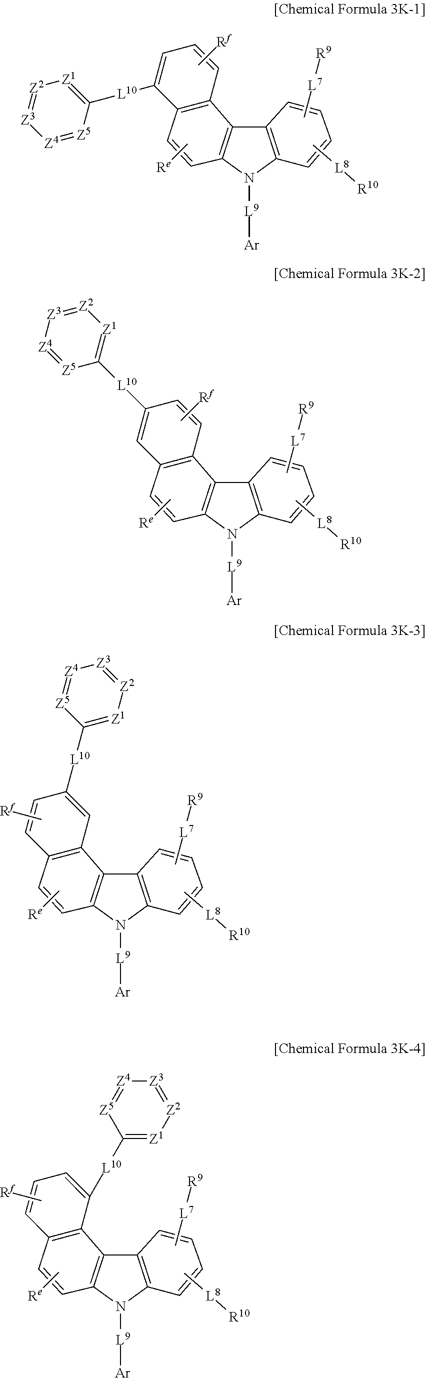

In Chemical Formula 3K-1 to Chemical Formula 3K-4, L⁷ to L⁹, R⁹, R¹⁰, R^{e}, R^{f}, R^{g}, Z¹ to Z⁵, and Ar may be defined the same as those of Chemical Formula 3K.

In an implementation, the second compound represented by Chemical Formula 3L may be represented by, e.g., one of Chemical Formula 3L-1 to Chemical Formula 3L-4 (e.g., according to a specific bonding position of the group represented by Chemical Formula b).

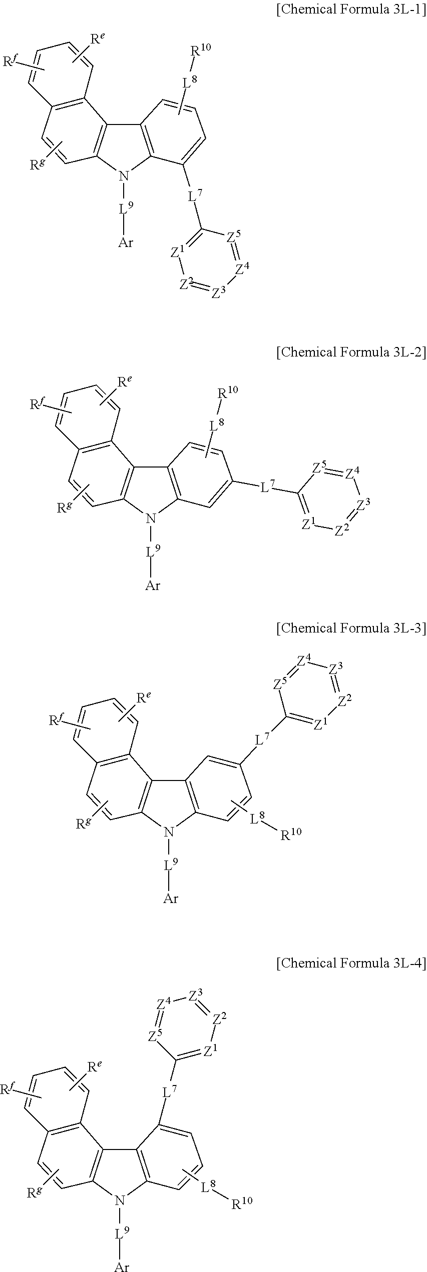

In Chemical Formula 3L-1 to Chemical Formula 3L-4, L⁷ to L⁹, R¹⁰, R^{e}, R^{f}, R^{g}, Z¹ to Z⁵, and Ar may be defined the same as those of Chemical Formula 3L.

In an implementation, the second compound represented by Chemical Formula 3L may be represented by Chemical Formula 3L-4.

In an implementation, the second compound represented by Chemical Formula 3L may be represented by Chemical Formula 3L-4. Formula 3J.

In an implementation, the second compound may be represented by, e.g., one of Chemical Formula 3A-2, Chemical Formula 3B, Chemical Formula 3E-2, and Chemical Formula 3J.

In an implementation, the second compound may be, e.g., a compound of the following Group 2.

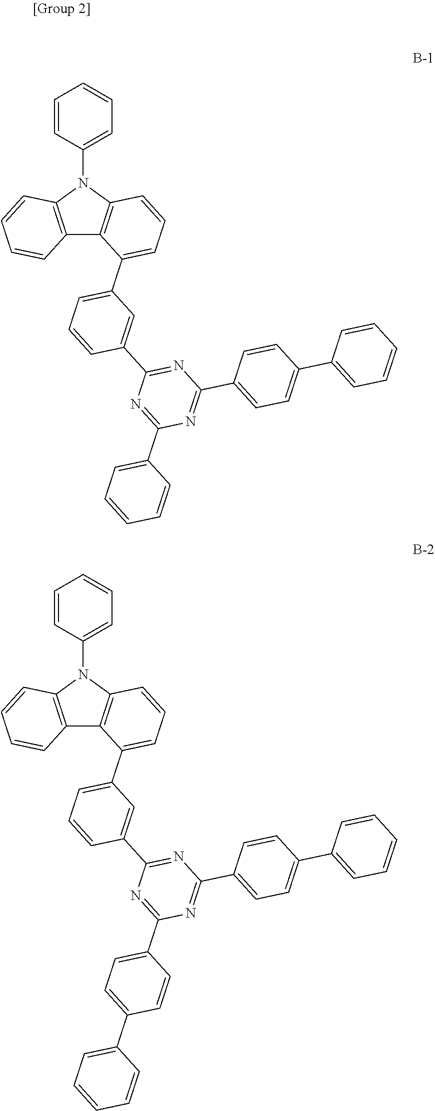 [SMILES: c1ccc(-c2ccc(-c3nc(-c4ccccc4)nc(-c4cccc(-c5cccc6c5c5ccccc5n6-c5ccccc5)c4)n3)cc2)cc1 ; c1ccc(-c2ccc(-c3nc(-c4ccc(-c5ccccc5)cc4)nc(-c4cccc(-c5cccc6c5c5ccccc5n6-c5ccccc5)c4)n3)cc2)cc1]

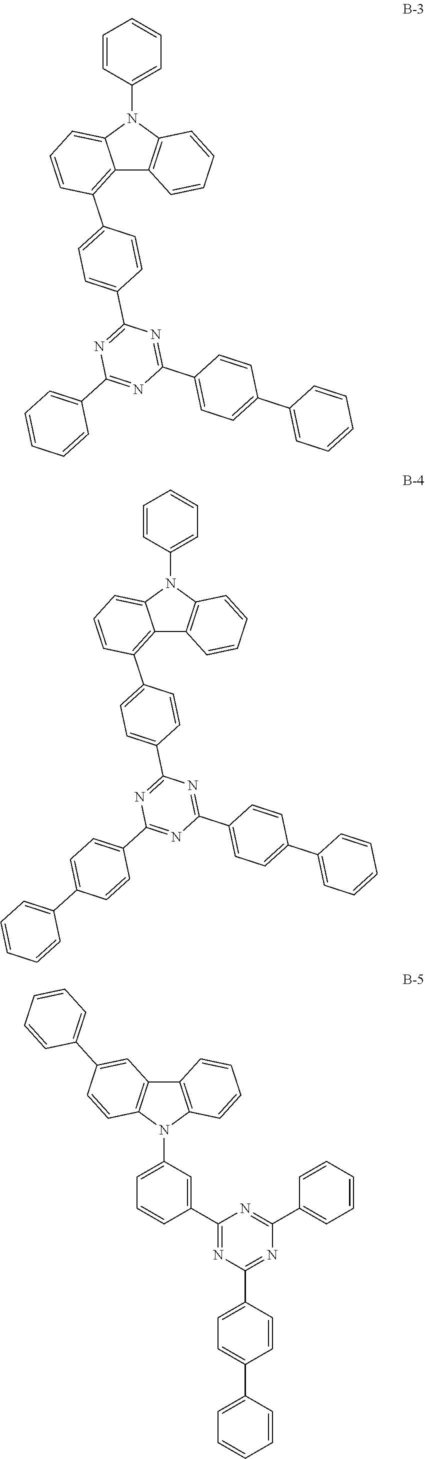

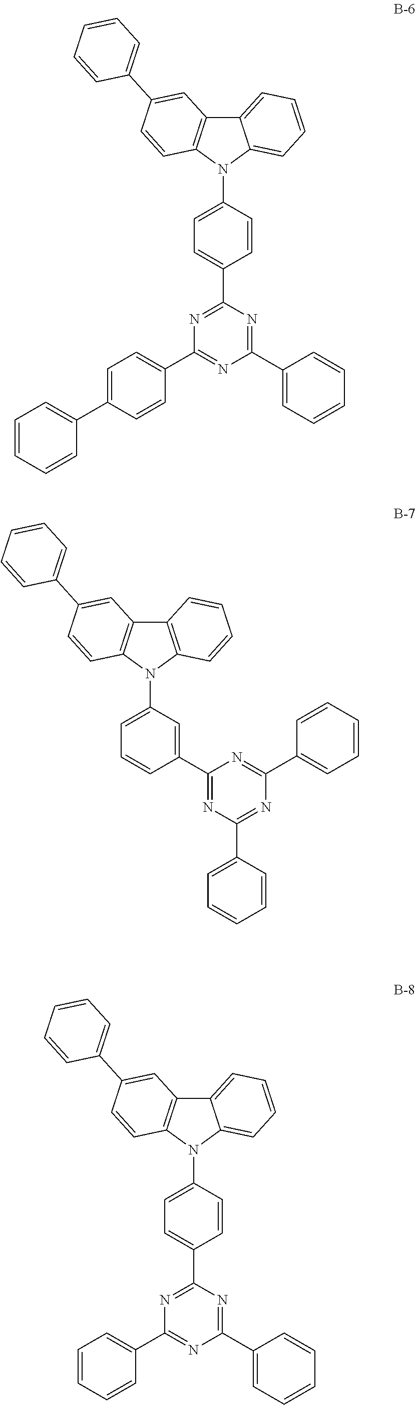 [SMILES: c1ccc(-c2ccc(-c3nc(-c4ccccc4)nc(-c4ccc(-n5c6ccccc6c6cc(-c7ccccc7)ccc65)cc4)n3)cc2)cc1 ; c1ccc(-c2ccc3c(c2)c2ccccc2n3-c2cccc(-c3nc(-c4ccccc4)nc(-c4ccccc4)n3)c2)cc1 ; c1ccc(-c2ccc3c(c2)c2ccccc2n3-c2ccc(-c3nc(-c4ccccc4)nc(-c4ccccc4)n3)cc2)cc1]

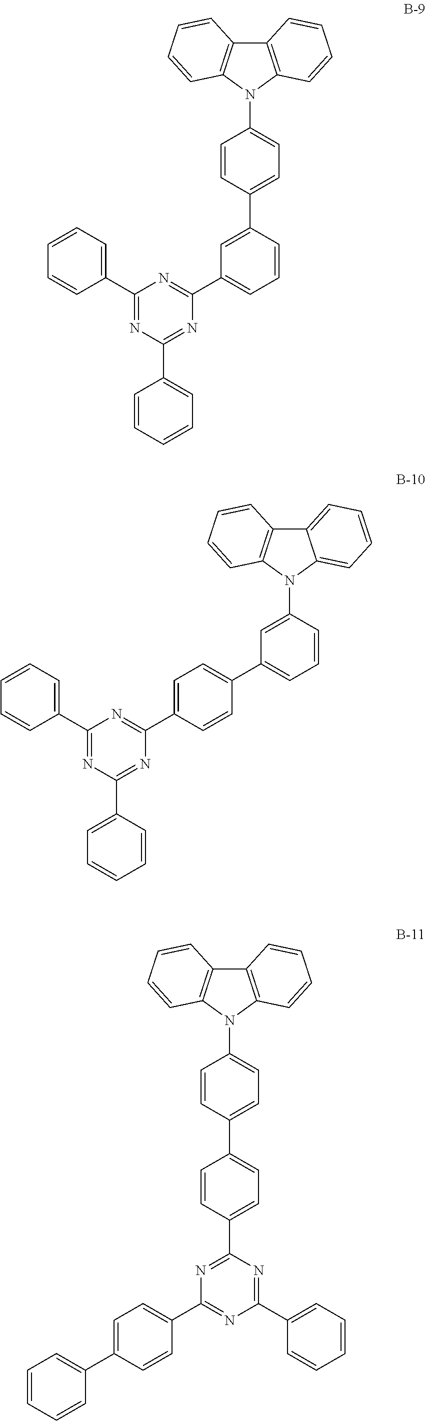 [SMILES: c1ccc(-c2nc(-c3ccccc3)nc(-c3cccc(-c4ccc(-n5c6ccccc6c6ccccc65)cc4)c3)n2)cc1 ; c1ccc(-c2nc(-c3ccccc3)nc(-c3ccc(-c4cccc(-n5c6ccccc6c6ccccc65)c4)cc3)n2)cc1 ; c1ccc(-c2ccc(-c3nc(-c4ccccc4)nc(-c4ccc(-c5ccc(-n6c7ccccc7c7ccccc76)cc5)cc4)n3)cc2)cc1]

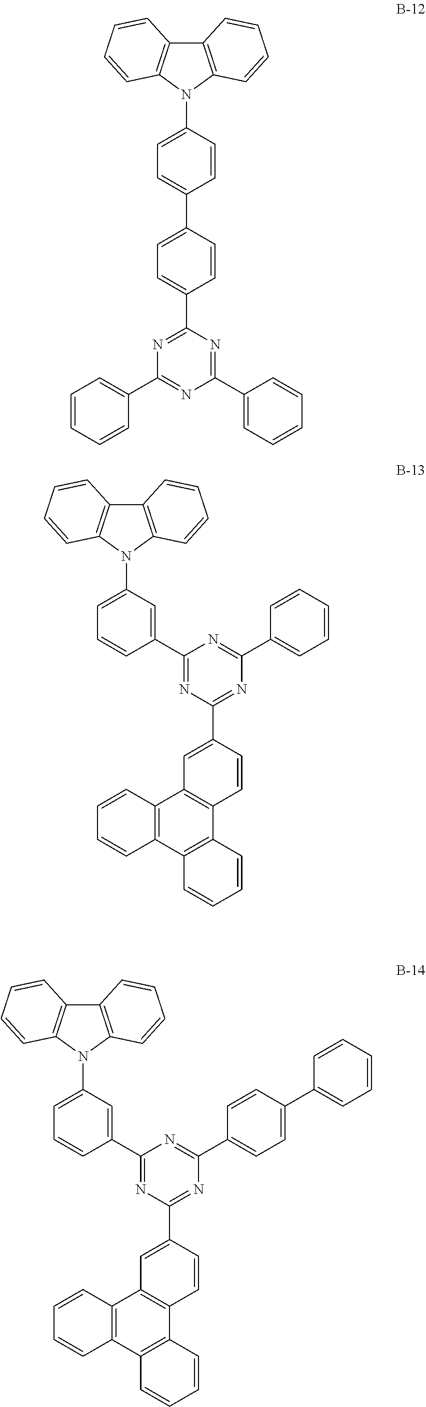

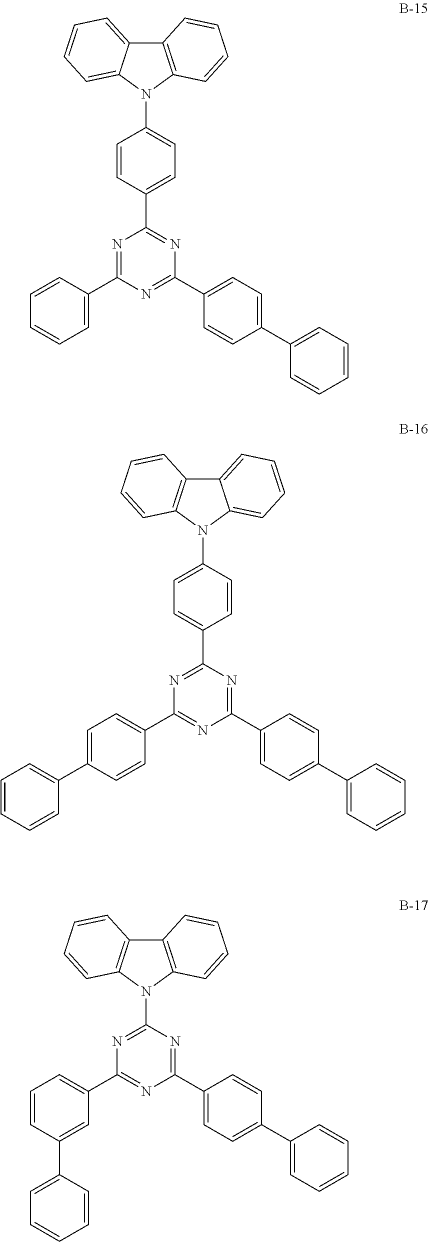 [SMILES: c1ccc(-c2ccc(-c3nc(-c4ccccc4)nc(-c4ccc(-n5c6ccccc6c6ccccc65)cc4)n3)cc2)cc1 ; c1ccc(-c2ccc(-c3nc(-c4ccc(-c5ccccc5)cc4)nc(-c4ccc(-n5c6ccccc6c6ccccc65)cc4)n3)cc2)cc1 ; c1ccc(-c2ccc(-c3nc(-c4cccc(-c5ccccc5)c4)nc(-n4c5ccccc5c5ccccc54)n3)cc2)cc1]

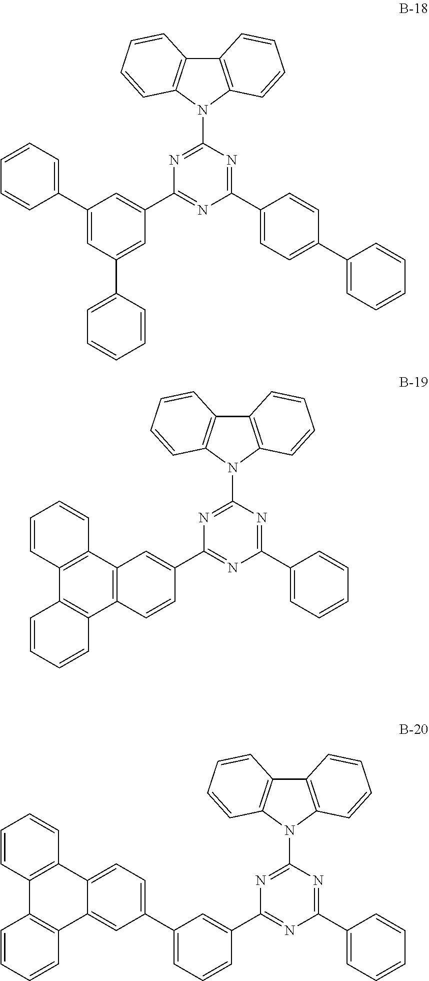 [SMILES: c1ccc(-c2ccc(-c3nc(-c4cc(-c5ccccc5)cc(-c5ccccc5)c4)nc(-n4c5ccccc5c5ccccc54)n3)cc2)cc1 ; c1ccc(-c2nc(-c3ccc4c5ccccc5c5ccccc5c4c3)nc(-n3c4ccccc4c4ccccc43)n2)cc1 ; c1ccc(-c2nc(-c3cccc(-c4ccc5c6ccccc6c6ccccc6c5c4)c3)nc(-n3c4ccccc4c4ccccc43)n2)cc1]

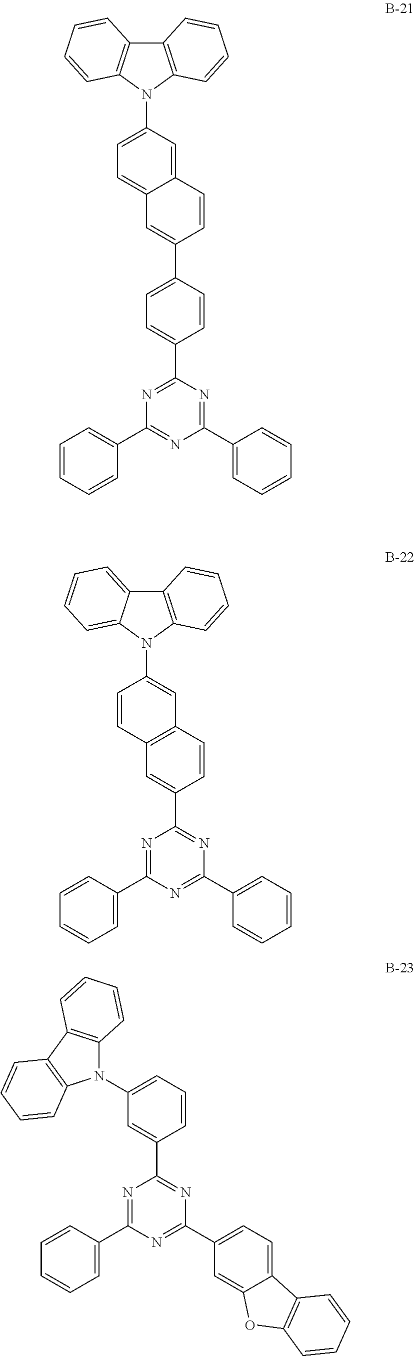

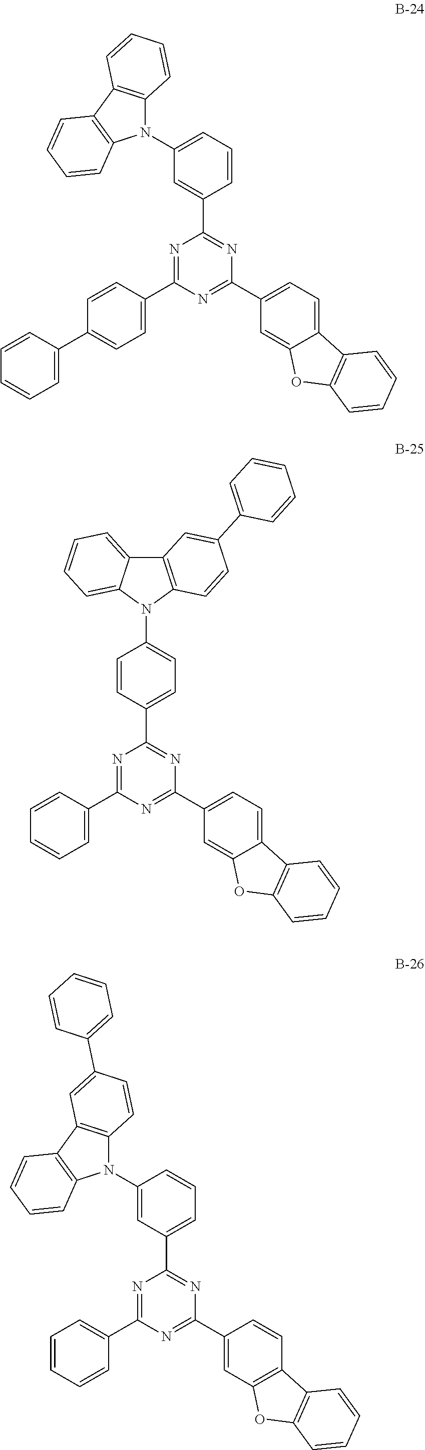 [SMILES: c1ccc(-c2ccc(-c3nc(-c4cccc(-n5c6ccccc6c6ccccc65)c4)nc(-c4ccc5c(c4)oc4ccccc45)n3)cc2)cc1 ; c1ccc(-c2ccc3c(c2)c2ccccc2n3-c2ccc(-c3nc(-c4ccccc4)nc(-c4ccc5c(c4)oc4ccccc45)n3)cc2)cc1 ; c1ccc(-c2ccc3c(c2)c2ccccc2n3-c2cccc(-c3nc(-c4ccccc4)nc(-c4ccc5c(c4)oc4ccccc45)n3)c2)cc1]

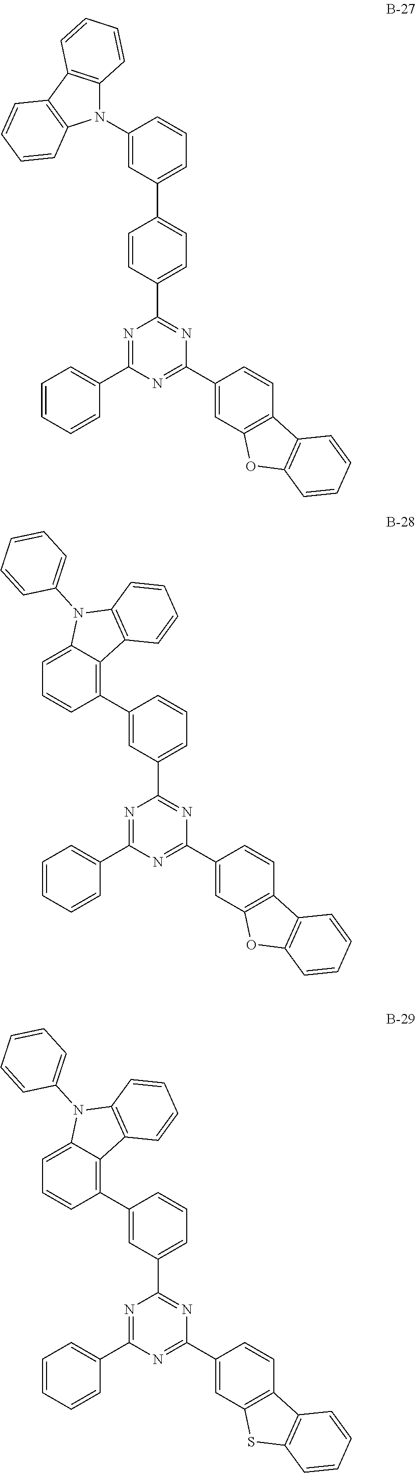 [SMILES: c1ccc(-c2nc(-c3ccc(-c4cccc(-n5c6ccccc6c6ccccc65)c4)cc3)nc(-c3ccc4c(c3)oc3ccccc34)n2)cc1 ; c1ccc(-c2nc(-c3cccc(-c4cccc5c4c4ccccc4n5-c4ccccc4)c3)nc(-c3ccc4c(c3)oc3ccccc34)n2)cc1 ; c1ccc(-c2nc(-c3cccc(-c4cccc5c4c4ccccc4n5-c4ccccc4)c3)nc(-c3ccc4c(c3)sc3ccccc34)n2)cc1]

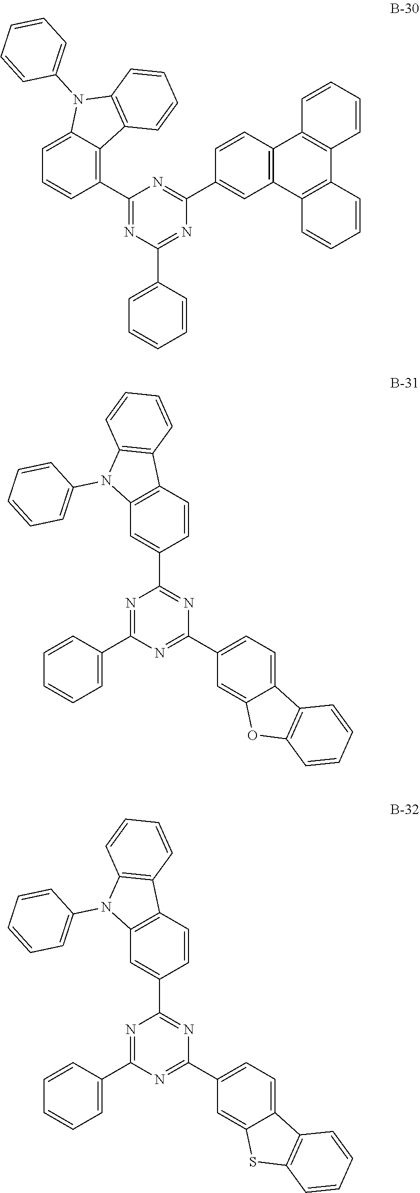 [SMILES: c1ccc(-c2nc(-c3ccc4c5ccccc5c5ccccc5c4c3)nc(-c3cccc4c3c3ccccc3n4-c3ccccc3)n2)cc1 ; c1ccc(-c2nc(-c3ccc4c(c3)oc3ccccc34)nc(-c3ccc4c5ccccc5n(-c5ccccc5)c4c3)n2)cc1 ; c1ccc(-c2nc(-c3ccc4c(c3)sc3ccccc34)nc(-c3ccc4c5ccccc5n(-c5ccccc5)c4c3)n2)cc1]

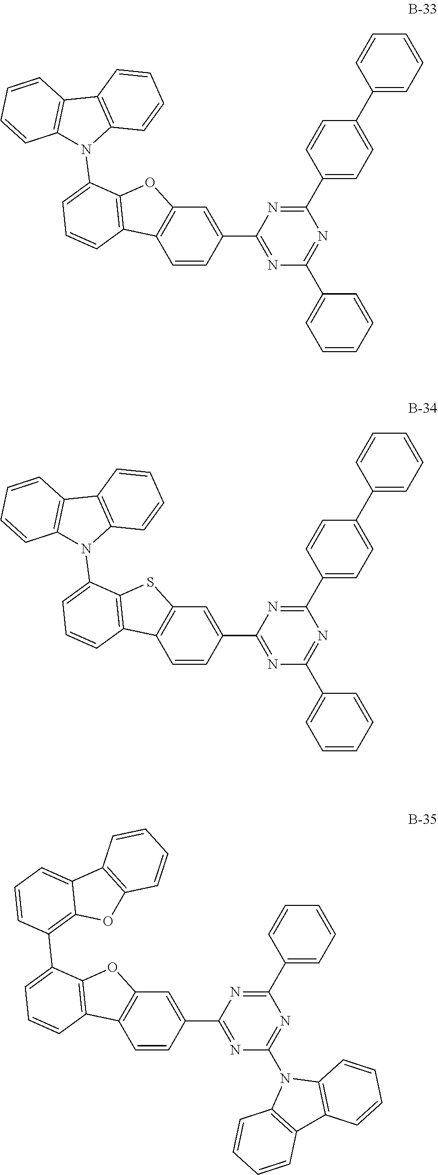 [SMILES: c1ccc(-c2ccc(-c3nc(-c4ccccc4)nc(-c4ccc5c(c4)oc4c(-n6c7ccccc7c7ccccc76)cccc45)n3)cc2)cc1 ; c1ccc(-c2ccc(-c3nc(-c4ccccc4)nc(-c4ccc5c(c4)sc4c(-n6c7ccccc7c7ccccc76)cccc45)n3)cc2)cc1 ; c1ccc(-c2nc(-c3ccc4c(c3)oc3c(-c5cccc6c5oc5ccccc56)cccc34)nc(-n3c4ccccc4c4ccccc43)n2)cc1]

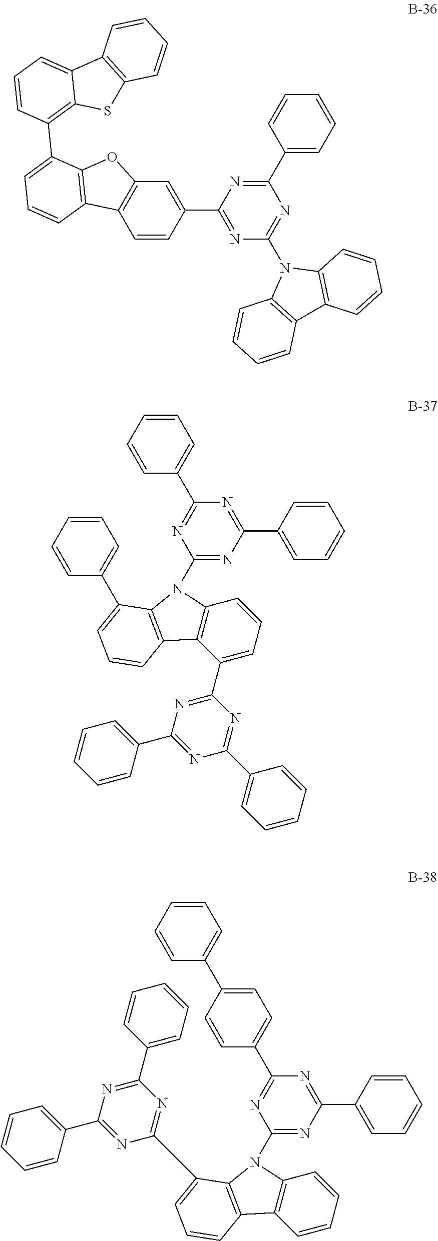 [SMILES: c1ccc(-c2nc(-c3ccc4c(c3)oc3c(-c5cccc6c5sc5ccccc56)cccc34)nc(-n3c4ccccc4c4ccccc43)n2)cc1 ; c1ccc(-c2nc(-c3ccccc3)nc(-c3cccc4c3c3cccc(-c5ccccc5)c3n4-c3nc(-c4ccccc4)nc(-c4ccccc4)n3)n2)cc1 ; c1ccc(-c2ccc(-c3nc(-c4ccccc4)nc(-n4c5ccccc5c5cccc(-c6nc(-c7ccccc7)nc(-c7ccccc7)n6)c54)n3)cc2)cc1]

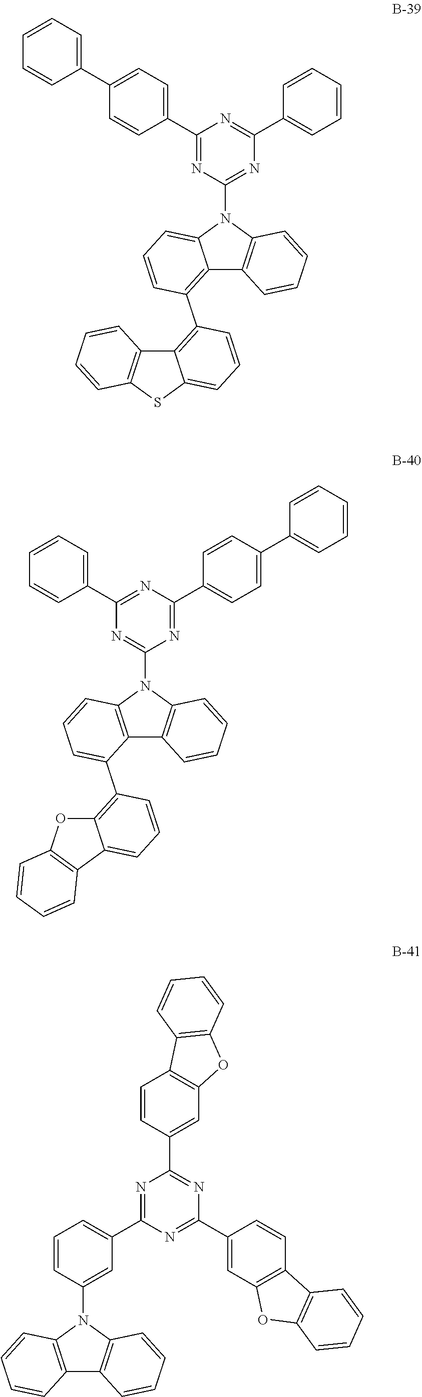 [SMILES: c1ccc(-c2ccc(-c3nc(-c4ccccc4)nc(-n4c5ccccc5c5c(-c6cccc7sc8ccccc8c67)cccc54)n3)cc2)cc1 ; c1ccc(-c2ccc(-c3nc(-c4ccccc4)nc(-n4c5ccccc5c5c(-c6cccc7c6oc6ccccc67)cccc54)n3)cc2)cc1 ; c1cc(-c2nc(-c3ccc4c(c3)oc3ccccc34)nc(-c3ccc4c(c3)oc3ccccc34)n2)cc(-n2c3ccccc3c3ccccc32)c1]

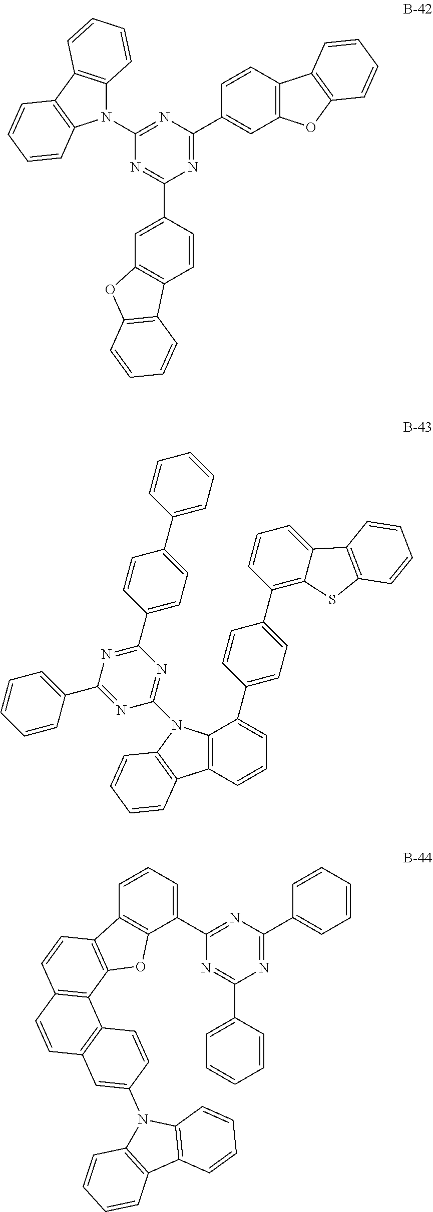 [SMILES: c1ccc2c(c1)oc1cc(-c3nc(-c4ccc5c(c4)oc4ccccc45)nc(-n4c5ccccc5c5ccccc54)n3)ccc12 ; c1ccc(-c2ccc(-c3nc(-c4ccccc4)nc(-n4c5ccccc5c5cccc(-c6ccc(-c7cccc8c7sc7ccccc78)cc6)c54)n3)cc2)cc1 ; c1ccc(-c2nc(-c3ccccc3)nc(-c3cccc4c3oc3c4ccc4ccc5cc(-n6c7ccccc7c7ccccc76)ccc5c43)n2)cc1]

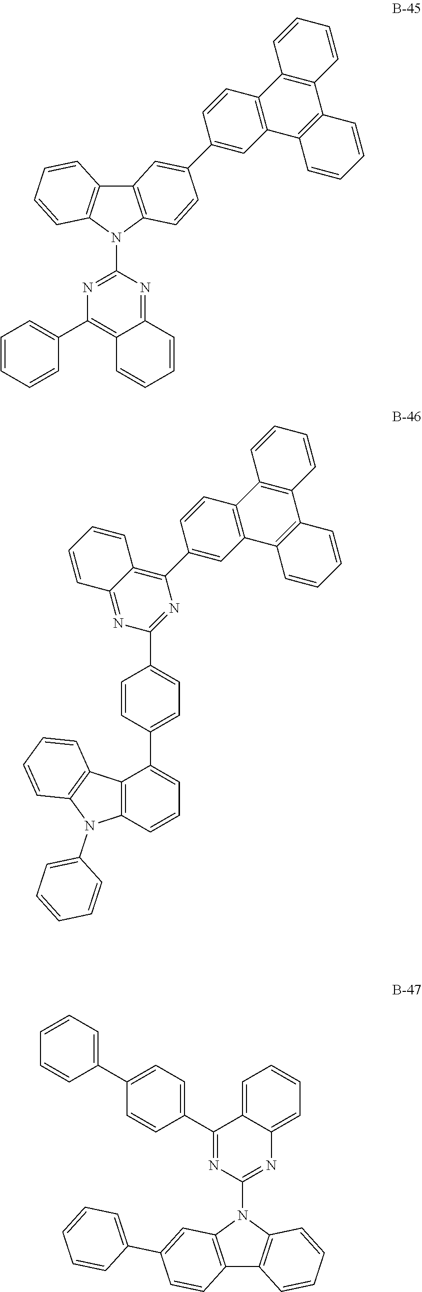 [SMILES: c1ccc(-c2nc(-n3c4ccccc4c4cc(-c5ccc6c7ccccc7c7ccccc7c6c5)ccc43)nc3ccccc23)cc1 ; c1ccc(-n2c3ccccc3c3c(-c4ccc(-c5nc(-c6ccc7c8ccccc8c8ccccc8c7c6)c6ccccc6n5)cc4)cccc32)cc1 ; c1ccc(-c2ccc(-c3nc(-n4c5ccccc5c5ccc(-c6ccccc6)cc54)nc4ccccc34)cc2)cc1]

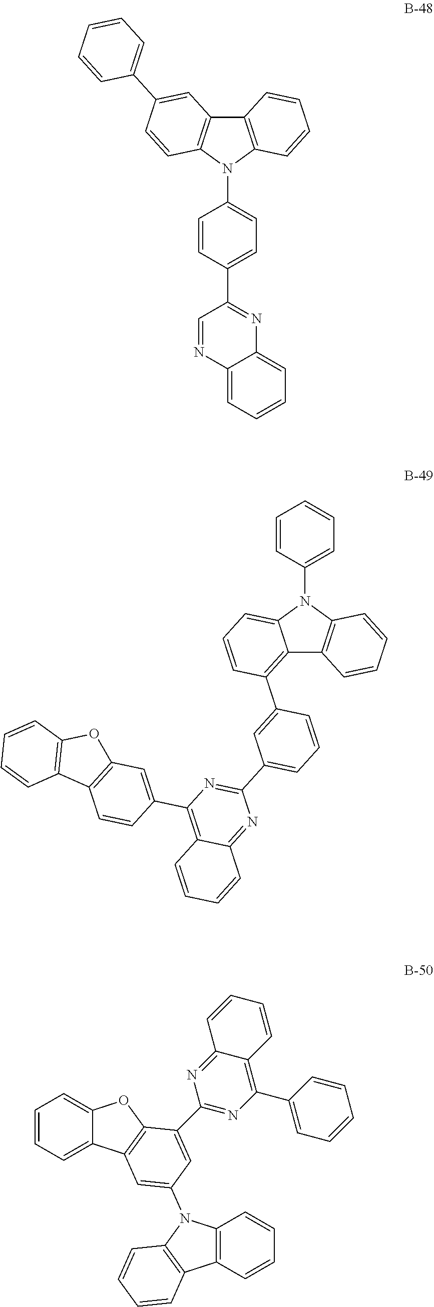 [SMILES: c1ccc(-c2ccc3c(c2)c2ccccc2n3-c2ccc(-c3cnc4ccccc4n3)cc2)cc1 ; c1ccc(-n2c3ccccc3c3c(-c4cccc(-c5nc(-c6ccc7c(c6)oc6ccccc67)c6ccccc6n5)c4)cccc32)cc1 ; c1ccc(-c2nc(-c3cc(-n4c5ccccc5c5ccccc54)cc4c3oc3ccccc34)nc3ccccc23)cc1]

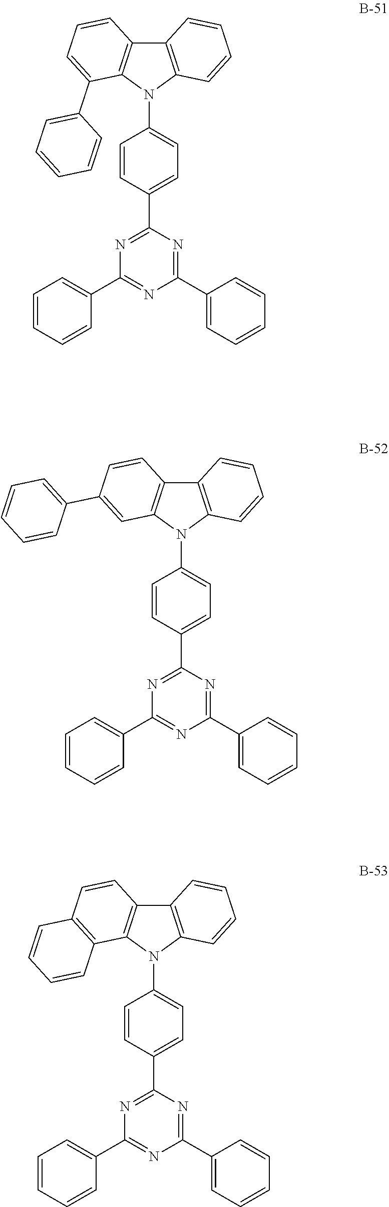 [SMILES: c1ccc(-c2nc(-c3ccccc3)nc(-c3ccc(-n4c5ccccc5c5cccc(-c6ccccc6)c54)cc3)n2)cc1 ; c1ccc(-c2ccc3c4ccccc4n(-c4ccc(-c5nc(-c6ccccc6)nc(-c6ccccc6)n5)cc4)c3c2)cc1 ; c1ccc(-c2nc(-c3ccccc3)nc(-c3ccc(-n4c5ccccc5c5ccc6ccccc6c54)cc3)n2)cc1]

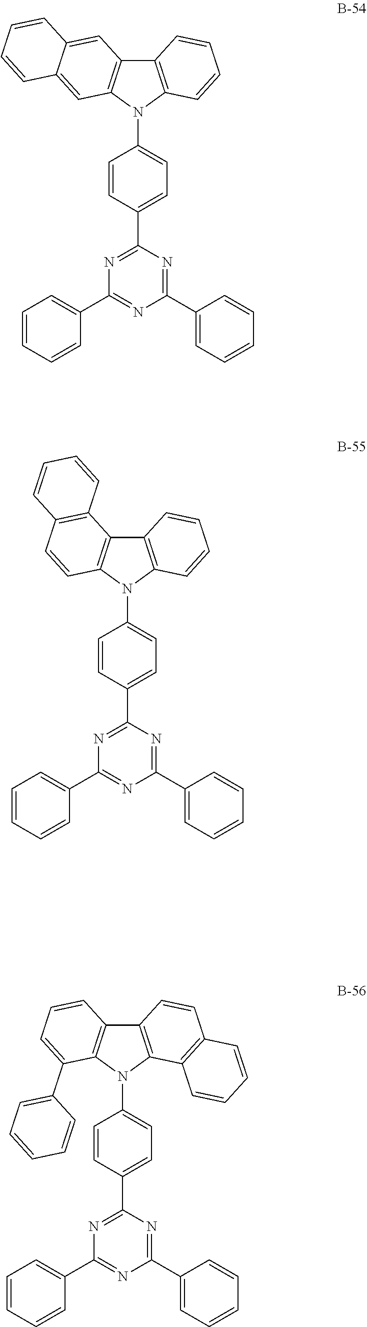 [SMILES: c1ccc(-c2nc(-c3ccccc3)nc(-c3ccc(-n4c5ccccc5c5cc6ccccc6cc54)cc3)n2)cc1 ; c1ccc(-c2nc(-c3ccccc3)nc(-c3ccc(-n4c5ccccc5c5c6ccccc6ccc54)cc3)n2)cc1 ; c1ccc(-c2nc(-c3ccccc3)nc(-c3ccc(-n4c5c(-c6ccccc6)cccc5c5ccc6ccccc6c54)cc3)n2)cc1]

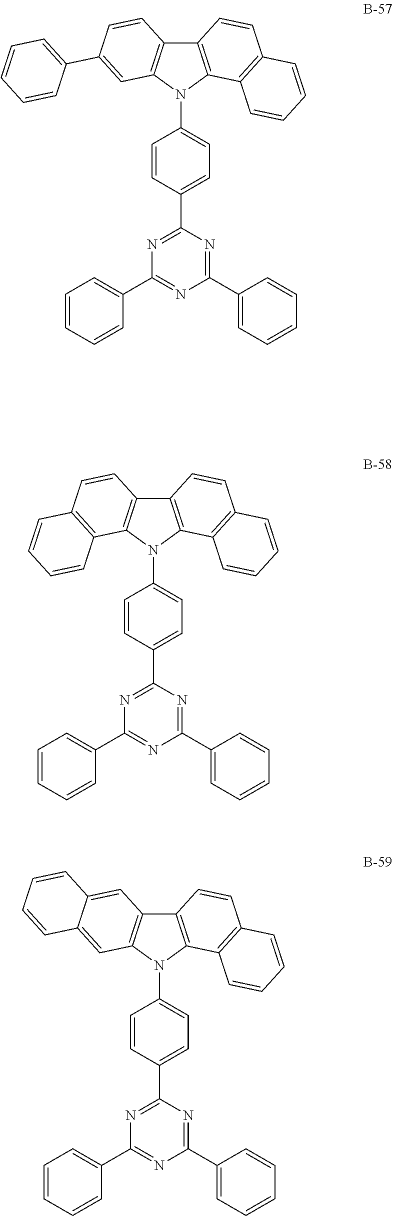 [SMILES: c1ccc(-c2ccc3c4ccc5ccccc5c4n(-c4ccc(-c5nc(-c6ccccc6)nc(-c6ccccc6)n5)cc4)c3c2)cc1 ; c1ccc(-c2nc(-c3ccccc3)nc(-c3ccc(-n4c5c6ccccc6ccc5c5ccc6ccccc6c54)cc3)n2)cc1 ; c1ccc(-c2nc(-c3ccccc3)nc(-c3ccc(-n4c5cc6ccccc6cc5c5ccc6ccccc6c54)cc3)n2)cc1]

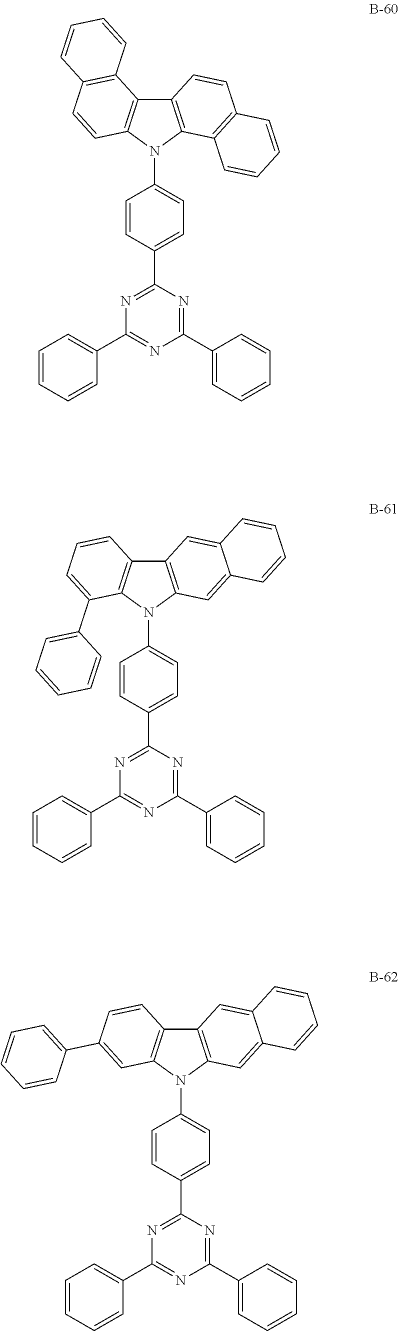 [SMILES: c1ccc(-c2nc(-c3ccccc3)nc(-c3ccc(-n4c5ccc6ccccc6c5c5ccc6ccccc6c54)cc3)n2)cc1 ; c1ccc(-c2nc(-c3ccccc3)nc(-c3ccc(-n4c5cc6ccccc6cc5c5cccc(-c6ccccc6)c54)cc3)n2)cc1 ; c1ccc(-c2ccc3c4cc5ccccc5cc4n(-c4ccc(-c5nc(-c6ccccc6)nc(-c6ccccc6)n5)cc4)c3c2)cc1]

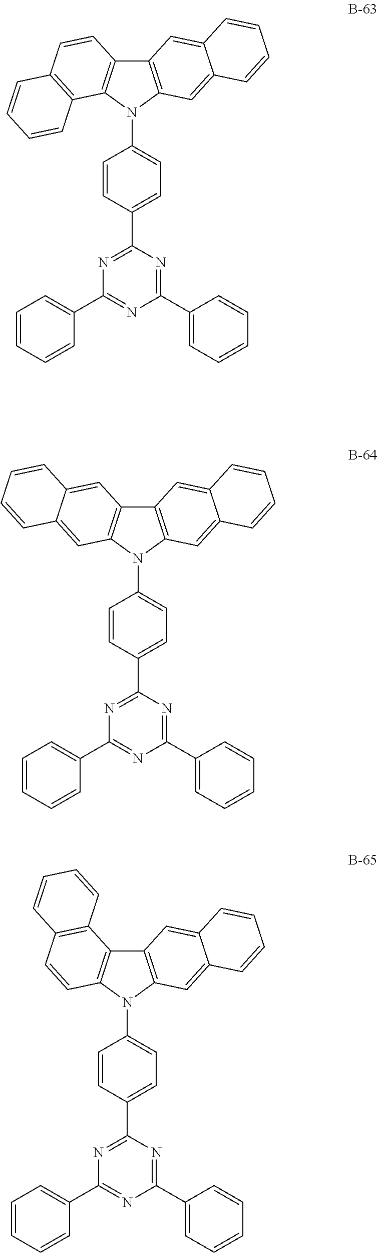 [SMILES: c1ccc(-c2nc(-c3ccccc3)nc(-c3ccc(-n4c5cc6ccccc6cc5c5ccc6ccccc6c54)cc3)n2)cc1 ; c1ccc(-c2nc(-c3ccccc3)nc(-c3ccc(-n4c5cc6ccccc6cc5c5cc6ccccc6cc54)cc3)n2)cc1 ; c1ccc(-c2nc(-c3ccccc3)nc(-c3ccc(-n4c5cc6ccccc6cc5c5c6ccccc6ccc54)cc3)n2)cc1]

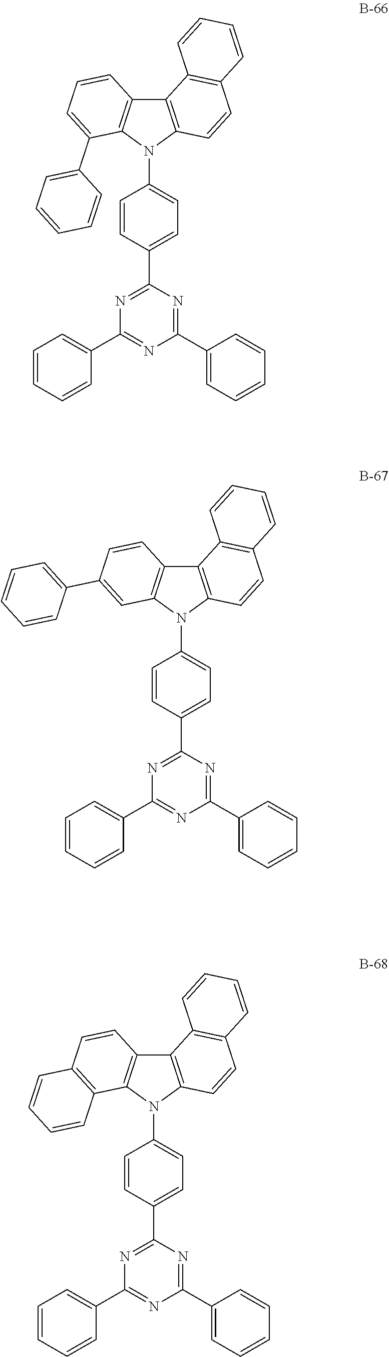 [SMILES: c1ccc(-c2nc(-c3ccccc3)nc(-c3ccc(-n4c5ccc6ccccc6c5c5cccc(-c6ccccc6)c54)cc3)n2)cc1 ; c1ccc(-c2ccc3c4c5ccccc5ccc4n(-c4ccc(-c5nc(-c6ccccc6)nc(-c6ccccc6)n5)cc4)c3c2)cc1 ; c1ccc(-c2nc(-c3ccccc3)nc(-c3ccc(-n4c5ccc6ccccc6c5c5ccc6ccccc6c54)cc3)n2)cc1]

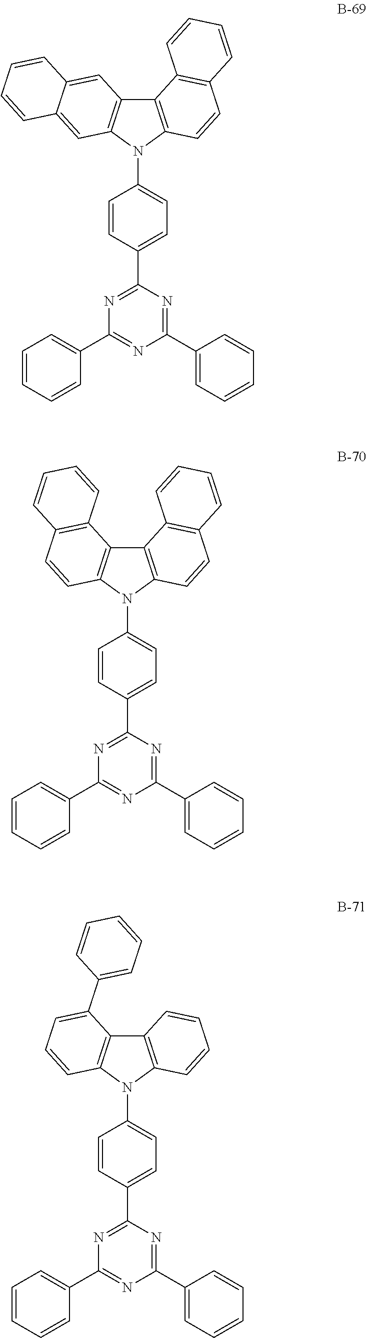 [SMILES: c1ccc(-c2nc(-c3ccccc3)nc(-c3ccc(-n4c5cc6ccccc6cc5c5c6ccccc6ccc54)cc3)n2)cc1 ; c1ccc(-c2nc(-c3ccccc3)nc(-c3ccc(-n4c5ccc6ccccc6c5c5c6ccccc6ccc54)cc3)n2)cc1 ; c1ccc(-c2nc(-c3ccccc3)nc(-c3ccc(-n4c5ccccc5c5c(-c6ccccc6)cccc54)cc3)n2)cc1]

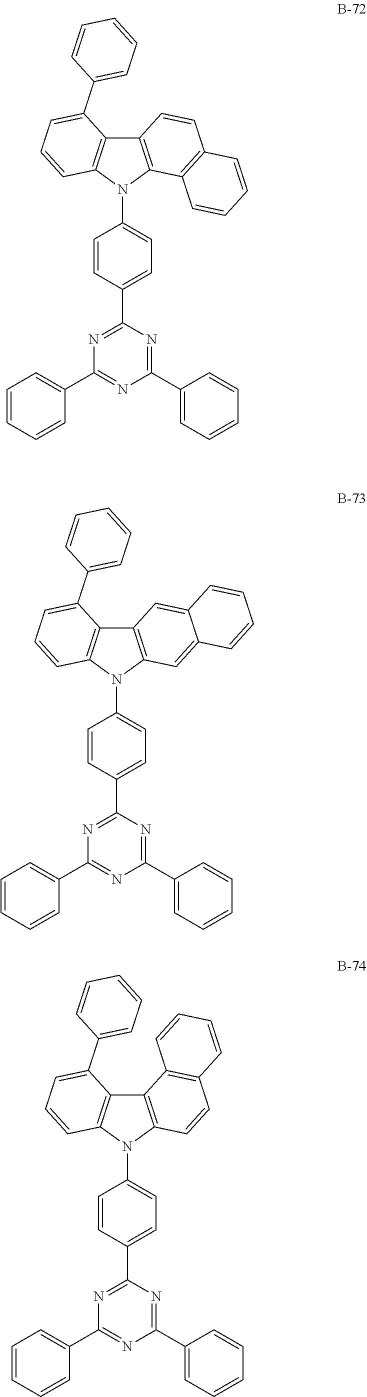 [SMILES: c1ccc(-c2nc(-c3ccccc3)nc(-c3ccc(-n4c5cccc(-c6ccccc6)c5c5ccc6ccccc6c54)cc3)n2)cc1 ; c1ccc(-c2nc(-c3ccccc3)nc(-c3ccc(-n4c5cc6ccccc6cc5c5c(-c6ccccc6)cccc54)cc3)n2)cc1 ; c1ccc(-c2nc(-c3ccccc3)nc(-c3ccc(-n4c5cccc(-c6ccccc6)c5c5c6ccccc6ccc54)cc3)n2)cc1]

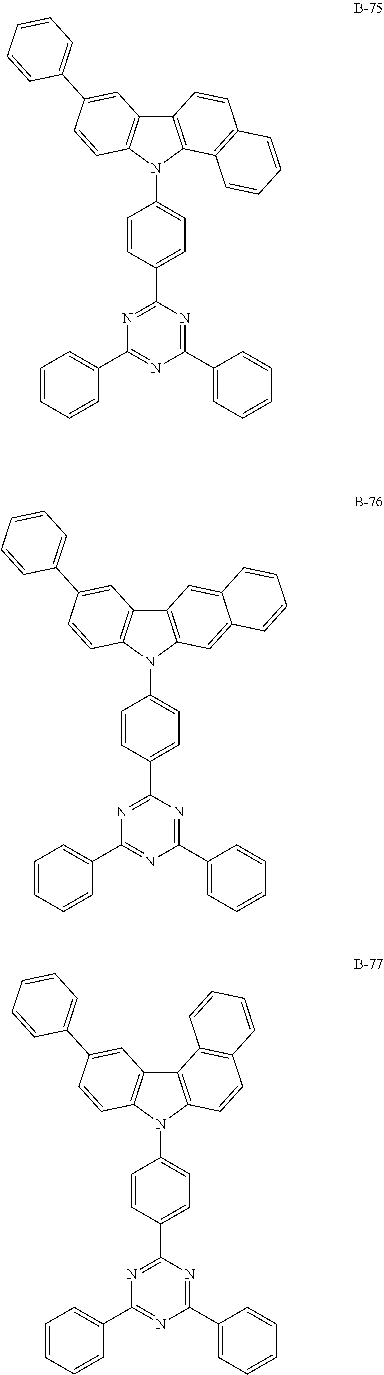 [SMILES: c1ccc(-c2ccc3c(c2)c2ccc4ccccc4c2n3-c2ccc(-c3nc(-c4ccccc4)nc(-c4ccccc4)n3)cc2)cc1 ; c1ccc(-c2ccc3c(c2)c2cc4ccccc4cc2n3-c2ccc(-c3nc(-c4ccccc4)nc(-c4ccccc4)n3)cc2)cc1 ; c1ccc(-c2ccc3c(c2)c2c4ccccc4ccc2n3-c2ccc(-c3nc(-c4ccccc4)nc(-c4ccccc4)n3)cc2)cc1]

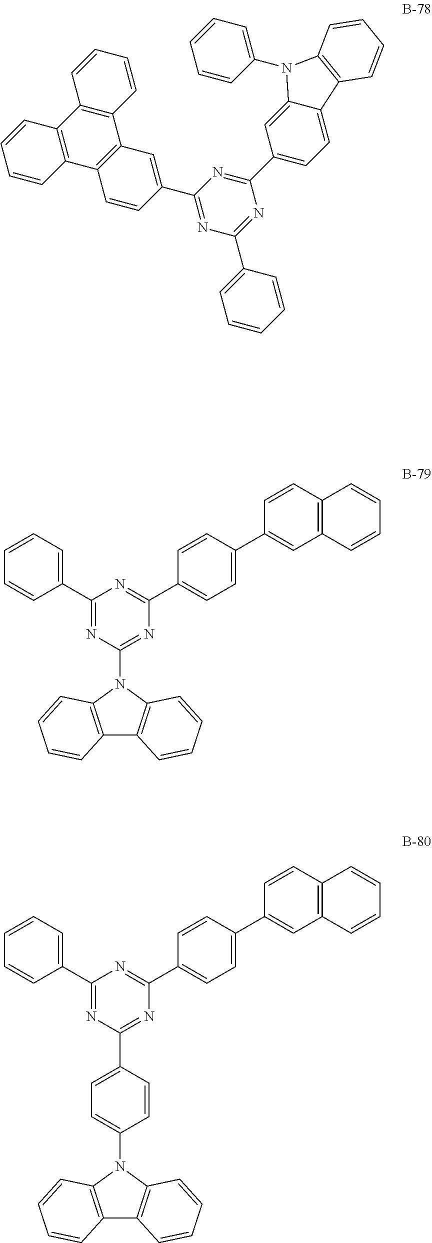 [SMILES: c1ccc(-c2nc(-c3ccc4c5ccccc5c5ccccc5c4c3)nc(-c3ccc4c5ccccc5n(-c5ccccc5)c4c3)n2)cc1 ; c1ccc(-c2nc(-c3ccc(-c4ccc5ccccc5c4)cc3)nc(-n3c4ccccc4c4ccccc43)n2)cc1 ; c1ccc(-c2nc(-c3ccc(-c4ccc5ccccc5c4)cc3)nc(-c3ccc(-n4c5ccccc5c5ccccc54)cc3)n2)cc1]

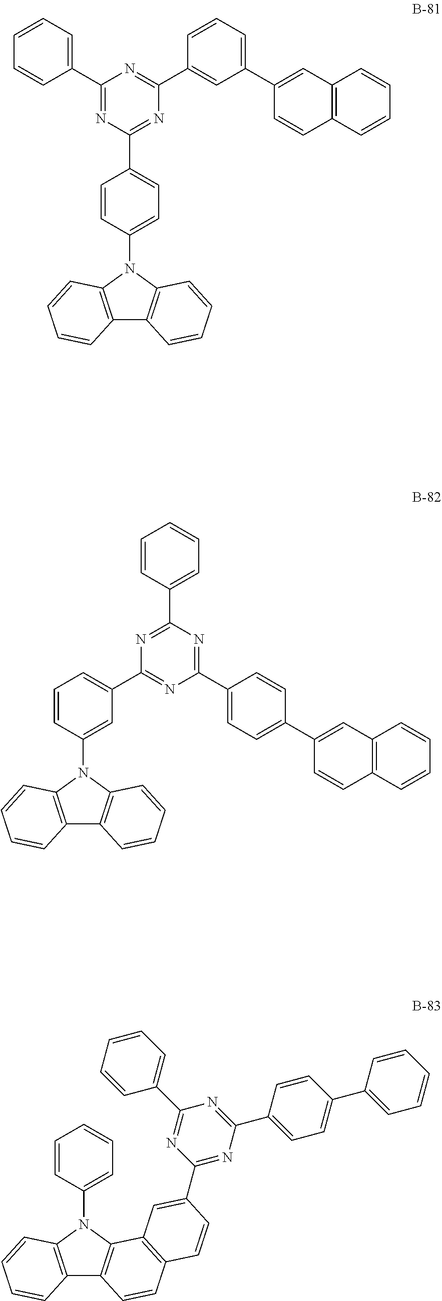 [SMILES: c1ccc(-c2nc(-c3ccc(-n4c5ccccc5c5ccccc54)cc3)nc(-c3cccc(-c4ccc5ccccc5c4)c3)n2)cc1 ; c1ccc(-c2nc(-c3ccc(-c4ccc5ccccc5c4)cc3)nc(-c3cccc(-n4c5ccccc5c5ccccc54)c3)n2)cc1 ; c1ccc(-c2ccc(-c3nc(-c4ccccc4)nc(-c4ccc5ccc6c7ccccc7n(-c7ccccc7)c6c5c4)n3)cc2)cc1]

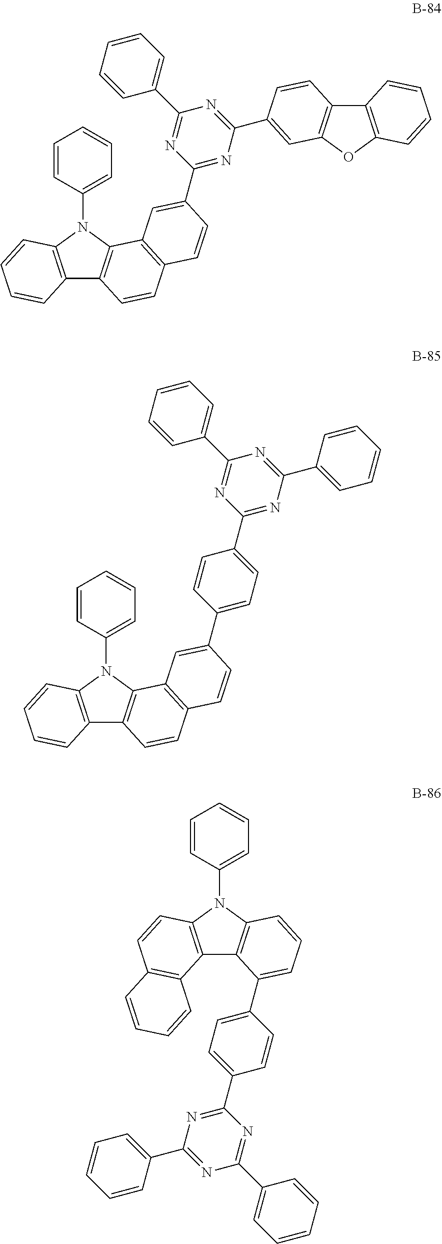 [SMILES: c1ccc(-c2nc(-c3ccc4c(c3)oc3ccccc34)nc(-c3ccc4ccc5c6ccccc6n(-c6ccccc6)c5c4c3)n2)cc1 ; c1ccc(-c2nc(-c3ccccc3)nc(-c3ccc(-c4ccc5ccc6c7ccccc7n(-c7ccccc7)c6c5c4)cc3)n2)cc1 ; c1ccc(-c2nc(-c3ccccc3)nc(-c3ccc(-c4cccc5c4c4c6ccccc6ccc4n5-c4ccccc4)cc3)n2)cc1]

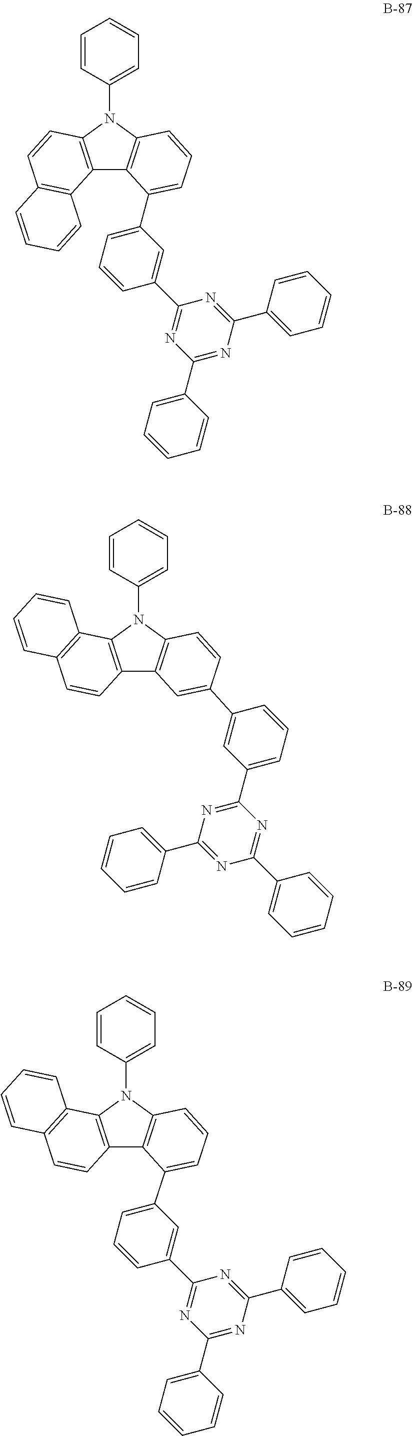 [SMILES: c1ccc(-c2nc(-c3ccccc3)nc(-c3cccc(-c4cccc5c4c4c6ccccc6ccc4n5-c4ccccc4)c3)n2)cc1 ; c1ccc(-c2nc(-c3ccccc3)nc(-c3cccc(-c4ccc5c(c4)c4ccc6ccccc6c4n5-c4ccccc4)c3)n2)cc1 ; c1ccc(-c2nc(-c3ccccc3)nc(-c3cccc(-c4cccc5c4c4ccc6ccccc6c4n5-c4ccccc4)c3)n2)cc1]

The first compound and the second compound may be included in a weight ratio of, e.g., about 1:99 to about 99:1. Within the range, a desirable weight ratio may be adjusted using a hole transport capability of the first compound and an electron transport capability of the second compound to realize bipolar characteristics and to help improve efficiency and life-span. Within the range, they may be included in a weight ratio of, e.g., about 10:90 to about 90:10, about 20:80 to about 80:20, about 30:70 to about 70:30, about 40:60 to about 60:40, or about 50:50. In an implementation, they may be included in a weight ratio of about 50:50 to about 60:40, e.g., about 50:50 or about 60:40.

In an implementation, the composition may include the compound represented by Chemical Formula 1E-2-2 as the first compound and the compound represented by one of Chemical Formula 3A-2, Chemical Formula 3B, Chemical Formula 3E-2, and Chemical Formula 3J as the second compound.

In an implementation, in Chemical Formula 1E-2-2, L^{a}, L^{b}, L^{c}, and L¹ to L⁴ may each independently be, e.g., a single bond, a substituted or unsubstituted phenylene group, a substituted or unsubstituted biphenylene group, a substituted or unsubstituted terphenylene group, or a substituted or unsubstituted naphthylene group, R^{a}, R¹, R², and R⁴ may each independently be, e.g., hydrogen, deuterium, a cyano group, a substituted or unsubstituted C1 to C10 alkyl group, or a substituted or unsubstituted C6 to C12 aryl group, R^{b} and R^{c} may each independently be, e.g., a substituted or unsubstituted phenyl group, a substituted or unsubstituted biphenyl group, a substituted or unsubstituted naphthyl group, a substituted or unsubstituted fluorenyl group, a substituted or unsubstituted carbazolyl group, a substituted or unsubstituted dibenzofuranyl group, a substituted or unsubstituted dibenzothiophenyl group, or a monovalent group of a fused ring compound represented by a combination of Chemical Formula 1 and Chemical Formula 2, and R⁵ and R⁶ may each independently be, e.g., a substituted or unsubstituted C1 to C10 alkyl group, or a substituted or unsubstituted C6 to C12 aryl group.

In an implementation, in Chemical Formula 3A-2, Chemical Formula 3B, Chemical Formula 3E-2, and Chemical Formula 3J, L⁵ to L¹⁰ may each independently be, e.g., a single bond, a substituted or unsubstituted phenylene group, a substituted or unsubstituted biphenylene group, a substituted or unsubstituted terphenylene group, or a substituted or unsubstituted naphthylene group, Ar may be, e.g., a substituted or unsubstituted phenyl group, a substituted or unsubstituted biphenyl group, or a substituted or unsubstituted naphthyl group, and R⁷ to R¹⁰, R^{e}, R^{f}, and R^{g} may each independently be, e.g., hydrogen, deuterium, a substituted or unsubstituted C1 to C10 alkyl group, or a substituted or unsubstituted C6 to C12 aryl group.

In an implementation, Z¹ to Z⁵ may each independently be, e.g., N or C-L^{d}-R^{d}, at least two of Z¹ to Z⁵ may be N, each L^{d} may independently be, e.g., a single bond, or a substituted or unsubstituted C6 to C12 arylene group, and each R^{d} may independently be, e.g., a substituted or unsubstituted phenyl group, a substituted or unsubstituted biphenyl group, a substituted or unsubstituted terphenyl group, a substituted or unsubstituted anthracenyl group, a substituted or unsubstituted naphthyl group, a substituted or unsubstituted phenanthrenyl group, a substituted or unsubstituted triphenylene group, a substituted or unsubstituted fluorenyl group, a substituted or unsubstituted carbazolyl group, a substituted or unsubstituted dibenzofuranyl group or a substituted or unsubstituted dibenzothiophenyl group.

In an implementation, the composition may further include one or more compounds in addition to the aforementioned first compound and second compound.

In an implementation, the composition may further include a dopant. The dopant may be, e.g., a phosphorescent dopant. The phosphorescent dopant may be, e.g., a red, green, or blue phosphorescent dopant. In an implementation, the dopant may be, e.g., a red phosphorescent dopant.

The dopant may be mixed with the first compound and the second compound in a small amount to cause light emission, and may be a material such as a metal complex that emits light by multiple excitation into a triplet or more. The dopant may be, e.g., an inorganic, organic, or organic/inorganic compound, and one or more types thereof may be used.

The dopant may be a phosphorescent dopant. Examples of the phosphorescent dopant may include an organometal compound including Ir, Pt, Os, Ti, Zr, Hf, Eu, Tb, Tm, Fe, Co, Ni, Ru, Rh, Pd, or a combination thereof. In an implementation, the phosphorescent dopant may be, e.g., a compound represented by Chemical Formula Z.

L¹¹MX [Chemical Formula Z]

In Chemical Formula Z, M may be, e.g., a metal, and L¹¹ and X may each independently be, e.g., a ligand to form a complex compound with M.

The M may be, e.g., Ir, Pt, Os, Ti, Zr, Hf, Eu, Tb, Tm, Fe, Co, Ni, Ru, Rh, Pd, or a combination thereof. L¹¹ and X may be, e.g., a bidendate ligand.

The composition may be formed into a film using a dry film-forming method such as chemical vapor deposition.

Hereinafter, an organic optoelectronic device to which the aforementioned composition is applied is described.

The organic optoelectronic device may be a suitable device to convert electrical energy into photoenergy and vice versa, and may be, e.g., an organic photoelectric device, an organic light emitting diode, an organic solar cell, and an organic photo-conductor drum.

Herein, an organic light emitting diode as one example of an organic optoelectronic device is described referring to drawings.

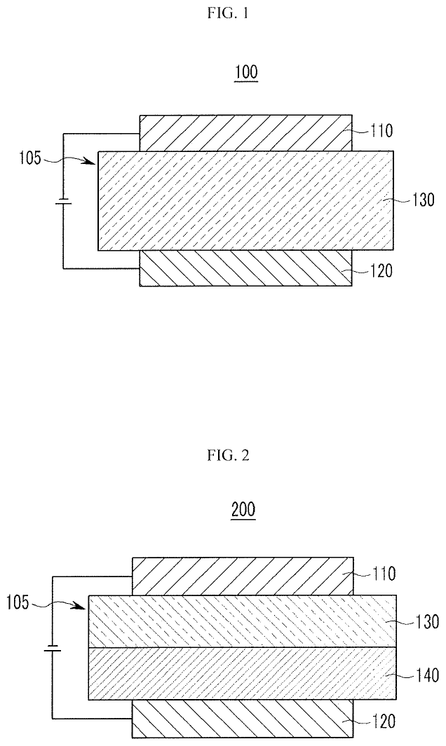

FIGS. 1 and 2 illustrate cross-sectional views of each organic light emitting diode according to an embodiment.

Referring to FIG. 1, an organic light emitting diode 100 according to one embodiment may include an anode 120 and a cathode 110 facing each other and an organic layer 105 between the anode 120 and cathode 110.

The anode 120 may be made of a conductor having a large work function to help hole injection, and may include e.g., a metal, a metal oxide and/or a conductive polymer. In an implementation, the anode 120 may include, e.g., a metal such as nickel, platinum, vanadium, chromium, copper, zinc, gold, and the like or an alloy thereof; a metal oxide such as zinc oxide, indium oxide, indium tin oxide (ITO), indium zinc oxide (IZO), and the like; a combination of a metal and an oxide such as ZnO and Al or SnO₂ and Sb; a conductive polymer such as poly(3-methylthiophene), poly(3,4-(ethylene-1,2-dioxy)thiophene) (PEDOT), polypyrrole, and polyaniline.

The cathode 110 may be made of a conductor having a small work function to help electron injection, and may include, e.g., a metal, a metal oxide and/or a conductive polymer. In an implementation, the cathode 110 may include, e.g., a metal such as magnesium, calcium, sodium, potassium, titanium, indium, yttrium, lithium, gadolinium, aluminum silver, tin, lead, cesium, barium, and the like, or an alloy thereof; a multi-layer structure material such as LiF/Al, LiO₂/Al, LiF/Ca, LiF/Al, and BaF₂/Ca.

The organic layer 105 may include a light emitting layer 130 including the aforementioned composition for the organic optoelectronic device.

The aforementioned composition for the organic optoelectronic device may be, e.g., a red light emitting composition.

The light emitting layer 130 may include, e.g., the aforementioned first compound and second compound as a phosphorescent host.

Referring to FIG. 2, an organic light emitting diode 200 may further include a hole auxiliary layer 140 as well as a light emitting layer 130. The hole auxiliary layer 140 may further increase hole injection and/or hole mobility between the anode 120 and light emitting layer 130 and block electrons. The hole auxiliary layer 140 may be, e.g., a hole transport layer, a hole injection layer, and/or an electron blocking layer, and may include at least one layer.

The hole auxiliary layer 140 may include e.g., a compound of the following Group E.

In an implementation, the hole auxiliary layer 140 may include a hole transport layer between the anode 120 and the light emitting layer 130 and a hole transport auxiliary layer between the light emitting layer 130 and the hole transport layer, and a compound of Group E may be included in the hole transport auxiliary layer.

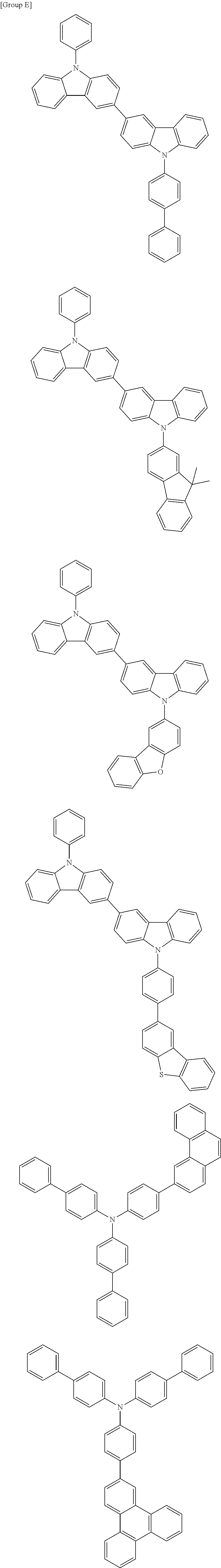

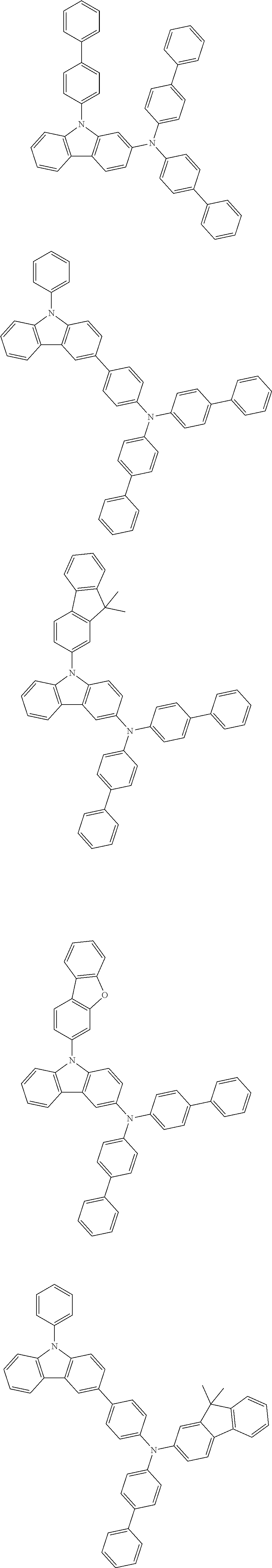

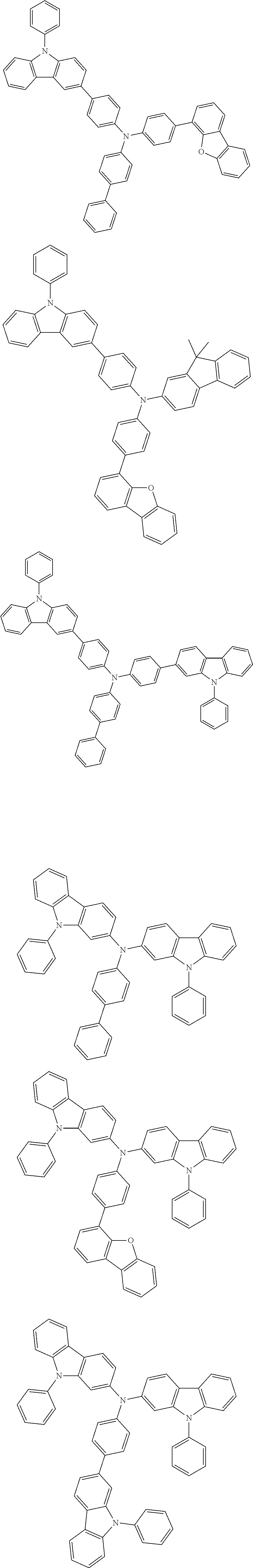

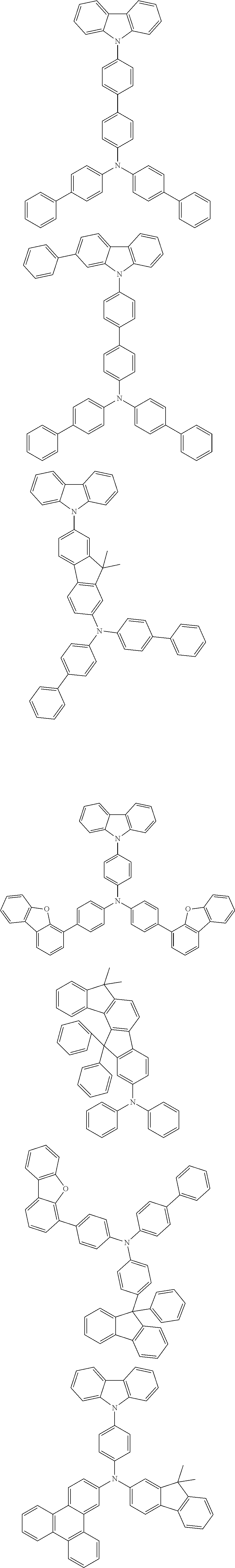

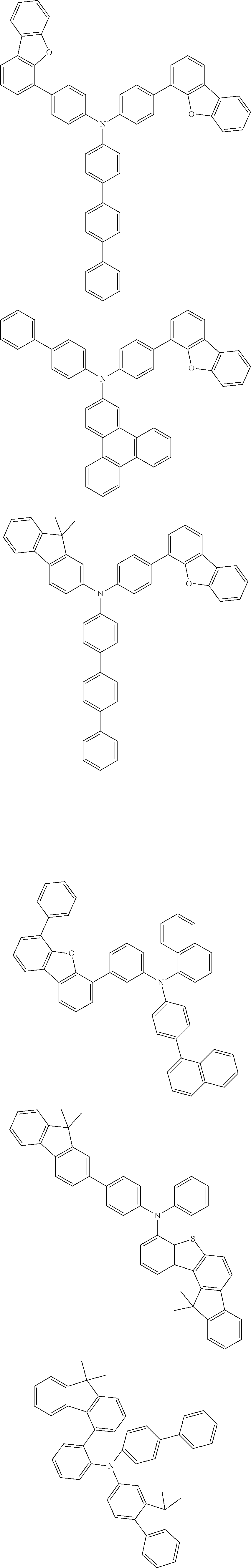

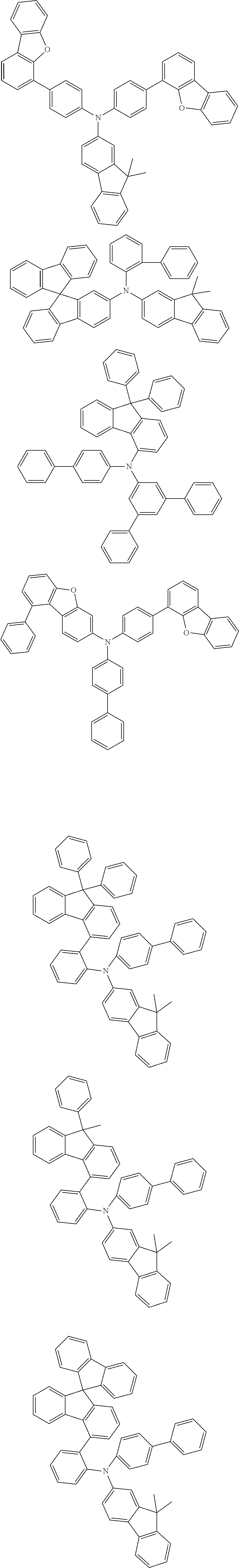

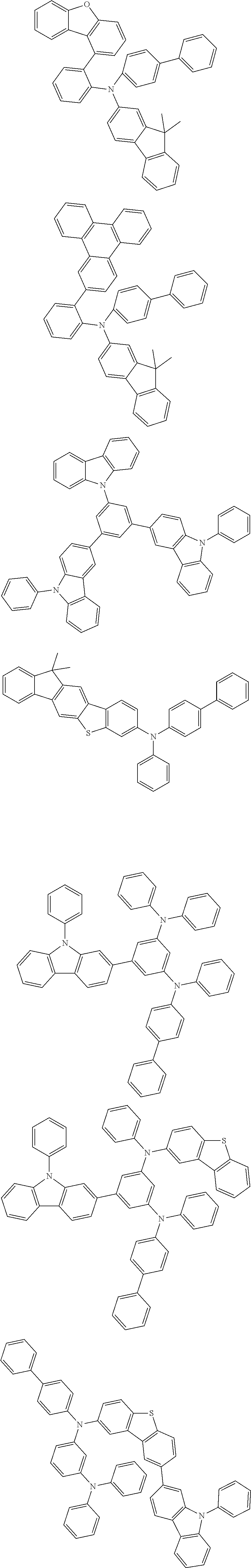

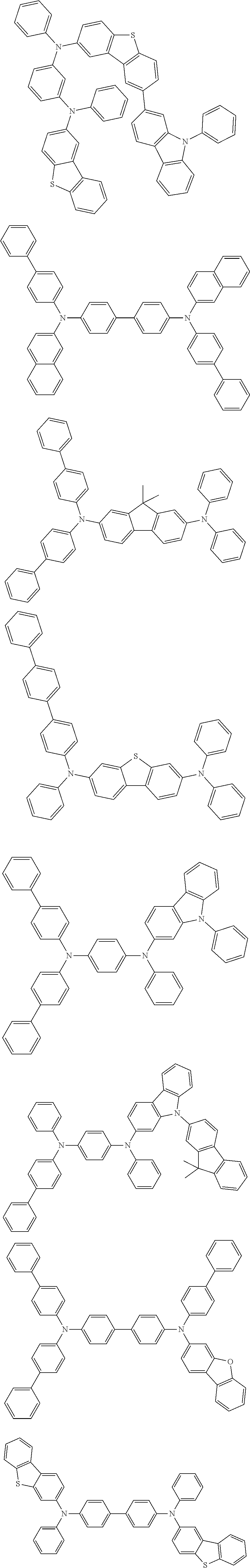

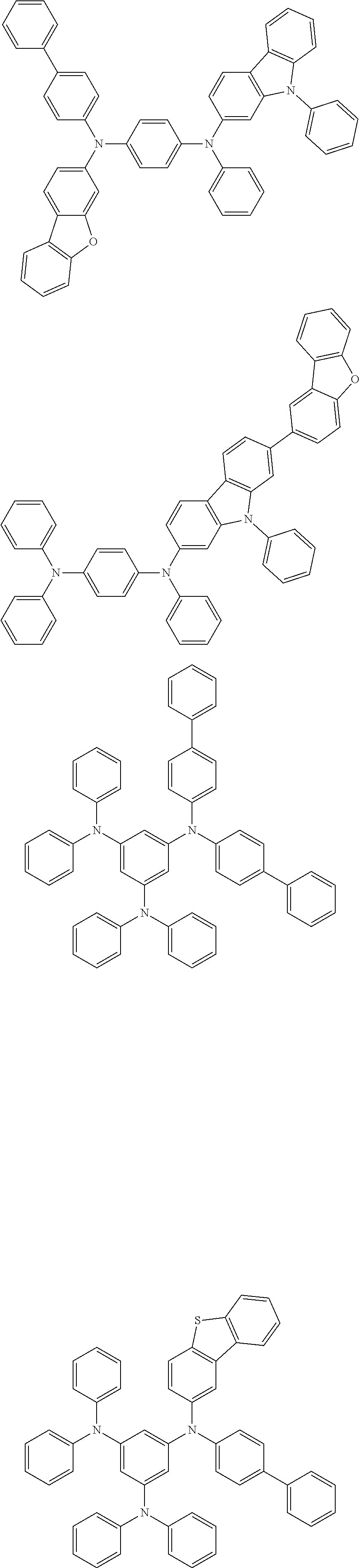

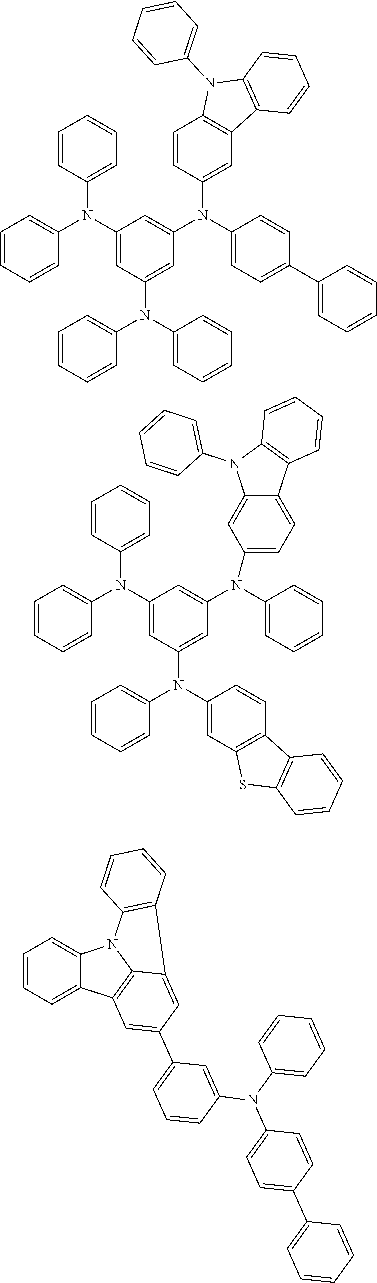

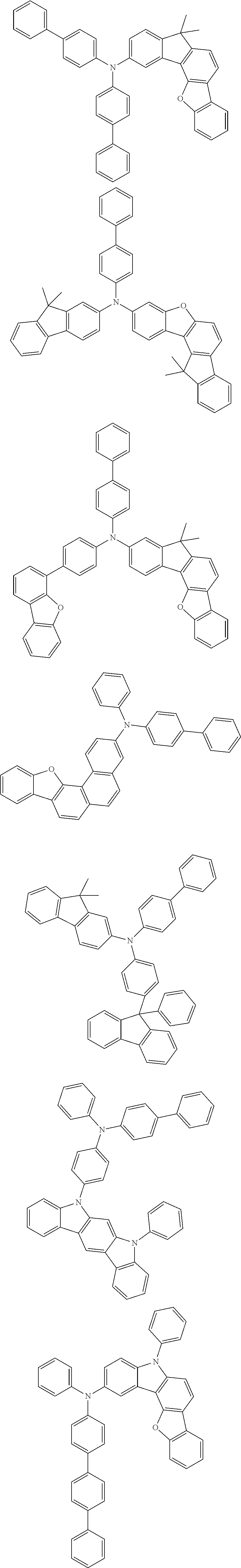

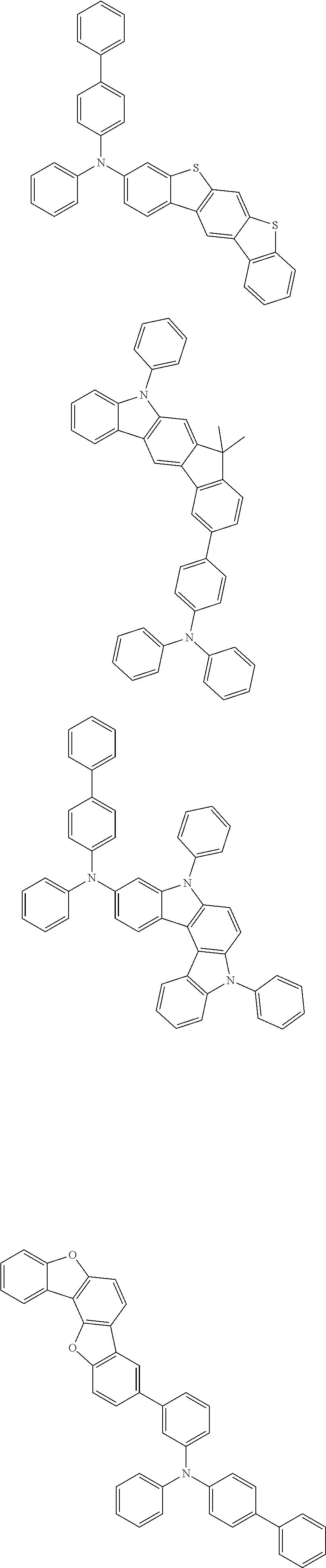 [SMILES: c1ccc(-c2ccc(N(c3ccccc3)c3ccc4c(c3)sc3cc5sc6ccccc6c5cc34)cc2)cc1 ; CC1(C)c2ccc(-c3ccc(N(c4ccccc4)c4ccccc4)cc3)cc2-c2cc3c4ccccc4n(-c4ccccc4)c3cc21 ; c1ccc(-c2ccc(N(c3ccccc3)c3ccc4c5c6c7ccccc7n(-c7ccccc7)c6ccc5n(-c5ccccc5)c4c3)cc2)cc1 ; c1ccc(-c2ccc(N(c3ccccc3)c3cccc(-c4ccc5oc6c(ccc7oc8ccccc8c76)c5c4)c3)cc2)cc1]

In the hole transport auxiliary layer, suitable compounds may be used in addition to the aforementioned compounds.

In an implementation, the organic light emitting diode may further include an electron transport layer, an electron injection layer, or a hole injection layer in the organic layer 105.

The organic light emitting diodes 100 and 200 may be manufactured by forming an anode or a cathode on a substrate, forming an organic layer using a dry film formation method such as a vacuum deposition method (evaporation), sputtering, plasma plating, and ion plating, and forming a cathode or an anode thereon.

The organic light emitting diode may be applied to an organic light emitting display device.

Hereinafter, starting materials and reactants used in Examples and Synthesis Examples were purchased from Sigma-Aldrich Co. Ltd., TCI Inc., Tokyo chemical industry or P&H tech as far as there in no particular comment or were synthesized by known methods.

The following Examples and Comparative Examples are provided in order to highlight characteristics of one or more embodiments, but it will be understood that the Examples and Comparative Examples are not to be construed as limiting the scope of the embodiments, nor are the Comparative Examples to be construed as being outside the scope of the embodiments. Further, it will be understood that the embodiments are not limited to the particular details described in the Examples and Comparative Examples.

The following synthesis intermediates were synthesized according to suitable methods.

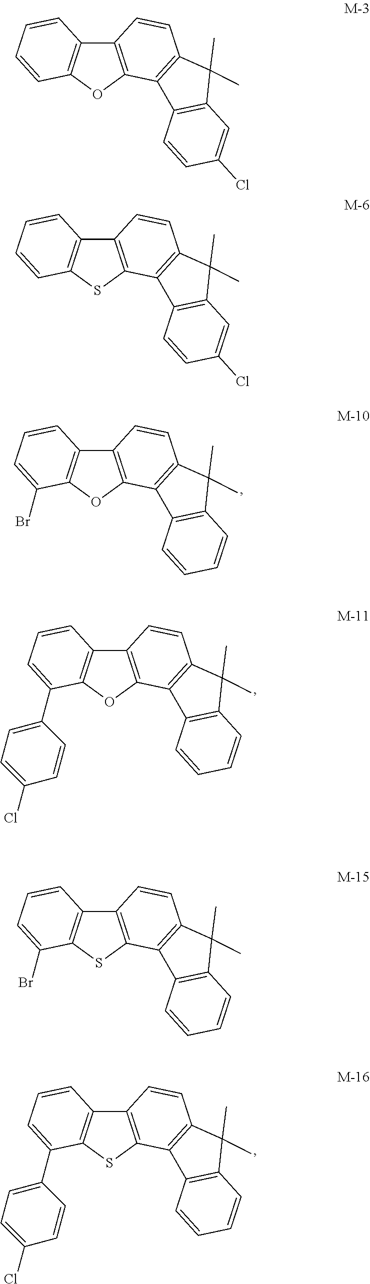 [SMILES: CC1(C)c2cc(Cl)ccc2-c2c1ccc1c2oc2ccccc21 ; CC1(C)c2cc(Cl)ccc2-c2c1ccc1c2sc2ccccc21 ; CC1(C)c2ccccc2-c2c1ccc1c2oc2c(Br)cccc21 ; CC1(C)c2ccccc2-c2c1ccc1c2oc2c(-c3ccc(Cl)cc3)cccc21 ; CC1(C)c2ccccc2-c2c1ccc1c2sc2c(Br)cccc21 ; CC1(C)c2ccccc2-c2c1ccc1c2sc2c(-c3ccc(Cl)cc3)cccc21]

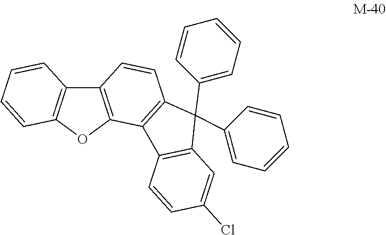 [SMILES: Clc1ccc2c(c1)C(c1ccccc1)(c1ccccc1)c1ccc3c(oc4ccccc43)c1-2]

### Preparation of First Compound for Organic Optoelectronic Device

### Synthesis Example 1: Synthesis of Compound A-51

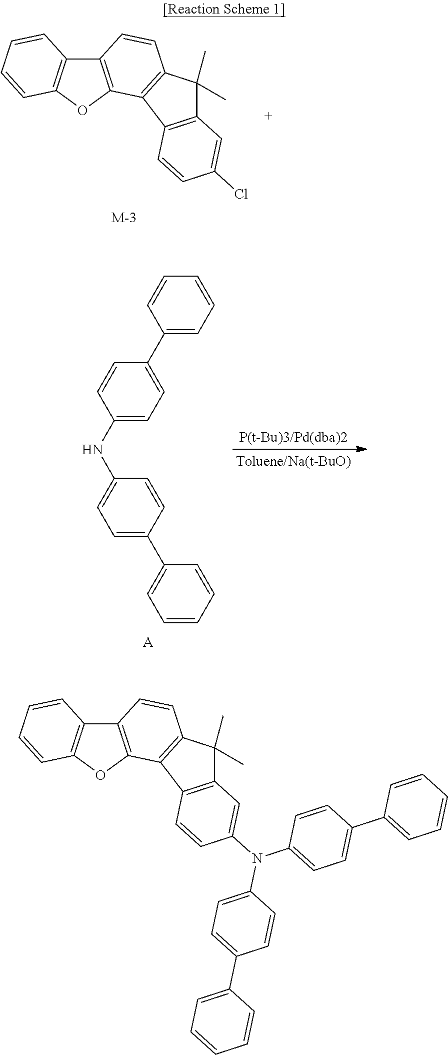

5.0 g (15.68 mmol) of Intermediate M-3, 5.04 g (15.68 mmol) of Intermediate A, 4.52 g (47.95 mmol) of sodium t-butoxide, and 0.1 g (0.47 mmol) of tri-tert-butylphosphine were dissolved in 200 ml of toluene, and 0.27 g (0.47 mmol) of Pd(dba)₂ was added thereto. The mixture was refluxed and stirred under a nitrogen atmosphere for 12 hours. When the reaction was complete, the resultant was extracted with toluene and distilled water. The obtained organic layer was dried with anhydrous magnesium sulfate, filtered, and concentrated under a reduced pressure. The resulting product was purified through silica gel column chromatography using n-hexane/dichloromethane mixed in a volume ratio of 2:1, obtaining 7.8 g (Yield: 82.3%) of a desired compound A-51 as a white solid.

Calculated value: C, 89.52; H, 5.51; N, 2.32; O, 2.65

Analyzed value: C, 89.51; H, 5.52; N, 2.32; O, 2.65

### Synthesis Example 2: Synthesis of Compound A-81

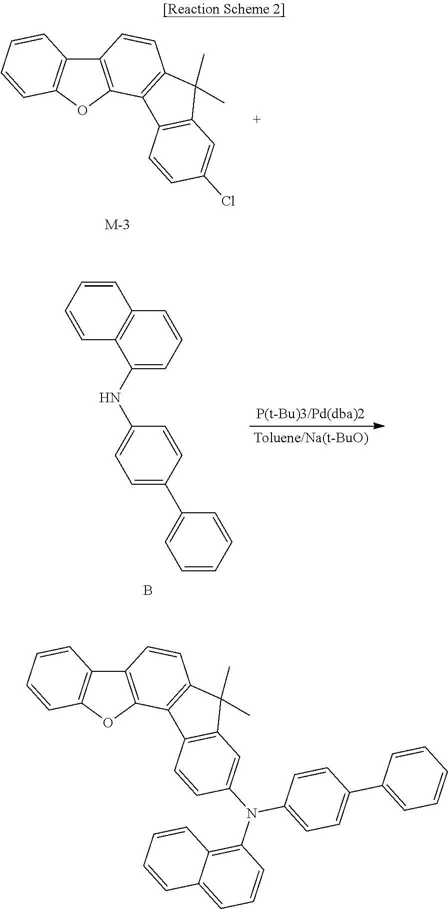

Compound A-81 was synthesized according to the same method as Synthesis Example 1 except that Intermediate M-3 and Intermediate B were used in a ratio of 1:1 (7.8 g, Yield: 80.5%).

Calculation value: C, 89.40; H, 5.41; N, 2.42; O, 2.77

Analyzed value: C, 89.42; H, 5.39; N, 2.42; O, 2.77

### Synthesis Example 3: Synthesis of Compound A-82

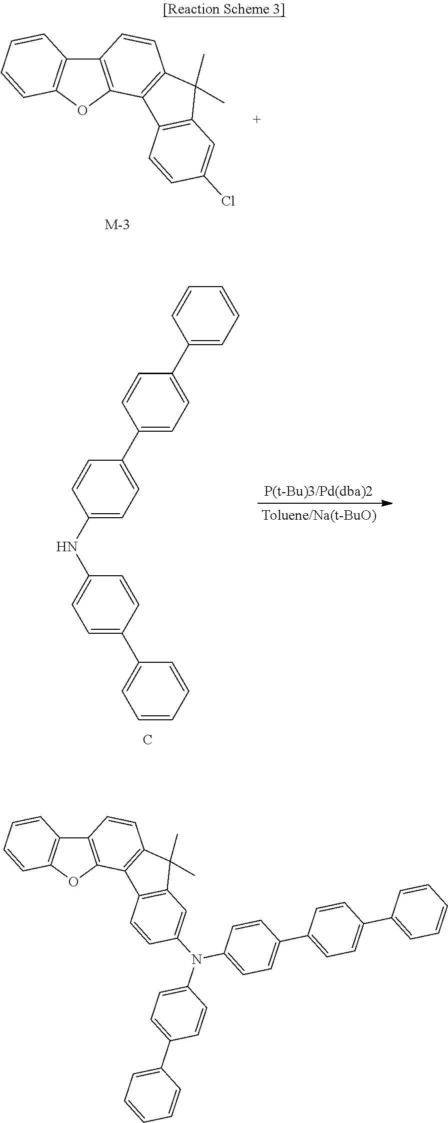

Compound A-82 was synthesized according to the same method as Synthesis Example 1 except that Intermediate M-3 and Intermediate C were used in a ratio of 1:1 (9.2 g, Yield: 86.2%).

Calculation value: C, 90.10; H, 5.49; N, 2.06; O, 2.35

Analyzed value: C, 90.12; H, 5.47; N, 2.06; O, 2.35

### Synthesis Example 4: Synthesis of Compound A-55

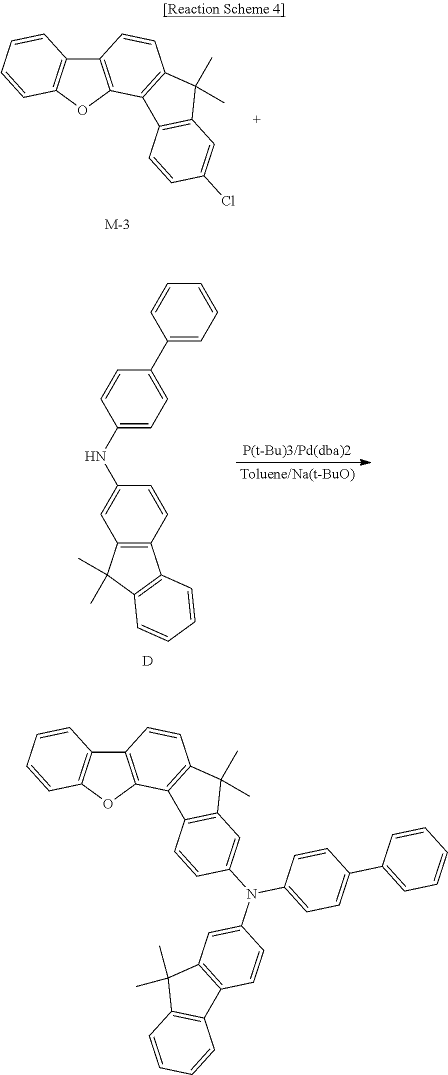

Compound A-55 was synthesized according to the same method as Synthesis Example 1 except that Intermediate M-3 and Intermediate D were used in an equivalence ratio of 1:1 (8.6 g, Yield: 85.1%).

Calculation value: C, 89.55; H, 5.79; N, 2.18; O, 2.49

Analyzed value: C, 89.56; H, 5.78; N, 2.18; O, 2.49

### Synthesis Example 5: Synthesis of Compound A-69

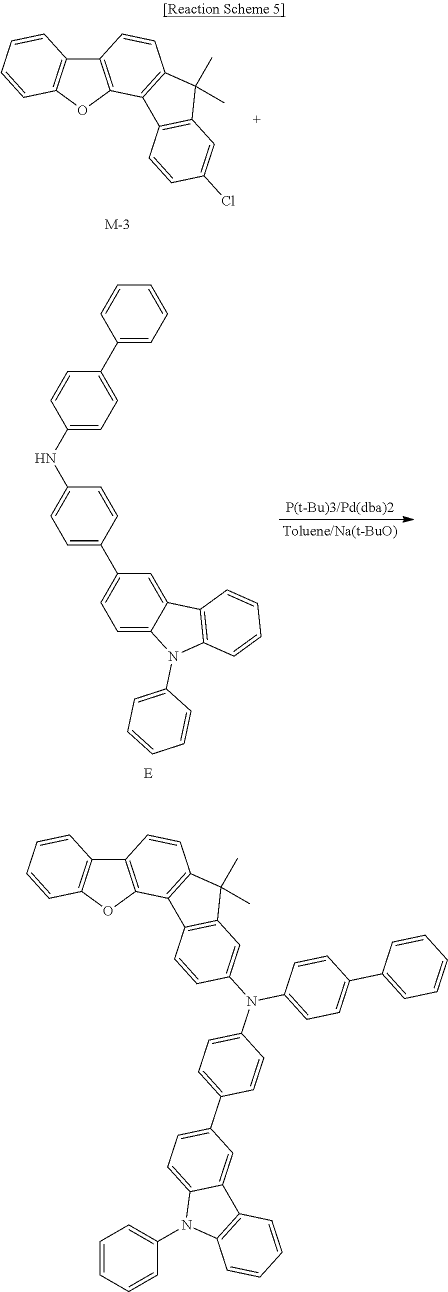

Compound A-69 was synthesized according to the same method as Synthesis Example 1 except that Intermediate M-3 and Intermediate E were used in an equivalence ratio of 1:1 (10.5 g, Yield: 87%).

Calculation value: C, 89.03; H, 5.24; N, 3.64; O, 2.08

Analyzed value: C, 89.01; H, 5.26; N, 3.64; O, 2.08

### Synthesis Example 6: Synthesis of Compound A-75

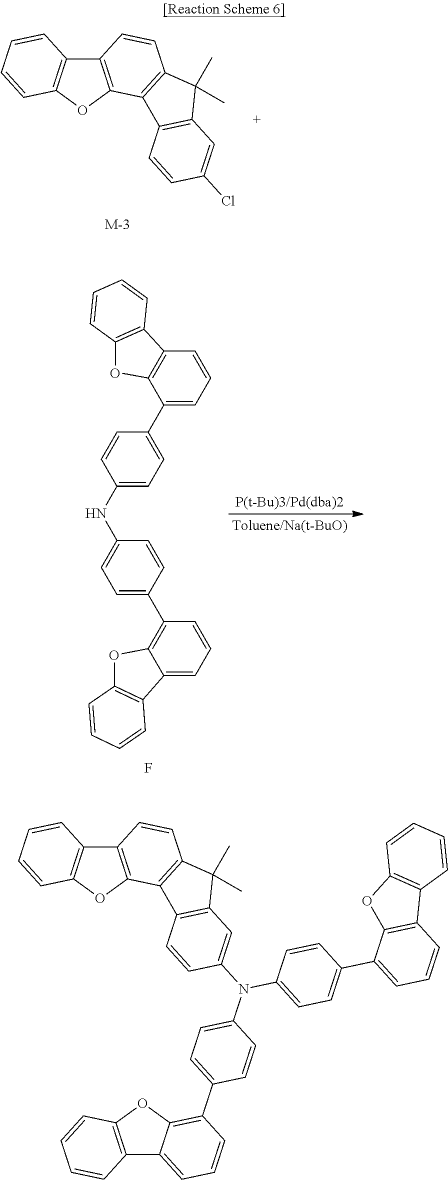

Compound A-75 was synthesized according to the same method as Synthesis Example 1 except that Intermediate M-3 and Intermediate F were used in an equivalence ratio of 1:1 (10.7 g, Yield: 87%).

Calculation value: C, 87.33; H, 4.76; N, 1.79; O, 6.12

Analyzed value: C, 87.31; H, 4.78; N, 1.79; O, 6.12

### Synthesis Example 7: Synthesis of Compound A-77

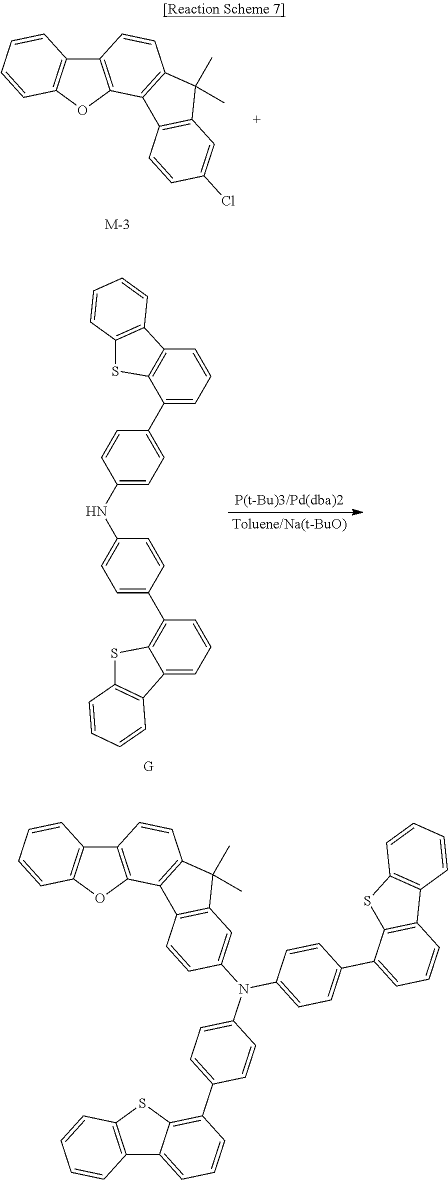

Compound A-77 was synthesized according to the same method as Synthesis Example 1 except that Intermediate M-3 and Intermediate G were used in an equivalence ratio of 1:1 (10.4 g, Yield: 81.2%).

Calculation value: C, 83.89; H, 4.57; N, 1.72; O, 1.96; S, 7.86

Analyzed value: C, 83.86; H, 4.59; N, 1.72; O, 1.96; S, 7.86

### Synthesis Example 8: Synthesis of Compound A-79

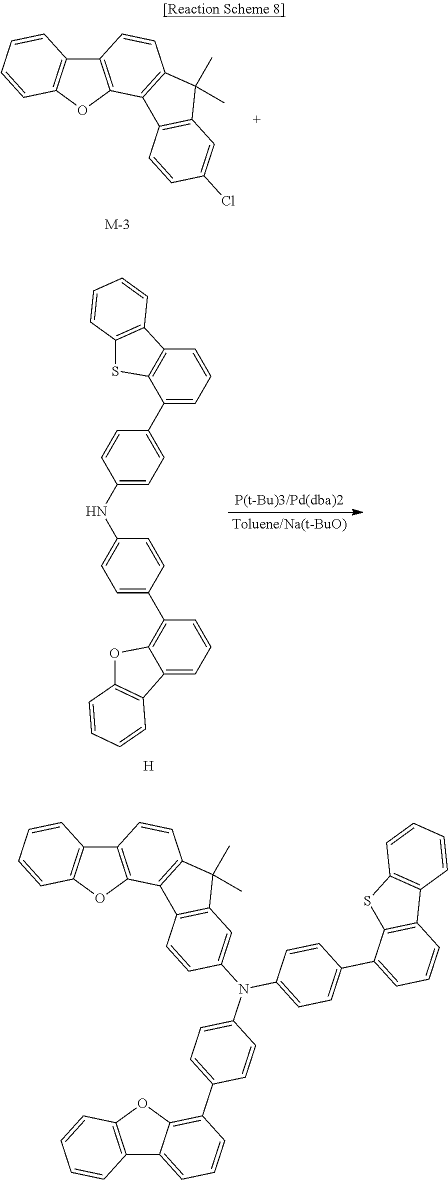

Compound A-79 was synthesized according to the same method as Synthesis Example 1 except that Intermediate M-3 and Intermediate H were used in an equivalence ratio of 1:1 (10.8 g, Yield: 86%).

Calculation value: C, 85.58; H, 4.66; N, 1.75; O, 4.00; S, 4.01

Analyzed value: C, 85.59; H, 4.67; N, 1.75; O, 4.00; S, 4.01

### Synthesis Example 9: Synthesis of Compound A-83

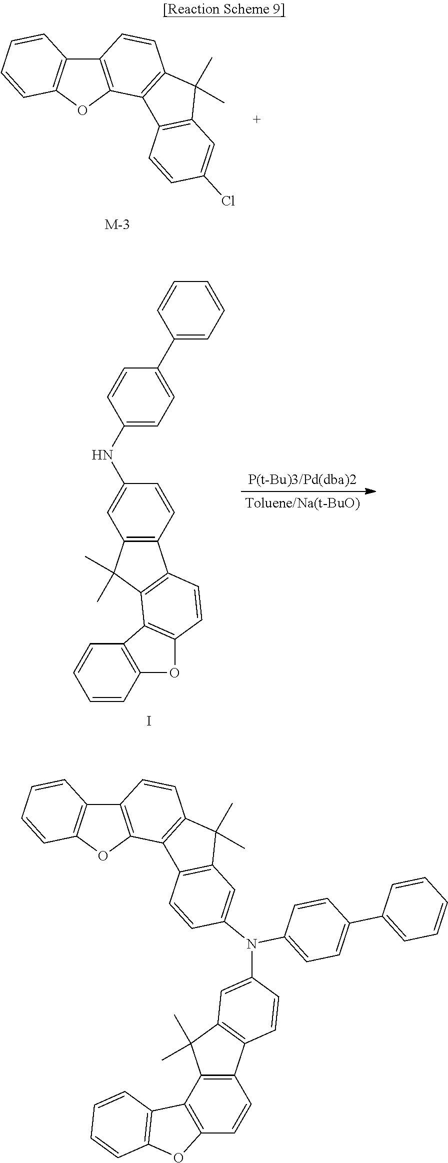

Compound A-83 was synthesized according to the same method as Synthesis Example 1 except that Intermediate M-3 and Intermediate I were used in an equivalence ratio of 1:1 (9.4 g, Yield: 81.6%).

Calculation value: C, 88.37; H, 5.36; N, 1.91; O, 4.36

Analyzed value: C, 88.35; H, 5.38; N, 1.91; O, 4.36

### Synthesis Example 10: Synthesis of Compound A-84

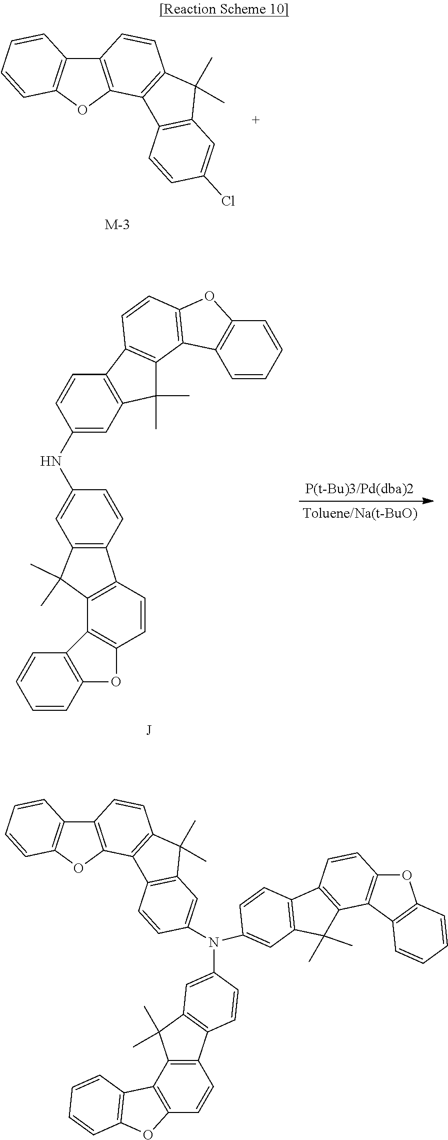 [SMILES: CC1(C)c2cc(Cl)ccc2-c2c1ccc1c2oc2ccccc21 ; "M-3" ; CC1(C)c2cc(Nc3ccc4c(c3)C(C)(C)c3c-4ccc4oc5ccccc5c34)ccc2-c2ccc3oc4ccccc4c3c21 ; CC1(C)c2cc(N(c3ccc4c(c3)C(C)(C)c3c-4ccc4oc5ccccc5c34)c3ccc4c(c3)C(C)(C)c3c-4ccc4oc5ccccc5c34)ccc2-c2c1ccc1c2oc2ccccc21]

Compound A-84 was synthesized according to the same method as Synthesis Example 1 except that Intermediate M-3 and Intermediate J were used in an equivalence ratio of 1:1 (10.4 g, Yield: 76.7%).

Calculation value: C, 87.57; H, 5.25; N, 1.62; O, 5.56

Analyzed value: C, 87.59; H, 5.23; N, 1.62; O, 5.56

### Synthesis Example 11: Synthesis of Compound A-52

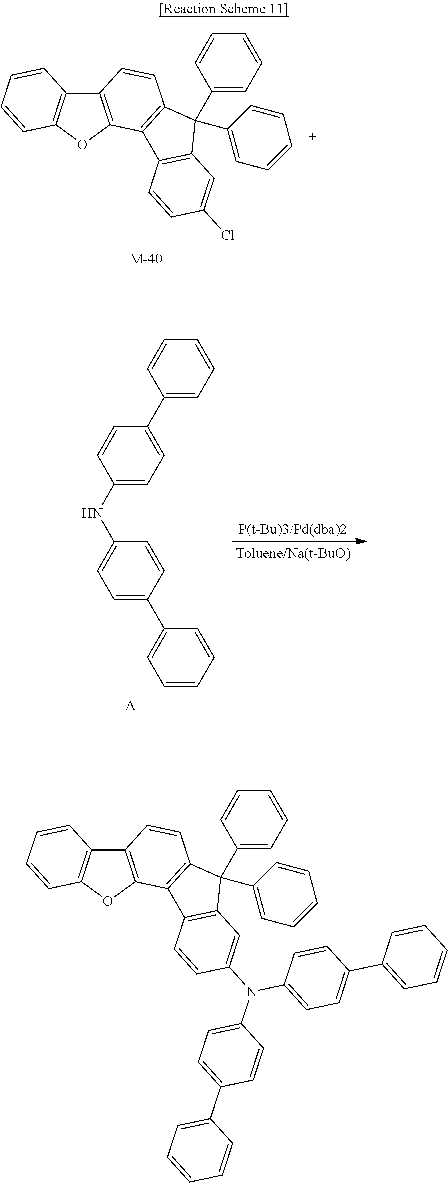

Compound A-52 was synthesized according to the same method as Synthesis Example 1 except that Intermediate M-40 and Intermediate A were used in an equivalence ratio of 1:1 (7.3 g, Yield: 88.8%).

Calculation value: C, 90.75; H, 5.12; N, 1.92; O, 2.20

Analyzed value: C, 90.73; H, 5.14; N, 1.92; O, 2.20

### Synthesis Example 12: Synthesis of Compound A-53

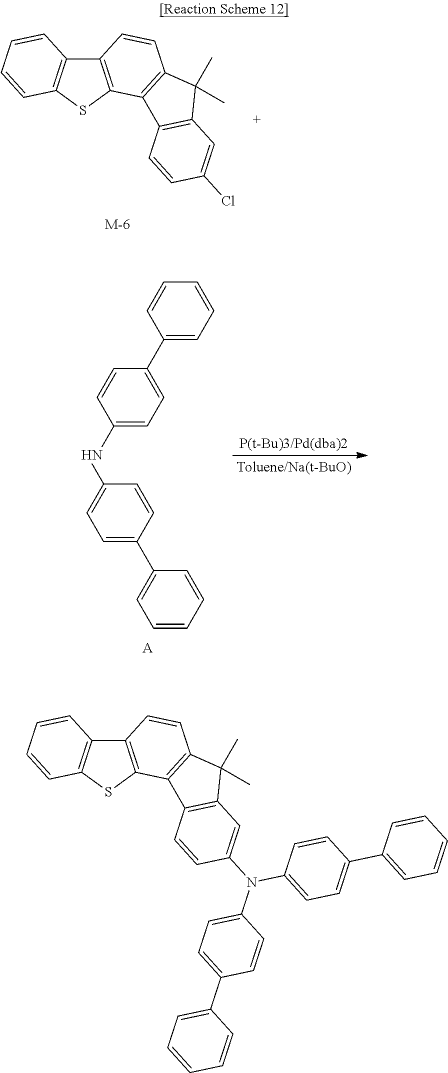

Compound A-53 was synthesized according to the same method as Synthesis Example 1 except that Intermediate M-6 and Intermediate A were used in an equivalence ratio of 1:1 (7.5 g, Yield: 81%).

Calculation value: C, 87.20; H, 5.37; N, 2.26; S, 5.17

Analyzed value: C, 87.22; H, 5.35; N, 2.26; S, 5.17

### Synthesis Example 13: Synthesis of Compound A-86

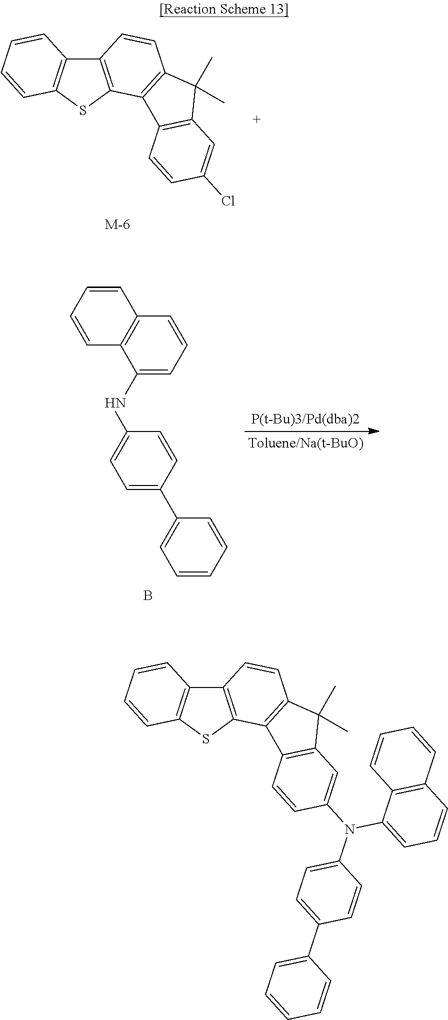

Compound A-86 was synthesized according to the same method as Synthesis Example 1 except that Intermediate M-6 and Intermediate B were used in an equivalence ratio of 1:1 (7.6 g, Yield: 85.7%).

Calculation value: C, 86.98; H, 5.26; N, 2.36; S, 5.40

Analyzed value: C, 86.99; H, 5.25; N, 2.36; S, 5.40

### Synthesis Example 14: Synthesis of Compound A-87

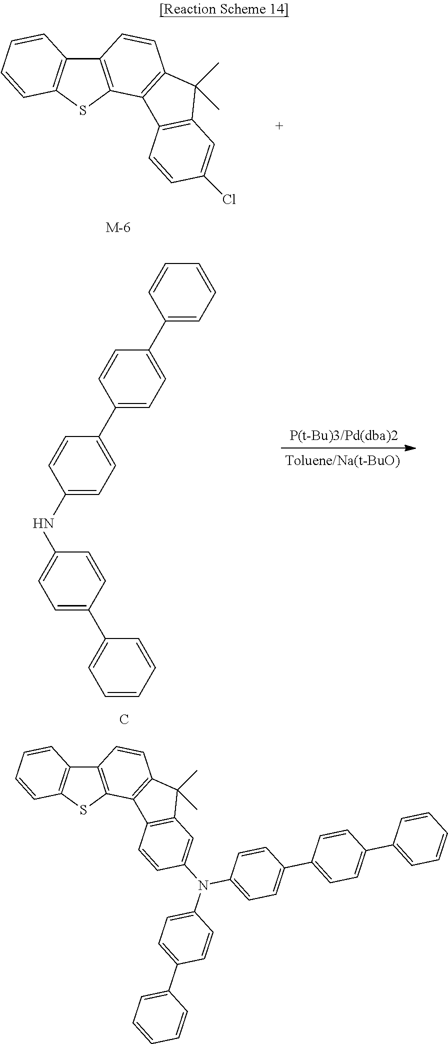

Compound A-87 was synthesized according to the same method as Synthesis Example 1 except that Intermediate M-6 and Intermediate C were used in an equivalence ratio of 1:1 (8.2 g, Yield: 78.9%).

Calculation value: C, 88.02; H, 5.36; N, 2.01; S, 4.61

Analyzed value: C, 88.00; H, 5.38; N, 2.01; S, 4.61

### Synthesis Example 15: Synthesis of Compound A-58

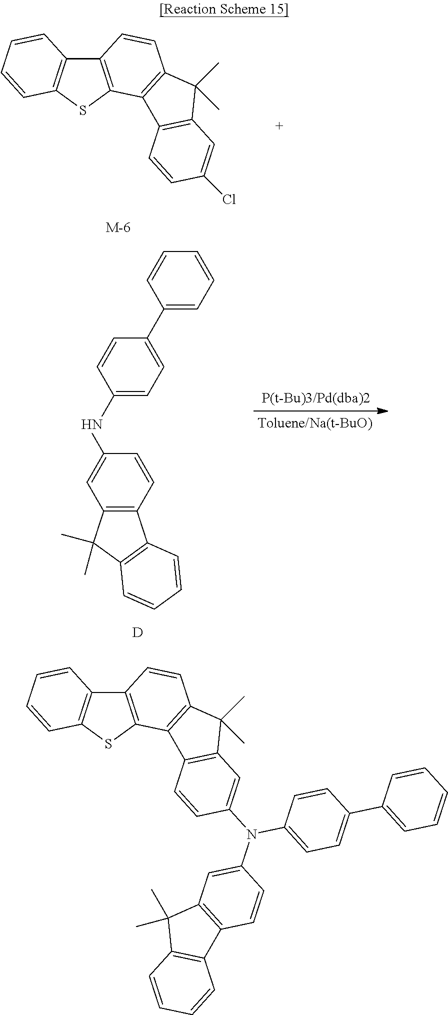

Compound A-58 was synthesized according to the same method as Synthesis Example 1 except that Intermediate M-6 and Intermediate D were used in an equivalence ratio of 1:1 (8.4 g, Yield: 85.2%).

Calculation value: C, 87.37; H, 5.65; N, 2.12; S, 4.86

Analyzed value: C, 87.35; H, 5.67; N, 2.12; S, 4.86

### Synthesis Example 16: Synthesis of Compound A-27

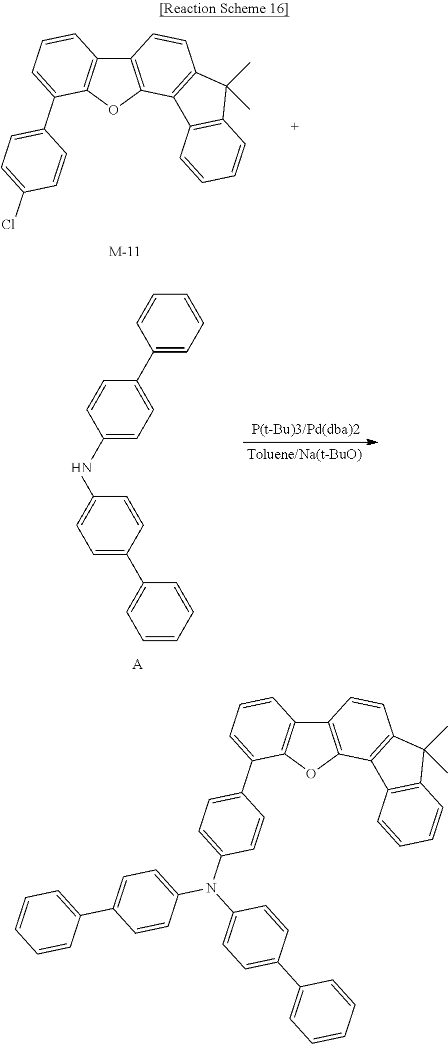

Compound A-27 was synthesized according to the same method as Synthesis Example 1 except that Intermediate M-1 and Intermediate A were used in an equivalence ratio of 1:1 (7.3 g, Yield: 84.8%).

Calculation value: C, 90.10; H, 5.49; N, 2.06; O, 2.35

Analyzed value: C, 90.12; H, 5.47; N, 2.06; O, 2.35

### Synthesis Example 17: Synthesis of Compound A-29

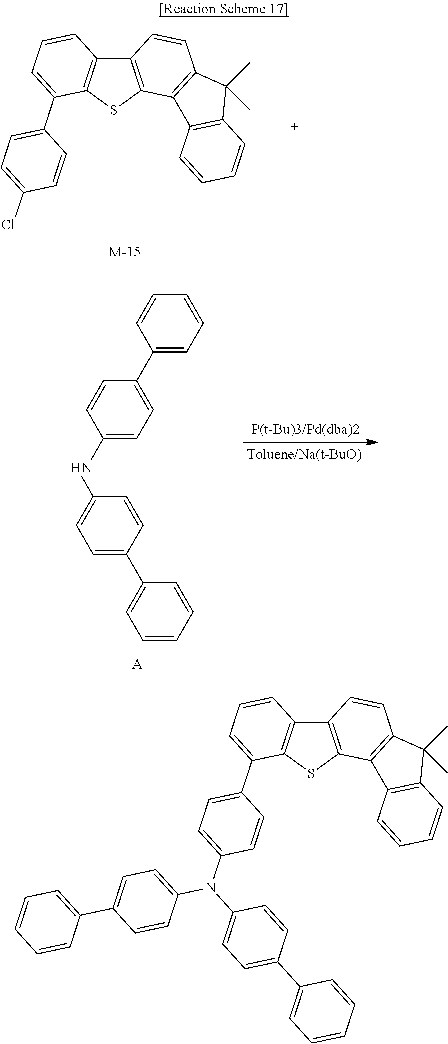

Compound A-29 was synthesized according to the same method as Synthesis Example 1 except that Intermediate M-16 and Intermediate A were used in an equivalence ratio of 1:1 (7.1 g, Yield: 83.8%).

Calculation value: C, 88.02; H, 5.36; N, 2.01; S, 4.61

Analyzed value: C, 88.04; H, 5.34; N, 2.01; S, 4.61

### Synthesis Example 18: Synthesis of Compound A-92

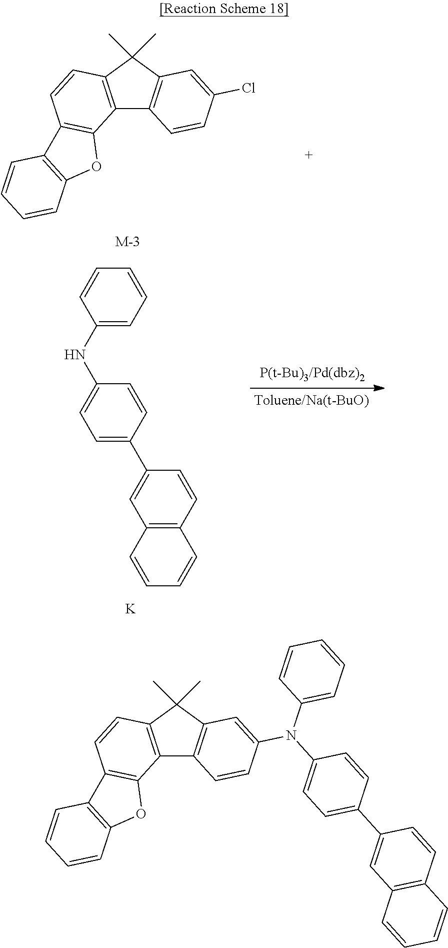

Compound A-92 was synthesized according to the same method as Synthesis Example 1 except that Intermediate M-3 and Intermediate K were used in an equivalence ratio of 1:1.

LC/MS calculated for: C43H31NO Exact Mass: 577.24 found for 577.77 [M+H].

### Synthesis Example 19: Synthesis of Compound A-93

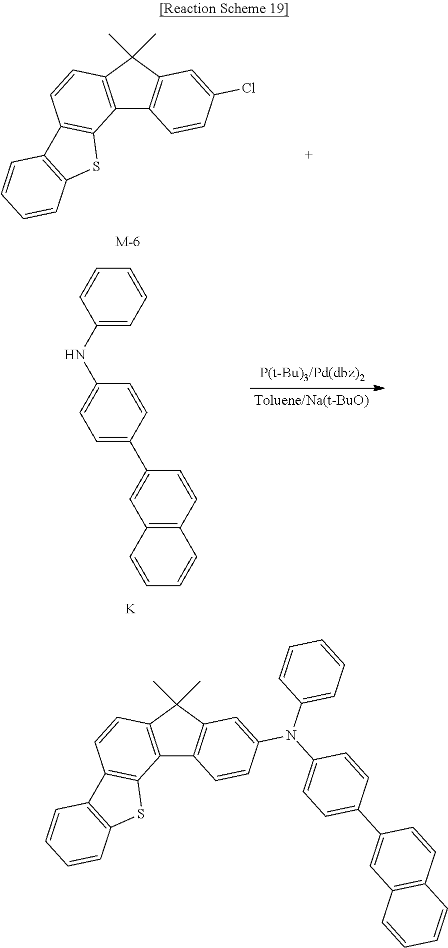

Compound A-93 was synthesized according to the same method as Synthesis Example 1 except that Intermediate M-6 and Intermediate K were used in an equivalence ratio of 1:1.

LC/MS calculated for: C43H31NS Exact Mass: 593.22 found for 593.78 [M+H].

### Comparative Synthesis Example 1: Synthesis of Comparative Compound 1

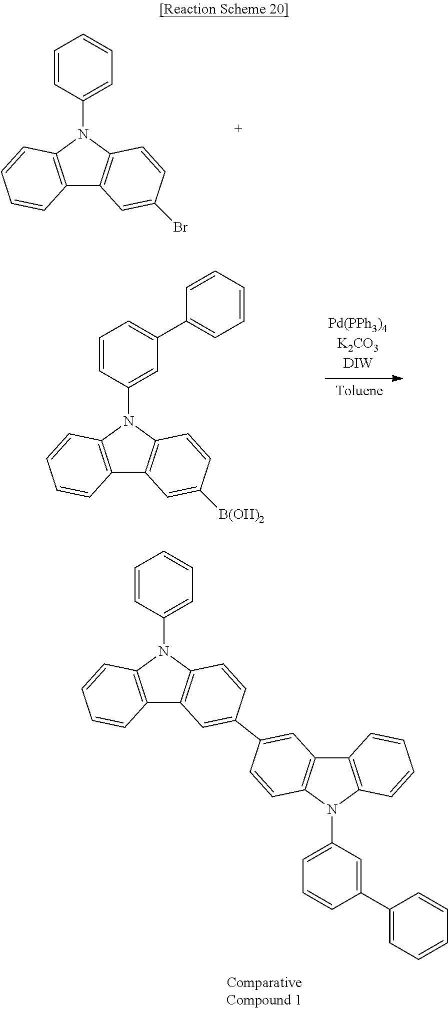 [SMILES: Brc1ccc2c(c1)c1ccccc1n2-c1ccccc1 ; OB(O)c1ccc2c(c1)c1ccccc1n2-c1cccc(-c2ccccc2)c1 ; c1ccc(-c2cccc(-n3c4ccccc4c4cc(-c5ccc6c(c5)c5ccccc5n6-c5ccccc5)ccc43)c2)cc1]

3-Bromo-9-phenylcarbazole (9.97 g, 30.95 mmol) was dissolved in 200 mL of toluene in an nitrogen environment, biphenylcarbazolylboronic acid (12.37 g, 34.05 mmol) and tetrakis(triphenylphosphine)palladium (1.07 g, 0.93 mmol) were added thereto, and the obtained mixture was stirred. Potassium carbonate saturated in water (12.83 g, 92.86 mmol) was added thereto, and the obtained mixture was heated and refluxed at 90° C. for 12 hours. When a reaction was complete, water was added to the reaction solution, and an extract was obtained by using dichloromethane (DCM), filtered after removing moisture therefrom by using anhydrous MgSO₄, and concentrated under a reduced pressure. A residue obtained therefrom was separated and purified through flash column chromatography to obtain Comparative Compound 1 (16 g, 92%).

LC/MS calculated for: C42H28N2 Exact Mass: 560.69 found for 560.73 [M+H].

### Comparative Synthesis Example 2: Synthesis of Comparative Compound 2

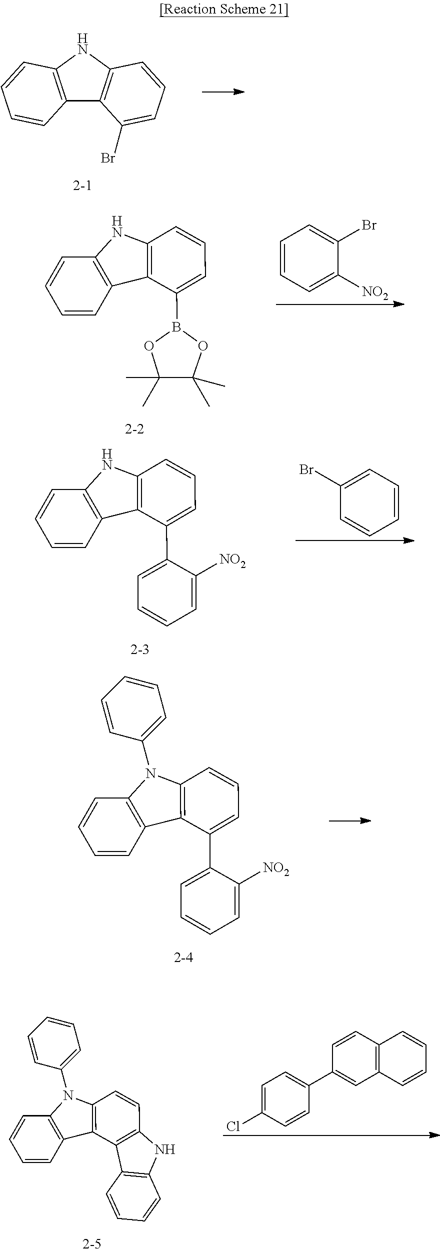

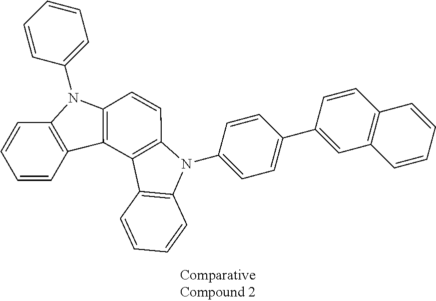 [SMILES: c1ccc(-n2c3ccccc3c3c4c5ccccc5n(-c5ccc(-c6ccc7ccccc7c6)cc5)c4ccc32)cc1]

Comparative Compound 2 was synthesized according to Reaction Scheme 21.

### Preparation of Second Compound for Organic Optoelectronic Device

### Synthesis Example 20: Synthesis of Compound B-12

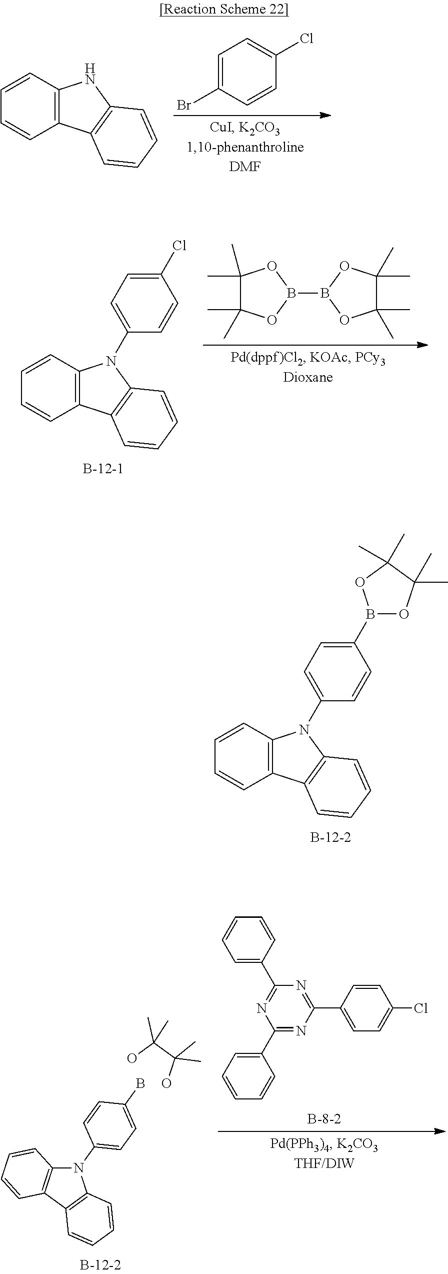

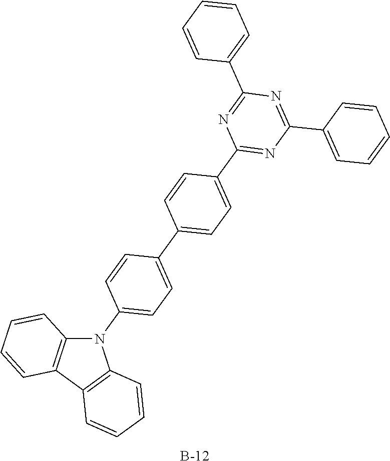

a) Synthesis of Intermediate B-12-1

Carbazole (35 g, 209.3 mmol), 1-bromo-4-chloro-benzene (60.11 g 313.98 mmol), CuI (3.99 g, 20.9 mmol), K₂CO₃ (43.39 g, 313.98 mmol), and 1,10-phenanthroline (3.77 g, 20.9 mmol) were put in a round-bottomed flask and dissolved in DMF (700 ml). The solution was stirred at 180° C. for 18 hours. When a reaction was complete, after removing a reaction solvent under a reduced pressure, a product therefrom was dissolved in dichloromethane and filtered through silica gel. After concentrating the dichloromethane, the product was recrystallized with hexane to obtain 40.0 g (68.8%) of Intermediate B-12-1.

b) Synthesis of Intermediate B-12-2

Intermediate B-12-1 (40 g, 144 mmol), bis(pinacolato)diboron (54.86 g, 216 mmol), Pd(dppf)C12 (7.1 g, 8.64 mmol), tricyclohexylphosphine (8.08 g, 28.8 mmol), and potassium acetate (42.4 g, 432.04 mmol) were put in a round-bottomed flask and dissolved in DMF (720 ml). The mixture was refluxed and stirred at 120° C. for 12 hours. When a reaction was complete, the mixture was poured into an excess of distilled water and then, stirred for one hour. A solid therein was filtered and dissolved in DCM. After removing moisture therefrom with MgSO₄, an organic solvent was filtered through a silica gel pad and removed under a reduced pressure. The solid therefrom was recrystallized with EA and hexane to obtain 31.3 g (58.9%) of Intermediate B-12-2.

c) Synthesis of Compound B-12

Intermediate B-12-2 (31 g, 83.95 mmol) was dissolved in 0.3 L of tetrahydrofuran (THF) in a 1 L round-bottomed flask, and Intermediate B-8-2 (28.86 g, 83.95 mmol) and tetrakis(triphenylphosphine)palladium (4.85 g, 4.2 mmol) were added thereto and stirred. Subsequently, potassium carbonate saturated in water (29.01 g, 209.9 mmol) was added thereto and then, heated and refluxed at 80° C. for 12 hours. When a reaction was complete, water was added to the reaction solution and then, stirred for 30 minutes, and a solid therefrom was dissolved in monochlorobenzene at 133° C. and then filtered by using silica gel after removing moisture with anhydrous magnesium sulfate, and a filtrate therefrom was cooled down to room temperature and filtered. The obtained solid was repetitively purified by using monochlorobenzene to obtain 31.0 g (67.1%) of Compound B-12.

### Synthesis Example 21: Synthesis of Compound B-79

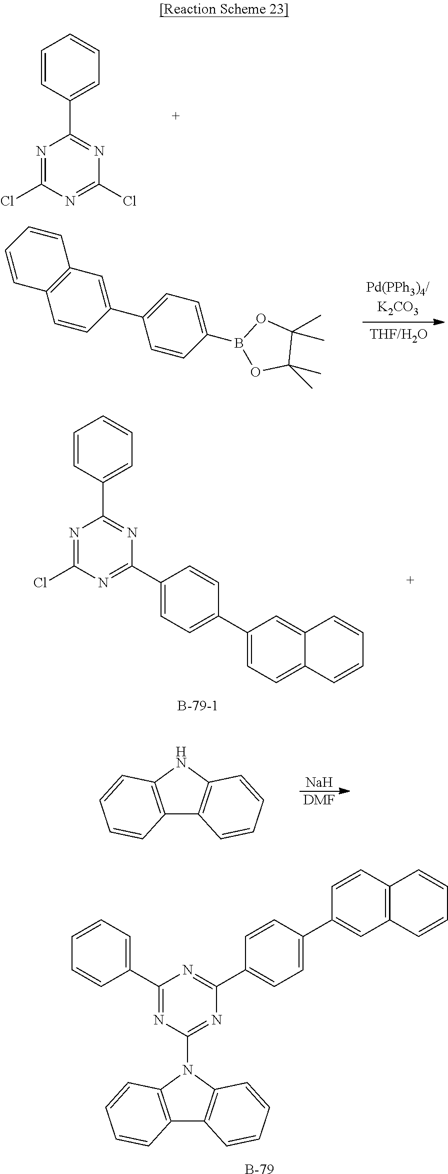

a) Synthesis of Intermediate B-79-1

Intermediate B-79-1 (18 g, 74%) was synthesized according to the same method as the c) of Synthesis Example 20 except that 2,4-chloro-6-phenyl-1,3,5-triazine (21 g, 93 mmol) and 4,4,5,5-tetramethyl-2-(4-naphthalen-2-yl-phenyl)-[1,3,2]dioxaborolane (20.5 g, 62 mmol) were used.

b) Synthesis of Compound B-79

Intermediate B-79-1 (22.5 g 57.2 mmol) and a carbazole (7.9 g, 47.6 mmol) were dissolved in 200 mL of DMF, and NaH was added thereto. The reaction solution was stirred at ambient temperature for 4 hours, and then, added to 500 ml of water to form a precipitate. A solid formed therein was filtered and washed with water and methanol. The solid was recrystallized in 500 mL of chlorobenzene to obtain 22.8 g (91%) of Compound B-79.

### Synthesis Example 22: Synthesis of Compound B-55

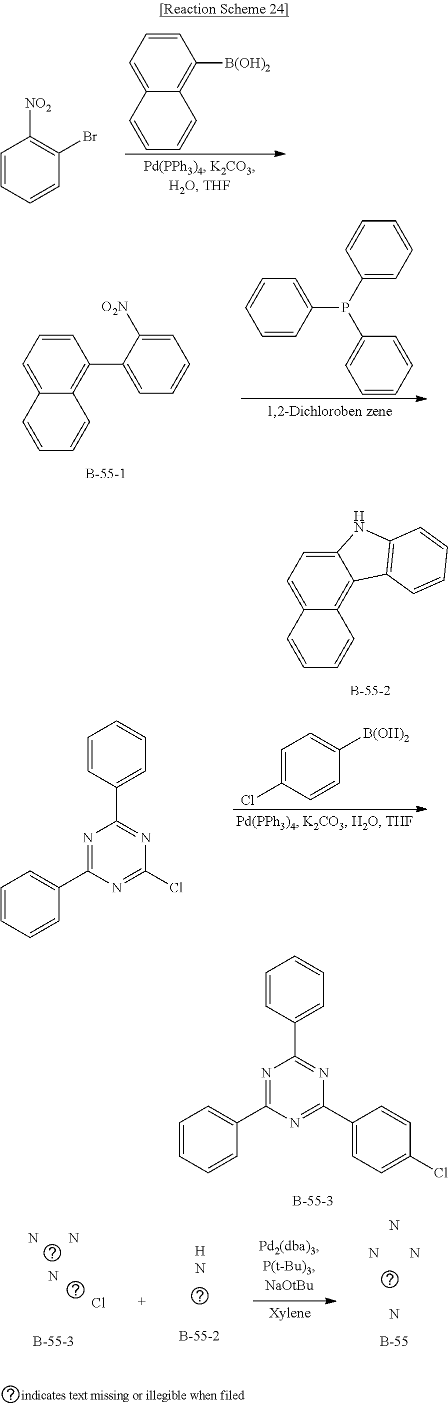

a) Synthesis of Intermediate B-55-1

28 g (64.8%) of Intermediate B-55-1 was synthesized according to the same method as c) of Synthesis Example 20 except that 1-bromo-2-nitro-benzene (35 g, 173.2 mmol) and 1-naphthalene-boronic acid (32.78 g, 190.6 mmol) were used.

b) Synthesis of Intermediate B-55-2

Intermediate B-55-1 (28.0 g, 112 mmol) and triphenylphosphine (88.4 g, 337 mmol) were put in a round-bottomed flask and dissolved in 1,2-dichlorobenzene (300 ml) and then, stirred at 180° C. for 24 hours. When a reaction was complete, after removing a solvent therefrom, a product therefrom was obtained through column chromatography to obtain 17.7 g (72.5%) of Intermediate B-55-2.

c) Synthesis of Intermediate B-55-3

Intermediate B-55-3 was synthesized according to the same method as c) of Synthesis Example 20.

d) Synthesis of Compound B-55

Intermediate B-55-3 (22 g, 63.9 mmol), Intermediate B-55-2 (13.9 g, 63.9 mmol), sodium t-butoxide (NaOtBu) (9.2 g, 95.9 mmol), Pd₂(dba)₃ (3.5 g, 3.8 mmol), and tri t-butylphosphine (P(tBu)₃) (4.6 g, 50% in toluene) were put in xylene (300 ml) and then, heated and refluxed under a nitrogen flow for 12 hours. After removing the xylene, 200 mL of methanol was added to a mixture obtained therefrom, a solid crystallized therein was filtered, dissolved in toluene, and filtered with silica gel/Celite, and an appropriate amount of an organic solvent was concentrated therefrom to obtain 21.0 g (62.6%) of Compound B-55.

### Synthesis Example 23: Synthesis of Compound B-31

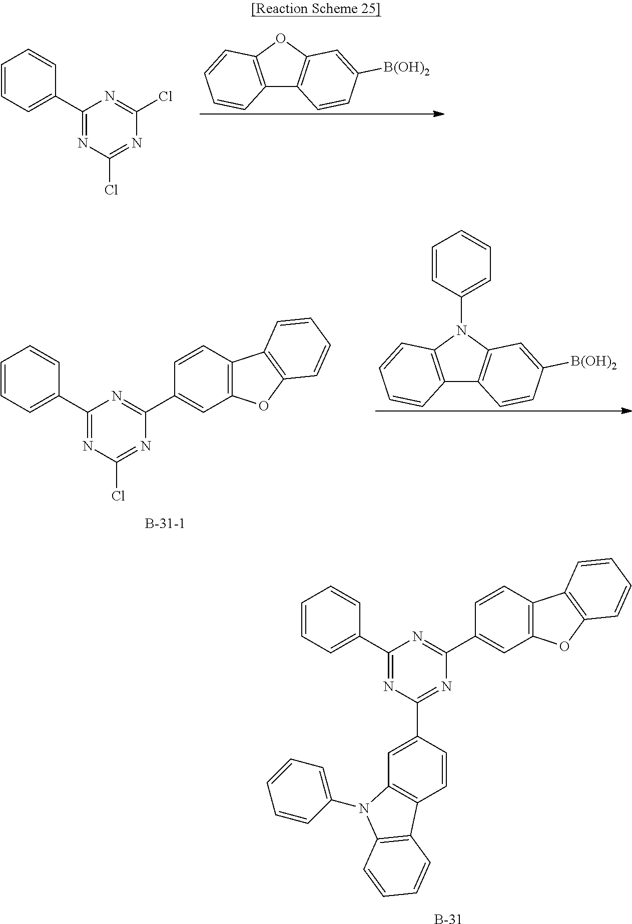

a) Synthesis of Intermediate B-31-1

21.4 g (60%) of Intermediate B-31-1 was synthesized according to the same method as the c) of Synthesis Example 20 except that 22.6 g (100 mmol) of 2,4-dichloro-6-phenyltriazine and 0.9 equivalent of dibenzofuran-3-boronic acid were used.

b) Synthesis of Compound B-31

25 g (74%) of Compound B-31 was synthesized according to the same method as c) of Synthesis Example 20 except that Intermediate B-31-1 (21.4 g, 59.8 mmol) and 1 equivalent of N-phenyl-carbazole-2-boronic acid were used.

### Synthesis Example 24: Synthesis of Compound B-83

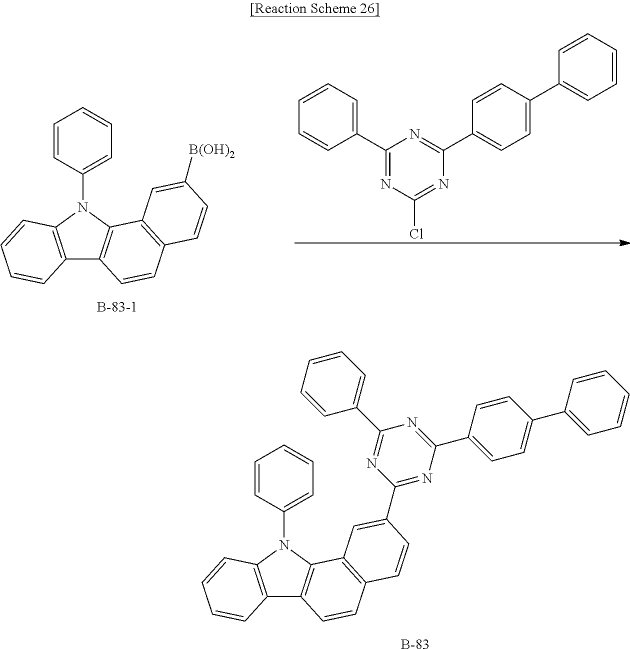

a) Synthesis of Compound B-83

24 g (67.4%) of Compound B-83 was synthesized according to the same method as the c) of Synthesis Example 20 except that Intermediate B-83-1 (20 g, 59.3 mmol) and 2-chloro-4-phenyl-6-(4-biphenyl)-1,3,5-triazine (20.39 g, 59.3 mmol) were used.

### Synthesis Example 25: Synthesis of Compound B-84

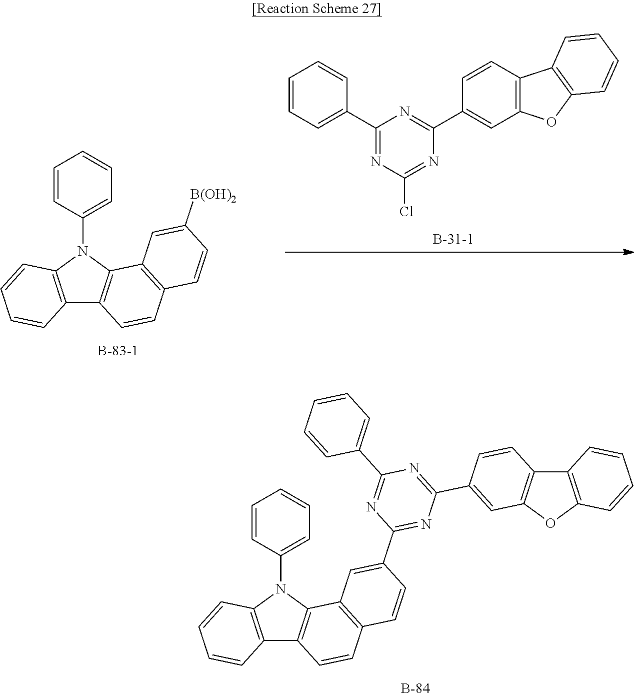

a) Synthesis of Compound B-84

26 g (71.3%) of Compound B-84 was synthesized according to the same method as the c) of Synthesis Example 20 except that Intermediate B-83-1 (20 g, 59.3 mmol) and Intermediate B-31-1 (21.22 g, 59.3 mmol) were used.

### Synthesis Example 26: Synthesis of Compound B-85

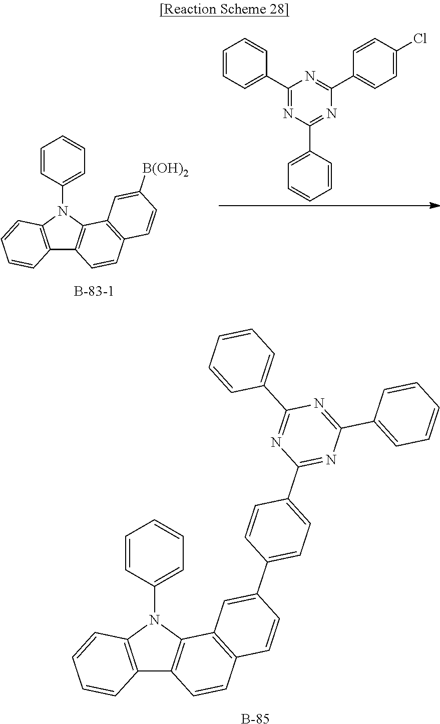

a) Synthesis of Compound B-85

23 g (64.6%) of Compound B-85 was synthesized according to the same method as the c) of Synthesis Example 20 except that Intermediate B-83-1 (20 g, 59.3 mmol) and 2-(4-chlorophenyl)-4,6-diphenyl-1,3,5-triazine (20.4 g, 59.3 mmol) were used.

### Manufacture of Organic Light Emitting Diode

### Example 1

A glass substrate coated with ITO (indium tin oxide) as a 1,500 Å-thick thin film was washed with distilled water. After washing with the distilled water, the glass substrate was ultrasonic wave-washed with isopropyl alcohol, acetone, or methanol, and dried and then, moved to a plasma cleaner, cleaned by using oxygen plasma for 10 minutes, and moved to a vacuum depositor. This obtained ITO transparent electrode was used as an anode, Compound A was vacuum-deposited on the ITO substrate to form a 700 Å-thick hole injection layer, and Compound B was deposited to be 50 Å-thick on the injection layer, and then Compound C was deposited to be 700 Å-thick to form a hole transport layer. On the hole transport layer, a 400 Å-thick light emitting layer was formed by vacuum-depositing Compound C-1 to form a hole transport auxiliary layer. On the hole transport auxiliary layer, 400 Å-thick light emitting layer was formed by using Compounds A-93 and B-12 simultaneously as a host and doping 2 wt % of [Ir(piq)₂acac] as a dopant by a vacuum-deposition. Herein Compound A-93 and Compound B-12 were used in a weight ratio of 6:4 (the ratios in the other Examples and Comparative Examples are separately provided in Table 1, below). Subsequently, on the light emitting layer, a 300 Å-thick electron transport layer was formed by simultaneously vacuum-depositing Compound D and Liq in a ratio of 1:1, and on the electron transport layer, Liq and Al were sequentially vacuum-deposited to be 15 Å-thick and 1,200 Å-thick, manufacturing an organic light emitting diode.

The organic light emitting diode had a five-layered organic thin layer, and specifically the following structure.

ITO/Compound A (700 Å)/Compound B (50 Å)/Compound C (700 Å)/Compound C-1 (400 Å)/EML [Compound A-93: B-12: [Ir(piq)₂acac] (2 wt %)] (400 Å)/Compound D: Liq (300 Å)/Liq (15 Å)/Al (1,200 Å).

Compound A: N4,N4′-diphenyl-N4,N4′-bis(9-phenyl-9H-carbazol-3-yl)biphenyl-4,4′-diamine

Compound B: 1,4,5,8,9,11-hexaazatriphenylene-hexacarbonitrile (HAT-CN)

Compound C: N-(biphenyl-4-yl)-9,9-dimethyl-N-(4-(9-phenyl-9H-carbazol-3-yl)phenyl)-9H-fluoren-2-amine

Compound C-1: N,N-di([1,1′-biphenyl]-4-yl)-7,7-dimethyl-7H-fluoreno[4,3-b]benzofuran-10-amine

Compound D: 8-(4-(4,6-di(naphthalen-2-yl)-1,3,5-triazin-2-yl)phenyl)quinoline

### Example 2 to Example 10, Comparative Example 1 and Comparative Example 2

Each organic light emitting diode was manufactured according to the same method as Example 1 except for changing compositions as shown in Table 1.

Evaluation

Luminous efficiency of the organic light emitting diodes according to Examples 1 to 10 and Comparative Examples 1 and 2 were evaluated.

Specific measurement methods are as follows, and the results are shown in Table 1.

(1) Measurement of Current Density Change Depending on Voltage Change

The obtained organic light emitting diodes were measured regarding a current value flowing in the unit device, while increasing the voltage from 0 V to 10 V using a current-voltage meter (Keithley 2400), and the measured current value was divided by area to provide the results.

(2) Measurement of Luminance Change Depending on Voltage Change

Luminance was measured by using a luminance meter (Minolta Cs-1000A), while the voltage of the organic light emitting diodes was increased from 0 V to 10 V.

(3) Measurement of Luminous Efficiency

Luminous efficiency (cd/A) at the same current density (10 mA/cm²) were calculated by using the luminance, current density, and voltages (V) from the items (1) and (2).

(4) Measurement of Life-Span

The results were obtained by measuring a time when luminous efficiency (cd/A) was decreased down to 97%, while luminance (cd/m²) was maintained to be 9,000 cd/m².

(5) Measurement of Driving Voltage

A driving voltage of each diode was measured using a current-voltage meter (Keithley 2400) at 15 mA/cm².

TABLE 1

| | | | Ratio of First | | | |
|---|---|---|---|---|---|---|
| | | Second | host:Second | | Driving | Life-span |
| | First host | host | host (wt:wt) | Color | voltage (V) | T97 (h) |
| Example 1 | A-93 | B-12 | 6:4 | red | 3.93 | 49 |
| Example 2 | A-93 | B-79 | 6:4 | red | 4 | 80 |
| Example 3 | A-93 | B-31 | 6:4 | red | 3.97 | 43 |
| Example 4 | A-93 | B-83 | 6:4 | red | 3.78 | 83 |
| Example 5 | A-93 | B-83 | 5:5 | red | 3.77 | 65 |
| Example 6 | A-93 | B-85 | 6:4 | red | 3.96 | 68 |
| Example 7 | A-93 | B-85 | 5:5 | red | 3.87 | 72 |
| Example 8 | A-93 | B-84 | 6:4 | red | 3.9 | 50 |
| Example 9 | A-92 | B-55 | 6:4 | red | 3.95 | 131 |
| Example 10 | A-92 | B-55 | 5:5 | red | 3.85 | 117 |
| Comparative | Comparative | B-12 | 6:4 | red | 4.14 | 25 |
| Example 1 | Compound 1 | | | | | |
| Comparative | Comparative | B-12 | 6:4 | red | 4.64 | 11 |
| Example 2 | Compound 2 | | | | | |

Referring to Table 1, organic light emitting diodes according to Examples 1 to 10 exhibited greatly improved driving voltage, efficiency, and life-span, when compared with the organic light emitting diodes according to Comparative Examples 1 and 2.

One or more embodiments may provide a composition for an organic optoelectronic device capable of realizing a high-efficiency and long life-span organic optoelectronic device.

Example embodiments have been disclosed herein, and although specific terms are employed, they are used and are to be interpreted in a generic and descriptive sense only and not for purpose of limitation. In some instances, as would be apparent to one of ordinary skill in the art as of the filing of the present application, features, characteristics, and/or elements described in connection with a particular embodiment may be used singly or in combination with features, characteristics, and/or elements described in connection with other embodiments unless otherwise specifically indicated. Accordingly, it will be understood by those of skill in the art that various changes in form and details may be made without departing from the spirit and scope of the present invention as set forth in the following claims.

What is claimed is:

1. A composition for an organic optoelectronic device, the composition comprising:
a first compound represented by a combination of Chemical Formula 1 and Chemical Formula 2, and
a second compound represented by Chemical Formula 3: 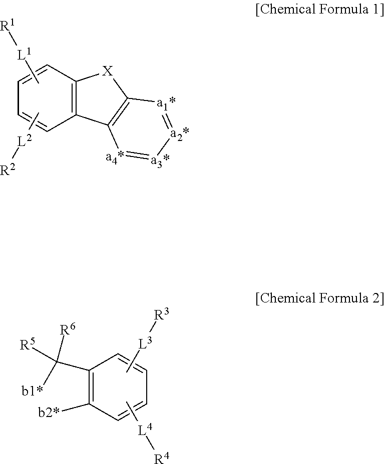
wherein, in Chemical Formula 1 and Chemical Formula 2,
X is O or S,
adjacent two of a₁* to a₄* are C linked with b₁* and b₂* respectively,
the other two of a₁* to a₄* not linked with b₁* and b₂* are each independently C-L^{a}-R^{a},
L^{a} and L¹ to L⁴ are each independently a single bond, a substituted or unsubstituted C6 to C20 arylene group, a substituted or unsubstituted C2 to C20 heterocyclic group, or a combination thereof,
R^{a} and R¹ to R⁶ are each independently hydrogen, deuterium, a cyano group, a substituted or unsubstituted amine group, a substituted or unsubstituted C1 to C30 alkyl group, a substituted or unsubstituted C6 to C30 aryl group, a substituted or unsubstituted C2 to C30 heterocyclic group, or a combination thereof, and
at least one of R¹ to R⁴ is a substituted amine group represented by Chemical Formula a, 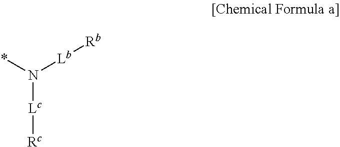
wherein, in Chemical Formula a,
L^{b} and L^{c} are each independently a single bond, a substituted or unsubstituted C6 to C20 arylene group, a substituted or unsubstituted C2 to C20 heterocyclic group, or a combination thereof,
R^{b} and R^{c} are each independently a substituted or unsubstituted C6 to C30 aryl group, a substituted or unsubstituted C2 to C30 heterocyclic group, or a combination thereof, and
is a linking point with L¹, L², L³, or L⁴; 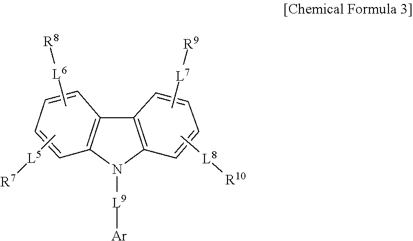
wherein, in Chemical Formula 3,
L⁵ to L⁹ are each independently a single bond, a substituted or unsubstituted C6 to C20 arylene group, a substituted or unsubstituted C2 to C20 heterocyclic group, or a combination thereof,
Ar is a substituted or unsubstituted C6 to C30 aryl group, a substituted or unsubstituted C2 to C30 heterocyclic group, or a combination thereof,
R⁷ to R¹⁰ are each independently hydrogen, deuterium, a substituted or unsubstituted C1 to C30 alkyl group, a substituted or unsubstituted C6 to C30 aryl group, a substituted or unsubstituted C2 to C30 heterocyclic group, a substituted or unsubstituted silyl group, a substituted or unsubstituted amine group, a halogen, a cyano group, or a combination thereof,
R⁷ to R¹⁰ are separately present or adjacent ones thereof are linked with each other to form a substituted or unsubstituted aliphatic monocyclic ring or a substituted or unsubstituted aliphatic polycyclic ring, a substituted or unsubstituted aromatic monocyclic ring or a substituted or unsubstituted aromatic polycyclic ring, or a substituted or unsubstituted heteroaromatic monocyclic ring or a substituted or unsubstituted heteroaromatic polycyclic ring, and
at least one of Ar and R⁷ to R¹⁰ is a group represented by Chemical Formula b, 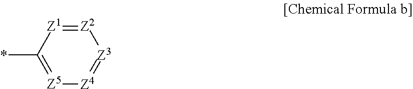
wherein, in Chemical Formula b,
Z¹ to Z⁵ are each independently N or C-L^{d}-R^{d},
at least two of Z¹ to Z⁵ are N,
each L^{d} is independently a single bond, a substituted or unsubstituted C6 to C20 arylene group, a substituted or unsubstituted C2 to C20 heterocyclic group, or a combination thereof,
each R^{d} is independently hydrogen, deuterium, a substituted or unsubstituted C1 to C30 alkyl group, a substituted or unsubstituted C6 to C30 aryl group, a substituted or unsubstituted C2 to C30 heterocyclic group, a substituted or unsubstituted silyl group, a substituted or unsubstituted amine group, a halogen, a cyano group, or a combination thereof,
each R^{d} is separately present or adjacent ones thereof are linked with each other to form a substituted or unsubstituted aliphatic monocyclic ring or a substituted or unsubstituted aliphatic polycyclic ring, a substituted or unsubstituted aromatic monocyclic ring or a substituted or unsubstituted aromatic polycyclic ring, or a substituted or unsubstituted heteroaromatic monocyclic ring or a substituted or unsubstituted heteroaromatic polycyclic ring, and
is a linking point with L⁵, L⁶, L⁷, L⁸, or L⁹.

2. The composition as claimed in claim 1, wherein the first compound is represented by one of Chemical Formula 1A to Chemical Formula 1F: 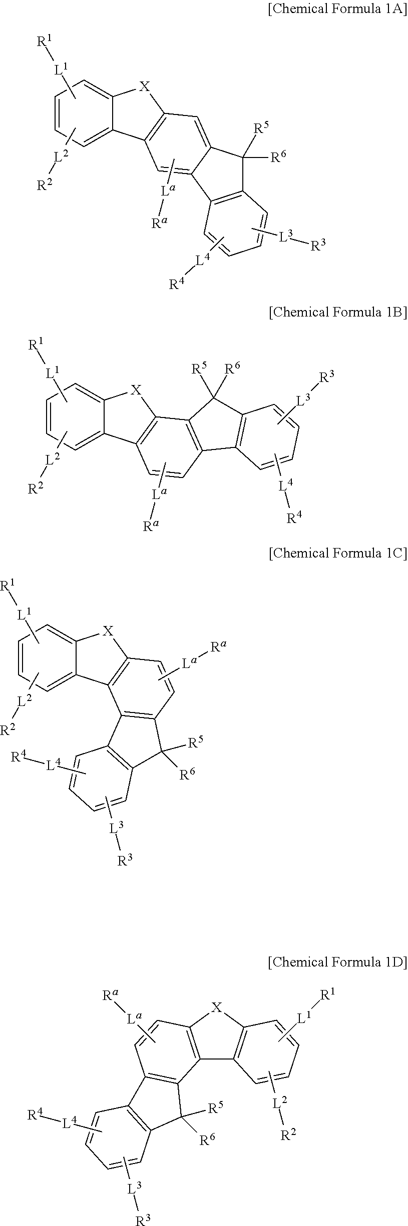 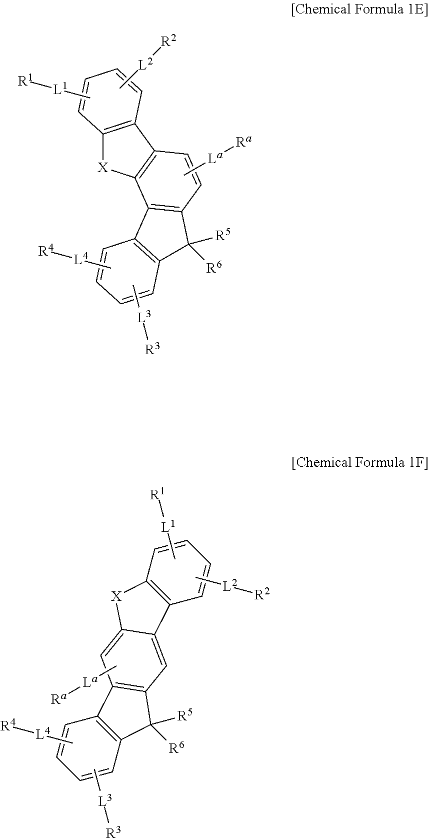
wherein, in Chemical Formula 1A to Chemical Formula 1F,
X is O or S,
L^{a} and L¹ to L⁴ are each independently a single bond, a substituted or unsubstituted C6 to C20 arylene group, a substituted or unsubstituted C2 to C20 heterocyclic group, or a combination thereof,
R^{a} and R¹ to R⁶ are each independently hydrogen, deuterium, a cyano group, a substituted or unsubstituted amine group, a substituted or unsubstituted C1 to C30 alkyl group, a substituted or unsubstituted C6 to C30 aryl group, a substituted or unsubstituted C2 to C30 heterocyclic group, or a combination thereof, and
at least one of R¹ to R⁴ is a substituted amine group represented by Chemical Formula a, 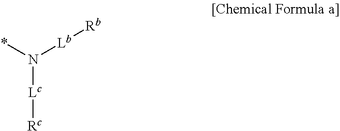
wherein, in Chemical Formula a,
L^{b} and L^{c} are each independently a single bond, a substituted or unsubstituted C6 to C20 arylene group, a substituted or unsubstituted C2 to C20 heterocyclic group, or a combination thereof,
R^{b} and R^{c} are each independently a substituted or unsubstituted C6 to C30 aryl group, a substituted or unsubstituted C2 to C30 heterocyclic group, or a combination thereof, and
is a linking point with L¹, L², L³, or L⁴.

3. The composition as claimed in claim 1, wherein the first compound is represented by Chemical Formula 1E-1-1 or Chemical Formula 1E-2-2: 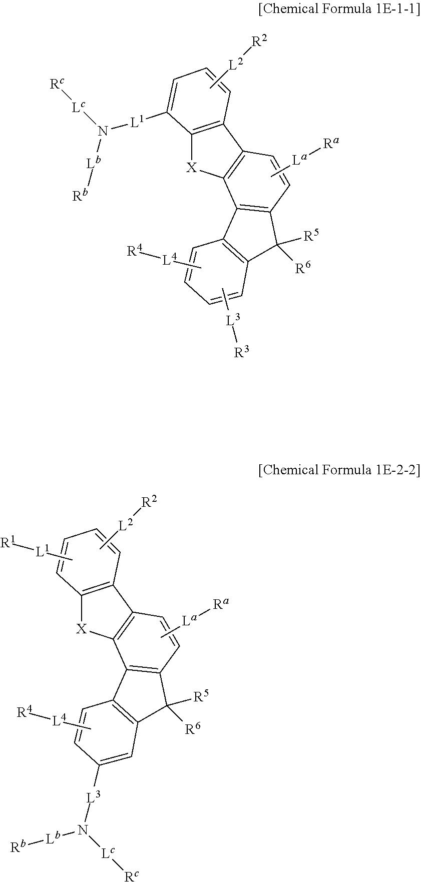
wherein, in Chemical Formula 1E-1-1 and Chemical Formula 1E-2-2,
X is O or S,
L^{a} and L¹ to L⁴ are each independently a single bond, a substituted or unsubstituted C6 to C20 arylene group, a substituted or unsubstituted C2 to C20 heterocyclic group, or a combination thereof,
R^{a} and R¹ to R⁶ are each independently hydrogen, deuterium, a cyano group, a substituted or unsubstituted amine group, a substituted or unsubstituted C1 to C30 alkyl group, a substituted or unsubstituted C6 to C30 aryl group, a substituted or unsubstituted C2 to C30 heterocyclic group, or a combination thereof,
L^{b} and L^{c} are each independently a single bond, a substituted or unsubstituted C6 to C20 arylene group, a substituted or unsubstituted C2 to C20 heterocyclic group, or a combination thereof, and
R^{b} and R^{c} are each independently a substituted or unsubstituted phenyl group, a substituted or unsubstituted biphenyl group, a substituted or unsubstituted terphenyl group, a substituted or unsubstituted anthracenyl group, a substituted or unsubstituted naphthyl group, a substituted or unsubstituted phenanthrenyl group, a substituted or unsubstituted triphenylene group, a substituted or unsubstituted fluorenyl group, a substituted or unsubstituted carbazolyl group, a substituted or unsubstituted dibenzofuranyl group, a substituted or unsubstituted dibenzothiophenyl group, or a monovalent fused ring group of a compound represented by a combination of Chemical Formula 1 and Chemical Formula 2.

4. The composition as claimed in claim 1, wherein the second compound is represented by one of Chemical Formula 3A to Chemical Formula 3R: 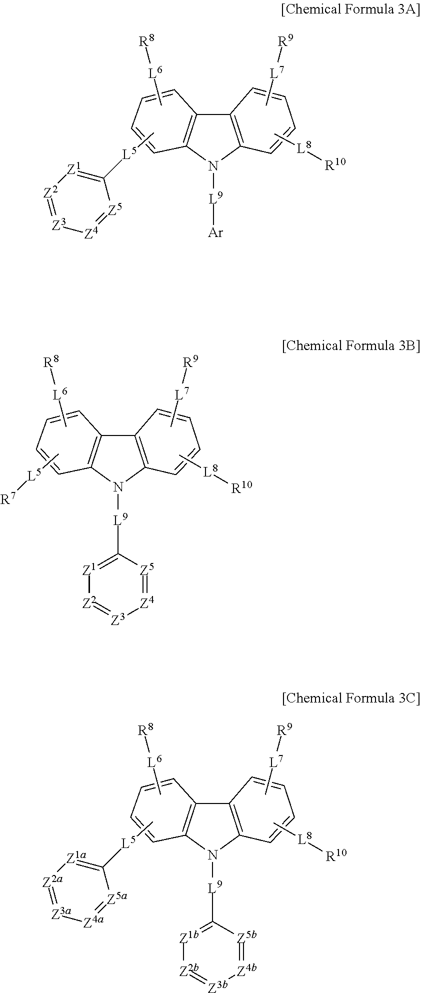 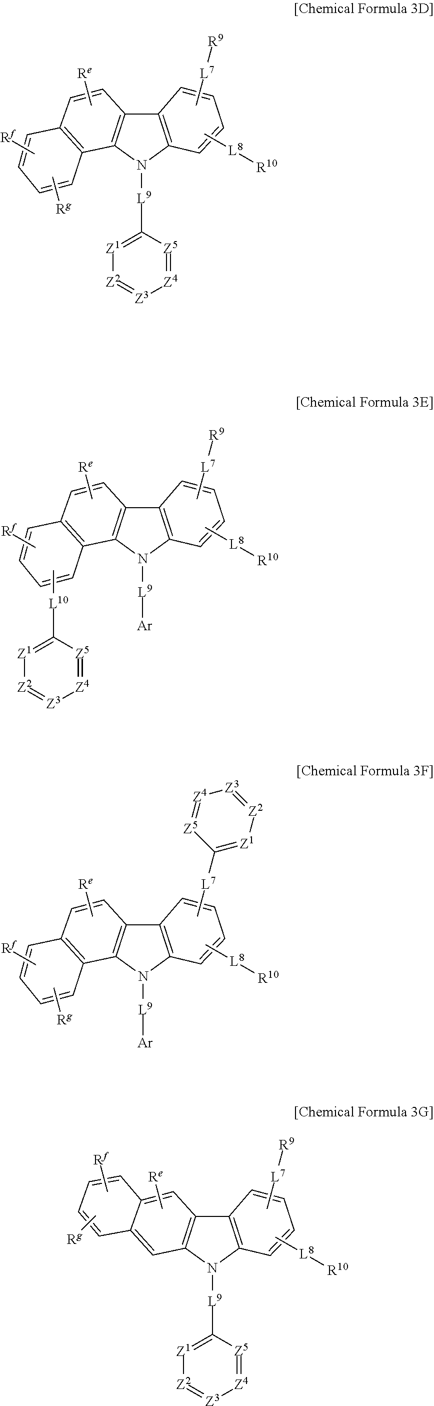 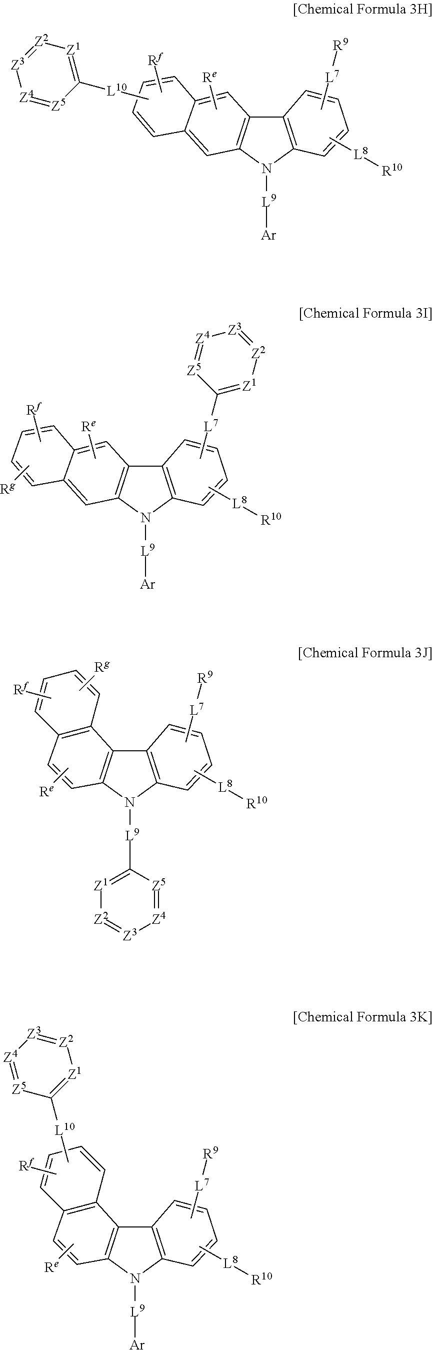 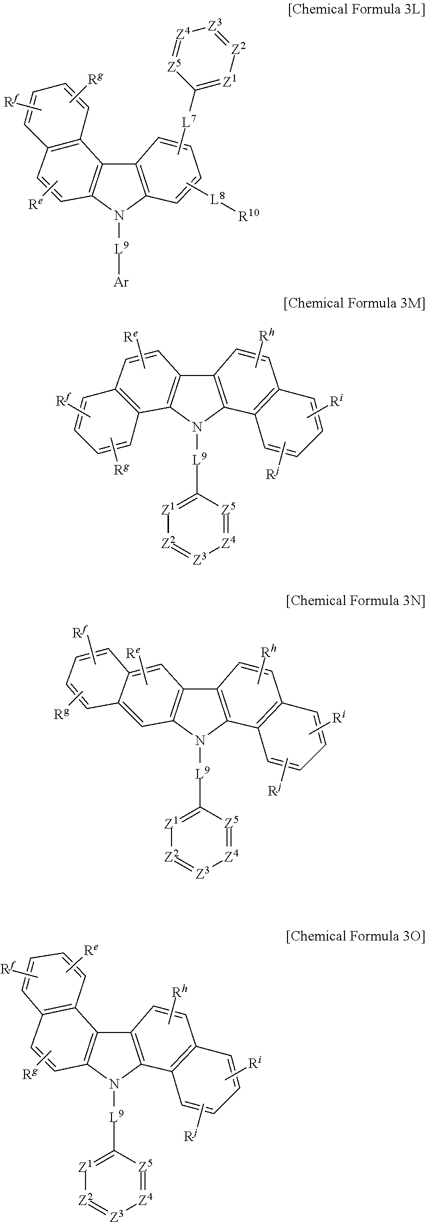 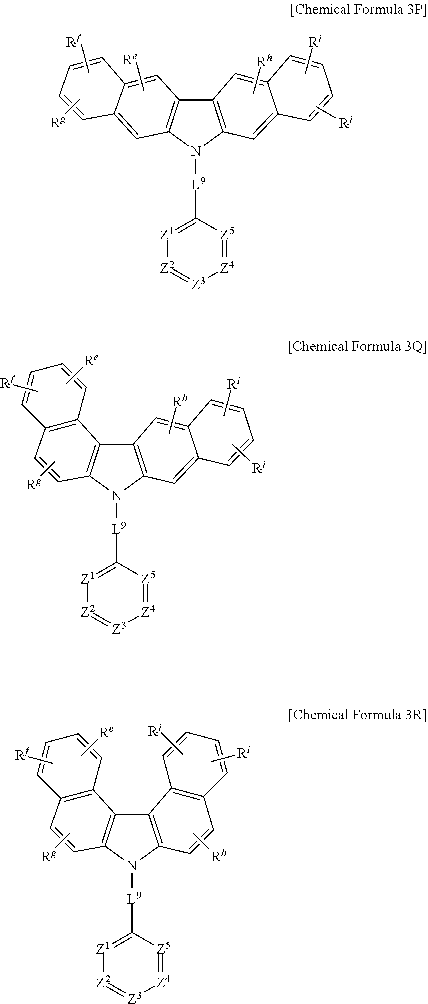
wherein, in Chemical Formula 3A to Chemical Formula 3R,
L⁵ to L¹⁰ are each independently a single bond, a substituted or unsubstituted C6 to C20 arylene group, a substituted or unsubstituted C2 to C20 heterocyclic group, or a combination thereof,
Ar is a substituted or unsubstituted C6 to C30 aryl group,
R⁷ to R¹⁰, R^{e}, R^{f}, R^{g}, R^{h}, Rⁱ, and R^{j} are each independently hydrogen, deuterium, a substituted or unsubstituted C1 to C30 alkyl group, a substituted or unsubstituted C6 to C30 aryl group, a substituted or unsubstituted C2 to C30 heterocyclic group, a substituted or unsubstituted silyl group, a substituted or unsubstituted amine group, a halogen, a cyano group, or a combination thereof,
Z¹ to Z⁵, Z^{1a} to Z^{5a}, and Z^{1b} to Z^{5b} are each independently N or C-L^{d}-R^{d},
at least two of Z¹ to Z⁵ are N,
at least two of Z^{1a} to Z^{5a} are N,
at least two of Z^{1b} to Z^{5b} are N,
each L^{d} is independently a single bond, a substituted or unsubstituted C6 to C20 arylene group, a substituted or unsubstituted C2 to C20 heterocyclic group, or a combination thereof,
each R^{d} is independently hydrogen, deuterium, a substituted or unsubstituted C1 to C30 alkyl group, a substituted or unsubstituted C6 to C30 aryl group, a substituted or unsubstituted C2 to C30 heterocyclic group, a substituted or unsubstituted silyl group, a substituted or unsubstituted amine group, a halogen, a cyano group, or a combination thereof, and
each R^{d} is separately present or adjacent ones thereof are linked with each other to form a substituted or unsubstituted aliphatic monocyclic ring or a substituted or unsubstituted aliphatic polycyclic ring, a substituted or unsubstituted aromatic monocyclic ring or a substituted or unsubstituted aromatic polycyclic ring, or a substituted or unsubstituted heteroaromatic monocyclic ring or a substituted or unsubstituted heteroaromatic polycyclic ring.

5. The composition as claimed in claim 4, wherein the second compound is represented by one of Chemical Formula 3A, Chemical Formula 3B, Chemical Formula 3E, and Chemical Formula 3J.

6. The composition as claimed in claim 4, wherein:
the second compound is represented by Chemical Formula 3A or Chemical Formula 3E,
the compound represented by Chemical Formula 3A is represented by Chemical Formula 3A-2 or Chemical Formula 3A-4, and
the compound represented by Chemical Formula 3E is represented by Chemical Formula 3E-2: 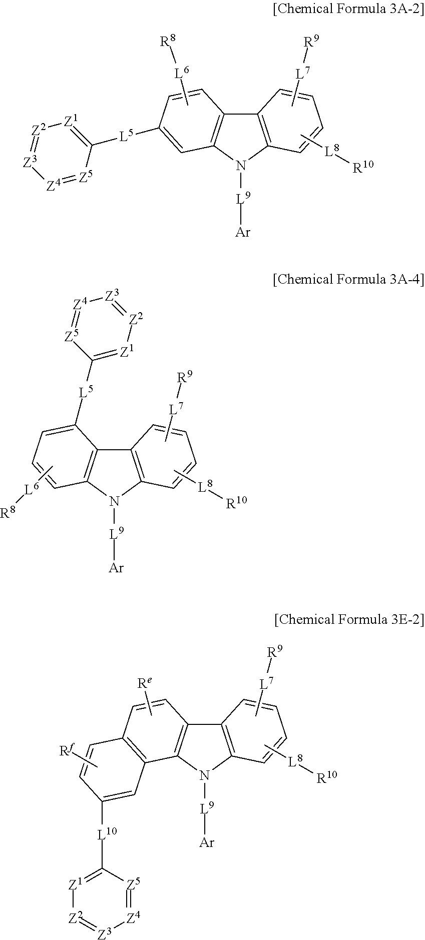
wherein, in Chemical Formula 3A-2, Chemical Formula 3A-4, and Chemical Formula 3E-2,
L⁵ to L¹⁰ are each independently a single bond, a substituted or unsubstituted C6 to C20 arylene group, a substituted or unsubstituted C2 to C20 heterocyclic group, or a combination thereof,
Ar is a substituted or unsubstituted C6 to C30 aryl group,
R⁸ to R¹⁰, R^{e} and R^{f} are each independently hydrogen, deuterium, a substituted or unsubstituted C1 to C30 alkyl group, a substituted or unsubstituted C6 to C30 aryl group, a substituted or unsubstituted C2 to C30 heterocyclic group, a substituted or unsubstituted silyl group, a substituted or unsubstituted amine group, a halogen, a cyano group, or a combination thereof,
Z¹ to Z⁵ are each independently N or C-L^{d}-R^{d},
at least two of Z¹ to Z⁵ are N,
each L^{d} is independently a single bond, a substituted or unsubstituted C6 to C20 arylene group, a substituted or unsubstituted C2 to C20 heterocyclic group, or a combination thereof,
each R^{d} is independently hydrogen, deuterium, a substituted or unsubstituted C1 to C30 alkyl group, a substituted or unsubstituted C6 to C30 aryl group, a substituted or unsubstituted C2 to C30 heterocyclic group, a substituted or unsubstituted silyl group, a substituted or unsubstituted amine group, a halogen, a cyano group, or a combination thereof, and
each R^{d} is separately present or adjacent ones thereof are linked with each other to form a substituted or unsubstituted aliphatic monocyclic ring or a substituted or unsubstituted aliphatic polycyclic ring, a substituted or unsubstituted aromatic monocyclic ring or a substituted or unsubstituted aromatic polycyclic ring, or a substituted or unsubstituted heteroaromatic monocyclic ring or a substituted or unsubstituted heteroaromatic polycyclic ring.

7. The composition as claimed in claim 1, wherein the group represented by Chemical Formula b is a substituted or unsubstituted pyrimidinyl group, a substituted or unsubstituted triazinyl group, a substituted or unsubstituted quinazolinyl group, a substituted or unsubstituted quinoxalinyl group, or a substituted or unsubstituted naphthyridinyl group.

8. The composition as claimed in claim 1, wherein the group represented by Chemical Formula b is represented by one of Chemical Formula b-1 to Chemical Formula b-5: 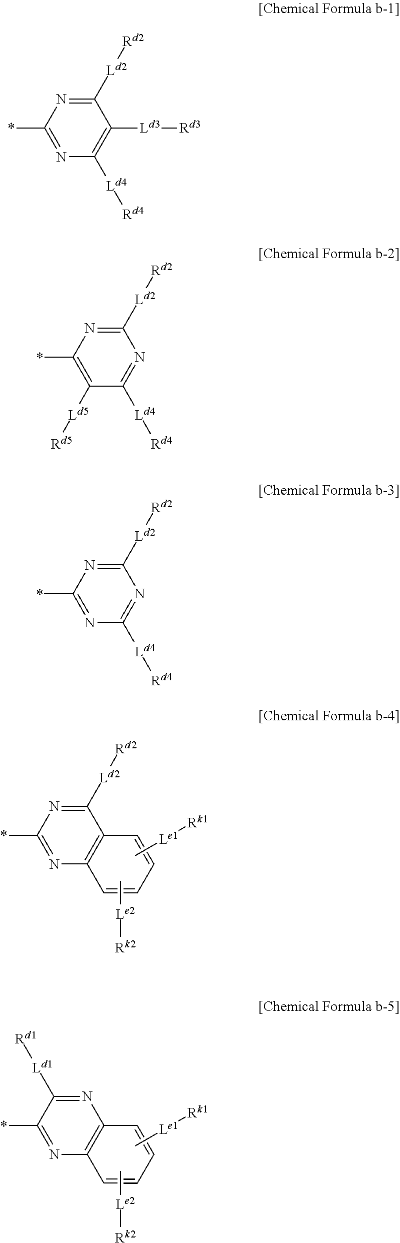
wherein, in Chemical Formula b-1 to Chemical Formula b-5,
L^{d1} to L^{d5}, L^{e1}, and L^{e2} are each independently a single bond, a substituted or unsubstituted C6 to C20 arylene group, a substituted or unsubstituted C2 to C20 heterocyclic group, or a combination thereof,
R^{d2} to R^{d5}, R^{k1}, and R^{k2} are each independently hydrogen, deuterium, a cyano group, a substituted or unsubstituted amine group, a substituted or unsubstituted C1 to C10 alkyl group, a substituted or unsubstituted phenyl group, a substituted or unsubstituted biphenyl group, a substituted or unsubstituted naphthyl group, a substituted or unsubstituted terphenyl group, a substituted or unsubstituted anthracenyl group, a substituted or unsubstituted phenanthrenyl group, a substituted or unsubstituted fluorenyl group, a substituted or unsubstituted triphenylene group, a substituted or unsubstituted dibenzofuranyl group, or a substituted or unsubstituted dibenzothiophenyl group, and
is a linking point with L⁵, L⁶, L⁷, L⁸, or L⁹.

9. The composition as claimed in claim 1, wherein the group represented by Chemical Formula b is a group of the following Group I: 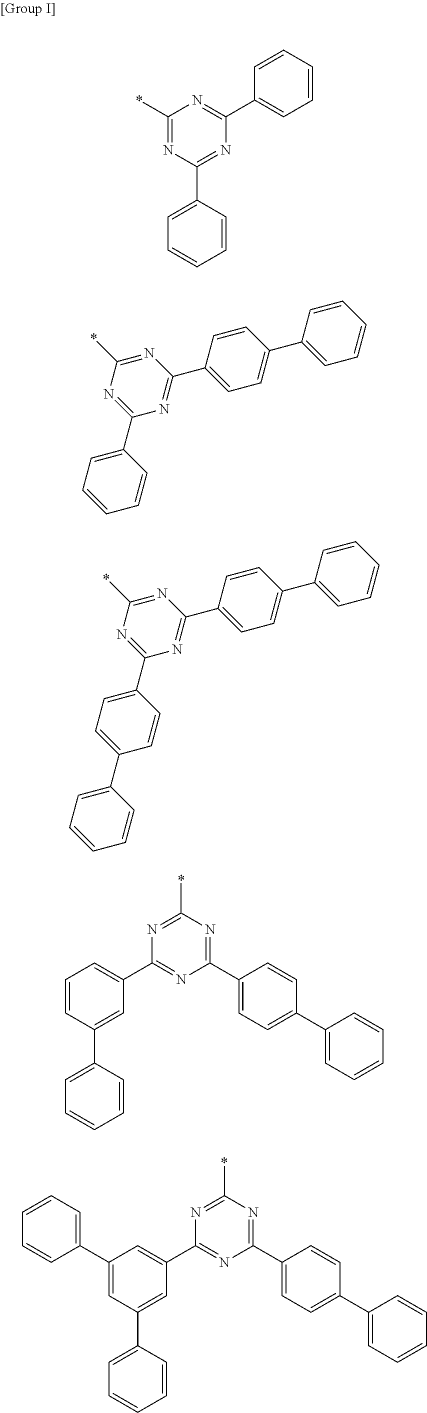 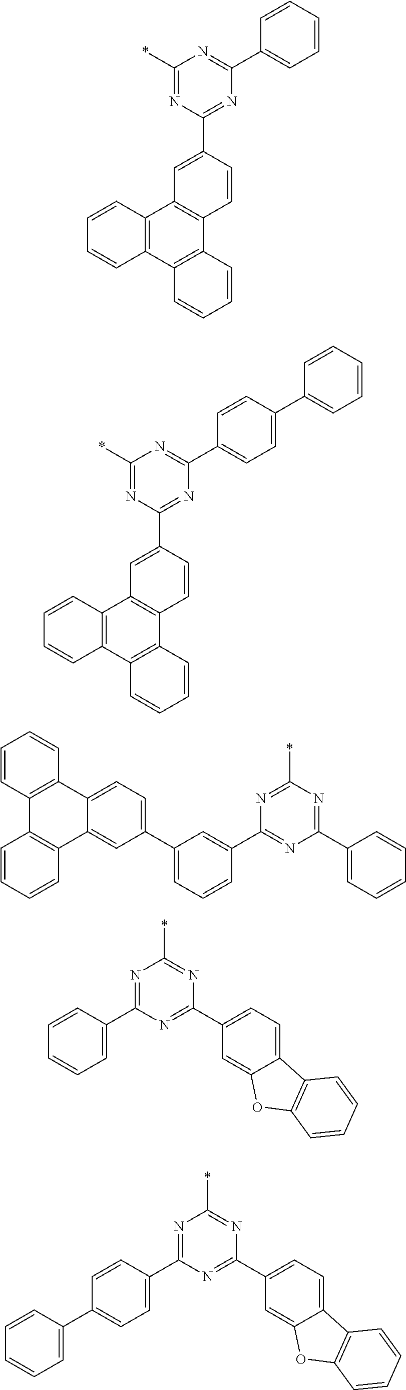 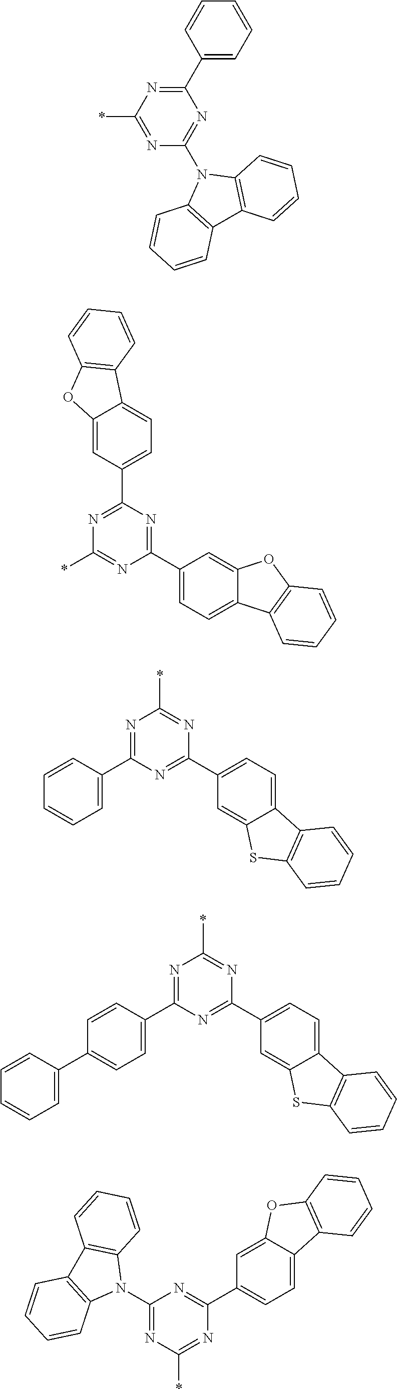 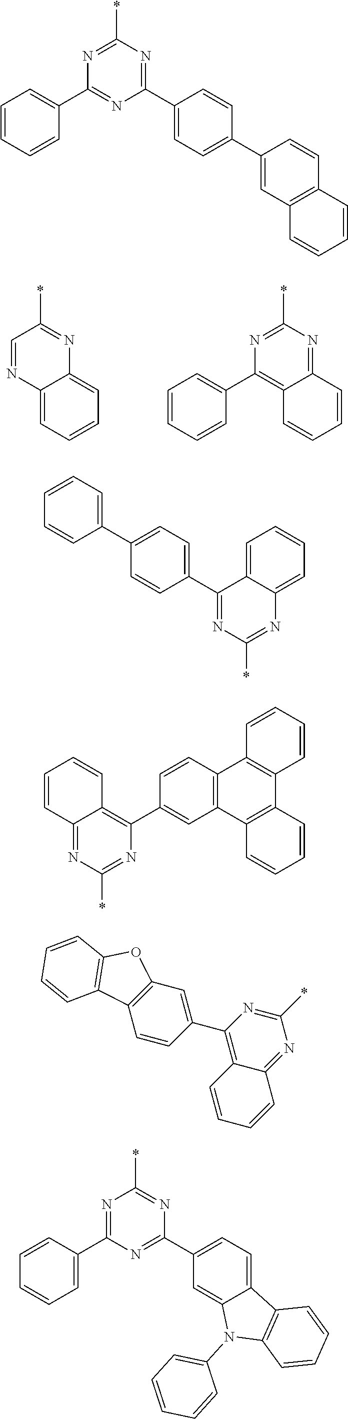 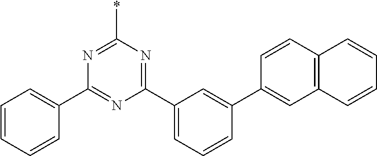
wherein, in Group I, * is a linking point.

10. The composition as claimed in claim 1, wherein:
the first compound is represented by Chemical Formula 1E-2-2, and
the second compound is represented by one of Chemical Formula 3A-2, Chemical Formula 3B, Chemical Formula 3E-2, and Chemical Formula 3J: 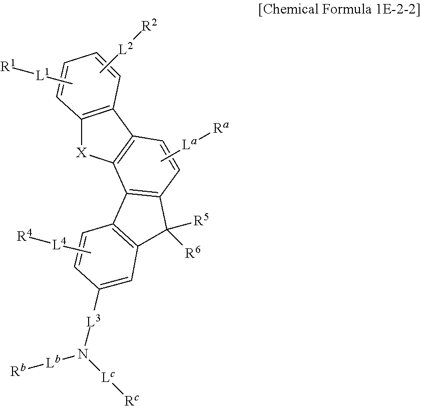
wherein, in Chemical Formula 1E-2-2,
X is O or S,
L^{a}, L^{b}, L^{c}, and L¹ to L⁴ are each independently a single bond, a substituted or unsubstituted phenylene group, a substituted or unsubstituted biphenylene group, a substituted or unsubstituted terphenylene group, or a substituted or unsubstituted naphthylene group,
R^{a}, R¹, R², and R⁴ are each independently hydrogen, deuterium, a cyano group, a substituted or unsubstituted C1 to C30 alkyl group, a substituted or unsubstituted C6 to C30 aryl group, a substituted or unsubstituted C2 to C30 heterocyclic group, or a combination thereof,
R⁵ and R⁶ are each independently a substituted or unsubstituted C1 to C10 alkyl group or a substituted or unsubstituted C6 to C20 aryl group, and
R^{b} and R^{c} are each independently a substituted or unsubstituted phenyl group, a substituted or unsubstituted biphenyl group, a substituted or unsubstituted anthracenyl group, a substituted or unsubstituted naphthyl group, a substituted or unsubstituted phenanthrenyl group, a substituted or unsubstituted triphenylene group, a substituted or unsubstituted fluorenyl group, a substituted or unsubstituted carbazolyl group, a substituted or unsubstituted dibenzofuranyl group, a substituted or unsubstituted dibenzothiophenyl group, or a monovalent fused ring group of a compound represented by a combination of Chemical Formula 1 and Chemical Formula 2; 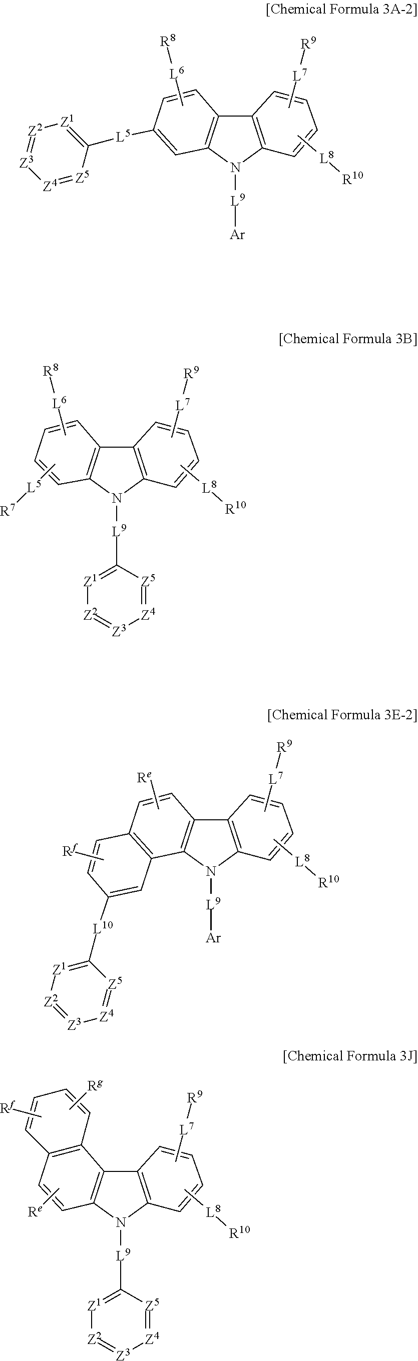
wherein, in Chemical Formula 3A-2, Chemical Formula 3B, Chemical Formula 3E-2, and Chemical Formula 3J,
L⁵ to L¹⁰ are each independently a single bond, a substituted or unsubstituted phenylene group, a substituted or unsubstituted biphenylene group, a substituted or unsubstituted terphenylene group, a substituted or unsubstituted naphthylene group, a substituted or unsubstituted dibenzofuranylene group, a substituted or unsubstituted dibenzothiophenylene group, a substituted or unsubstituted carbazolylene group, a substituted or unsubstituted fused dibenzofuranylene group, a substituted or unsubstituted fused dibenzothiophenylene group, or a combination thereof,
Ar is a substituted or unsubstituted phenyl group, a substituted or unsubstituted biphenyl group, or a substituted or unsubstituted naphthyl group, or a substituted or unsubstituted fluorenyl group,
R⁷ to R¹⁰, R^{e}, R^{f} and R^{g} are each independently hydrogen, deuterium, a cyano group, a substituted or unsubstituted phenyl group, a substituted or unsubstituted biphenyl group, a substituted or unsubstituted terphenyl group, a substituted or unsubstituted naphthyl group, a substituted or unsubstituted triphenylene group, a substituted or unsubstituted dibenzofuranyl group, a substituted or unsubstituted dibenzothiophenyl group, or a combination thereof,
Z¹ to Z⁵ are each independently N or C-L^{d}-R^{d},
at least two of Z¹ to Z⁵ are N,
each L^{d} is independently a single bond or a substituted or unsubstituted C6 to C20 arylene group,
each R^{d} is independently a substituted or unsubstituted phenyl group, a substituted or unsubstituted biphenyl group, a substituted or unsubstituted terphenyl group, a substituted or unsubstituted anthracenyl group, a substituted or unsubstituted naphthyl group, a substituted or unsubstituted phenanthrenyl group, a substituted or unsubstituted triphenylene group, a substituted or unsubstituted fluorenyl group, a substituted or unsubstituted carbazolyl group, a substituted or unsubstituted dibenzofuranyl group or a substituted or unsubstituted dibenzothiophenyl group, and
each R^{d} is separately present or adjacent ones thereof are linked with each other to form a substituted or unsubstituted quinazolinyl group or a substituted or unsubstituted quinoxalinyl group.

11. The composition as claimed in claim 1, further comprising a dopant.

12. An organic optoelectronic device, comprising:
an anode and a cathode facing each other,
at least one organic layer between the anode and the cathode,
wherein the organic layer includes the composition for an organic optoelectronic device as claimed in claim 1.

13. The organic optoelectronic device as claimed in claim 12, wherein:
the organic layer includes a light emitting layer, and
the light emitting layer includes the composition.

14. The organic optoelectronic device as claimed in claim 13, wherein the first compound and the second compound are phosphorescent hosts of the light emitting layer.

15. The organic optoelectronic device as claimed in claim 12, wherein the composition is a red light emitting composition.

16. A display device comprising the organic optoelectronic device as claimed in claim 12.
